SEQUENCE LISTING (375 sequences)

SEQ ID NO: 1 - protein, 9 aa, Artificial Sequence
  Description of Artificial Sequence Synthetic peptide
  MOD_RES (7)..(7): L or V
  MOD_RES (8)..(8): V, L, I or F
VMAPRTXXL

SEQ ID NO: 2 - protein, 9 aa, Simian immunodeficiency virus
RMYNPTNIL

SEQ ID NO: 3 - protein, 9 aa, Human immunodeficiency virus 1
RMYSPTSIL

SEQ ID NO: 4 - protein, 9 aa, Artificial Sequence
  Description of Artificial Sequence Synthetic peptide
  MOD_RES (1)..(1): V, A, C, I, S, T, V, H or P
  MOD_RES (7)..(7): L or V
  MOD_RES (8)..(8): V, L, I or F
XMAPRTXXL

SEQ ID NO: 5 - protein, 9 aa, Unknown
  Description of Unknown VL9 peptide
VMAPRTLLL

SEQ ID NO: 6 - protein, 9 aa, Artificial Sequence
  Description of Artificial Sequence Synthetic peptide
RMAPRTLLL

SEQ ID NO: 7 - protein, 9 aa, Human immunodeficiency virus 1
RMYSPTSIL

SEQ ID NO: 8 - protein, 9 aa, Artificial Sequence
  Description of Artificial Sequence Synthetic peptide
VMYSPTSIL

SEQ ID NO: 9 - protein, 9 aa, Artificial Sequence
  Description of Artificial Sequence Synthetic peptide
  MOD_RES (1)..(1): V, A, C, I, S, T, V, H or P
  MOD_RES (2)..(2): M, L, Q or F
  MOD_RES (7)..(7): L or V
  MOD_RES (8)..(8): V, L, I or F
XXAPRTXXL

SEQ ID NO: 10 - protein, 9 aa, Artificial Sequence
  Description of Artificial Sequence Synthetic peptide
  MOD_RES (8)..(8): V or L
VMAPRTLXL

SEQ ID NO: 11 - protein, 9 aa, Artificial Sequence
  Description of Artificial Sequence Synthetic peptide
  MOD_RES (8)..(8): L, I or F
VMAPRTVXL

SEQ ID NO: 12 - protein, 9 aa, Artificial Sequence
  Description of Artificial Sequence Synthetic peptide
  MOD_RES (1)..(1): V, A, C, I, S or T
XMAPRTLLL

SEQ ID NO: 13 - protein, 9 aa, Artificial Sequence
  Description of Artificial Sequence Synthetic peptide
  MOD_RES (2)..(2): M or T
VXAPRTLLL

SEQ ID NO: 14 - protein, 9 aa, Artificial Sequence
  Description of Artificial Sequence Synthetic peptide
  MOD_RES (2)..(2): L, I, Q, T, V, S, A or F
VXAPRTLLL

SEQ ID NO: 15 - protein, 9 aa, Artificial Sequence
  Description of Artificial Sequence Synthetic peptide
  MOD_RES (9)..(9): L or F
VMAPRTLLX

SEQ ID NO: 16 - protein, 5 aa, Artificial Sequence
  Description of Artificial Sequence Synthetic peptide
LEWIG

SEQ ID NO: 17 - protein, 9 aa, Human betaherpesvirus 5
VMAPRTLIL

SEQ ID NO: 18 - protein, 9 aa, Unknown
  Description of Unknown VL9 peptide
VMAPRTLVL

SEQ ID NO: 19 - protein, 9 aa, Simian immunodeficiency virus
RMYNPTNIL

SEQ ID NO: 20 - protein, 9 aa, Escherichia coli
VMPPRALLL

SEQ ID NO: 21 - protein, 9 aa, Stenotrophomonas sp.
VMAGRTLLL

SEQ ID NO: 22 - protein, 9 aa, Pseudomonas formosensis
VMAPRTKLL

SEQ ID NO: 23 - protein, 9 aa, Unknown
  Description of Unknown AL9 peptide
AMAPRTLLL

SEQ ID NO: 24 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgataccgcaagatatagctacggctggtacttcgatgtctggggcacagggaccacggtcaccgtctcctca

SEQ ID NO: 25 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgattccgcacagtacagctacggctggtacttcgatgtctggggcacagggaccacggtcaccgtctcctca

SEQ ID NO: 26 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgatactggtcgcttcagctacggctggtacttcgatgtctggggcacagggaccacggtcaccgtctcctca

SEQ ID NO: 27 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgatggagcacagactagctacggctggtacttcgatgtctggggcacagggaccacggtcaccgtctcctca

SEQ ID NO: 28 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgatggagcacagtggagctacggctggtacttcgatgtctggggcacagggaccacggtcaccgtctcctca

SEQ ID NO: 29 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgatggtgcgagatggagctacggctggtacttcgatgtctggggcacagggaccacggtcaccgtctcctca

SEQ ID NO: 30 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgatgcagctcagcatagctacggctggtacttcgatgtctggggcacagggaccacggtcaccgtctcctca

SEQ ID NO: 31 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgatgcagctagatttagctacggctggtacttcgatgtctggggcacagggaccacggtcaccgtctcctca

SEQ ID NO: 32 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgatggtgctagacatagctacggctggtacttcgatgtctggggcacagggaccacggtcaccgtctcctca

SEQ ID NO: 33 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgatgctgcgacatacagctacggctggtacttcgatgtctggggcacagggaccacggtcaccgtctcctca

SEQ ID NO: 34 - DNA, 369 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgatagaggtccaccaagctacggctggtacttcgatgtctggggcacagggaccacggtcaccgtctcctca

SEQ ID NO: 35 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDTARYSYGWYFDVWGTGTTVTVSS

SEQ ID NO: 36 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDSAQYSYGWYFDVWGTGTTVTVSS

SEQ ID NO: 37 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDTGRFSYGWYFDVWGTGTTVTVSS

SEQ ID NO: 38 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDGAQTSYGWYFDVWGTGTTVTVSS

SEQ ID NO: 39 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDGAQWSYGWYFDVWGTGTTVTVSS

SEQ ID NO: 40 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDGARWSYGWYFDVWGTGTTVTVSS

SEQ ID NO: 41 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDAAQHSYGWYFDVWGTGTTVTVSS

SEQ ID NO: 42 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDAARFSYGWYFDVWGTGTTVTVSS

SEQ ID NO: 43 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDGARHSYGWYFDVWGTGTTVTVSS

SEQ ID NO: 44 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDAATYSYGWYFDVWGTGTTVTVSS

SEQ ID NO: 45 - protein, 123 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDRGPPSYGWYFDVWGTGTTVTVSS

SEQ ID NO: 46 - protein, 9 aa, Artificial Sequence
  Description of Artificial Sequence Synthetic peptide
VMAPRTVLL

SEQ ID NO: 47 - protein, 141 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYGSSYGWYFDVWGTGTTVTVSSASTKGPSVFLPAPSSKST

SEQ ID NO: 48 - protein, 9 aa, Artificial Sequence
  Description of Artificial Sequence Synthetic peptide
CMAPRTLLL

SEQ ID NO: 49 - protein, 9 aa, Artificial Sequence
  Description of Artificial Sequence Synthetic peptide
DMAPRTLLL

SEQ ID NO: 50 - protein, 9 aa, Artificial Sequence
  Description of Artificial Sequence Synthetic peptide
EMAPRTLLL

SEQ ID NO: 51 - protein, 9 aa, Artificial Sequence
  Description of Artificial Sequence Synthetic peptide
FMAPRTLLL

SEQ ID NO: 52 - protein, 9 aa, Artificial Sequence
  Description of Artificial Sequence Synthetic peptide
GMAPRTLLL

SEQ ID NO: 53 - protein, 9 aa, Artificial Sequence
  Description of Artificial Sequence Synthetic peptide
HMAPRTLLL

SEQ ID NO: 54 - protein, 9 aa, Artificial Sequence
  Description of Artificial Sequence Synthetic peptide
IMAPRTLLL

SEQ ID NO: 55 - protein, 9 aa, Artificial Sequence
  Description of Artificial Sequence Synthetic peptide
TMAPRTLLL

SEQ ID NO: 56 - protein, 9 aa, Artificial Sequence
  Description of Artificial Sequence Synthetic peptide
WMAPRTLLL

SEQ ID NO: 57 - protein, 9 aa, Artificial Sequence
  Description of Artificial Sequence Synthetic peptide
KMAPRTLLL

SEQ ID NO: 58 - protein, 9 aa, Artificial Sequence
  Description of Artificial Sequence Synthetic peptide
LMAPRTLLL

SEQ ID NO: 59 - protein, 9 aa, Artificial Sequence
  Description of Artificial Sequence Synthetic peptide
MMAPRTLLL

SEQ ID NO: 60 - protein, 9 aa, Artificial Sequence
  Description of Artificial Sequence Synthetic peptide
NMAPRTLLL

SEQ ID NO: 61 - protein, 9 aa, Artificial Sequence
  Description of Artificial Sequence Synthetic peptide
PMAPRTLLL

SEQ ID NO: 62 - protein, 9 aa, Artificial Sequence
  Description of Artificial Sequence Synthetic peptide
QMAPRTLLL

SEQ ID NO: 63 - protein, 9 aa, Artificial Sequence
  Description of Artificial Sequence Synthetic peptide
SMAPRTLLL

SEQ ID NO: 64 - protein, 9 aa, Artificial Sequence
  Description of Artificial Sequence Synthetic peptide
YMAPRTLLL

SEQ ID NO: 65 - protein, 9 aa, Mycobacterium bohemicum
RMSPRTLLL

SEQ ID NO: 66 - protein, 9 aa, Unknown
  Description of Unknown Proteobacteria sequence
KMAPRSLLL

SEQ ID NO: 67 - protein, 9 aa, Unknown
  Description of Unknown Lentisphaerae sequence
LMAPKTLLL

SEQ ID NO: 68 - protein, 9 aa, Unknown
  Description of Unknown Microbiome-derived peptide
LMAPRNLLL

SEQ ID NO: 69 - protein, 9 aa, Massilia sp.
GMAPRSLLL

SEQ ID NO: 70 - DNA, 370 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgattactacggtagtagctacggctggtacttcgatgtctggggcacagggaccacggtcaccgtctcctcag

SEQ ID NO: 71 - DNA, 322 bp, Artificial Sequence
  Description of Artificial Sequence Synthetic polynucleotide
gatattgtgatcacacagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgctcacgttcggtgctgggaccaagctggagctgaaac

SEQ ID NO: 72 - DNA, 343 bp, Artificial Sequence
  Description of Artificial Sequence Synthetic polynucleotide
gaggtgcagctgcaggagtctggggctgagcttgtgaggccaggggcctcagtcaagttgtcctgcacagcttctggctttaacattaaagacgactatatgcactgggtgaagcagaggcctgaacagggcctggagtggattggatggattgatcctgagaatggtgatactgaatatgcctcgaagttccagggcaaggccactataacagcagacacatcctccaacacagcctacctgcagctcagcagcctgacatctgaggacactgccgtctattactgtactacgtacccgtttgcttactggggccaagggactctggtcactgtctctgcag

SEQ ID NO: 73 - DNA, 319 bp, Artificial Sequence
  Description of Artificial Sequence Synthetic polynucleotide
  modified_base (172)..(172): a, c, t, g, unknown or other
gatattgtgatcacacaatctccagcaatcatgtctgcatctctaggggagaaggtcaccatgagctgcagggccagctcaagtgtaaattacatgtactggtaccagcagaagtcagatgcctcccccaaactatggatttattacacatccaacctggctcctggagtcncagctcgcttcagtggcagtgggtctgggaactcttattctctcacaatcagcagcatggagggtgaagatgctgccacttattactgccagcagtttactagttccccatccacgttcggtgctgggaccaagctggagctgaaac

SEQ ID NO: 74 - DNA, 361 bp, Artificial Sequence
  Description of Artificial Sequence Synthetic polynucleotide
gaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagagggtacggtagtagctacgaatactttgactactggggccaaggcaccactctcacagtctcctcag

SEQ ID NO: 75 - DNA, 322 bp, Artificial Sequence
  Description of Artificial Sequence Synthetic polynucleotide
ggatattgtgatcacacagtctccaaattcatgtccacatcagtaggagacagggtcagcatcacctgcaaggccagtcaggatgtgagtactgctgtagcctggtatcaacaaaaaccagggcaatctcctaaactactgatttactgggcatccacccggcacactggagtccctgatcgcttcacaggcagtggatctgggacagattatactctcaccatcagcagtgtgcaggctgaagacctggcactttattactgtcagcaacattatagcactccattcacgttcggctcggggacaaagttggaaataaaac

SEQ ID NO: 76 - DNA, 358 bp, Artificial Sequence
  Description of Artificial Sequence Synthetic polynucleotide
gaggtgcagctgcaggagtctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactagctacaaccagaagttcaagggcaaggccacattgactgtagacaagtcctccagcacagcctacatggagctccgcagcctgacatctgaggactctgcagtctattactgtgcaagatacctcctatggtcctatgctatggactactggggtcaaggaacctcagtcaccgtctcctcag

SEQ ID NO: 77 - DNA, 319 bp, Artificial Sequence
  Description of Artificial Sequence Synthetic polynucleotide
gatattgtgatcacacagtctccagcaatcatgtctgcatctccaggggagaaggtcaccatgacctgcagtgccagctcaagtgtaagttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccgctcacgttcggtgctgggaccaagctggagctgaaac

SEQ ID NO: 78 - DNA, 358 bp, Artificial Sequence
  Description of Artificial Sequence Synthetic polynucleotide
gaggttcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgaggggatggcagcagttagcatattgggactactggggccagggaaccctggtcaccgtctcctcag

SEQ ID NO: 79 - DNA, 325 bp, Artificial Sequence
  Description of Artificial Sequence Synthetic polynucleotide
tcttatgagctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcaggaatccctgagcgattctctggctccaactctgggaacacggccaccctgaccatcagcagggtcgaagccggggatgaggccgactattactgtcaggtgtgggatagtagtagtgatcattatgtcttcggaactgggaccaaggtcaccgtcctag

SEQ ID NO: 80 - DNA, 364 bp, Artificial Sequence
  Description of Artificial Sequence Synthetic polynucleotide
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggtgactacgtgttccccacgtaccactttgactactggggccagggaaccctggtcaccgtctcctcag

SEQ ID NO: 81 - DNA, 331 bp, Artificial Sequence
  Description of Artificial Sequence Synthetic polynucleotide
cagcttgtgctgactcaatcaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtgtggtattcggcggagggaccaagctgaccgtcctag

SEQ ID NO: 82 - DNA, 358 bp, Artificial Sequence
  Description of Artificial Sequence Synthetic polynucleotide
caggtgcagctgcaggagtcggggggaggcttggtacagcctggggggtccctgagactctcctgtgtagcctctggattcaccttcagtacctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagccgaggacacggctgtgtattactgtgcgagagacccgggctggctgagctggttcgacccctggggccagggaaccctagtcaccgtctcctcag

SEQ ID NO: 83 - DNA, 325 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagtaacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtggctcacctaggacgttcggccaagggaccaaggtggaaatcaaac

SEQ ID NO: 84 - DNA, 367 bp, Artificial Sequence
  Description of Artificial Sequence Synthetic polynucleotide
caggttcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagttgatggctggtaccccaataatgatgcttttgatatctggggccaagggacaatggtcaccgtctcttcag

SEQ ID NO: 85 - DNA, 325 bp, Artificial Sequence
  Description of Artificial Sequence Synthetic polynucleotide
gaaattgtgctgactcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgcactttcggcggagggaccaaggtggagatcaaac

SEQ ID NO: 86 - DNA, 376 bp, Artificial Sequence
  Description of Artificial Sequence Synthetic polynucleotide
gaggtgcagctggtggagtctgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagagatcagggtagtaccagctgctattattgctactacggtatggacgtctggggccaagggaccacggtcaccgtctcctcag

SEQ ID NO: 87 - DNA, 322 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggaccaaggtggagatcaaac

SEQ ID NO: 88 - DNA, 358 bp, Artificial Sequence
  Description of Artificial Sequence Synthetic polynucleotide
gaagtgcagctggtgcagtctgggggaggcttggtcaagcctggagggtccctgagactctcctgtgcagcctctggattcaccttcagtgactactacatgagctggatacgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagcggattctcaagttggggagccgacgactactggggccagggaaccctggtcaccgtctcctcag

SEQ ID NO: 89 - DNA, 322 bp, Artificial Sequence
  Description of Artificial Sequence Synthetic polynucleotide
gacatcgtgatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccccctcacttttggccaggggaccaagctggagatcaaac

SEQ ID NO: 90 - DNA, 364 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagttgtggatacagctatgggtgatgcttttgatatctggggccaagggacaatggtcaccgtctcttcag

SEQ ID NO: 91 - DNA, 325 bp, Artificial Sequence
  Description of Artificial Sequence Synthetic polynucleotide
gacatcgtgatgacccagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctctctacacttttggccaggggaccaagctggagatcaaac

SEQ ID NO: 92 - DNA, 361 bp, Artificial Sequence
  Description of Artificial Sequence Synthetic polynucleotide
caggtgcagctggtacagtctgggggaggcttggtcaagcctggagggtccctgagactctcctgtgcagcctctggattcaccttcagtgactactacatgagctggatacgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagagatgggtggcgtttgggggagttatttgactactggggccagggaaccctggtcaccgtctcctcag

SEQ ID NO: 93 - DNA, 325 bp, Artificial Sequence
  Description of Artificial Sequence Synthetic polynucleotide
gaaattgtgatgactcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccactcactttcggcggagggaccaaggtggagatcaaac

SEQ ID NO: 94 - DNA, 361 bp, Artificial Sequence
  Description of Artificial Sequence Synthetic polynucleotide
caggtgcagctggtacagtctgggggaggcttggtcaagcctggagggtccctgagactctcctgtgcagcctctggattcaccttcagtgactactacatgagctggatacgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagagatagtgggagcttcgtaggggtctttgactactggggccagggaaccctggtcaccgtctcctcag

SEQ ID NO: 95 - DNA, 322 bp, Artificial Sequence
  Description of Artificial Sequence Synthetic polynucleotide
gacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccctattcactttcggccctgggaccaaagtggatatcaaac

SEQ ID NO: 96 - DNA, 364 bp, Artificial Sequence
  Description of Artificial Sequence Synthetic polynucleotide
caggtgcagctggtacagtctggggctgaggtgaagaagcctggggcctcagtgaaggtctcctgcaaggcttctggattcaccatcaccggctactatatgcactgggtgcgacaggcccctggacaaggccttgagtggatgggacggatcaaccctaacagaggtgacacaaattatgcacagaaatttcagggcagggtcaccatgaccgcggacacgtccatcagcacaatctacatggagctgagcagactgacatctgacgacacggccgtctattactgtgcgagactgaggcctttgggtagaagcaacagcttagactactggggccagggaaccctggtcaccgtctcctcgg

SEQ ID NO: 97 - DNA, 325 bp, Artificial Sequence
  Description of Artificial Sequence Synthetic polynucleotide
cagtctgccctgactcagcctgcctccgtgtctgggtctcctggacagtcgatcaccatctcctgcactggaactagcagtgacgttgatggtctctcgtggtaccaacagcacccaggcaaagcccccaaactcatgatttatgatgtcactaatcggccctcaggggtttctaatcgcttctctggctcgaagtctggcaacacggcctccctgaccatctctggtctccagactgacgacgcggctgactattactgtagcacatatacaagcagtaccactcctgtggtattcgccggagggaccaagctgaccgtcctag

SEQ ID NO: 98 - DNA, 358 bp, Artificial Sequence
  Description of Artificial Sequence Synthetic polynucleotide
caggtgcagctggtacagtctgggggaggcttggtacagcctggggggtccctgagactctcctgtgtagcctctggattcaccttcagtacctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagccgaggacacggctgtgtattactgtgcgagagacccgggctggctgagctggttcgacccctggggccagggaaccctagtcaccgtctcctcag

SEQ ID NO: 99 - DNA, 325 bp, Artificial Sequence
  Description of Artificial Sequence Synthetic polynucleotide
gaaattgtgttgacacagtctccaggcaccctgtctttgtctccaggggaaagagccaccctctcctgcagggccagtcagagtgttagtaacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctaggacgttcggccaagggaccaaggtggaaatcaaac

SEQ ID NO: 100 - DNA, 361 bp, Artificial Sequence
  Description of Artificial Sequence Synthetic polynucleotide
caggtgcagctgcaggagtcgggctcaggactggtgaagccttcacagaccctgtccctcacctgcgctgtctctggtggctccatcagcagtggtggttactcctggagctggatacggcagccaccagggaagggcctggagtggattgggtacatctatcatagtgggagcacctactacaacccgtccctcaagagtcgagtcaccatatcagtagacaggtccaagaaccagttctccctgaagctgagctctgtgaccgccgcggacacggccgtgtattactgtgccacagggggtggccacaaggaattgggaggatgggggccaaggggaaccctggtcaccgtctcctcag

SEQ ID NO: 101 - DNA, 322 bp, Artificial Sequence
  Description of Artificial Sequence Synthetic polynucleotide
gacatcgtgatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccactcactttcggcggagggaccaaggtggagatcaaac

SEQ ID NO: 102 - DNA, 358 bp, Artificial Sequence
  Description of Artificial Sequence Synthetic polynucleotide
gaagtgcagctggtgcagtctggaggaggcttggtacagcctggggggtccctgagactctcctgtgtagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagccgaggacacggctgtgtattactgtgcgagagacccgggctggctgagctggttcgacccctggggccagggaaccctggtcaccgtctcctcag

SEQ ID NO: 103 - DNA, 325 bp, Artificial Sequence
  Description of Artificial Sequence Synthetic polynucleotide
gaaattgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctaggacgttcggccaagggaccaaggtggaaatcaaac

SEQ ID NO: 104 - DNA, 364 bp, Artificial Sequence
  Description of Artificial Sequence Synthetic polynucleotide
gaggttcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagtattcggggagcagctggtatctttttttgactactggggccagggaaccctggtcaccgtctcctcag

SEQ ID NO: 105 - DNA, 325 bp, Artificial Sequence
  Description of Artificial Sequence Synthetic polynucleotide
gacatcgtgatgacccagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgtggacgttcggccaagggaccaaggtggaaatcaaac

SEQ ID NO: 106 - DNA, 358 bp, Artificial Sequence
  Description of Artificial Sequence Synthetic polynucleotide
gaagtgcagctggtgcagtctggaggaggcttggtacagcctggggggtccctgagactctcctgtgtagcctctggattcaccttcagtacctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagccgaggacacggctgtgtattactgtgcgagagacccgggctggctgagctggttcgacccctggggccagggaaccctagtcaccgtctcctcag

SEQ ID NO: 107 - DNA, 349 bp, Artificial Sequence
  Description of Artificial Sequence Synthetic polynucleotide
gaagtgcagctggtgcagtctggaggaggcttggtcaagcctggagggtccctgagactctcctgtgcagcctctaggttcaccttcagtgactactacatgagctggatacgccaggctccagggaagggactggagtgggtttcatccattaatagtagtagtagtactatatattatgcagactctgtgaagggccgattcaccatctccagggacaacgccaagaactcactggatctgcaaatgaacagcctgagagccgaggacacggccgtatattattgtgcgagagggcgtgacttctacatttactggggccagggaaccctggtcaccgtctcctcag

SEQ ID NO: 108 - DNA, 322 bp, Artificial Sequence
  Description of Artificial Sequence Synthetic polynucleotide
gacatcgtgatgacccagtctccttccaccctgtctgcatctgtaggagacagagtcaccatcacttgccgggccagtcagagtattagtaggtggatggcctggtatcagcagaaaccagggatagcccctaaactcctgatctataaggcgtctagtttagaaagtggggtcccttcaaggttcagcggcagtggatctgggacagaattcactctcaccatcagcagcctgcagcctgatgattttgcaacttattactgccaacagtataatgattacccctacacttttggccaggggaccaagctggagatcaaac

SEQ ID NO: 109 - DNA, 358 bp, Artificial Sequence
  Description of Artificial Sequence Synthetic polynucleotide
gaggttcagctggtggagtctgggggaggcttggtacagcctggggggtccctgagactctcctgtgtagcctctggattcaccttcagtacctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagccgaggacacggctgtgtattactgtgcgagagacccgggctggctgagctggttcgacccctggggccagggaaccctagtcaccgtctcctcag

SEQ ID NO: 110 - DNA, 385 bp, Artificial Sequence
  Description of Artificial Sequence Synthetic polynucleotide
caggtgcagctggtacagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatgtattactatggttcggggagctaggacccaactactactacggtatggacgtctggggccaagggaccacggtcaccgtctcctcag

SEQ ID NO: 111 - DNA, 337 bp, Artificial Sequence
  Description of Artificial Sequence Synthetic polynucleotide
gaaattgtgttgacacagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcacaccttcggccaagggacacgactggagattaaac

SEQ ID NO: 112 - DNA, 358 bp, Artificial Sequence
  Description of Artificial Sequence Synthetic polynucleotide
gaggttcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgagggggggttattacttttccgagtttgactactggggccagggaaccctggtcaccgtctcctcag

SEQ ID NO: 113 - DNA, 322 bp, Artificial Sequence
  Description of Artificial Sequence Synthetic polynucleotide
gacatcgtgatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccctctcactttcggcggagggaccaaggtggagatcaaac

SEQ ID NO: 114 - DNA, 358 bp, Artificial Sequence
  Description of Artificial Sequence Synthetic polynucleotide
gaggttcagctggtggagtctgggggaggcttggtacagcctggagggtccctgagactctcctgtgcagcctctggattcaccttcagtagttatgaaatgaactgggtccgccaggctccagggaaggggctggagtgggtttcatacattagtagtagtggtaataccatatactacgcaaactctgtgaagggccgattcaccatctccagagacaacgccaagaactcactctatctgcaaatgaacagcctgagagccgaggacacggctgtttattactgtgcgagatttttttttggtcgaggctggttcgacccctggggccagggaaccctggtcaccgtctcctcag

SEQ ID NO: 115 - DNA, 325 bp, Artificial Sequence
  Description of Artificial Sequence Synthetic polynucleotide
gaaattgtgttgacacagtctccagccaccctgtctttgtctccaggggaaagagccaccctctcctgcagggccagtcagagtgttagcagctacttagcctggtatcaacagaaacctggccaggctccccggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgctcactttcggcggagggaccaaggtggagatcaaac

SEQ ID NO: 116 - DNA, 361 bp, Artificial Sequence
  Description of Artificial Sequence Synthetic polynucleotide
caggtgcagctgcaggagtcggggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagcctcagaaaacggagatgatgcttttgatatctggggccaagggacaatggtcaccgtctcttcag

SEQ ID NO: 117 - DNA, 325 bp, Artificial Sequence
  Description of Artificial Sequence Synthetic polynucleotide
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgtacacttttggccaggggaccaagctggagatcaaac

SEQ ID NO: 118 - DNA, 361 bp, Artificial Sequence
  Description of Artificial Sequence Synthetic polynucleotide
caggtgcagctggtacagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatagtcttccagctggcgactactttgactactggggccagggaaccctggtcaccgtctcctcag

SEQ ID NO: 119 - DNA, 325 bp, Artificial Sequence
  Description of Artificial Sequence Synthetic polynucleotide
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctcagaccactttcggcggagggaccaaggtggagatcaaac

SEQ ID NO: 120 - DNA, 361 bp, Artificial Sequence
  Description of Artificial Sequence Synthetic polynucleotide
caggtgcagctggtacagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatcgggtagagatggcttttcactttgactactggggccagggaaccctggtcaccgtctcctcag

SEQ ID NO: 121 - DNA, 325 bp, Artificial Sequence
  Description of Artificial Sequence Synthetic polynucleotide
gaaattgtgttgacacagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgtggacgttcggccaagggaccaaggtggaaatcaaac

SEQ ID NO: 122 - DNA, 361 bp, Artificial Sequence
  Description of Artificial Sequence Synthetic polynucleotide
gaggttcagctggtggagtctgggggaggcttggtcaagcctggagggtccctgagactctcctgtgcagcctctggattcaccttcagtgactactacatgagctggatacgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagagatgtactggaactacggtggtggttcgacccctggggccagggaaccctggtcaccgtctcctcag

SEQ ID NO: 123 - DNA, 322 bp, Artificial Sequence
  Description of Artificial Sequence Synthetic polynucleotide
gacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgacgttcggccaagggaccaaggtggaaatcaaac

SEQ ID NO: 124 - DNA, 373 bp, Artificial Sequence
  Description of Artificial Sequence Synthetic polynucleotide
gaagtgcagctggtgcagtctggaggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatcggtattcaatagtgggagctacctcgaacggtatggacgtctggggccaagggaccacggtcaccgtctcctcag

SEQ ID NO: 125 - DNA, 322 bp, Artificial Sequence
  Description of Artificial Sequence Synthetic polynucleotide
gacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtggacgttcggccaagggaccaaggtggaaatcaaac

SEQ ID NO: 126 - DNA, 367 bp, Artificial Sequence
  Description of Artificial Sequence Synthetic polynucleotide
gaagtgcagctggtgcagtctggaggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaatcttcagtttgggactactactactacggtatggacgtctggggccaagggaccacggtcaccgtctcctcag

SEQ ID NO: 127 - DNA, 322 bp, Artificial Sequence
  Description of Artificial Sequence Synthetic polynucleotide
gaaattgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcactcactttcggcggagggaccaaggtggagatcaaac

SEQ ID NO: 128 - protein, 123 aa, Artificial Sequence [sequence omitted]

SEQ ID NO: 129 - protein, 107 aa, Artificial Sequence
  Description of Artificial Sequence Synthetic polypeptide
DIVITQSPSSMYASLGERVTITCKASQDINSYLSWFQQKPGKSPKTLIYRANRLVDGVPSRFSGSGSGQDYSLTISSLEYEDMGIYYCLQYDEFPLTFGAGTKLELK

SEQ ID NO: 130 - protein, 114 aa, Artificial Sequence
  Description of Artificial Sequence Synthetic polypeptide
EVQLQESGAELVRPGASVKLSCTASGFNIKDDYMHWVKQRPEQGLEWIGWIDPENGDTEYASKFQGKATITADTSSNTAYLQLSSLTSEDTAVYYCTTYPFAYWGQGTLVTVSA

SEQ ID NO: 131 - protein, 106 aa, Artificial Sequence
  Description of Artificial Sequence Synthetic polypeptide
  MOD_RES (58)..(58): Any amino acid
DIVITQSPAIMSASLGEKVTMSCRASSSVNYMYWYQQKSDASPKLWIYYTSNLAPGVXARFSGSGSGNSYSLTISSMEGEDAATYYCQQFTSSPSTFGAGTKLELK

SEQ ID NO: 132 - protein, 120 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARGYGSSYEYFDYWGQGTTLTVSS

SEQ ID NO: 133 - protein, 107 aa, Artificial Sequence
  Description of Artificial Sequence Synthetic polypeptide
GYCDHTVSKFMSTSVGDRVSITCKASQDVSTAVAWYQQKPGQSPKLLIYWASTRHTGVPDRFTGSGSGTDYTLTISSVQAEDLALYYCQQHYSTPFTFGSGTKLEIK

SEQ ID NO: 134 - protein, 119 aa, Artificial Sequence
  Description of Artificial Sequence Synthetic polypeptide
EVQLQESGPELVKPGASVKISCKASGYTFTDYYMNWVKQSHGKSLEWIGDINPNNGGTSYNQKFKGKATLTVDKSSSTAYMELRSLTSEDSAVYYCARYLLWSYAMDYWGQGTSVTVSS

SEQ ID NO: 135 - protein, 106 aa, Artificial Sequence
  Description of Artificial Sequence Synthetic polypeptide
DIVITQSPAIMSASPGEKVTMTCSASSSVSYMHWYQQKSGTSPKRWIYDTSKLASGVPARFSGSGSGTSYSLTISSMEAEDAATYYCQQWSSNPLTFGAGTKLELK

SEQ ID NO: 136 - protein, 119 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGWQQLAYWDYWGQGTLVTVSS

SEQ ID NO: 137 - protein, 108 aa, Artificial Sequence
  Description of Artificial Sequence Synthetic polypeptide
SYELTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVL

SEQ ID NO: 138 - protein, 121 aa, Artificial Sequence
  Description of Artificial Sequence Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKGDYVFPTYHFDYWGQGTLVTVSS

SEQ ID NO: 139 - protein, 110 aa, Artificial Sequence
  Description of Artificial Sequence Synthetic polypeptide
QLVLTQSPSASGTPGQRVTISCSGSSSNIGSNTVNWYQQLPGTAPKLLIYSNNQRPSGVPDRFSGSKSGTSASLAISGLQSEDEADYYCAAWDDSLNGVVFGGGTKLTVL

SEQ ID NO: 140 - protein, 119 aa, Artificial Sequence
  Description of Artificial Sequence Synthetic polypeptide
QVQLQESGGGLVQPGGSLRLSCVASGFTFSTYSMNWVRQAPGKGLEWVSYISSSSSTIYYADSVKGRFTISRDNAKNSLYLQMNSLRAEDTAVYYCARDPGWLSWFDPWGQGTLVTVSS

SEQ ID NO: 141 - protein, 108 aa, Artificial Sequence
  Description of Artificial Sequence Synthetic polypeptide
EIVLTQSPGTLSLSPGERATLSCRASQSVSNSYLAWYQQKPGQAPRLLIYGASSRATGIPDRFSGSGSGTDFTLTISRLEPEDFAVYYCQQYGGSPRTFGQGTKVEIK

SEQ ID NO: 142 - protein, 122 aa, Artificial Sequence
  Description of Artificial Sequence Synthetic polypeptide
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVDGWYPNNDAFDIWGQGTMVTVSS

SEQ ID NO: 143 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGGGTKVEIK

SEQ ID NO: 144 - protein, 125 aa, Artificial Sequence
  Description of Artificial Sequence Synthetic polypeptide
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARDQGSTSCYYCYYGMDVWGQGTTVTVSS

SEQ ID NO: 145 - protein, 107 aa, Artificial Sequence [sequence omitted]

SEQ ID NO: 146 - protein, 119 aa, Artificial Sequence
  Description of Artificial Sequence Synthetic polypeptide
EVQLVQSGGGLVKPGGSLRLSCAASGFTFSDYYMSWIRQAPGKGLEWVSYISSSGSTIYYADSVKGRFTISRDNAKNSLYLQMNSLRAEDTAVYYCASGFSSWGADDYWGQGTLVTVSS

SEQ ID NO: 147 - protein, 107 aa, Artificial Sequence
  Description of Artificial Sequence Synthetic polypeptide
DIVMTQSPSSLSASVGDRVTITCRASQGISNYLAWFQQKPGKAPKSLIYAASSLQSGVPSKFSGSGSGTDFTLTISSLQPEDFATYYCQQYNSYPLTFGQGTKLEIK

SEQ ID NO: 148 - protein, 121 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARVVDTAMGDAFDIWGQGTMVTVSS

SEQ ID NO: 149 - protein, 108 aa, Artificial Sequence
  Description of Artificial Sequence Synthetic polypeptide
DIVMTQSPATLSVSPGERATLSCRASQSVSSNLAWYQQKPGQAPRLLIYGASTRATGIPARFSGSGSGTEFTLTISSLQSEDFAVYYCQQYNNWPLYTFGQGTKLEIK

SEQ ID NO: 150 - protein, 120 aa, Artificial Sequence
  Description of Artificial Sequence Synthetic polypeptide
QVQLVQSGGGLVKPGGSLRLSCAASGFTFSDYYMSWIRQAPGKGLEWVSYISSSGSTIYYADSVKGRFTISRDNAKNSLYLQMNSLRAEDTAVYYCARDGWRLGELFDYWGQGTLVTVSS

SEQ ID NO: 151 - protein, 108 aa, Artificial Sequence
  Description of Artificial Sequence Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYNNWPPLTFGGGTKVEIK

SEQ ID NO: 152 - protein, 120 aa, Artificial Sequence
  Description of Artificial Sequence Synthetic polypeptide
QVQLVQSGGGLVKPGGSLRLSCAASGFTFSDYYMSWIRQAPGKGLEWVSYISSSGSTIYYADSVKGRFTISRDNAKNSLYLQMNSLRAEDTAVYYCARDSGSFVGVFDYWGQGTLVTVSS

SEQ ID NO: 153 - protein, 107 aa, Artificial Sequence
  Description of Artificial Sequence Synthetic polypeptide
DIVMTQSPSSLSASVGDRVTITCRASQSISSYLNWYQQKPGKAPKLLIYAASSLQSGVPSRFSGSGSGTDFTLTISSLQPEDFATYYCQQSYSTLFTFGPGTKVDIK

SEQ ID NO: 154 - protein, 121 aa, Artificial Sequence
  Description of Artificial Sequence Synthetic polypeptide
QVQLVQSGAEVKKPGASVKVSCKASGFTITGYYMHWVRQAPGQGLEWMGRINPNRGDTNYAQKFQGRVTMTADTSISTIYMELSRLTSDDTAVYYCARLRPLGRSNSLDYWGQGTLVTVSS

SEQ ID NO: 155 - protein, 108 aa, Artificial Sequence
  Description of Artificial Sequence Synthetic polypeptide
QSALTQPASVSGSPGQSITISCTGTSSDVDGLSWYQQHPGKAPKLMIYDVTNRPSGVSNRFSGSKSGNTASLTISGLQTDDAADYYCSTYTSSTTPVVFAGGTKLTVL

SEQ ID NO: 156 - protein, 119 aa, Artificial Sequence
  Description of Artificial Sequence Synthetic polypeptide
QVQLVQSGGGLVQPGGSLRLSCVASGFTFSTYSMNWVRQAPGKGLEWVSYISSSSSTIYYADSVKGRFTISRDNAKNSLYLQMNSLRAEDTAVYYCARDPGWLSWFDPWGQGTLVTVSS

SEQ ID NO: 157 - protein, 108 aa, Artificial Sequence
  Description of Artificial Sequence Synthetic polypeptide
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPRTFGQGTKVEIK

SEQ ID NO: 158 - protein, 120 aa, Artificial Sequence
  Description of Artificial Sequence Synthetic polypeptide
QVQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCATGGGHKELGGWGPRGTLVTVSS

SEQ ID NO: 159 - protein, 107 aa, Artificial Sequence
  Description of Artificial Sequence Synthetic polypeptide
DIVMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 160 - protein, 119 aa, Artificial Sequence
  Description of Artificial Sequence Synthetic polypeptide
EVQLVQSGGGLVQPGGSLRLSCVASGFTFSSYSMNWVRQAPGKGLEWVSYISSSSSTIYYADSVKGRFTISRDNAKNSLYLQMNSLRAEDTAVYYCARDPGWLSWFDPWGQGTLVTVSS

SEQ ID NO: 161 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 162 - protein, 121 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARVFGEQLVSFFDYWGQGTLVTVSS

SEQ ID NO: 163 - protein, 108 aa, Artificial Sequence
  Description of Artificial Sequence Synthetic polypeptide
DIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIK

SEQ ID NO: 164 - protein, 119 aa, Artificial Sequence
  Description of Artificial Sequence Synthetic polypeptide
EVQLVQSGGGLVQPGGSLRLSCVASGFTFSTYSMNWVRQAPGKGLEWVSYISSSSSTIYYADSVKGRFTISRDNAKNSLYLQMNSLRAEDTAVYYCARDPGWLSWFDPWGQGTLVTVSS

SEQ ID NO: 165 - protein, 116 aa, Artificial Sequence
  Description of Artificial Sequence Synthetic polypeptide
EVQLVQSGGGLVKPGGSLRLSCAASRFTFSDYYMSWIRQAPGKGLEWVSSINSSSSTIYYADSVKGRFTISRDNAKNSLDLQMNSLRAEDTAVYYCARGRDFYIYWGQGTLVTVSS

SEQ ID NO: 166 - protein, 107 aa, Artificial Sequence
  Description of Artificial Sequence Synthetic polypeptide
DIVMTQSPSTLSASVGDRVTITCRASQSISRWMAWYQQKPGIAPKLLIYKASSLESGVPSRFSGSGSGTEFTLTISSLQPDDFATYYCQQYNDYPYTFGQGTKLEIK

SEQ ID NO: 167 - protein, 119 aa, Artificial Sequence
  Description of Artificial Sequence Synthetic polypeptide
EVQLVESGGGLVQPGGSLRLSCVASGFTFSTYSMNWVRQAPGKGLEWVSYISSSSSTIYYADSVKGRFTISRDNAKNSLYLQMNSLRAEDTAVYYCARDPGWLSWFDPWGQGTLVTVSS

SEQ ID NO: 168 - protein, 128 aa, Artificial Sequence
  Description of Artificial Sequence Synthetic polypeptide
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKDVLLWFGELGPNYYYGMDVWGQGTTVTVSS

SEQ ID NO: 169 - protein, 112 aa, Artificial Sequence
  Description of Artificial Sequence Synthetic polypeptide
EIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGQGTRLEIK

SEQ ID NO: 170 - protein, 119 aa, Artificial Sequence
  Description of Artificial Sequence Synthetic polypeptide
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARGGYYFSEFDYWGQGTLVTVSS

SEQ ID NO: 171 - protein, 107 aa, Artificial Sequence
  Description of Artificial Sequence Synthetic polypeptide
DIVMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIK

SEQ ID NO: 172 - protein, 119 aa, Artificial Sequence
  Description of Artificial Sequence Synthetic polypeptide
EVQLVESGGGLVQPGGSLRLSCAASGFTFSSYEMNWVRQAPGKGLEWVSYISSSGNTIYYANSVKGRFTISRDNAKNSLYLQMNSLRAEDTAVYYCARFFFGRGWFDPWGQGTLVTVSS

SEQ ID NO: 173 - protein, 108 aa, Artificial Sequence [sequence omitted]

SEQ ID NO: 174 - protein, 120 aa, Artificial Sequence
  Description of Artificial Sequence Synthetic polypeptide
QVQLQESGGGLVKPGGSLRLSCAASGFTFSSYSMNWVRQAPGKGLEWVSSISSSSSYIYYADSVKGRFTISRDNAKNSLYLQMNSLRAEDTAVYYCARASENGDDAFDIWGQGTMVTVSS

SEQ ID NO: 175 - protein, 108 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPYTFGQGTKLEIK

SEQ ID NO: 176 - protein, 120 aa, Artificial Sequence
  Description of Artificial Sequence Synthetic polypeptide
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARDSLPAGDYFDYWGQGTLVTVSS

SEQ ID NO: 177 - protein, 108 aa, Artificial Sequence
  Description of Artificial Sequence Synthetic polypeptide
DIQLTQSPSFLSASVGDRVTITCRASQGISSYLAWYQQKPGKAPKLLIYAASTLQSGVPSRFSGSGSGTEFTLTISSLQPEDFATYYCQQLNSYPQTTFGGGTKVEIK

SEQ ID NO: 178 - protein, 120 aa, Artificial Sequence
  Description of Artificial Sequence Synthetic polypeptide
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARDRVEMAFHFDYWGQGTLVTVSS

SEQ ID NO: 179 - protein, 108 aa, Artificial Sequence
  Description of Artificial Sequence Synthetic polypeptide
EIVLTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIK

SEQ ID NO: 180 - protein, 120 aa, Artificial Sequence
  Description of Artificial Sequence Synthetic polypeptide
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARDVLELRWWFDPWGQGTLVTVSS

SEQ ID NO: 181 - protein, 107 aa, Artificial Sequence
  Description of Artificial Sequence Synthetic polypeptide
DIVMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 182 - protein, 124 aa, Artificial Sequence
  Description of Artificial Sequence Synthetic polypeptide
EVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKDRYSIVGATSNGMDVWGQGTTVTVSS

SEQ ID NO: 183 - protein, 107 aa, Artificial Sequence
  Description of Artificial Sequence Synthetic polypeptide
DIVMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIK

SEQ ID NO: 184 - protein, 122 aa, Artificial Sequence
  Description of Artificial Sequence Synthetic polypeptide
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKSSVWDYYYYGMDVWGQGTTVTVSS

SEQ ID NO: 185 - protein, 107 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSLTFGGGTKVEIK

SEQ ID NO: 186 - protein, 122 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSVQLWDEYNWFDPWGQGTLVTVSS

SEQ ID NO: 187 - protein, 108 aa, Artificial Sequence
  Description of Artificial Sequence Synthetic polypeptide
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLVVFGGGTKLTVL

SEQ ID NO: 188 - protein, 255 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYGSSYGWYFDVWGTGTTVTVSSGSGGGGSGGGGSGGGGSGGGGSGGGDIVITQSPSSMYASLGERVTITCKASQDINSYLSWFQQKPGKSPKTLIYRANRLVDGVPSRFSGSGSGQDYSLTISSLEYEDMGIYYCLQYDEFPLTFGAGTKLELK

SEQ ID NO: 189 - protein, 255 aa, Artificial Sequence; identical to SEQ ID NO: 188
  Description of Artificial Sequence Synthetic polypeptide

SEQ ID NO: 190 - DNA, 358 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgaggggatggcagcagttagcatattgggactactggggccagggaaccctggtcaccgtctcctcag

SEQ ID NO: 191 - DNA, 358 bp, Artificial Sequence
  Description of Artificial Sequence Synthetic polynucleotide
gaggtgcagctggtggagtctgggggaggcttggtacagcctggggggtccctgagactctcctgtgtagcctctggattcaccttcagtacctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagccgaggacacggctgtgtattactgtgcgagagacccgggctggctgagctggttcgacccctggggccagggaaccctagtcaccgtctcctcag

SEQ ID NO: 192 - DNA, 367 bp, Artificial Sequence
  Description of Artificial Sequence Synthetic polynucleotide
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagttgatggctggtaccccaataatgatgcttttgatatctggggccaagggacaatggtcaccgtctcttcag

SEQ ID NO: 193 - DNA, 375 bp, Artificial Sequence
  Description of Artificial Sequence Synthetic polynucleotide
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagagatcagggtagtaccagctgctattattgctactacggtatggacgtctggggccaagggaccacggtcaccgtctcctca

SEQ ID NO: 194 - DNA, 358 bp, Artificial Sequence
  Description of Artificial Sequence Synthetic polynucleotide
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagcggattctcgagttggggagccgacgactactggggccagggaaccctggtcaccgtctcctcag

SEQ ID NO: 195 - DNA, 361 bp, Artificial Sequence
  Description of Artificial Sequence Synthetic polynucleotide
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagagatagtgggagcttcgtaggggtctttgactactggggccagggaaccctggtcaccgtctcctcag

SEQ ID NO: 196 - DNA, 358 bp, Artificial Sequence
  Description of Artificial Sequence Synthetic polynucleotide
cagctgcagctgcaggagtccggctcaggactggtgaagccttcacagaccctgtccctcacctgcgctgtctctggtggctccatcagcagtggtggttactcctggagctggatccggcagccaccagggaagggcctggagtggattgggtacatctatcatagtgggagcacctactacaacccgtccctcaagagtcgagtcaccatatcagtagacaggtccaagaaccagttctccctgaagctgagctctgtgaccgccgcggacacggccgtgtattactgtgccacagggggtggccacaaggaattgggaggatggggccagggaaccctggtcaccgtctcctcag

SEQ ID NO: 197 - DNA, 358 bp, Artificial Sequence
  Description of Artificial Sequence Synthetic polynucleotide
gaggtgcagctggtggagtctgggggaggcttggtacagcctggggggtccctgagactctcctgtgtagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagccgaggacacggctgtgtattactgtgcgagagacccgggctggctgagctggttcgacccctggggccagggaaccctggtcaccgtctcctcag

SEQ ID NO: 198 - DNA, 364 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagtattcggggagcagctggtatctttttttgactactggggccagggaaccctggtcaccgtctcctcag

SEQ ID NO: 199 - DNA, 384 bp, Artificial Sequence
  Description of Artificial Sequence Synthetic polynucleotide
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatgtattactatggttcggggagctaggacccaactactactacggtatggacgtctggggccaagggaccacggtcaccgtctcctca

SEQ ID NO: 200 - DNA, 358 bp, Artificial Sequence
  Description of Artificial Sequence Synthetic polynucleotide
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgagggggggttattacttttccgagtttgactactggggccagggaaccctggtcaccgtctcctcag

SEQ ID NO: 201 - DNA, 361 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagcctcagaaaacggagatgatgcttttgatatctggggccaagggacaatggtcaccgtctcttcag

SEQ ID NO: 202 - DNA, 361 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatagtcttccagctggcgactactttgactactggggccagggaaccctggtcaccgtctcctcag

SEQ ID NO: 203 - DNA, 361 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatcgggtagagatggcttttcactttgactactggggccagggaaccctggtcaccgtctcctcag

SEQ ID NO: 204 - DNA, 361 bp, Artificial Sequence
  Description of Artificial Sequence Synthetic polynucleotide
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagagatgtactggaactacggtggtggttcgacccctggggccagggaaccctggtcaccgtctcctcag

SEQ ID NO: 205 - DNA, 372 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatcggtattcaatagtgggagctacctcgaacggtatggacgtctggggccaagggaccacggtcaccgtctcctca

SEQ ID NO: 206 - DNA, 366 bp, Artificial Sequence
  Description of Artificial Sequence Synthetic polynucleotide
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaatcttcagtttgggactactactactacggtatggacgtctggggccaagggaccacggtcaccgtctcctca

SEQ ID NO: 207 - DNA, 369 bp, Artificial Sequence
  Description of Artificial Sequence Synthetic polynucleotide
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagaacccccggagatgaatactactactactactacatggacgtctggggcaaagggaccacggtcaccgtctcctca

SEQ ID NO: 208 - DNA, 364 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagatgcgcagctggttttgtccctgatgcttttgatatctggggccaagggacaatggtcaccgtctcttcag

SEQ ID NO: 209 - DNA, 378 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagtttccctaattgtagtagtaccagctgctactactactactacatggacgtctggggcaaagggaccacggtcaccgtctcctca

SEQ ID NO: 210 - DNA, 364 bp, Artificial Sequence
  Description of Artificial Sequence Synthetic polynucleotide
caggtgcagctgcaggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagtttcacgcgtatgtccccgtatgcttttgatatctggggccaagggacaatggtcaccgtctcttcag

SEQ ID NO: 211 - DNA, 363 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaggacagtggtccccggggggctacggtatggacgtctggggccaagggaccacggtcaccgtctcctca

SEQ ID NO: 212 - DNA, 354 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagactgacttactactactacatggacgtctggggcaaagggaccacggtcaccgtctcctca

SEQ ID NO: 213 - DNA, 364 bp, Artificial Sequence
  Description of Artificial Sequence Synthetic polynucleotide
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagtcagggattaccagctccctgatgcttttgatgtctggggccaagggacaatggtcaccgtctcttcag

SEQ ID NO: 214 - DNA, 360 bp, Artificial Sequence
  Description of Artificial Sequence Synthetic polynucleotide
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtagtagttacacaaactacgcagactctgtgaagggccgattcaccatctccagagacaacgccaagaactcactgtatctgcaaatgaacagcctgagagccgaggacacggccgtgtattactgtgcgagaggtggtatttactactactactacatggacgtctggggcaaagggaccacggtcaccgtctcctca

SEQ ID NO: 215 - DNA, 364 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatttttcgggggatttttggagtggttatgactactggggccagggaaccctggtcaccgtctcctcag

SEQ ID NO: 216 - DNA, 364 bp, Artificial Sequence
  Description of Artificial Sequence Synthetic polynucleotide
gaggtgcagctggtggagtctgggggaggcttggtacagcctggagggtccctgagactctcctgtgcagcctctggattcaccttcagtagttatgaaatgaactgggtccgccaggctccagggaaggggctggagtgggtttcatacattagtagtagtggtagtaccatatactacgcagactctgtgaagggccgattcaccatctccagagacaacgccaagaactcactgtatctgcaaatgaacagcctgagagccgaggacacggctgtttattactgtgcgagagggtattgtagtagtaccagctgcccttttgactactggggccagggaaccctggtcaccgtctcctcag

SEQ ID NO: 217 - DNA, 355 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaatgtaccagctggccgccattgactactggggccagggaaccctggtcaccgtctcctcag

SEQ ID NO: 218 - DNA, 357 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagctagttactactactactacatggacgtctggggcaaagggaccacggtcaccgtctcctca

SEQ ID NO: 219 - DNA, 349 bp, Artificial Sequence
  Description of Artificial Sequence Synthetic polynucleotide
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcaggtattagtggtagtggtggtagcacatacaacgcagactccgtgaagggccggttcaccatctccagagacaattccaagaacacgctgtatctgcaaatgaacagcctgagagccgaggacacggccgtatattactgtgcgaaagggtcagacatccttgactactggggccagggaaccctggtcaccgtctcctcag

SEQ ID NO: 220 - DNA, 358 bp, Artificial Sequence
  Description of Artificial Sequence Synthetic polynucleotide
caggtgcagctggtggagtctgggggaggcgtggtccagcctgagaggtccctgagactctcctgtgcagcctctggattcaccttcagttcctatgatatgcactgggtccgccaggctccaggcaaggggctggagtgggtggcaattatatcatatggtggcgccaatagatattacgcagactccgtgaagggccgattcaccatctccagagacaattcgaagaacaccctgtatctgcagatgaacagcctaaaaactgaggacacggctgtttattactgtgcgaggcctgataagtgggagctgccacctgactactggggccagggaaccctggtcaccgtctcctcag

SEQ ID NO: 221 - DNA, 388 bp, Artificial Sequence
  Description of Artificial Sequence Synthetic polynucleotide
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtagtagttacacaaactacgcagactctgtgaagggccgattcaccatctccagagacaacgccaagaactcactgtatctgcaaatgaacagcctgagagccgaggacacggctgtgtattactgtgcgagaggccgcactatgttcaagaattactatgatagtagtggttattacggctactttgactactggggccagggaaccctggtcaccgtctcctcag

SEQ ID NO: 222 - DNA, 367 bp, Artificial Sequence
  Description of Artificial Sequence Synthetic polynucleotide
caggttcagctggtgcagtctggagctggggcctcagtgaaggtctcctgcaaggcttctggcaacacctttaccagctatggtatcagctgggtgcgacaggcccctggacaagggcttgagtggatgggatggatcatcccttacaatggtaacacaaactatgcacagaagttccagggcagagtcaccatgaccacagacacatccacgagcacagcctacatggagctgaggagcctgagatctgacgacacggccgtgtattactgtgcgagagatggggaggttggtgcttttgacaactactactactactgcggctacggcgtcatcgtcaccaccgcctccaccaccaccgccaccc

SEQ ID NO: 223 - DNA, 361 bp, Artificial Sequence
  Description of Artificial Sequence Synthetic polynucleotide
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtagtagttacacaaactacgcagactctgtgaagggccgattcaccatctccagagacaacgccaagaactcactgtatctgcaaatgaacagcctgagagccgaggacacggctgtgtattactgtgcgagagttggtgtgggagccacgtactactttgactactggggccagggaaccctggtcaccgtctcctcag

SEQ ID NO: 224 - DNA, 364 bp, Artificial Sequence
  Description of Artificial Sequence Synthetic polynucleotide
gaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccaagtattacgatattttgaccggtcatgactactggggccagggaaccctggtcaccgtctcctcag

SEQ ID NO: 225 - DNA, 358 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagttatgattacgtttgggggagctgactactggggccagggaaccctggtcaccgtctcctcag

SEQ ID NO: 226 - DNA, 363 bp, Artificial Sequence
  Description of Artificial Sequence Synthetic polynucleotide
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtagtagttacacaaactacgcagactctgtgaagggccgattcaccatctccagagacaacgccaagaactcactgtatctgcaaatgaacagcctgagagccgaggacacggctgtgtattactgtgcgagagtaggggaggatgactacgataacggtatggacgtctggggccaagggaccacggtcaccgtctcctca

SEQ ID NO: 227 - DNA, 369 bp, Artificial Sequence
  Description of Artificial Sequence Synthetic polynucleotide
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgagagccctgacgtattacgattttccggactacggtatggacgtctggggccaagggaccacggtcaccgtctcctca

SEQ ID NO: 228 - DNA, 355 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagtggtgtcttactggaagcctttgactactggggccagggaaccctggtcaccgtctcctcag

SEQ ID NO: 229 - DNA, 358 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagatcgtgacctacttgatgcttttgatatctggggccaagggacaatggtcaccgtctcttcag

SEQ ID NO: 230 - DNA, 364 bp, Artificial Sequence
  Description of Artificial Sequence Synthetic polynucleotide
gaggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaacgccaagaactcactgtatctgcaaatgaacagcctgagagccgaggacacggctgtgtattactgtgcgaggaccttctacagtaactacctcgatgcttttgatatctggggccaagggacaatggtcaccgtctcttcag

SEQ ID NO: 231 - DNA, 358 bp, Artificial Sequence
  Description of Artificial Sequence Synthetic polynucleotide
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtagtagttacacaaactacgcagactctgtgaagggccgattcaccatctccagagacaacgccaagaactcactgtatctgcaaatgaacagcctgagagccgaggacacggccgtgtattactgtgcgagagctttacgatattttgactggtatgactactggggccagggaaccctggtcaccgtctcctcag

SEQ ID NO: 232 - DNA, 376 bp, Artificial Sequence
  Description of Artificial Sequence Synthetic polynucleotide
gaggtgcagctggtggagtctgggggaggcctggtcaagcctggggggtccctgagactctcctgtgcagcctctggattcaccttcagtagctatagcatgaactgggtccgccaggctccaggggaggggctggagtgggtctcatccattagtagtagtagtacttacatatactacgcagactcagtgaagggccgattcaccatctccagagacaacgccaagaactcactgtatctacaaatgaacagcctgagagccgaggacacggctgtgtattactgtgcgagagatcggcccgcgggcagctcgtcgttgggttcgggatacttcgatctctggggccgtggcaccctggtcactgtctcctcag

SEQ ID NO: 233 - DNA, 364 bp, Artificial Sequence
  Description of Artificial Sequence Synthetic polynucleotide
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaggagaccagaggttactatggacggaaattgactactggggccagggaaccctggtcaccgtctcctcag

SEQ ID NO: 234 - DNA, 354 bp, Artificial Sequence
  Description of Artificial Sequence Synthetic polynucleotide
gaggtgcagctggtggagtctgggggaggcttggtacagcctggggggtccctgagactctcctgtgcagcctctggattcaccttcagtacctatcccatgaactgggtccgccaggctccagggaaggggctggagtgggtttcatacattagtagtagtagtagtaccatatactacgcagactgtgtgaagggccgattcaccatctccagagacaatgccaagaactcactgtatctgcaaatgaacagcctgagagacgaggacacggctgtgtattactgtgcgagatactatattgcgtacggtatggacgtctggggccaagggaccacggtcaccgtctcctca

SEQ ID NO: 235 - DNA, 379 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaagatgaagggagtagcgatttttggagtggttattatgcctcctttgactactggggccagggaaccctggtcaccgtctcctcag

SEQ ID NO: 236 - DNA, 355 bp, Artificial Sequence
  Description of Artificial Sequence Synthetic polynucleotide
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaaaaaccagttctccctgaagctgagctctgtgaccgctgcggacacggccgtgtattactgtgcgagtcatgactacactgaccacgcggcaggatactggggccagggaaccctggtcaccgtctcctcag

SEQ ID NO: 237 - DNA, 367 bp, Artificial Sequence
  Description of Artificial Sequence Synthetic polynucleotide
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagattacggtgggagctactattatgaatacttccagcactggggccagggcaccctggtcaccgtctcctcag

SEQ ID NO: 238 - DNA, 361 bp, Artificial Sequence
  Description of Artificial Sequence Synthetic polynucleotide
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtagtagttacacaaactacgcagactctgtgaagggccgattcaccatctccagagacaacgccaagaactcactgtatctgcaaatgaacagcctgagagccgaggacacggccgtgtattactgtgcgagaggacgtggatacagctatggttattctgactactggggccagggaaccctggtcaccgtctcctcag

SEQ ID NO: 239 - DNA, 376 bp, Artificial Sequence
  Description of Artificial Sequence Synthetic polynucleotide
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagacctggggtatagcagcagctggtacacctcaaactggttcgacccctggggccagggaaccctggtcaccgtctcctcag

SEQ ID NO: 240 - DNA, 306 bp, Artificial Sequence
  Description of Artificial Sequence Synthetic polynucleotide
tcctatgtgctgactcagccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcattatgtcttcggaactgggaccaaggtcaccgtcctag

SEQ ID NO: 241 - DNA, 333 bp, Artificial Sequence
  Description of Artificial Sequence Synthetic polynucleotide
gacatcgtgatgacccagtctcaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatgacagcctgaatggtgtggtattcggcggagggaccaagctgaccgtcctag

SEQ ID NO: 242 - DNA, 325 bp, Artificial Sequence
  Description of Artificial Sequence Synthetic polynucleotide
gaaattgtgttgacgcagtctcccggcaccctgtctttgtctccaggggaaagagccaccctctcctgcagggccagtcagagtgttagtaacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtggctcacctaggacgttcggccaagggaccaaggtggaaatcaaac

SEQ ID NO: 243 - DNA, 324 bp, Artificial Sequence
  Description of Artificial Sequence Synthetic polynucleotide
gaaattgtgttgacgcagtctccaggcaccctgtctttgtctcaaggtggagatcaaaccaactgtggctgcaccatctgacttcagcttcccactacttaacctggtaccagcagaaacctggccaggatcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcgcactttcggcggagggaccaaggtggagatcaaac

SEQ ID NO: 244 - DNA, 322 bp, Artificial Sequence
  Description of Artificial Sequence Synthetic polynucleotide
gacatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccccctcacttttggccaggggaccaagctggagatcaaac

SEQ ID NO: 245 - DNA, 325 bp, Artificial Sequence
  Description of Artificial Sequence Synthetic polynucleotide
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctctctacacttttggccaggggaccaagctggagatcaaac

SEQ ID NO: 246 - DNA, 316 bp, Artificial Sequence
  Description of Artificial Sequence Synthetic polynucleotide
gacatccagatgacccagtctcccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccctattcactttcggccctgggaccaaagtggatatcaaac

SEQ ID NO: 247 - DNA, 310 bp, Artificial Sequence
  Description of Artificial Sequence Synthetic polynucleotide
gaaattgtgttgacgcagtctctgtctccaggggaaagagccaccctctcctgcagggccagtcagagtgttagtaacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctaggacgttcggccaagggaccaaggtggaaatcaaac

SEQ ID NO: 248 - DNA, 307 bp, Artificial Sequence
  Description of Artificial Sequence Synthetic polynucleotide
gaaattgtgttgacgcagtctcctccaggggaaagagccaccctctcctgcagggccagtcagagtgttagtaacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtggctcacctaggacgttcggccaagggaccaaggtggaaatcaaac

SEQ ID NO: 249 - DNA, 325 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctaggacgttcggccaagggaccaaggtggaaatcaaac

SEQ ID NO: 250 - DNA, 307 bp, Artificial Sequence
  Description of Artificial Sequence Synthetic polynucleotide
gaaatagtgatgacgcagtctcctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgtggacgttcggccaagggaccaaggtggaaatcaaac

SEQ ID NO: 251 - DNA, 325 bp, Artificial Sequence
  Description of Artificial Sequence Synthetic polynucleotide
gaaattgtgttgacgcagtctcagggcaccctgtctttgtctccaggggaaagagccaccctctcctgcagggccagtcagagtgttagtaacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctaggacgttcggccaagggaccaaggtggaaatcaaac

SEQ ID NO: 252 - DNA, 325 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagtaacagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctaggacgttcggccaagggaccaaggtggaaatcaaac

SEQ ID NO: 253 - DNA, 337 bp, Artificial Sequence
  Description of Artificial Sequence Synthetic polynucleotide
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcacaccttcggccaagggacacgactggagattaaac

SEQ ID NO: 254 - DNA, 322 bp, Artificial Sequence
  Description of Artificial Sequence Synthetic polynucleotide
gacatccagatgacccagtctccatcctcactgtctgcatctgtaggagacagagtcaccatcacttgtcgggcgagtcagggcattagcaattatttagcctggtttcagcagaaaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacagtataatagttaccctctcactttcggcggagggaccaaggtggagatcaaac

SEQ ID NO: 255 - DNA, 325 bp, Artificial Sequence
  Description of Artificial Sequence Synthetic polynucleotide
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgtggacgttcggccaagggaccaaggtggaaatcaaac

SEQ ID NO: 256 - DNA, 322 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgacgttcggccaagggaccaaggtggaaatcaaac

SEQ ID NO: 257 - DNA, 322 bp, Artificial Sequence
  Description of Artificial Sequence Synthetic polynucleotide
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtggacgttcggccaagggaccaaggtggaaatcaaac

SEQ ID NO: 258 - DNA, 322 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcactcactttcggcggagggaccaaggtggagatcaaac

SEQ ID NO: 259 - DNA, 322 bp, Artificial Sequence
  Description of Artificial Sequence Synthetic polynucleotide
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctcggacttttggccaggggaccaagctggagatcaaac

SEQ ID NO: 260 - DNA, 337 bp, Artificial Sequence
  Description of Artificial Sequence Synthetic polynucleotide
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacccgtgatgtggtattcggcggagggaccaagctgaccgtcctag

SEQ ID NO: 261 - DNA, 322 bp, Artificial Sequence
  Description of Artificial Sequence Synthetic polynucleotide
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgccaacagtataatagttatccgtggacgttcggccaagggaccaaggtggaaatcaaac

SEQ ID NO: 262 - DNA, 337 bp, Artificial Sequence
  Description of Artificial Sequence Synthetic polynucleotide
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcggacttttggccaggggaccaagctggagatcaaac

SEQ ID NO: 263 - DNA, 325 bp, Artificial Sequence
  Description of Artificial Sequence Synthetic polynucleotide
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgccaacagtataatagttattcatcgtggacgttcggccaagggaccaaggtggaaatcaaac

SEQ ID NO: 264 - DNA, 322 bp, Artificial Sequence
  Description of Artificial Sequence Synthetic polynucleotide
gccatccggatgacccagtctccatcctcattctctgcatctacaggagacagagtcaccatcacttgtcgggcgagtcagggtattagcagttatttagcctggtatcagcaaaaaccagggaaagcccctaagctcctgatctatgctgcatccactttgcaaagtggggtcccatcaaggttcagcggcagtggatctgggacagatttcactctcaccatcagctgcctgcagtctgaagattttgcaacttattactgtcaacagtattatagttacgcgctcactttcggcggagggaccaaggtggagatcaaac

SEQ ID NO: 265 - DNA, 325 bp, Artificial Sequence
  Description of Artificial Sequence Synthetic polynucleotide
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgctcactttcggcggagggaccaaggtggagatcaaac

SEQ ID NO: 266 - DNA, 325 bp, Artificial Sequence
  Description of Artificial Sequence Synthetic polynucleotide
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgtggacgttcggccaagggaccaaggtggaaatcaaac

SEQ ID NO: 267 - DNA, 346 bp, Artificial Sequence
  Description of Artificial Sequence Synthetic polynucleotide
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccggaattcactttcggccctgggaccaaagtggatatcaaac

SEQ ID NO: 268 - DNA, 322 bp, Artificial Sequence
  Description of Artificial Sequence Synthetic polynucleotide
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgccaacagtataatagttacccattcactttcggccctgggaccaaagtggatatcaaac

SEQ ID NO: 269 - DNA, 328 bp, Artificial Sequence
  Description of Artificial Sequence Synthetic polynucleotide
cagtctgtgctgactcagccaccctcagcgtctgggacccccgggcagagggtcaccatctcttgttctggaagcagctccaacatcggaagcaatactgtaaactggtaccagcagctcccaggaacggcccccaaactcctcatctatggtaatgataagcggccctcaggggtccctgaccgattctctggctccaagtctggcacctcagcctccctggccatcagtgggctccagtctgaggatgaggctgattattactgtgcagcatgggatgacagtgtgaatggggtgttcggcggagggaccaagctgaccgtcctag

SEQ ID NO: 270 - DNA, 325 bp, Artificial Sequence
  Description of Artificial Sequence Synthetic polynucleotide
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgatcaccttcggccaagggacacgactggagattaaac

SEQ ID NO: 271 - DNA, 329 bp, Artificial Sequence
  Description of Artificial Sequence Synthetic polynucleotide
gaaattgtgttgacgcagtctccagtcaccctgtctttgtctccaggggaaagagccaccctctcctgcagggccagtcagaggattagcacctactacttatgctggcaacagaaaaatgctgagcatcctccgatactcatcaattatgcatcctacaggaccgcggctgtcatcccaaagatgttcggtggtgggggtgggacacaaatactttcttctcatcatctccctacagcagcctgatgaggattcagtgtaatattgtcatcaacatgatagttcacatttattcactttcggccatgggaccaaactggatatcaaac

SEQ ID NO: 272 - DNA, 319 bp, Artificial Sequence
  Description of Artificial Sequence Synthetic polynucleotide
gacatccagatgacccagtctccatcttctgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcactatcagcagcctgcagcctgaagattttgcaacttactattgtcaacaggctaacagccctctcactttcggcggagggaccaaggtggagatcaaac

SEQ ID NO: 273 - DNA, 328 bp, Artificial Sequence
  Description of Artificial Sequence Synthetic polynucleotide
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctcctatgtacacttttggccaggggaccaagctggagatcaaac

SEQ ID NO: 274 - DNA, 368 bp, Artificial Sequence
  Description of Artificial Sequence Synthetic polynucleotide
cagcctgtgctgactcagccaacctccctctcagcatctctcggagcatcagccagattcacctgcaccttgcgcagtggcatcaatgttggtacctacaggatatactggtaccagcagaagccagggagtcttccccggtatctcctgaggtacaaatcagactcagataagcagcagggctctggagtccccagccgcttctctggatccaaagatgcttcaaccaatgcaggccttttactcatctctgggctccagtctgaagatgaggctgactattactgtgccatttggtacagcagcacttctcacagtgacacacacacatggggaagtgggacaaaaacctcaccctgctctcactc

SEQ ID NO: 275 - DNA, 322 bp, Artificial Sequence
  Description of Artificial Sequence Synthetic polynucleotide
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgctcactttcggcggagggaccaaggtggagatcaaac

SEQ ID NO: 276 - DNA, 322 bp, Artificial Sequence
  Description of Artificial Sequence Synthetic polynucleotide
gacatccagatgacccagtctccatcctccctgtctgcatctgtaggagacagagtcaccatcacttgccgggcaagtcagaacattaacacctatttaaattggtatcagaagagaccagggatagcccctaagctcctcatatatggtgcatccagtttgcaaagtggggtcccatcaaggttcagtggcagtggatctgggacagatttcactctcaccatcagcagcctgcagcctgaagattttgcaacttattactgtcagcaatatcacacttcccctctcactttcggcggagggaccccagtggacatcaaac

SEQ ID NO: 277 - DNA, 322 bp, Artificial Sequence
  Description of Artificial Sequence Synthetic polynucleotide
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattcgtggacgttcggccaagggaccaaggtggaaatcaaac

SEQ ID NO: 278 - DNA, 331 bp, Artificial Sequence
  Description of Artificial Sequence Synthetic polynucleotide
cagtctgccctgactcagcctccctccgcgtccgggtctcctggacagtcagtcaccatctcctgcactggaaccagcagtgacgttggtggttataactatgtctcctggtaccaacaacacccaggcaaagtccccaaactcatgatttatgaggtcactaagcggccctcaggggtccctgatcgcttctctggctccaagtctggcaacacggcctccctgaccgtctctgggctccaggctgaggatgaggctgattattactgcagctcatatgcaggcagcaacaatttggtattcggcggagggaccaagctgaccgtcctag

SEQ ID NO: 279 - DNA, 325 bp, Artificial Sequence
  Description of Artificial Sequence Synthetic polynucleotide
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatgtggtattcggcggagggaccaagctgaccgtcctag

SEQ ID NO: 280 - DNA, 328 bp, Artificial Sequence
  Description of Artificial Sequence Synthetic polynucleotide
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatccttgggtgttcggcggagggaccaagctgaccgtcctag

SEQ ID NO: 281 - DNA, 328 bp, Artificial Sequence
  Description of Artificial Sequence Synthetic polynucleotide
gaaattgtgttgacacagtctccagacaccctgtctatgtgtccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggctttccatgtacacttttggccaggggaccaagctggagatcaaac

SEQ ID NO: 282 - DNA, 325 bp, Artificial Sequence
  Description of Artificial Sequence Synthetic polynucleotide
gaaattgtgctgactcagtctccagactttcagtctgtgactccaaaggggaaagtcaccatcacctgccgggccagtcagagcattggtagtagcttacactggtaccagcagaaaccagatcagtctccaaagctcctcatcaagtatgcttcccagtccttctcaggggtcccctcgaggttcagtggcagtggatctgggacagaattcactctcacaatcagcagcctgcagcctgaagattttgcaacttattactgtcaacagcttaatagttattctccgatcaccttcggccaagggacacgactggagattaaac

SEQ ID NO: 283 - DNA, 322 bp, Artificial Sequence
  Description of Artificial Sequence Synthetic polynucleotide
gacatccagatgacccagtctccttccaccctgtctgcatctgtaggagacagagtcaccatcacttgccgggccagtcagagtattagtagttggttggcctggtatcagcagaaaccagggaaagcccctaagctcctgatctataaggcgtctaatttagaaagtggggtcccatcaaggttcagcggcagtggatctgggacagaattcactctcaccatcagcagcctgcagcctgatgattttgcaacttattactgccaacagtataatagttacccgtggacgttcggccaagggaccaaggtggaaatcaaac

SEQ ID NO: 284 - DNA, 319 bp, Artificial Sequence
  Description of Artificial Sequence Synthetic polynucleotide
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattcaacttttggccaggggaccaagctggagatcaaac

SEQ ID NO: 285 - DNA, 322 bp, Artificial Sequence
  Description of Artificial Sequence Synthetic polynucleotide
gacatccagatgacccagtctccatcttccatgtctgcatctgtaggcgacagagtcaccatcacttgtcgggcgagtcagggcattagcaattatttagcctggtttcagcagaaaccagggaaagcccctaagcgcctgatctatggtgcatccagtttgcaccgtggggtcccatcaaggttcagcggcagcggatctgggacagaattcactctcacaatcaccagcctgcagcctgaagattttgcaacttattactgtctacagcataatacttaccctcggacgttcggccaagggaccaaggtggaaatcaaac

SEQ ID NO: 286 - DNA, 322 bp, Artificial Sequence
  Description of Artificial Sequence Synthetic polynucleotide
gacatccagatgacccagtctcccatctccctgtctgcatctgtaggagacagagtcaccatcacttgccgggcaagtcaggacattagcaattatttaacttggtatcagcagaaaccagggaaagcccctaagctcctgatctatgctgcatccagtttgcaaagtggggtcccatcaagattcagcggcagtggatctgggacagatttcactctcaccatcagcaatctgcagcctgaagattttgcaacttattactgtcaacagaattacagttacccgtgcacgttcggccaagggaccaaagtggagatcaaac

SEQ ID NO: 287 - DNA, 340 bp, Artificial Sequence
  Description of Artificial Sequence Synthetic polynucleotide
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgtggacgttcggccaagggaccaaggtggaaatcaaac

SEQ ID NO: 288 - DNA, 328 bp, Artificial Sequence
  Description of Artificial Sequence Synthetic polynucleotide
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccaccgatcaccttcggccaagggacacgactggagattaaac

SEQ ID NO: 289 - DNA, 368 bp, Artificial Sequence
  Description of Artificial Sequence Synthetic polynucleotide
cagcctgtgctgactcagcccacctccctctcagcatctcctggagcatcagccagattcacctgcaccttgcgcagtggtatcaatgttggtacctacaggatatactggtaccagcagaagccagggagtcttccccggtatctcctgaggtacaaatcagactcagataagcagcagggctctggagtccccagccgcttctctggatccaaagatgcttcaaccaatgcaggccttttactcatctctgggctccagtctgaagatgaggctgactattactgtgccatttggtacagcagcacttctcacagtgacacacacacatggggaagtgggacaaaaacctcaccctgctctcactc

SEQ ID NO: 290 - DNA, 331 bp, Artificial Sequence
  Description of Artificial Sequence Synthetic polynucleotide
cagtctgccctgactcagcctgcctccgtgtctgggtctcctggacagtcgatcaccatctcctgcactggaaccagcagtgatgttgggagttataaccttgtctcctggtaccaacagcacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgctgctcatatgcaggtagtagcactgtggtattcggcggagggaccaagctgaccgtcctag

SEQ ID NO: 291 - protein, 9 aa, Haliangium ochraceum
SLPPRTLLL

SEQ ID NO: 292 - protein, 9 aa, Unknown
  Description of Unknown Acidobacteria bacterium sequence
VMAPRTLQL

SEQ ID NO: 293 - protein, 9 aa, Gluconobacter japonicus
IMAPRALLL

SEQ ID NO: 294 - protein, 9 aa, Methylocystis sp.
VMAPRTLAL

SEQ ID NO: 295 - protein, 9 aa, Unknown
  Description of Unknown Rhodospirillales bacterium sequence
KMAPRTVLL

SEQ ID NO: 296 - protein, 9 aa, Mycobacterium tuberculosis
VMPPVSPML

SEQ ID NO: 297 - protein, 9 aa, Unknown
  Description of Unknown Rhizobiales bacterium sequence
RMAPRTLIL

SEQ ID NO: 298 - protein, 9 aa, Streptomyces sp.
VMAPRATLL

SEQ ID NO: 299 - protein, 9 aa, Halomonas salina
SMAPRSLLL

SEQ ID NO: 300 - protein, 9 aa, Mesorhizobium sp.
VMSPRALLL

SEQ ID NO: 301 - protein, 9 aa, Unknown
  Description of Unknown Acidobacteria bacterium sequence
SMAPRTLSL

SEQ ID NO: 302 - protein, 9 aa, Unknown
  Description of Unknown Rhodospirillaceae bacterium sequence
VMAPRFLLL

SEQ ID NO: 303 - protein, 9 aa, Unknown
  Description of Unknown bacterium HR22 sequence
AMAPRILLL

SEQ ID NO: 304 - protein, 9 aa, Pseudomonas syringae
VMPAPRTLL

SEQ ID NO: 305 - protein, 9 aa, Pseudomonas guariconensis
SLAPRTLLL

SEQ ID NO: 306 - protein, 9 aa, Unknown
  Description of Unknown Rhizobiales bacterium sequence
YLAPRTLLL

SEQ ID NO: 307 - protein, 9 aa, Unknown
  Description of Unknown Rhodocabterales bacterium sequence
LMAPRTILL

SEQ ID NO: 308 - protein, 9 aa, Unknown
  Description of Unknown Anaerolineae bacterium sequence
VMEPRILLL

SEQ ID NO: 309 - protein, 9 aa, Unknown
  Description of Unknown Puniceicoccaceae bacterium sequence
IIAPRTLLL

SEQ ID NO: 310 - protein, 9 aa, Mycobacterium tuberculosis
VMPARFLIL

SEQ ID NO: 311 - protein, 9 aa, Acidiphilium sp.
VMAPRVLLM

SEQ ID NO: 312 - protein, 9 aa, Streptomyces viridochromogenes
VMRPRTLLL

SEQ ID NO: 313 - protein, 9 aa, Unknown
  Description of Unknown Orrella dioscoreae sequence
VMAPITLLL

SEQ ID NO: 314 - protein, 9 aa, Unknown
  Description of Unknown Candidatus Lindowbacteria bacterium sequence
VMAPVTLLL

SEQ ID NO: 315 - protein, 9 aa, Sedimenticola sp.
VMAPRTILI

SEQ ID NO: 316 - protein, 9 aa, Unknown
  Description of Unknown Candidatus Entotheonella sequence
VTAPRTLLL

SEQ ID NO: 317 - protein, 9 aa, Desulfurococcus amylolyticus
LMNPRTLLL

SEQ ID NO: 318 - protein, 9 aa, Paenibacillus catalpae
LMAPRALLL

SEQ ID NO: 319 - protein, 9 aa, Petrotoga sp.
VMSSRTLLL

SEQ ID NO: 320 - protein, 9 aa, Unknown
  Description of Unknown Acidobacteria bacterium sequence
SMAPRTLGL

SEQ ID NO: 321 - protein, 9 aa, Unknown
  Description of Unknown Candidatus Aminicenantes bacterium sequence
VMARRTLLL

SEQ ID NO: 322 - protein, 9 aa, Achromobacter xylosoxidans
VAAPRTLLL

SEQ ID NO: 323 - protein, 9 aa, Phenylobacterium sp.
AMAARTLLL

SEQ ID NO: 324 - protein, 9 aa, Cystobacter fuscus
VMAPMARTL

SEQ ID NO: 325 - protein, 9 aa, Desulfarculus sp.
VMDPRTLLV

SEQ ID NO: 326 - protein, 9 aa, Rhodopirellula islandica
MAMPRTLLL

SEQ ID NO: 327 - protein, 9 aa, Rhodopirellula islandica
SMAMPRTLL

SEQ ID NO: 328 - protein, 9 aa, Rhodanobacter sp.
QMAPWTLLL

SEQ ID NO: 329 - protein, 9 aa, Unknown
  Description of Unknown Sphingomonadales bacterium sequence
PMPPRTLLL

SEQ ID NO: 330 - protein, 9 aa, Acidovorax avenae
MGAPRTLLL

SEQ ID NO: 331 - protein, 9 aa, Desulfarculus baarsii
VMEPRTLLV

SEQ ID NO: 332 - protein, 9 aa, Acidovorax avenae
TMGAPRTLL

SEQ ID NO: 333 - protein, 9 aa, Cystobacter fuscus
MAPMARTLL

SEQ ID NO: 334 - protein, 9 aa, Unknown
  Description of Unknown Acidobacteria bacterium sequence
MAPRTLSLL

SEQ ID NO: 335 - DNA, 1439 bp, Artificial Sequence
  Description of Artificial Sequence Synthetic polynucleotide
tctagaccaccatgggatggtcatgtatcatcctttttctagtagcaactgcaaccggtgtacattccgaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgattactacggtagtagctacggctggtacttcgatgtctggggcacagggaccacggtcaccgtctcctcagcgtcgaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgacgaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggggctctgcacaaccactacacgcagaagtacctctccctgtccccgggtaaatgatgagaatcc

SEQ ID NO: 336 - DNA, 1436 bp, Artificial Sequence
  Description of Artificial Sequence Synthetic polynucleotide
tctagaccaccatgggatggtcatgtatcatcctttttctagtagcaactgcaaccggtgtacattccgaagtgcagctggtgcagtctggaggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaatcttcagtttgggactactactactacggtatggacgtctggggccaagggaccacggtcaccgtctcctcagcgtcgaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgacgaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggggctctgcacaaccactacacgcagaagtacctctccctgtccccgggtaaatgatgagaatcc

SEQ ID NO: 337 - protein, 472 aa, Artificial Sequence
  Description of Artificial Sequence Synthetic polypeptide
MGWSCIILFLVATATGVHSEVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYGSSYGWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 338 - protein, 453 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYGSSYGWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 339 - protein, 471 aa, Artificial Sequence
  Description of Artificial Sequence Synthetic polypeptide
MGWSCIILFLVATATGVHSEVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKSSVWDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 340 - protein, 452 aa, Artificial Sequence
  Description of Artificial Sequence Synthetic polypeptide
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKSSVWDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 341 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSPGK

SEQ ID NO: 342 - protein, 254 aa, Artificial Sequence
  Description of Artificial Sequence Synthetic polypeptide
MGWSCIILFLVATATGVHSGGGGGSGEVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYGSSYGWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 343 - protein, 235 aa, Artificial Sequence
  Description of Artificial Sequence Synthetic polypeptide
GGGGGSGEVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYGSSYGWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 344 - protein, 233 aa, Artificial Sequence
  Description of Artificial Sequence Synthetic polypeptide
MGWSCIILFLVATATGVHSDIVITQSPSSMYASLGERVTITCKASQDINSYLSWFQQKPGKSPKTLIYRANRLVDGVPSRFSGSGSGQDYSLTISSLEYEDMGIYYCLQYDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - protein, 214 aa, Artificial Sequence
  Description of Artificial Sequence Synthetic polypeptide
DIVITQSPSSMYASLGERVTITCKASQDINSYLSWFQQKPGKSPKTLIYRANRLVDGVPSRFSGSGSGQDYSLTISSLEYEDMGIYYCLQYDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 346 - protein, 120 aa, Artificial Sequence
  Description of Artificial Sequence Synthetic polypeptide
  MOD_RES (103)..(106): Any amino acid
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYXXXXGWYFDVWGTGTTVT

SEQ ID NO: 347 - protein, 120 aa, Artificial Sequence
  Description of Artificial Sequence Synthetic polypeptide
  MOD_RES (59)..(60): Any amino acid
  MOD_RES (62)..(62): Any amino acid
  MOD_RES (65)..(65): Any amino acid
EVQLQESGPELVKPGASVKIPCKASGYTFTDYNMDWVKQSHGKSLEWIGDINPNNGGTXXNXKFXGKATLTVDKSSSTAYMELRSLTSEDTAVYYCARPDYYGSSYGWYFDVWGTGTTVT

SEQ ID NO: 348 - protein, 257 aa, Artificial Sequence
  Description of Artificial Sequence Synthetic polypeptide
MGWSCIILFLVATATGVHSEVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKSSVWDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKSGGSGLPETGG

SEQ ID NO: 349 - protein, 238 aa, Artificial Sequence
  Description of Artificial Sequence Synthetic polypeptide
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKSSVWDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKSGGSGLPETGG

SEQ ID NO: 350 - protein, 233 aa, Artificial Sequence
  Description of Artificial Sequence Synthetic polypeptide
MGWSCIILFLVATATGVHSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 351 - protein, 214 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 352 - protein, 471 aa, Artificial Sequence; identical to SEQ ID NO: 339
  Description of Artificial Sequence Synthetic polypeptide

SEQ ID NO: 353 - protein, 472 aa, Artificial Sequence; identical to SEQ ID NO: 337
  Description of Artificial Sequence Synthetic polypeptide

SEQ ID NO: 354 - protein, 120 aa, Artificial Sequence
  Description of Artificial Sequence Synthetic polypeptide
  MOD_RES (50)..(50): Any amino acid
  MOD_RES (52)..(52): Any amino acid
  MOD_RES (55)..(55): Any amino acid
  MOD_RES (57)..(57): Any amino acid
EVQLQESGPELVKPGASVKIPCKASGYTFTDYNMDWVKQSHGKSLEWIGXIXPNXGXTIYNQKFKGKATLTVDKSSSTAYMELRSLTSEDTAVYYCARPDYYGSSYGWYFDVWGTGTTVT

SEQ ID NO: 355 - protein, 107 aa, Artificial Sequence
  Description of Artificial Sequence Synthetic polypeptide
  MOD_RES (27)..(28): Any amino acid
  MOD_RES (30)..(30): Any amino acid
  MOD_RES (32)..(32): Any amino acid
DIVITQSPSSMYASLGERVTITCKASXXIXSXLSWFQQKPGKSPKTLIYRANRLVDGVPSRFSGSGSGQDYSLTISSLEYEDMGIYYCLQYDEFPLTFGAGTKLELK

SEQ ID NO: 356 - DNA, 785 bp, Artificial Sequence
  Description of Artificial Sequence Synthetic polynucleotide
tctagaccaccatgggatggtcatgtatcatcctttttctagtagcaactgcaaccggtgtacattccggcggaggcggaggatctggcgaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgattactacggtagtagctacggctggtacttcgatgtctggggcacagggaccacggtcaccgtctcctcagcgtcgaccaagggcccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaatgatgaggatcc

SEQ ID NO: 357 - DNA, 721 bp, Artificial Sequence
  Description of Artificial Sequence Synthetic polynucleotide
tctagaccaccatgggatggtcatgtatcatcctttttctagtagcaactgcaaccggtgtacattcagatattgtgatcacacagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgctcacgttcggtgctgggaccaagctggagctgaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctgataggatcc

SEQ ID NO: 358 - protein, 107 aa, Artificial Sequence
  Description of Artificial Sequence Synthetic polypeptide
  MOD_RES (92)..(95): Any amino acid
DIVITQSPSSMYASLGERVTITCKASQDINSYLSWFQQKPGKSPKTLIYRANRLVDGVPSRFSGSGSGQDYSLTISSLEYEDMGIYYCLQYXXXXLTFGAGTKLELK

SEQ ID NO: 359 - protein, 9 aa, Unknown
  Description of Unknown Mtb44 Control sequence
RLPAKAPLL

SEQ ID NO: 360 - DNA, 794 bp, Artificial Sequence
  Description of Artificial Sequence Synthetic polynucleotide
tctagaccaccatgggatggtcatgtatcatcctttttctagtagcaactgcaaccggtgtacattccgaagtgcagctggtgcagtctggaggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaatcttcagtttgggactactactactacggtatggacgtctggggccaagggaccacggtcaccgtctcctcagcgtcgaccaagggcccatcggtcttccccctggcaccctcctccaagagcacctctgggggcacagcggccctgggctgcctggtcaaggactacttccccgaacccgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaagcggcggcagcggcctgcctgagaccggaggatgatgaggatcc

SEQ ID NO: 361 - DNA, 721 bp, Artificial Sequence
  Description of Artificial Sequence Synthetic polynucleotide
tctagaccaccatgggatggtcatgtatcatcctttttctagtagcaactgcaaccggtgtacattcagaaattgtgttgacacagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcactcactttcggcggagggaccaaggtggagatcaaacgtacggtggctgcaccatctgtcttcatcttcccgccatctgatgagcagttgaaatctggaactgcctctgttgtgtgcctgctgaataacttctaccccagagaagccaaagtgcagtggaaggtggacaacgccctgcagagcggaaacagccaggaaagcgtgacagagcaggattccaaggattccacatacagcctgagcagcacactgacactgtccaaggccgactacgagaagcacaaggtgtacgcctgcgaagtgacacaccagggactgtcctcccctgtgacaaagagcttcaacagaggagaatgctgataggatcc

SEQ ID NO: 362 - DNA, 1436 bp, Artificial Sequence
  Description of Artificial Sequence Synthetic polynucleotide
tctagaccaccatgggatggtcatgtatcatcctttttctagtagcaactgcaaccggtgtacattccgaagtgcagctggtgcagtctggaggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaatcttcagtttgggactactactactacggtatggacgtctggggccaagggaccacggtcaccgtctcctcagcgtcgaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgacgaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggggctctgcacaaccactacacgcagaagtacctctccctgtccccgggtaaatgatgaggatcc

SEQ ID NO: 363 - DNA, 1439 bp, Artificial Sequence
  Description of Artificial Sequence Synthetic polynucleotide
tctagaccaccatgggatggtcatgtatcatcctttttctagtagcaactgcaaccggtgtacattccgaggtgcagctgcaggagtctggacctgagctggtgaagcctggggcttcagtgaagataccctgcaaggcttctggatacacattcactgactacaacatggactgggtgaagcagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagacctgattactacggtagtagctacggctggtacttcgatgtctggggcacagggaccacggtcaccgtctcctcagcgtcgaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgacgaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatggggctctgcacaaccactacacgcagaagtacctctccctgtccccgggtaaatgatgaggatcc

SEQ ID NO: 364 - protein, 125 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYGSSYGWYFDVWGTGTTVTVSSGS

SEQ ID NO: 365 - protein, 125 aa, Artificial Sequence
  Description of Artificial Sequence Synthetic polypeptide
  MOD_RES (101)..(104): Any amino acid
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDXXXXSYGWYFDVWGTGTTVTVSSGS

SEQ ID NO: 366 - protein, 125 aa, Artificial Sequence
  Description of Artificial Sequence Synthetic polypeptide
  MOD_RES (104)..(106): Any amino acid
  MOD_RES (108)..(108): Any amino acid
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYGXXXGXYFDVWGTGTTVTVSSGS

SEQ ID NO: 367 - protein, 125 aa, Artificial Sequence
  Description of Artificial Sequence Synthetic polypeptide
  MOD_RES (103)..(106): Any amino acid
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYXXXXGWYFDVWGTGTTVTVSSGS

SEQ ID NO: 368 - protein, 125 aa, Artificial Sequence
  Description of Artificial Sequence Synthetic polypeptide
  MOD_RES (59)..(60): Any amino acid
  MOD_RES (62)..(62): Any amino acid
  MOD_RES (65)..(65): Any amino acid
EVQLQESGPELVKPGASVKIPCKASGYTFTDYNMDWVKQSHGKSLEWIGDINPNNGGTXXNXKFXGKATLTVDKSSSTAYMELRSLTSEDTAVYYCARPDYYGSSYGWYFDVWGTGTTVTVSSGS

SEQ ID NO: 369 - protein, 125 aa, Artificial Sequence
  Description of Artificial Sequence Synthetic polypeptide
  MOD_RES (50)..(50): Any amino acid
  MOD_RES (52)..(52): Any amino acid
  MOD_RES (55)..(55): Any amino acid
  MOD_RES (57)..(57): Any amino acid
EVQLQESGPELVKPGASVKIPCKASGYTFTDYNMDWVKQSHGKSLEWIGXIXPNXGXTIYNQKFKGKATLTVDKSSSTAYMELRSLTSEDTAVYYCARPDYYGSSYGWYFDVWGTGTTVTVSSGS

SEQ ID NO: 370 - protein, 110 aa, Artificial Sequence
  Description of Artificial Sequence Synthetic polypeptide
  MOD_RES (30)..(31): Any amino acid
  MOD_RES (33)..(33): Any amino acid
  MOD_RES (35)..(35): Any amino acid
GGGDIVITQSPSSMYASLGERVTITCKASXXIXSXLSWFQQKPGKSPKTLIYRANRLVDGVPSRFSGSGSGQDYSLTISSLEYEDMGIYYCLQYDEFPLTFGAGTKLELK

SEQ ID NO: 371 - protein, 110 aa, Artificial Sequence
  Description of Artificial Sequence Synthetic polypeptide
  MOD_RES (95)..(98): Any amino acid
GGGDIVITQSPSSMYASLGERVTITCKASQDINSYLSWFQQKPGKSPKTLIYRANRLVDGVPSRFSGSGSGQDYSLTISSLEYEDMGIYYCLQYXXXXLTFGAGTKLELK

SEQ ID NO: 372 - protein, 110 aa, Artificial Sequence
  Description of Artificial Sequence Synthetic polypeptide
GGGDIVITQSPSSMYASLGERVTITCKASQDINSYLSWFQQKPGKSPKTLIYRANRLVDGVPSRFSGSGSGQDYSLTISSLEYEDMGIYYCLQYDEFPLTFGAGTKLELK

SEQ ID NO: 373 - protein, 120 aa, Artificial Sequence
  Description of Artificial Sequence Synthetic polypeptide
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYGSSYGWYFDVWGTGTTVT

SEQ ID NO: 374 - protein, 120 aa, Artificial Sequence
  Description of Artificial Sequence Synthetic polypeptide
  MOD_RES (101)..(104): Any amino acid
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDXXXXSYGWYFDVWGTGTTVT

SEQ ID NO: 375 - protein, 120 aa, Artificial Sequence
  Description of Artificial Sequence Synthetic polypeptide
  MOD_RES (104)..(106): Any amino acid
  MOD_RES (108)..(108): Any amino acid
EVQLQESGPELVKPGASVKIPCKASGYTFTDYNMDWVKQSHGKSLEWIGDINPNNGGTIYNQKFKGKATLTVDKSSSTAYMELRSLTSEDTAVYYCARPDYYGXXXGXYFDVWGTGTTVT

(19) United States
(12) Patent Application Publication
(10) Pub. No.: US 2025/0051454 A1
(43) Pub. Date: Feb. 13, 2025

(54) ANTIBODIES THAT TARGET HLA-E-HOST PEPTIDE COMPLEXES AND USES THEREOF

(71) Applicants: Duke University, Durham, NC (US); The Chancellor, Masters and Scholars of the University of Oxford, Oxford (GB)

(72) Inventors: Barton F. HAYNES, Durham, NC (US); Kevin O. SAUNDERS, Durham, NC (US); Dapeng LI, Durham, NC (US); Mihai AZOITEI, Durham, NC (US); Lucy C. WALTERS, Oxford (GB); Geraldine GILLESPIE, Oxford (GB); Simon BRACKENRIDGE; Andrew James MCMICHAEL

(21) Appl. No.: 18/245,540

(22) PCT Filed: Sep. 15, 2021
(86) PCT No.: PCT/US2021/050537

§ 371 Date: Mar. 15, 2023

Related U.S. Application Data
(60) Provisional application No. 63/235,535, filed on Aug. 20, 2021.
(60) Provisional application No. 63/121,031, filed on Dec. 3, 2020.
(60) Provisional application No. 63/121,036, filed on Dec. 3, 2020.
(60) Provisional application No. 63/078,780, filed on Sep. 15, 2020.

(51) Int. Cl. C07K 16/28

(52) U.S. Cl. CPC C07K 16/2833; C07K 2317/24; C07K 2317/32; C07K 2317/92

(57) ABSTRACT

Recombinant monoclonal antibodies (mAbs) and fragments that bind specifically to an HLA-E-peptide complex, including HLA-E-VL9 complexes, and regulate the cytotoxicity effector cell function of NK and/or CD8+ T-cells positive for cell-surface expression of NKG2A (“NKG2A+”). Herein, monoclonal antibodies were recombinantly derived from isolated functional HLA-E-VL9-specific mAbs from HLA-E-VL9 peptide-immunized HLA-B transgenic mice and from the naive human B cell repertoire. Such antibodies are capable of regulating effector cell cytotoxicity and can preferentially recognize HLA-E-VL9 peptide complexes expressed on the surface of tumor cells. The invention provides methods for using HLA-E-VL9 m Abs to modulate NK and/or CD8+ T-cell function as part of immunotherapeutic strategies.

This International Patent Application claims the benefit of and priority to U.S. Application No. 63/078,780, filed Sep. 15, 2020, entitled “Antibodies That Target HLA-E-Host Peptide Complexes And Uses Thereof,” U.S. Application No. 63/121,031, filed Dec. 3, 2020, entitled “Antibodies That Target HLA-E-Host Peptide Complexes and Uses Thereof,” U.S. Application No. 63/121,036, filed Dec. 3, 2020, entitled “Antibodies That Target HLA-E-Host Peptide Complexes and Uses Thereof,” and U.S. Application No. 63/235,535, filed Aug. 20, 2021, entitled “Antibodies That Target HLA-E-Host Peptide Complexes And Uses Thereof,” the contents of each of which are hereby incorporated by reference in their entireties.

This invention was made with government support under the 5UM1AI126619-05 awarded by NIH, UM1-AI100645 awarded by NIH/NIAID and UM1AI144371 awarded by NIH/NIAID. The government has certain rights in the invention.

### SEQUENCE LISTING

The instant application contains a Sequence Listing which has been filed electronically in ASCII format and is hereby incorporated by reference in its entirety. Said ASCII copy, created on Sep. 13, 2021, is named 1234300_00376W01_SL.txt and is 297,004 bytes in size.

### TECHNICAL FIELD

The invention relates to antibodies that bind to HLA-E-peptide complexes thereby preventing the complex from binding to the NKG2A/CD94 heterodimeric receptor expressed on subsets of Natural Killer (NK), CD8+ T-cells, and other immune cells. By preventing this interaction that normally works to inhibit NK and T-cell effector functions, the antibodies are useful for at least NK and T-cell-based immunotherapeutic, diagnostic, and research tool strategies.

### BACKGROUND

Killer Immunoglobulin like receptors (KIRs) recognize classical HLA class I molecules (Colonna and Samaridis, 1995; Karlhofer et al., 1992; Pende et al., 2019), and the NKG2A/CD94 heterodimeric inhibitory receptor interacts with the non-classical HLA class Ib molecule, HLA-E in humans and the HLA-E ortholog Qa-1b in mice. This inhibitory receptor is balanced by an activating receptor NKG2C/CD94. (Braud et al., 1997; Braud et al., 1998; Brooks et al., 1997.)

While KIRs are heterogeneously expressed, NKG2A/CD94 is expressed on about 40-50% of peripheral blood NK cells. (Andre et al., 1999; Mahapatra et al., 2017; Manser and Uhrberg, 2016; Pende et al., 2019.) About 5% of human peripheral blood CD8+ T cells express cell-surface NKG2A at steady state, but this expression can be upregulated by chronic antigenic stimulation. (Bertone et al., 1999; McMahon et al., 2002; Braud et al., 2003; Sheu et al., 2005.) NKG2A-expressing-CD8+ T-cells can form a distinct population of early activated tumor resident T cells. (van Montfoort et al., 2018.)

NKG2A is an ITIM-bearing receptor. Intracytoplasmic tyrosine based inhibitory motifs (ITIMs) are phosphorylated and recruit the phosphatases (SHP-1/2 or SHIP), which are responsible for transmitting the inhibition signal to immune effector cells. Binding of NKG2A/CD94 to its cognate ligand inhibits T and NK cell effector functions. This inhibition appears dependent on the recruitment of the SHP-1 tyrosine phosphatase to the tyrosine-phosphorylated form of the ITIM in NKG2A.

Unlike classical HLA class I molecules, HLA-E has limited polymorphism with only two predominant expressed variants HLA-E*01:01 and HLA-E*01:03 that differ only in residue 107 that is outside the peptide binding groove. (Kraemer et al., 2014.)

Natural killer (NK) cells play critical roles in immune surveillance by discriminating non-self from self, and function as effector cells by killing non-self malignant or pathogen-infected cells and producing inflammatory cytokines. (Chiossone et al., 2018; Raulet, 2006; Yokoyama and Kim, 2006.) Specific recognition of non-self by NK cells relies on a series of inhibitory receptors, including the killer immunoglobulin-like receptor (KIR) family and the NKG2A/CD94 heterodimeric receptor. (Chiossone et al., 2018; Guia et al., 2018.) NK cell inhibitory receptors promote NK sensing of human lymphocyte antigen (HLA) or major histocompatibility (MHC) class I molecules expressed on healthy cells as self. Conversely, cells lacking MHC class I are recognized by NK cells as “missing-self” and are sensitive to NK cell-mediated killing. (Ljunggren and Karre, 1990.)

HLA-E engages with NKG2A/CD94 via a restricted subset of peptides VMAPRT(L/V)(V/L/I/F)L (designated VL9) that derive from the leader sequence of HLA A, C, G and a third of B molecules. (Braud et al., 1997; Braud et al., 1998.) HLA-E binds VL9 peptides that stabilize HLA-E surface expression (Braud et al., 1997; Braud et al., 1998) and initiate the recognition by NKG2A/CD94 or NKG2C/CD94 on NK cells. The binding affinity of the HLA-E-VL9 peptide complex is greater for NKG2A/CD94 so that the inhibitory signal dominates to suppress aberrant NK cell-mediated cytotoxicity as well as cytokine production. (Aldrich et al., 1994; Braud et al., 1998; Kaiser et al., 2008; Llano et al., 1998; Rolle et al., 2018.) In addition, HLA-E or its murine and rhesus macaque homologs, is also capable of binding to a range of other host peptides and pathogen-derived peptides, including heat shock protein 60 (Hsp60)-derived peptides (Michaelsson et al., 2002), Mycobacterium tuberculosis (Mtb) peptides (Joosten et al., 2010; van Meijgaarden et al., 2015), Simian immunodeficiency virus (SIV) Gag peptides, including the RMYNPTNIL peptide (RL9SIV) (Hansen et al., 2016) and its homolog in human immunodeficiency virus (HIV) Gag, RMYSPTSIL peptide (RL9HIV) (Walters et al., 2018). However, it is believed that only VL9 peptide-loaded HLA-E can protect cells from NK cell cytotoxicity. (Kraemer et al., 2015; Michaelsson et al., 2002; Sensi et al., 2009.) Hence, the leader sequence VL9 peptides are essential not only for stabilizing HLA-E surface expression but also for determining the role of HLA-E/NKG2A/CD94 pathway in regulating NK cell self-recognition.

Although HLA-E has a broad tissue distribution, it is expressed at low surface levels in normal cells but at higher levels in tumor tissues, including melanoma and carcinomas of lung, cervix, ovarium, vulva, and head/neck (Andersson et al., 2016; de Kruijf et al., 2010; Gooden et al., 2011; Levy et al., 2008; Seliger et al., 2016; Wei and Orr, 1990) and aged cells (Pereira et al., 2019). In the tumor microenvironment, the high expression of HLA-E renders tumor cells resistant to NK cell lysis (Gustafson and Ginder, 1996; Malmberg et al., 2002; Nguyen et al., 2009) and CD8+ tumor-infiltrating lymphocytes (TILs) responses (Abd Hamid et al., 2019; Eugene et al., 2019). Increased HLA-E expression also contributes to the persistence of senescent cells during aging by inhibiting NK cell- and CD8+ T cell-mediated clearance (Pereira et al., 2019). Moreover, negative correlations between HLA-E expression and prognosis have been reported in different tumor types (Andersson et al., 2016; de Kruijf et al., 2010; Gooden et al., 2011; Seliger et al., 2016), suggesting an inhibitory role of HLA-E/NKG2A/CD94 pathway in anti-tumor immune responses.

The NKG2A/CD94/HLA-E pathway is considered an important immune checkpoint target and immunotherapy strategies including antibodies targeting NKG2A have been developed. (Andre et al., 2018; van Montfoort et al., 2018; Hu et al., 2019; Kim et al., 2019; Souza-Fonseca-Guimaraes et al., 2019; Kamiya et al., 2019.) However, it remained unknown if interruption of this pathway by targeting HLA-E-peptide complexes can enhance NK cell and other effector cell activities.

Here, the invention provides novel antibodies that can interrupt this inhibitory pathway by targeting HLA-E-peptide complexes and have the ability to enhance NK and CD8+ T-cell effector activities.

### SUMMARY OF THE INVENTION

The present invention provides monoclonal antibodies (mAbs) and fragments that bind to an HLA-E-peptide complex. As used herein, the term “antibody” is used broadly, and can refer to a full-length antibody, a fragment, or synthetic forms. In certain aspects, the antibody binds preferentially, or specifically, to an HLA-E-VL9 peptide complex. In certain aspects, the antibody binds specifically to an HLA-E-peptide complex where the peptide is a VL9 peptide or variant thereof. In certain aspects, the peptide of said complex is a nine-mer (9 amino acids) viral peptide or a nine-mer microbiome peptide. In certain aspects, the antibody binds preferentially to a HLA-E-VL9 peptide or variant complex and is also cross-reactive to complexes presenting viral peptides or microbiome peptides. In certain aspects, the antibody can regulate the cytotoxicity effector cell function of NK and/or CD8+ T-cells positive for cell-surface expression of NKG2A (“NKG2A+”). Herein, monoclonal antibodies were recombinantly derived from isolated functional HLA-E-VL9-binding mAbs from HLA-E-RL9 peptide-immunized HLA-B transgenic mice and from the naïve human B cell repertoire. Such antibodies are capable of regulating effector cell cytotoxicity and can recognize HLA-E-VL9 peptide complexes expressed on the surface of tumor cells. The invention provides methods for using HLA-E-VL9 mAbs to modulate NK and/or CD8+ T-cell function as part of immunotherapeutic strategies.

In certain aspects, provided are antibodies and fragments comprising VH and VL (as used herein, VH and VL can also be referred to as Vh or Vl and VH or VL, respectively) sequences of the antibodies described in Table 1 or FIG. 22A-E. As used herein, “Table 1” may implicitly refer to FIGS. 7 and 8 that provide the nucleotide and amino acid sequences of the antibodies listed in Table 1. Furthermore, FIG. 22E provide nucleotide sequences for Vh and Vl domains; the amino acid sequences are readily derived from the nucleotide sequences, such as by IMGT and other online tools as cited herein, which tools not only provide amino acid translations but also predicted CDR and framework boundaries. See, e.g., http://www.imgt.org/IMGT_vquest/. More specifically, for example, a nucleotide sequence for a Vh or Vl domain in FIG. 22E can be input at http://www.imgt.org/IMGT_vquest/input and results include “V-REGION translation” that provides the nucleotide sequence and amino acid translation along with the framework and CDR boundaries according to the IMGT scheme.

In certain aspects, the antibodies or fragments have a binding specificity that is dependent on an HLA-E-peptide complex where the peptide has an amino acid sequence according to the VL9 motif: (V/A/C/I/S/T/V/H/P)MAPRT(L/V)(V/L/I/F)L. In certain aspects, the binding specificity of the antibody or the fragment is dependent on an HLA-E-peptide complex where the peptide has an amino acid sequence according to the VL9 motif: VMAPRT(L/V)(V/L/I/F)L. As used herein, both motifs are referred to as “VL9” motifs, although the artisan might consider the first motif to be a VL9 variant motif.

In certain aspects, the antibodies can cause an increase in cytotoxic cell numbers or activity, which can be measured by counting the number of activated cytotoxic cells in biological samples or by in vitro effector cell assays as known in the art.

In certain aspects, the antibodies are recombinant antibodies having an IgG or IgM Fc domain, or a portion thereof.

In certain aspects, provided are recombinant antibodies and fragments comprising HCDR1-3 and LCDR1-3 (as used herein, the Vh CDRs can be referred to as HCDR1-3 or CDRH1-3; likewise the Vl CDRs can be referred to as LCDR1-3 or CDRL1-3) from the pairs of Vh and Vl sequences as described in Table 1 or FIG. 22A-E, wherein, in some embodiments, affinity maturation could lead to enhanced VL9 peptide/HLA-E complex binding or enhanced blocking of inhibitory NKG2A binding to VL9/HLA-E complexes. In certain aspects, such antibodies and fragments are humanized from a murine antibody or fragment listed in Table 1 and comprise the HCDR1-3 and LCDR1-3 regions from the murine antibodies or fragments. In one aspect, the antibody comprises HCDR1-3 and LCDR1-3 of antibody 3H4.

In certain aspects, an antibody that comprises HCDR1-3 and LCDR1-3 of an antibody of Table 1 or FIG. 22A-E is affinity matured by testing mutations in one or more of the CDRs. In one aspect, an antibody that comprises HCDR1-3 and LCDR1-3 of an antibody of Table 1 or FIG. 22A-E is affinity matured or further affinity matured by testing mutations only in HCDR3. In one aspect, an antibody that comprises HCDR1-3 and LCDR1-3 of an antibody of Table 1 or FIG. 22A-E is affinity matured by testing residues which contact the HLA-E-VL9 complex, including but not limited to residues outside HCDR3. In certain aspects, mutations are favorable when the antibody maintains binding specificity but improves affinity or avidity for the antigen, e.g., HLA-E-VL9.

In certain aspects the invention provides optimized sequences, including without limitation affinity matured sequences, as described herein. In non-limiting embodiments, the optimized sequences which are based on a murine antibody are humanized.

In non-limiting embodiments, the optimized sequences comprise changes at one, two, three, four residues, or a combination of changes at any of the residues as described in the VH chain of an optimized variant 3H4 SD1, 3H4 SD2, 3H4 SD3, 3H4 SD4, 3H4 SD5, 3H4 SD6 or 3H4 SD6. Provided are also non-limiting embodiments of optimized sequences which comprise changed residues, or a combination thereof, selected from any of the residues as described in the VH chain of an optimized variant 3H4 SD1, 3H4 SD2, 3H4 SD3, 3H4 SD4, 3H4 SD5, 3H4 SD6 or 3H4 SD6. In certain embodiments the affinity matured 3H4 antibody is 3H4 Gv2, 3H4 Gv3, 3H4 Gv4, 3H4 Gv5, 3H4 Gv6, 3H4 Gv7, 3H4 Gv8, 3H4 Gv9, 3H4 Gv10, 3H4 Gv11, or 3H4 Gv12, a multimer thereof or a fragment thereof.

In certain aspects, the invention provides a pharmaceutical composition comprising the recombinant antibodies of the invention.

In certain aspects, the invention provides nucleic acids comprising sequences encoding anti-HLA-9-VL9 peptide complex antibodies comprising Vh and Vl sequences of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap. In some embodiments, the antibodies could be administered using mRNAs without encapsulation into LNPs, particularly when applied to mucosal surfaces (Lindsday et al. Molecular Therapy Vol. 28 No 3 Mar. 2020^{a} 2020, p. 805)

In certain aspects, the invention provides a kit comprising: a composition comprising an antibody of the invention, a syringe, needle, or applicator for administration of the antibody to a subject; and instructions for use.

In certain aspects, the invention provides prophylactic methods comprising administering the pharmaceutical composition of the invention.

In certain aspects, the invention provides methods of treatment comprising administering the pharmaceutical composition of the invention. Generally, the methods are applicable to infectious diseases, malignant diseases or other conditions that would benefit from an increase in the number of stimulated effector immune cells such as NK cells and CD8+ T-cells. Exemplary diseases or conditions include, but are not limited to, cancer and viral or intracellular bacterial infections. In some cases, the cancer comprises tumor cells that express, or overexpress HLA-E, including melanoma and carcinomas of lung, kidney, skin, prostate, stomach, rectum, cervix, ovarium, vulva, breast and head/neck. (See, e.g., Kamiya et al., J. Clin. Invest., 2019, 129(5):2094-2106.) Such methods of treatment can relate to methods of immunostimulation comprising the step of administering a therapeutically effective amount of an antibody of the invention, which antibody specifically binds to at least an HLA-E-VL9 peptide or variant complex and increases the number of activated NK cells or activated CD8+ cells or other cells with cytotoxic functions such as γδ T-cells.

In some aspects, the therapeutic compositions and methods not only involve blocking the inhibitory HLA-E-VL9-NKG2A pathway in NK cells and CD8+ T-cells with an antibody or fragment of the invention, but also: (1) blocking other inhibitory receptors on these NK cells and CD8+ T-cells, and/or (2) promoting the activation of stimulatory receptors on these NK cells and CD8+ T-cells. The targeting of multiple receptors on NK cell and CD8+ T-cell sub-populations can be accomplished, for example, by the use of combination of different antibodies or agents each targeting a different receptor, or by recombinant multi-specific antibodies as described herein.

In other aspects, the administration of the anti-HLA-E-peptide complex antibodies or fragments of the invention is part of a vaccine regimen, whether the vaccine is a viral vaccine or a cancer vaccine. In certain respects, anti-HLA-E-VL9 antibodies or fragments is part of a vaccine regimen.

In other aspects, provided are methods for making and/or screening recombinant antibodies specific to an HLA-E-peptide complex from single circulating B-cells, the method including steps of folding a VL9 peptide with HLA-E to make a stable complex and assembling the folded HLA-E-peptide as a tetramer, such that the labeled tetramers can be used to identify B-cells that express antibodies that specifically bind to an HLA-E-peptide of interest complex.

In certain aspects the invention provides a recombinant HLA-E-VL9 monoclonal antibody, or an antigen binding fragment thereof, which binds to an HLA-E-VL9 complex and comprises a variable heavy (Vh) domain and a variable light (Vl) domain that have amino acid sequences that have an overall 80% sequence identity to the Vh and Vl domains of an antibody listed in Table 1, or wherein the Vh domain and Vl domain each have at least 80% sequence identity to the Vh and Vl domains, respectively, of an antibody listed in Table 1 or an antibody encoded by a nucleic acid sequence in FIG. 22E. In non-limiting embodiments, the antibody is any one of 3H4, affinity matured form of 3H4 (Example 4), a fragment or a humanized version thereof. In non-limiting embodiments, the antibody is CA147, affinity matured form of CA147 or a fragment thereof. In non-limiting embodiments, the antibody is designed such that it forms hexamers. In non-limiting embodiments, the antibody is designed such that it displays full antibody or functional fragments on a nanoparticle. In certain non-limiting embodiments, the antibody or antigen binding fragment preferentially or specifically binds to an HLA-E-VL9 complex.

In certain non-limiting embodiments, the Vh domain and Vl domain each have at least 90% sequence identity to the Vh and Vl domains, respectively, of an antibody listed in Table 1 or an antibody encoded by a nucleic acid sequence in FIG. 22E.

In certain non-limiting embodiments, (a) Vl domain CDRL1-3 regions together have no more than 10 amino acid variations as compared to the corresponding CDRL1-3 regions of an antibody listed in Table 1, and (b) Vh domain CDRH1-3 regions together have no more than 10 amino acid variations as compared to the corresponding CDRH1-3 regions of an antibody listed in Table 1 or an antibody encoded by a nucleic acid sequence in FIG. 22E.

In certain non-limiting embodiments, the antibody or fragment is humanized or fully human.

In certain non-limiting embodiments, the Vh domain and Vl domain of the antibody or fragment comprises framework regions that each have sufficient number of, e.g. no more than 20 or 10, amino acid variations derived from framework regions of a human antibody. In certain embodiments, the framework regions are from human antibodies listed in Table 1.

In certain non-limiting embodiments, (a) Vl domain CDRL1-3 regions together have no more than 10 amino acid variations as compared to the corresponding CDRL1-3 regions of an antibody listed in Table 1, (b) Vh domain CDRH1-3 regions together have no more than 10 amino acid variations as compared to the corresponding CDRH1-3 regions of the antibody listed in Table 1, and (c) the Vl domain and Vh domain framework regions are derived from a human antibody.

In certain non-limiting embodiments, the antibody or fragment is chimeric or humanized.

In certain aspects the invention provides a humanized HLA-E-VL9 monoclonal antibody, or an antigen binding fragment thereof, which specifically binds to an HLA-E-VL9 complex and comprises: (1) a variable heavy (Vh) domain with CDRH1-3 regions derived from a murine parental antibody listed in Table 1; (2) a variable light (Vl) domain with CDRL1-3 regions derived from said murine parental antibody listed in Table 1.

In certain non-limiting embodiments of the humanized antibodies or fragments thereof of the invention, the CDRH1-3 and CDRL1-3 regions collectively have an amino acid sequence that has no more than twenty variations as compared to the CDRH1-3 and CDRL1-3 regions of the parental murine antibody. In certain non-limiting embodiments, the murine antibody listed in Table 1 is 3H4 or any one of the affinity matured variants of 3H4: 3H4 Gv2, 3H4 Gv3, 3H4 Gv4, 3H4 Gv5, 3H4 Gv6, 3H4 Gv7, 3H4 Gv8, 3H4 Gv9, 3H4 Gv10, 3H4 Gv11, or 3H4 Gv12.

In certain non-limiting embodiments of the humanized antibodies or fragments thereof of the invention have a paratope comprising the same contact residues as 3H4, or any one of the affinity matured variants of 3H4: 3H4 Gv2, 3H4 Gv3, 3H4 Gv4, 3H4 Gv5, 3H4 Gv6, 3H4 Gv7, 3H4 Gv8, 3H4 Gv9, 3H4 Gv10, 3H4 Gv11, or 3H4 Gv12.

In certain non-limiting embodiments of the humanized antibodies or fragments thereof of the invention the Vh domain framework regions are derived from a human antibody having a Vl domain amino acid sequence that is most similar or identical to the Vl domain amino acid sequence of the murine antibody; and wherein the Vh domain framework regions are derived from a human antibody having a Vh domain amino acid sequence that is most similar or identical to the Vh domain amino acid sequence of the murine antibody.

In certain non-limiting embodiments of the humanized antibodies or fragments thereof of the invention the Vh domain framework regions are derived from a human antibody having a Vh domain that has the most similar three-dimensional structure to the Vh domain of the murine antibody; and wherein the Vl domain framework regions are derived from a human antibody having a Vl domain that has the most similar three-dimensional structure to the Vl domain of the murine antibody.

In certain non-limiting embodiments of the human or humanized antibodies or fragments thereof of the invention, the Vh domain framework regions are derived from IGHV3-21, IGHV3-11, IGHV3-23, IGHV1-69, or IGHV3-48. In non-limiting embodiments of the humanized or human antibodies or fragments thereof of the invention, the Vh domain framework region is derived from any one of the IGHV genes listed in FIG. 22C.

In certain non-limiting embodiments of the human or humanized antibodies or fragments thereof of the invention, the Vl domain framework regions are derived from IGKV3-15, IGKV3-20, IGKV1-39, IGKV3-11, or IGKV1-5. In non-limiting embodiments of the humanized or human antibodies or fragments thereof of the invention, the Vl domain framework region is derived from any one of the IGKV or IGLV genes listed in FIG. 22D.

In non-limiting embodiment, the binding specificity of the antibody or the fragment thereof requires the peptide of the HLA-E-VL9 complex to have an amino acid sequence according to the following motif: (V/A/C/I/S/T/V/H/P)MAPRT(L/V)(V/L/I/F)L.

In non-limiting embodiment, the binding specificity of the antibody or the fragment thereof requires the peptide of the HLA-E-VL9 complex to have an amino acid sequence according to the following motif: VMAPRT(L/V)(V/L/I/F)L.

In non-limiting embodiment, the antibody or fragment specifically binds to epitopes on both the HLA-E α2 domain and the amino terminal end of the VL9 peptide.

In non-limiting embodiment, the antibody, or the antigen binding fragment thereof, has an affinity or avidity for the HLA-E-VL9 complex that is greater than the affinity or avidity between the HLA-E-VL9 complex and NKG2A.

In non-limiting embodiment, the antibody or fragment thereof increases the cytotoxic activity of NKG2A+NK cells, NKG2A+CD8+ T-cells, or NKG2A+γδ T-cells, in vitro or in vivo.

In non-limiting embodiment, the antibody, or the antigen-binding fragment thereof comprises an Fc moiety. In non-limiting embodiment, the antibody, or antigen-binding fragment thereof, comprises a mutation(s) in the Fc moiety that reduces binding of the antibody to an Fc receptor and/or increases the half-life of the antibody.

In non-limiting embodiment, the antigen binding fragment thereof, is a purified antibody, a single chain antibody, Fab, Fab′, F(ab′)2, Fv or scFv.

In non-limiting embodiment, the antibody or antigen fragment thereof is multimerized in any suitable form. Non-limiting embodiments include hexamers formed via the Fc portion of the antibody. Non-limiting embodiments include antibodies or antigen binding fragments thereof comprised in a nanoparticle. In non-limiting embodiments, these multimers increase binding avidity and/or affinity.

In non-limiting embodiment, the antibody is of any isotype.

In certain aspects the invention provides an antibody or antigen binding fragment of the for use as a medicament. In certain aspect the use is in the prevention and/or treatment of a tumor comprising tumor cells that overexpress HLA-E.

In certain aspects, the invention provides a nucleic acid molecule comprising a polynucleotide encoding the antibody, or the antigen-binding fragment thereof. In non-limiting embodiments, the polynucleotide sequence comprises, consists essentially of or consists of a nucleic acid sequence according to any one of the sequences in FIG. 6, FIG. 7, FIG. 22E, FIG. 24 or FIG. 26; or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional variation is 80%, 81%, 82%, 83%, 84%, 85%, 86%, 87%, 88%, 89%, 90%, 91%, 92%, 93%, 94%, 95%, 96%, 97%, 98% or 99% identity. In non-limiting embodiments, the nucleic acid is a ribonucleic acid (RNA) based on a nucleic acid or protein as shown in FIG. 6, FIG. 7, FIG. 22E, FIG. 24 or FIG. 26.

In certain aspects the invention provides a vector comprising a nucleic acid molecule encoding an antibody or antigen binding fragment of the invention.

In certain aspects the invention provides a cell expressing the antibody, or the antigen binding fragment of the invention; or comprising a vector comprising a nucleic acid molecule encoding an antibody or antigen binding fragment of the invention.

In certain aspects the invention provides a pharmaceutical composition comprising the antibody, or the antigen binding fragment thereof, a nucleic acid of the invention, a vector comprising a nucleic acid molecule encoding an antibody or antigen binding fragment of the invention and/or a cell expressing the antibody, or the antigen binding fragment of the invention; or comprising a vector comprising a nucleic acid molecule encoding an antibody or antigen binding fragment of the invention, and optionally a pharmaceutically acceptable carrier.

In non-limiting embodiments, the pharmaceutical composition comprises a pharmaceutically acceptable excipient, diluent or carrier.

In certain aspect the invention provides method of administering antibodies or antigen fragments thereof of the invention in an amount sufficient to modulation NKG2A+ NK cells or T-cells in a subject in need thereof, for example to achieve a desired therapeutic effect. In certain aspects the invention provides methods of treating or preventing a condition that would benefit from an increase in the activation of NKG2A+ NK cells or T-cells in a subject in need thereof, comprising administering the recombinant antibody or antigen binding fragment thereof of the invention, a nucleic acid encoding these, a vector comprising a nucleic acid of the invention, or a pharmaceutical composition comprising any of these in an amount suitable to increase the number of activated cytotoxic NK cells or T-cells in the subject. In non-limited embodiments, the methods comprise administering any combination of antibodies or antigen binding fragments of the invention. In non-limiting embodiments, the methods comprise administering any additional antibody.

In certain aspects, the methods further comprise administering an additional agent that is an antagonist to an inhibitory receptor on NK cells or cytotoxic T-cells and/or an additional agent that is an agonist to a stimulatory receptor on NK cells or cytotoxic T-cells.

In certain aspects, the invention provides an in vitro transcription system to synthesize ribonucleic acids (RNAs) encoding antibodies of the invention, comprising: a reaction vessel, a DNA vector template comprising nucleic acid sequence encoding an antibody of the invention as described in any of the preceding claims, and reagents for carrying out an in vitro transcription reaction that produces mRNA encoding an antibody or fragment thereof of the invention. In certain embodiments the mRNA is modified mRNA.

In certain aspects, the invention provides methods for manufacturing an mRNA encoding an antibody or antigen binding fragment thereof, comprising:
a. providing an in vitro transcription reaction vessel comprising a DNA template encoding an antibody or fragment thereof according to any of the preceding claims and reagents under conditions suitable for in vitro transcription of the nucleic acid template, thereby producing an mRNA template encoding the antibody or fragment thereof according to any of the preceding claims, and
b. isolating the mRNA by any suitable method of purification and separating reaction reagents, the DNA template, and/or mRNA product related impurities.
   In certain embodiments, the mRNA comprises modified nucleotides. In certain embodiments, the mRNA comprises 5′-CAP, and/or any other suitable modification.

In certain aspects, the invention provides methods manufacturing an antibody or antigen binding fragment thereof, comprising culturing a host cell comprising a nucleic acid according to any of the preceding claims under conditions suitable for expression of the antibody or fragment thereof and isolating said antibody or antigen binding fragment thereof.

In certain aspects, the invention provides methods of screening for an antibody or antigen binding fragment thereof that specifically binds to an HLA-E-VL9 peptide complex, comprising: (a) providing either a substrate with immobilized HLA-E-VL9 single chain trimers or HLA-E-non-VL9 peptide control single chain trimers or with cells expressing on their surface HLA-E-VL9 single chain trimers or HLA-E-non-VL9 peptide control single chain trimers, and (b) selecting an antibody or antigen binding fragment thereof for its ability to specifically bind to the HLA-E-VL9 single chain trimers but not the HLA-E-non-VL9 peptide control single chain trimers. In certain embodiments, the antigen-binding fragments are expressed on phage, and wherein said phage are incubated with the single chain trimers prior to the step of selecting an antigen binding fragment for its ability to specifically bind to the HLA-E-VL9 single chain trimers.

In certain aspects, the invention provides methods for identifying a non-human antibody that specifically binds to an HLA-E-VL9 peptide complex, comprising: (a) immunizing a non-human mammal with either soluble HLA-E-VL9 single chain trimers or with cells expressing HLA-E-VL9 single chain trimers, (b) isolating B-cells from the non-human mammal that express antibodies that can bind to HLA-E-VL9 single chain trimers but not HLA-E-non-VL9 control peptide single chain trimers.

In non-limiting embodiments, certain methods further comprise recombinantly expressing the selected antibody or antigen-binding fragment and further selecting the antibody or antigen-binding fragment if it can increase the cytotoxic activity of NK cells or CD8+ T-cells when such cells are co-cultured with HLA-E-VL9 expressing cells and the antibody or antigen-binding fragment, but not when such cells are co-cultured with HLA-E-non-VL9 peptide expressing cells and the antibody or antigen-binding fragment thereof.

In certain aspects, the invention provides methods for making recombinant antibodies specific to an HLA-E-peptide complex from single circulating B-cells, the method comprising: (1) folding a VL9 peptide, or other test peptide, with HLA-E to make a stable complex; (2) assembling the folded HLA-E-peptide as a tetramer; (3) staining B cells from peripheral blood of a human donor or an animal with the tetramer; (4) sorting tetramer binding B cells as single cells and cloning DNA or mRNA for antibody heavy and light chains; (4) expressing full length DNA for heavy and light chains in a suitable cell or cell-line (e.g., HEK293T) so that antibody is expressed and secreted; (5) testing specificity of the antibody or antibodies expressed and secreted from step (4) for binding to HLA-E-peptide protein complexes expressed on cells or immobilized on a substrate; and (6) purifying antibodies with requisite binding specificity, and wherein in certain embodiments step (5) further comprises selecting antibodies that can specifically bind to HLA-E-VL9 single chain trimers but not HLA-E-non-VL9 control peptide single chain trimers.

In certain aspects, the invention provides a recombinant HLA-E-VL9 monoclonal antibody, or an antigen binding fragment thereof, which binds to an HLA-E-VL9 complex and comprises:
a. Vh domain CDRH1-3 regions from an antibody listed in Table 1; and/or Vl domain CDRL1-3 regions from an antibody listed in Table 1, wherein the Vh and Vl are from the same antibody; and
b. Wherein the framework portions of the variable heavy (Vh) domain comprises amino acid sequences that have at least 80%, 85%, 90%, 91%, 92%, 93%, 94%, 95%, 96%, 97%, 98%, or 99% sequence identity to the V gene, D gene and J gene making up the Vh gene of the corresponding antibody from which the CDRs are derived and wherein the framework portions of the variable light (V1) domain comprises amino acid sequences that have at least 80%, 85%, 90%, 91%, 92%, 93%, 94%, 95%, 96%, 97%, 98%, or 99% sequence identity to the V and J genes making up the Vl gene from the corresponding antibody from which the CDRs are derived.

In certain aspects, the invention provides a recombinant HLA-E-VL9 monoclonal antibody, or an antigen binding fragment thereof, which binds to an HLA-E-VL9 complex and comprises:
a. Vh domain CDRH1-3 regions from an antibody listed in Table 1; and/or Vl domain CDRL1-3 regions from an antibody listed in Table 1, wherein the Vh and Vl are from the same antibody; and
b. Wherein the framework portions of the variable heavy (Vh) domain comprises amino acid sequences that have 80%, 85%, 90%, 91%, 92%, 93%, 94%, 95%, 96%, 97%, 98%, or 99% sequence identity to the V gene, D gene and J gene making up the Vh gene of the corresponding antibody from which the CDRs are derived and wherein the framework portions of the variable light (Vl) domain comprises amino acid sequences that have 80%, 85%, 90%, 91%, 92%, 93%, 94%, 95%, 96%, 97%, 98%, or 99% sequence identity to the V and J genes making up the Vl gene from the corresponding antibody from which the CDRs are derived.

In certain aspects, the invention provides a recombinant HLA-E-VL9 monoclonal antibody, or an antigen binding fragment thereof, which binds to an HLA-E-VL9 complex and comprises:
a. Vh domain CDRH1-3 regions from an antibody listed in Table 1; and/or Vl domain CDRL1-3 regions from an antibody listed in Table 1, wherein the Vh and Vl are from the same antibody; and
b. Wherein the framework portions of the variable heavy (Vh) domain comprises amino acid sequences that have 90%-99%, 95%-99% sequence identity to the V gene, D gene and J gene making up the Vh gene of the corresponding antibody from which the CDRs are derived and wherein the framework portions of the variable light (Vl) domain comprises amino acid sequences that have 90%-99%, 95%-99% sequence identity to the V and J genes making up the Vl gene from the corresponding antibody from which the CDRs are derived.

FIGS. 22C and 22D, respectively show the immunogenetics of the Vh V/D/J genes and the immunogenetics of Vl V and J genes, along with mutation frequency of certain human antibodies. Table 5 shows the immunogenetics of mouse antibodies. In non-limiting embodiments, the CDRs are from any one of the following antibodies: CA147, 3H4, 3H4 Gv2, 3H4 Gv3, 3H4 Gv4, 3H4 Gv5, 3H4 Gv6, 3H4 Gv7, 3H4 Gv8, 3H4 Gv9, 3H4 Gv10, 3H4 Gv11, or 3H4 Gv12. The immunogenetics of the 3H4, 3H4 Gv2, 3H4 Gv3, 3H4 Gv4, 3H4 Gv5, 3H4 Gv6, 3H4 Gv7, 3H4 Gv8, 3H4 Gv9, 3H4 Gv10, 3H4 Gv11, or 3H4 Gv12 are as for 3H4.

In non-limiting embodiments, the antibody is CA147 and the framework portions of the variable heavy (Vh) domain comprises, consists essentially of, consists of or has amino acid sequences derived from IGHV3-23, IGHD4-4 and IGHJ6 human Ig genes (See FIG. 22C line 25, CA147) and wherein the framework portions of the variable light (Vl) domain comprises, consists essentially of, consists of or has amino acid sequences derived from IGKV3-20 and IGKJ4 (FIG. 22D line 25, CA147).

In non-limiting embodiments, the antibody is CA147 and the framework portions of the variable heavy (Vh) domain comprises, consists essentially of, consists of or has amino acid sequences derived from IGHV3-23*04, IGHD4-4*01 and IGHJ6*02 human Ig genes (See FIG. 22C line 25, CA147) and wherein the framework portions of the variable light (Vl) domain comprises, consists essentially of, consists of or has amino acid sequences derived from IGKV3-20*01 and IGKJ4*01 (FIG. 22D line 25, CA147). In non-limiting embodiments CA147 Vh and Vl are shown in FIGS. 8H and 26B.

In non-limiting embodiments, the antibody is 3H4 or an affinity matured 3H4 variant thereof, and the framework portions of the variable heavy (Vh) domain comprises, consists essentially of, consists of or has amino acid sequences derived from Vh gene 1-18 (Table 5) and wherein the framework portions of the variable light (Vl) domain comprises, consists essentially of, consists of or has amino acid sequences derived from Vk gene 14-111 (Table 5).

In non-limiting embodiments, the recombinant antibody or the antigen binding fragment thereof of any of the invention is a multimer.

In non-limiting embodiments, the multimer is a hexameric IgG. In non-limiting embodiments, each IgG monomer comprises a heavy chain comprising a Vh sequence and constant heavy chain sequence comprising mutations E345R, E430G and S440Y in the Fc region of a human gamma immunoglobulin gene, and a Vl sequence from the same antibody, wherein in certain embodiments the IgG is G1m3 allotype. These Vh and constant heavy chain sequences are linked consecutively to express a heavy chain sequence.

In non-limiting embodiments of the hexameric IgG, each IgG monomer comprises mutations E345R, E430G and S440Y in Fc region of human gamma immunoglobulin (G1m3 allotype) and the Vh and Vl chain are from antibody CA147. In certain embodiments, the heavy chain sequence is “>CA147_G1m3_E345R/E430G/S440Y without signal peptide” shown in FIG. 24B and light chain sequence is “>CA147_G1m3_E345R_E430G_S440Y VL (Same as CA147_FabH VL)”/“>CA147_FabH VL without signal peptide” shown in FIG. 26A. In non-limiting embodiments of the hexamer, the Vl and Vh are corresponding Vh and Vl chains from any one of the antibodies 3H4, 3H4 Gv2, 3H4 Gv3, 3H4 Gv4, 3H4 Gv5, 3H4 Gv6, 3H4 Gv7, 3H4 Gv8, 3H4 Gv9, 3H4 Gv10, 3H4 Gv11, or 3H4 Gv12.

In non-limiting embodiments, the antibody or the antigen binding fragment thereof forms a multimer displayed on a nanoparticle. In certain embodiments, the nanoparticle is ferritin based nanoparticle.

In certain embodiments, the antigen binding fragment is a Fab fragment from any one of the antibodies 3H4, 3H4 Gv2, 3H4 Gv3, 3H4 Gv4, 3H4 Gv5, 3H4 Gv6, 3H4 Gv7, 3H4 Gv8, 3H4 Gv9, 3H4 Gv10, 3H4 Gv11, or 3H4 Gv12.

In certain embodiments, the antigen binding fragment is a Fab fragment from CA147 antibody, wherein in certain embodiments the Fab heavy chain sequence comprises CA147VH (FIG. 8H) immediately followed by CH1 amino acid sequence immediately followed by sortase donor sequence and wherein the Fab light chain fragment comprise CA147VL (FIG. 8H) immediately followed by CL1 amino acid sequence.

In non-limiting embodiments, the antigen binding fragment of CA147 comprises, consists essentially of, consists of or has the Fab heavy chain sequence: EVQLVQSGGGLVQPGGSLRLSCAASGFTFSSYAMSWVRQAPGKGLEWVSAISGSGG STYYADSVKGRFTISRDNSKNTLYLQMNSLRAEDTAVYYCAKSSVWDYYYYGMDV WGQGTTVTVSSASTKGPSVFPLAPSSKSTSGGTAALGCLVKDYFPEPVTVSWNSGAL TSGVHTFPAVLQSSGLYSLSSVVTVPSSSLGTQTYICNVNHKPSNTKVDKKVEPKSCD KSGGSG-[sortase donor sequence](FIG. 26). In a non-limiting embodiment, the sortase donor sequence is LPXTG(G) and the Fab light chain sequence is “>CA147_FabH VL without signal peptide” (FIG. 26A).

Further, the invention encompasses the aspects described in the claims herein.

### BRIEF DESCRIPTION OF DRAWINGS

The patent or application file contains at least one drawing executed in color. To conform to the requirements for PCT patent applications, many of the figures presented herein are black and white representations of images originally created in color.

FIGS. 1A-G. Isolation of monoclonal antibody 3114 specifically targeting HLA-E-VL9 complex. (A) 3114 bound HLA-E-VL9 single chain trimer (SCT)-transfected 293T cells. All SCT constructs express EGFP to indicate transfection efficiency. Transfected cells were stained with test antibody and then an Alexa fluor 555 (AF555)-anti-mouse Ig(H+L) secondary antibody. A control mouse IgM TE4 was used as a negative control. Anti-pan-HLA-E antibody 3D12 was used as a positive control. Representative data from one of five independent experiments are shown. (B) 3114 bound VL9 peptide pulsed K562-HLA-E cells. RL9HIV, RL9SIV, Mtb44 peptides served as peptide controls. TE4 and 3D12 were used as antibody controls. Peptide-pulsed cells were stained with test antibody and then an Alexa fluor 647 (AF647)-anti-mouse Ig(H+L) secondary antibody. Mean fluorescence intensity (MFI) of each sample is shown. Representative data from one of three independent experiments are shown. (C-D) 3114 specifically bound to soluble HLA-E-VL9 complexes as measured by ELISA and SPR. (C) ELISA plates were coated with 3H4 or control IgM TE4 in serial dilution, blocked, and incubated with C-trap-stabilized HLA-E-VL9, HLA-E-RL9HIV, HLA-E-RL9SIV antigens. After washing, antigen binding was detected by adding HRP-conjugated anti-human β2M antibody. (D) For SPR, biotinylated HLA-E-peptide complexes (HLA-E-VL9, HLA-E-RL9SIV, HLA-E-RL9HIV and mock control) were bound to the immobilized streptavidin. Antibody 3H4 and control TE4 were flowed over sensor chips and antibody binding was monitored in real-time. Representative data from one of two independent experiments are shown. (E) 3114 recognized the α2 domain of HLA-E. Flow cytometry analysis of 3H4 and 2M2 (a control β2M mAb) binding to 293T cells transfected with VL9 presented by HLA-E, Mamu-E, and two HLA-E/Mamu-E hybrids—one with HLA-E α1/Mamu-E α2 (Hα1/Mα2), the other with Mamu-E α1/HLA-E α2 (Mα1/Hα2) (green). Transfected cells were stained with test antibody and then an AF647-anti-mouse Ig(H+L) secondary antibody. Isotype control stained cells were used as negative controls (grey filled histograms). Representative data from one of three independent experiments are shown. (F) 3114 recognized position 1 (P1) of the VL9 peptide. 3H4 and 2M2 (a control β2M mAb) staining of 293T cells transfected with HLA-E-VL9 (VMAPRTLLL) or HLA-E-VL9 with a mutation at P1 (valine to arginine; RMAPRTLLL) (blue), and with HLA-E-RL9HIV (RMYSPTSIL) or HLA-E-RL9HIV with a mutation at P1 (arginine to valine; VMYSPTSIL) (red). Transfected cells were stained with test antibody and then an AF647-anti-mouse Ig(H+L) secondary antibody. Isotype control stained cells were used as negative controls (grey filled histograms). Representative data from one of three independent experiments are shown. (G) 3H4 recognized peptides with variants in P1. 293T cells were transfected with HLA-E SCTs with VL9 peptides with single amino acid mutations at P1, then stained with 3H4 antibody followed by AF647 conjugated anti-mouse IgG(H+L) secondary antibody. Cells were gated for EGFP positive subsets. MFI of 3H4 staining on wildtype VL9 peptide was set as 100%, and the percentages for binding to mutants calculated as (MFI of 3H4 binding on each P1 variant)/(MFI of 3H4 binding on wildtype VL9)×100%.

FIGS. 2A-J. 3H4 Fab-HLA-E-VL9 co-complex structural visualisation. (A-B) 3H4 Fab-HLA-E docking angles. The HLA-E heavy chain and β2M light chain are shown as a grey cartoon, the VL9 peptide as lime green sticks, the 3H4 HC as a light purple cartoon and the 3H4 light chain (LC) as a teal cartoon. (C-D) Superposition of 3H4 Fab and CD94/NKG2A docking sites on HLA-E. The HLA-E complex and 3H4 Fab are color-coded according to A and B. The CD94 subunit is shown as an orange surface and the NKG2A subunit as a marine blue surface. (E) Aerial view of the HLA-E-VL9 peptide binding groove surface. Non-interfacing residues of the HLA-E heavy chain are shaded light grey and non-interfacing peptide residues shaded lime green. VL9 peptide residues involved in the 3H4 interface are coloured marine blue. Interfacing HLA-E HC residues that contact the 3H4 VH are shaded orange whereas those that contact the 3H4 VL are shaded teal. HLA-E heavy chain residues involved in the interface with both the 3H4 VH and VL are shaded violet. Residue positions are numbered on the HLA-E surface view. (F) Aerial view of the overlapping 3H4 and CD94/NKG2A footprints on the HLA-E peptide binding groove. VL9 peptide residues involved in both the 3H4 and CD94/NKG2A interfaces are shaded marine blue whereas HLA-E heavy chain residues involved in both interfaces are shaded violet. Peptide and HLA-E heavy chain residues involved exclusively in the CD94/NKG2A interface are shaded in teal and orange, respectively. (G) Binding interface of 3H4 HC/VL9 peptide. Interfacing residues (Y97, S100, S100A and Y100B of the VH CDR3 loop and V1, M2, P4 and R5 of the VL9 peptide) are shown in ball and stick-form with non-interfacing residues in cartoon form. The VL9 peptide is shaded lime green, the HLA-E heavy chain in grey and the 3H4 HC in light purple. 3H4 is numbered according to the Kabat scheme whereby alternate insertion codes (letters after the residue number) are added to variable length regions of the antibody sequence. Kabat numbering is applied in all subsequent figures. (H-I) Binding interfaces of 3H4 HC/HLA-E heavy chain (H) and 3H4 LC/HLA-E HC (I). Interfacing residues are displayed in ball-and-stick form, non-interfacing residues are displayed in cartoon form and hydrogen bonds as dashed lines. In the 3H4 HC/HLA-E heavy chain interface (H), interfacing residues derived from the HLA-E heavy chain (grey) include G56, S57, E58, Y59, D61, R62, E63, R65, S66 and D69 of the α1-helix and E154, H155, A158, Y159, D162, T163 and W167 of the α2-helix. 3H4 VH (light purple) interfacing residues include N33 of the CDR1 region, N52, N54, G56 and T57 of the CDR2 region, Y97, G99, S100, S100A, Y100B and W100D of the CDR3 region and W47, D50, 158, Y59, N60, Q61 and K64 of the non-CDR VH domain. In the 3H4 LC/HLA-E heavy chain interface (I), HLA-E heavy chain (grey)-derived interfacing residues include E55, E58, Y59 and R62 of the α1-helix and D162, T163, E166, W167, K170 and K174 of the α2-helix. 3H4 LC (teal) interfacing residues include Q27, D28, N30 and Y32 of the CDR1 region, D92, E93, F94 and P95 of the CDR3 region in addition to D1 of the VL domain. (J) Key interfacing residues within the germline-encoded D-junction. 3H4 HC amino acid sequence with the VH segment in purple and the CDR1/2/3 regions shaded grey. Germline-encoded residues within the VH CDR3 D-junction are denoted. The 4 key interfacing residues (Y97, S100, S100A and Y100B) within this germline-encoded D-junction that make contacts with both the HLA-E heavy chain and VL9 peptide are highlighted magenta in the sequence and illustrated as magenta sticks in the PyMol visualisation. The HLA-E heavy chain and VL9 peptide are displayed as grey and green cartoons, respectively, with key interfacing residues in stick form. Hydrogen bonds are depicted as magenta dashed lines and residues of the 3H4 VH domain that are not germline-encoded key interfacing residues are displayed in light purple cartoon form.

FIGS. 3A-E. MAb 3H4 enhanced the cytotoxicity of the NKG2A+ NK cell line NK-92 against HLA-E-VL9 expressing 293T cells. (A) Schematic illustrating the hypothesis. Blockade of the inhibitory NKG2A/CD94/HLA-E pathway with anti-HLA-E-VL9 antibody (3H4) and/or anti-NKG2A antibody (Z199) could enhance target cell lysis by NK cells. (B-C) NK cell cytotoxicity against 3H4 IgM-treated target cells as assessed by 51Cr release assay. Antibody was incubated with HLA-E-VL9 transfected 293T cells (B) and untransfected 293T cells (C) at final concentration of 10 μg/ml or 3 μg/ml, and NK92 cells were added into the mixture as effector cells at effector: target (E:T) ratios of 20:1 and 6:1. Mouse IgM MM-30 was used as an isotype control. Dots represent the mean values of triplicate wells in eight independent experiments. Statistical analysis was performed using mixed effects models. Asterisks show the statistical significance between indicated groups: ns, not significant, *P<0.05, **P<0.01, ***P<0.001, ****P<0.0001. (D-E) NK cell cytotoxicity in the presence of anti-NKG2A mouse IgG Z199 in combination with TE4 control- or 3H4-treated target cells as assessed by 51Cr release assay. Antibody combinations of Z199+IgM control (D) or Z199+3H4 (E) were incubated with HLA-E-VL9 transfected 293T cells and untransfected 293T cells at a final concentration of 10 μg/ml, and NK92 cells were added into the mixture as effector cells. Dots represent the mean values of triplicate wells in three independent experiments.

FIGS. 4A-G. Affinity maturation of HLA-E-VL9-specific antibody 3H4 on human IgG1 backbone. (A) Schematic illustration of the affinity maturation strategy. Libraries of 3H4 mAb variants were transformed into S. cerevisiae and displayed on the surface of yeast cells as single-chain fragment variable (scFv). APC-conjugated HLA-E-VL9 tetramers were used for FACS sorting. (B) Sites at the 3H4/HLA-E-VL9 interface where sequence optimization by library screening provided the most significant affinity gains. 3H4: purple; HLA-E: green; VL9 peptide: orange. See Example 3 FIG. 6 for VH sequences. (C) Enrichment of HLA-E-VL9+ library clones after three rounds of selection by fluorescence-activated cell sorting (FACS). The yeast cells containing the scFv libraries were sorted sequentially for binding to decreasing concentrations of fluorescently labeled HLA-E-VL-9 (50 μg/ml, top; 10 μg/ml, middle; or 0.6 μg/ml bottom). (D) Mutations at positions 97-100 in the eleven 3H4 variants chosen for additional characterization upon library screening. (E) Binding of 3H4 Gwt and optimized variants to HLA-E-VL9 or HLA-E-Mtb44 transfected 293T cells. Representative flow cytometry data from one of three independent experiments are shown. (F) SPR sensorgrams showing binding kinetics of 3H4 Gwt and optimized variants. Rate constants (ka, kd) and dissociation constant KD were determined by curve fitting analysis of SPR data with a 1:1 binding model. Binding data are shown as black lines, and the best fits of a 1:1 binding model are shown as colored lines. Representative data from one of two independent experiments are shown. (G) Enhanced NK-92 cell cytotoxicity by optimized IgG 3H4 Gv3 and 3H4 Gv6 on HLA-E-VL9 transfected 293T cells and untransfected 293T cells, in compare with IgG 3H4 Gwt. Dots represent the mean values of triplicate wells in four or five independent 51Cr release assays. Statistical analysis was performed using mixed effects models. Asterisks show the statistical significance between indicated groups: ns, not significant, *P<0.05, **P<0.01, ***P<0.001, ****P<0.0001.

FIGS. 5A-L. HLA-E-VL9-specific antibodies exist in the B cell pool of healthy humans. (A) Scheme of isolating HLA-E-VL9-specific antibodies from healthy humans. Pan-B cells were first isolated by negative selection from human leukapheresis PBMCs. A three-color sorting strategy was used to sort single B cells that were positive for HLA-E-VL9 and negative for HLA-E-RL9HIV or HLA-E-RL9SIV. Flow cytometry data showing the sorting of HLA-E-VL9 double positive, HLA-E-RL9HIV negative, HLA-E-RL9SIV negative B cells in PBMCs from four donors (LP021, LP030, LP059 and LP060) are shown. Variable regions of antibody heavy and light chain genes were isolated from the sorted B cells by PCR and cloned into an expression backbone with a human IgG1 constant region. Antibodies were produced by transient transfection in 293i cells, and antibody binding specificities were analyzed by surface staining of transfected 293T cells and high throughput screening (HTS) flow cytometry. (B) Binding specificities of the HLA-E-VL9-specific antibodies (n=56) from four donors shown as a heatmap. The compensated MFIs of HLA-E-VL9-specific antibody staining on HLA-E-VL9, HLA-E-RL9HIV, or HLA-E-RL9SIV transfected 293T cells at a concentration of 1 μg/ml were shown. Representative data from one of two independent experiments are shown. (C) NK cell cytotoxicity against CA147 IgG-treated target cells as assessed by 51Cr release assay. Human antibody CA147 was incubated with HLA-E-VL9 transfected 293T cells and untransfected 293T cells at final concentration of 10 μg/ml or 3 μg/ml, and NK92 cells were added into the mixture as effector cells at effector: target (E:T) ratio of 20:1 and 6:1. Human antibody A32 was used as the isotype control. Dots represent the mean values of triplicate wells in five independent experiments. Statistical analysis was performed using mixed effects models. Asterisks show the statistical significance between indicated groups: ns, not significant, *P<0.05, **P<0.01, ***P<0.001, ****P<0.0001. (D) Percentage of HLA-E-VL9-specific B cells in CD19+ pan-B cells in four donors. (E) Phenotypes of HLA-E-VL9-specific B cells (n=56) shown as heatmap. Expression of markers in each single B cell were determined from index sorting data and are shown as MFIs after compensation. Compensated MFIs below zero were set as zero. Each row indicates one single cell. The rows were clustered by K-means Clustering in R. Four subsets were observed: CD10−CD27−CD38+/−naïve B cells, CD10+CD27−CD38++ transitional B cells, CD10−CD27+CD38− non-class-switched memory B cells, and CD10−CD27+CD38+ plasmablast cells. Detailed information for each single cell and antibody is shown in FIG. 22E. (F-G) Antibody gene usages. (F) Heavy chain viable (VH) region gene usage shown as a bar chart (left) and pie chart (right). The top five VH genes found in HLA-E-VL9-specific antibodies are colored in the pie charts. (G) Kappa chain variable (Vx) and lambda chain variable (VW) region gene usage shown as a bar chart (left) and pie chart (right). The top five Vx/VX genes found in HLA-E-VL9-specific antibodies are colored in the pie charts. Reference VH-VL repertoires (n=198,148) from three healthy human donors from a previous study (DeKosky et al., 2015) were used as a control. The chi-square test of independence was performed to test for an association between indicated gene usage and repertoire/antibody type in panels A-B. ****, p<0.0001; *, 0.01<p<0.05. (H-I) Comparison of heavy chain (H) and light chain (I) CDR3 (CDR-H3) length. HLA-E-VL9 antibody CDR-H3 length was compared with the reference (DeKosky et al., 2015) human antibody CDR-H3 length. (J-K) Violin plots showing the mutation rates of heavy chains (J) and light chains (K). HLA-E-VL9 antibody sequences (E-VL9) were compared with reference sequences from naïve and antigen-experienced (Ag-Exp) antibody repertoires (n=13,780 and 34,692, respectively) (DeKosky et al., 2016). (L) Mouse or human HLA-E-VL9-specific antibodies cross-react with microbiome-derived peptides presented by HLA-E. K562-E cells loaded with microbiome-derived peptides were detected with the indicated antibodies at a concentration of 10 μg/ml. Binding activities are shown in heatmap form with relative MFIs (MFI of peptide-loaded cells minus MFI of no peptide control cells) depicted on the scale shown. Representative data from one of three independent experiments are shown.

FIGS. 6A-C shows a summary of affinity matured antibodies (A), non-limiting embodiments of nucleic acids (B) and amino acids (C). In this figure, underlined are the four positions changed relative to the original 3H4 VH sequence. FIG. 6C discloses SEQ ID NOS 35-45, respectively, in order of appearance.

FIGS. 7A-K provide the nucleotide sequences of the Vh and Vl domains for antibodies listed in Table 1. The CDR regions are underlined according to IMGT convention, which is only one embodiment of possible CDR boundaries.

FIGS. 8A-H provide the amino acid sequences of the Vh and Vl domains for antibodies listed in Table 1. The CDR regions are underlined according to IMGT convention, which is only one embodiment of possible CDR boundaries. FIG. 8A discloses SEQ ID NOS 128-135, respectively, in order of appearance. FIG. 8B discloses SEQ ID NOS 136-143, respectively, in order of appearance. FIG. 8C discloses SEQ ID NOS 144-151, respectively, in order of appearance. FIG. 8D discloses SEQ ID NOS 152-159, respectively, in order of appearance. FIG. 8E discloses SEQ ID NOS 156, 141, 160-164, and 157, respectively, in order of appearance. FIG. 8F discloses SEQ ID NOS 165-167, 157, and 168-171, respectively, in order of appearance. FIG. 8G discloses SEQ ID NOS 172-179, respectively, in order of appearance. FIG. 8H discloses SEQ ID NOS 180-187, respectively, in order of appearance.

FIG. 9. A selection scheme of optimized libraries to affinity mature antibodies.

FIG. 10. Antibody residues to be optimized through site directed libraries mapped on the structure of the 3H4-HLA-E/VL9 complex. Antibody residues showed in spheres and displayed in the same color will be varied at the same time in the respective site directed libraries. 3H4 antibody is shown in purple (light chain) and magenta (light chain). HLA-E is shown in green and the VLP peptide in orange.

FIG. 11. Non-limiting embodiments of scFv variants (3H4 SD1-7) comprising contact residues which could be modified in an optimized variant. Based on structural analysis, seven scFv different directed libraries that sample all the amino acid variants at four sites were designed. The four sites are shown in bold and red in the sequence. Sequence changes identified from these libraries that improve the properties of 3H4 mAb will be further combined in additional libraries that will be screened as in FIG. 9 to identify sets of mutations that are cumulative towards the optimization 3H4. Each sequence shows one embodiment of a scFv fragment where VH is italicized and VL is double underlined, and HCDR3 sequence is underlined. The linker between the VH and VL sequence could be any suitable linker of varying length and/or sequence. See e.g. Chao et al. Nat Protoc. 2006; 1(2):755-68. doi: 10.1038/nprot.2006.94.

FIGS. 12A-K. Isolation and characterization of monoclonal antibody 3114. Related to FIG. 1. (A-B) Expression of human HLA-B27 and β2M in peripheral blood lymphocytes (PBLs) of the transgenic (TG) mice. HLA-B27/β2M TG mice were used to minimize the induction of antibodies to HLA class I and β2M. Mouse PBLs from TG mice and littermate control were isolated and stained with anti-mouse CD45, anti-human HLA class I (A/B/C) and anti-human β2m antibodies. Representative data (A) and the percentages of human HLA-B27+β2M+ cells in CD45+ PBLs from TG mice (n=6) and control mice (n=6) (B) were shown. (C) Schematic diagram of the immunization, splenocyte fusion and hybridoma screening strategy. HLA-B27/β2M TG mice (n=10) were immunized with cell surface-expressing HLA-E-RL9 peptide (a peptide derived from HIV-1; denoted RL9HIV hereafter) single-chain trimer (SCT)-transfected 293T cells (indicated by red arrows). After immunizations, spleen cells were harvested from the selected mouse and the fusion was performed using NS0 cells to generate hybridoma cells. Supernatants from the hybridoma cell candidates were screened for differential binding by surface staining on HLA-E-VL9, HLA-E-RL9HIV or HLA-E-RL9SIV transfected 293T cells. Hybridomas producing antibodies specific for HLA-E-VL9 but not others were selected for cloning and downstream analysis. Monoclonal cells were cloned for at least five rounds. (D) Serum antibody binding ELISA. Serum antibodies to HLA-E-VL9, HLA-E-RL9SIV, HLA-E-RL9HIV complexes were quantified by ELISA and shown as log AUC (area under curve). Antigens used for immunizations and ELISA assays are all cysteine (C)-trap stabilized. Each curve represents one animal, and the curve for animal that we used for splenocyte fusion are shown in red. (E) Affinity of 3H4 binding to soluble HLA-E-VL9 complex. 3H4 as a mouse IgM or as a recombinant human IgG1 were immobilized on CM5 sensor chips and soluble HLA-E-VL9 complex protein at the indicated concentrations was flowed over antibody immobilized sensor chips. Binding data are shown as black lines, and the best fits of a 1:1 binding model are shown as colored lines. Rate constants (ka, kd) and dissociation constant KD were measured following curve fitting analysis. (F) Purification of 3H4 by FPLC using Superose 6 size exclusion column. The arrowed peak was collected and analyzed by negative staining. (G) Representative class average images of 3H4 negative stain electron microscopy (NSEM). (H) Binding of 3H4 expressed in a human backbone G1.4A to a panel of autoantigens by AtheNA assays. HIV-1 gp41 antibody 4E10 was set as a positive control, and a Flu antibody Ab82 was used as a negative control. The dotted lines indicate the cutoff values ≥100 luminance units used to denote positivity. (I) Binding of 3H4 in human backbone G1.4A to HEp-2 epithelial cells in indirect immunofluorescence staining assays. HIV-1 gp41 antibody 2F5 was set as a positive control, and HIV-1 gp120 antibody 17B was used as a negative control. Antibody staining concentration was 50 μg/ml, and data were collected at 40× objective for 8 seconds. Data are representative from one of two independent experiments. (J) 3H4 does not cross-react with mouse Qa-1b-peptide complex. 293T cells were transfected with HLA-E-VL9 (VMAPRTLLL), HLA-E-AL9 (AMAPRTLLL), mouse Qa-1b-VL9, or mouse Qa-1b-AL9. Transfected cells were stained with 3H4 antibody or an anti-β2M control antibody 2M2 followed by AF647 conjugated anti-mouse IgG(H+L) secondary antibody. Data are representative from one of three independent experiments. (K) 3114 recognizes peptides with variants in P1. 293T cells were transfected with HLA-E SCTs with VL9 peptides with single amino acid mutations at P1, then stained with 3H4 antibody or an anti-β2M control antibody 2M2 followed by AF647 conjugated anti-mouse IgG(H+L) secondary antibody (dark blue). Cells were gated for EGFP positive subsets. Isotype control stained cells were used as a negative control (grey filled histograms), and the wildtype VL9 peptide was a positive control (pale blue filled histograms). Data are representative from one of three independent experiments.

FIG. 13. Phenotypic analysis of NK-92 cells. Related to FIG. 3. NKG2A and CD94 expression in NK-92 cell line detected by flow cytometry. NK-92 cells were stained with PE-CD94 antibody or FITC-NKG2A antibody and analyzed in flow cytometer. A PE-isotype and FITC-isotype antibodies were used as negative controls.

FIG. 14. Related to FIG. 3. Fc receptors CD16, CD32 and CD64 expression in NK-92 cell line detected by flow cytometry. NK-92 cells were stained with BV650-CD16 antibody, APC-CD32 antibody, or BV421-CD64 antibody and analyzed in flow cytometer. Peripheral blood mononuclear cells (PBMCs) were used as positive controls. Dot plots overlay of antibody stained cells (red) and unstained control cells (grey) were shown. NK-92 cells were negative for CD16, CD32 or CD64, while a subset of PBMC cells were positive for each antibody. Data from a single antibody phenotype experiment.

FIGS. 15A-C Crystallographic data for the 3114 Fab and VL9-bound HLA-E co-complex structure. Related to FIG. 3. (A) Crystallographic data collection and refinement statistics. AS: Ammonium sulphate. § r.m.s.d.: root mean square deviation from ideal geometry. Statistics for outer shell indicated in parentheses. AU: asymmetric unit. Rfree equals the R-factor against 5% of the data removed prior to refinement. Inter-chain RMSD and inter-molecular interfaces in the 3H4-HLA-E-VL9 structure. (B) Table detailing the total buried surface area of the interface in A2 between the 3H4 VH, VL and the VL9-bound HLA-E complex. (C) Table of RMSD (root mean square deviation) in Å between chains of the 3H4-HLA-E-VL9 co-complex structure. Two copies of the 3H4 Fab-HLA-E-VL9 co-complex were present in the asymmetric unit and thus RMSD between chains related by non-crystallographic symmetry was calculated via Ca atom pairwise alignment on the PDBePISA server. Average Ca atom RMSD following pairwise alignment is also reported for the HLA-E heavy chain (HC) of 1MHE (Chain A), a previously published non-receptor-bound VL9-loaded HLA-E complex, and the HLA-E HC from the 3H4-HLA-E-VL9 structure reported here (Chain A).

FIG. 16. Crystallographic data for the 3H4 Fab and VL9-bound HLA-E co-complex structure. Related to FIG. 3. Table listing residues involved in the interface between the 3H4 VH and the VL9 peptide.

FIGS. 17A-D. Crystallographic data for the 3H4 Fab and VL9-bound HLA-E co-complex structure. Related to FIG. 3. (A) Table of interacting residues of the 3H4 VH and HLA-E HC interface. (B) Table of interacting residues of the 3H4 VL and HLA-E HC interface.

(C-D) Hydrogen bonding and salt bridges in the 3H4-HLA-E-VL9 structure. Table of hydrogen bonds and salt bridges formed between the 3H4 heavy chain (HC) and the HLA-E HC (C) and the 3H4 light chain and the HLA-E HC (D). Hydrogen bonding cut-offs according to the PDBePISA default criteria. 3H4 chain numbering is according to the Kabat scheme whereby alternate insertion codes (letters after the residue number) are added to variable length regions of the antibody sequence. 3H4 residues within the CDRs are shaded green and labelled ‘CDR1/2/3’. The position of HLA-E HC residues either on the α1 or α2 helix is also noted. Amino acid atom abbreviations: C—mainchain Carbon atom, O—mainchain Oxygen atom, N—mainchain Nitrogen atom, CA—α-Carbon atom, CB—β-Carbon atom, CD—δ-Carbon atom, CE—ε-Carbon atom, CG—γ-Carbon atom, CH—η-Carbon atom, CZ—ζ-Carbon atom, OD—δ-Oxygen atom, OE—ε-Oxygen atom, OG—γ-Oxygen atom, OH—η-Oxygen atom, ND—δ-Nitrogen atom, NE—ε-Nitrogen atom, NH—η-Nitrogen atom, NZ—ζ-Nitrogen atom.

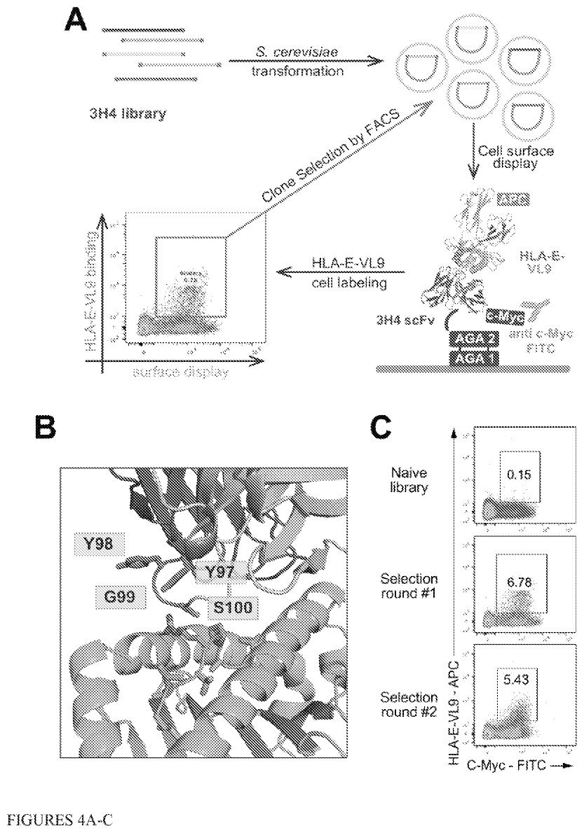

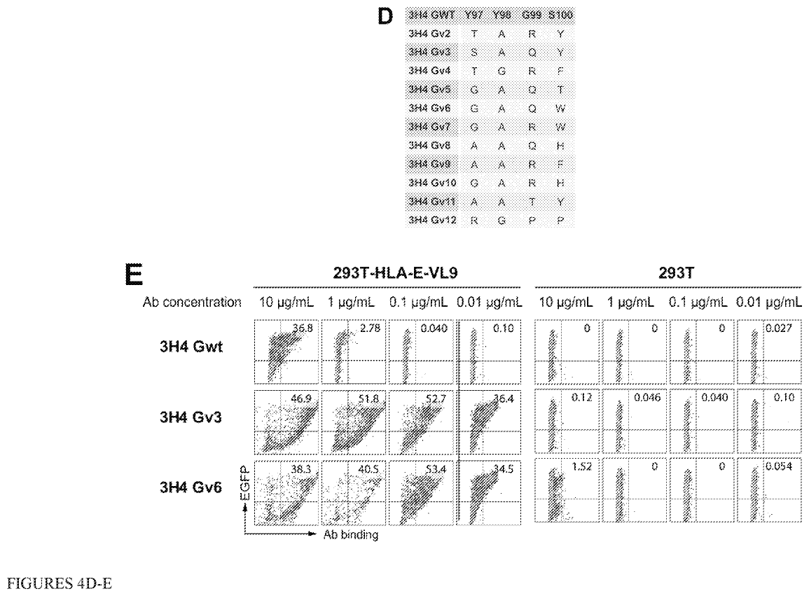

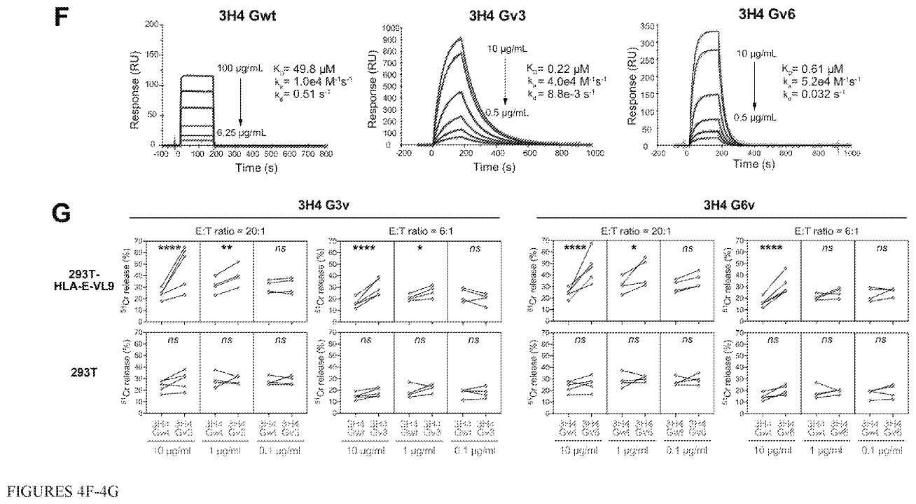

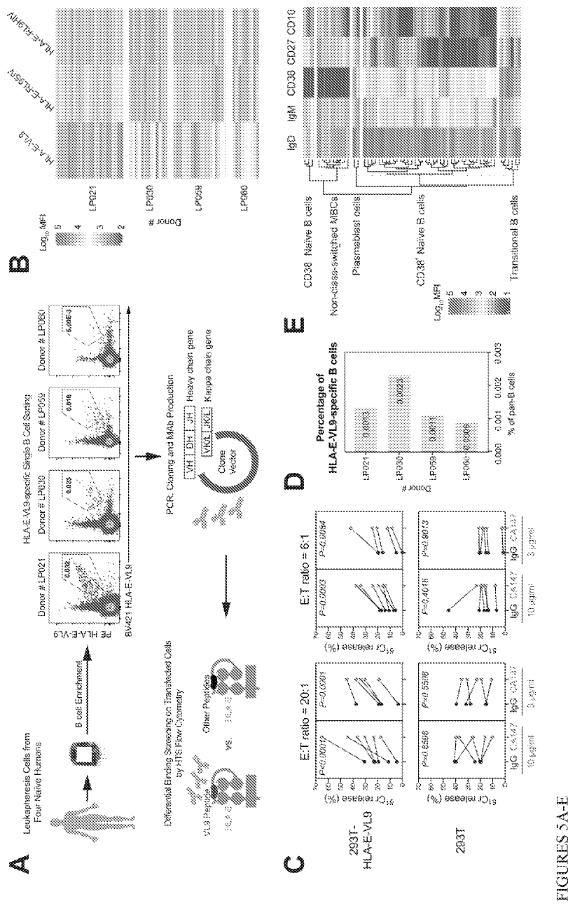

FIGS. 18A-E. Affinity optimization of 3H4 IgG. Related to FIG. 4. (A) NK cell cytotoxicity against wild-type 3H4 IgG-treated target cells measured by 51Cr release assay. Unoptimized, wild-type 3H4 on a human IgG1 backbone (3H4 Gwt) was incubated with HLA-E-VL9 transfected 293T cells and untransfected 293T cells at final concentration of 10 μg/ml, 1 μg/ml, or 0.1 μg/ml, and NK92 cells were added into the mixture as effector cells at effector: target (E:T) ratios of 20:1 and 6:1. Human IgG1 CH65 was used as an isotype control. Dots represent the mean values of triplicate wells in four or five independent experiments. Asterisks show the statistical significance between indicated groups: ns, not significant. (B) 3H4 residues optimized for affinity improvements by library screening. Seven different libraries were designed that simultaneously sampled group of 4 amino acids in the CDR loops of 3H4 that interact with HLA-E-VL-9 by structural analysis. Top two panels: Structural mapping of the amino acids (spheres) sampled together in the different libraries. Residues shown in the same color were randomized together. HLA-E backbone: green; VL-9 backbone: orange; (C) Binding of wild-type 3114 and variants on transfected 293T cells. Wild-type 3H4 and variants were titrated on HLA-E-VL9-transfected or untranfected 293T cells. Mean fluorescent intensity (MFI) from one of three independent experiments were shown. (D) and (E) Enhanced NK-92 cell cytotoxicity by optimized IgG 3114 Gv5 and 3114 Gv7 on HLA-E-VL9 transfected 293T cells and untransfected 293T cells, in comparison to IgG 3114 Gwt. Dots represent the mean values of triplicate wells in four or five independent 51Cr release assays. Statistical analysis was performed using mixed effects models. Asterisks show the statistical significance between indicated groups: ns, not significant, *P<0.05, **P<0.01, ***P<0.001, ****P<0.0001.

FIG. 19. Alignments of the 3H4 amino acid sequence indicating the groups of positions of amino acids randomized in each of the seven 3H4 libraries. Randomized residues are marked with ‘X’ and colored as in the structural panels above.

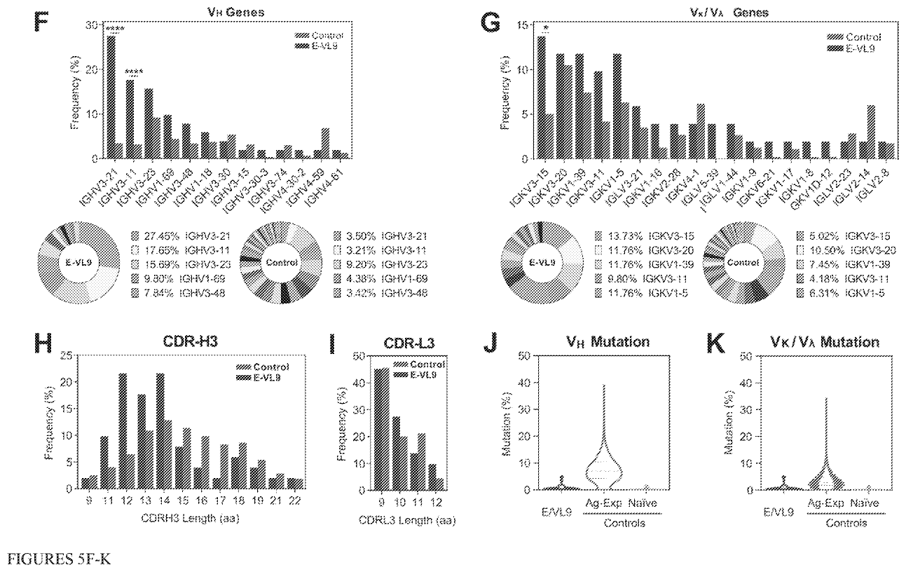

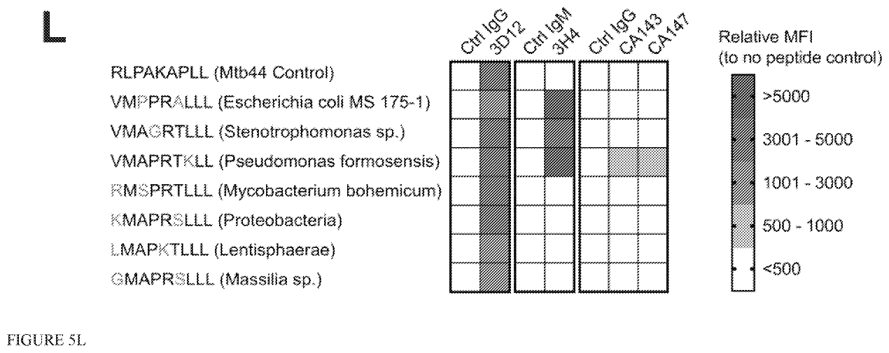

FIGS. 20A-J. Isolation and characterization of human HLA-E-VL9-specific antibodies. Related to FIG. 5. (A-B) HLA-E-VL9-specific B cell phenotyping from mice. Splenic cells isolated from HLA-B27/β2M TG mice (A) or C57BL/6 mice (B) were stained and analysed flow cytometry for B cells (B220+CD19+) that are HLA-E-VL9 double positive, HLA-E-RL9HIV negative and HLA-E-RL9SIV negative. (C) Gating strategy of the single cell sorting for HLA-E-VL9-specific B cells from a Cytomegalovirus (CMV)-negative, male human. Human B cells were first enriched from PBMCs by pan-B cell negative selection magnetic beads. The enriched cells were stained and gated on viable/CD14neg/CD16neg/CD3neg/CD235aneg/CD19pos/HLA-E-VL9pos/HLA-E-RL9HIVneg/HLA-E-RL9SIVneg subset as shown. Cells were single-cell sorted into 96-well plates for the downstream PCR cloning. Representative data from one of the four donors were shown. (D-E) Flow cytometry titration of purified HLA-E-VL9-specific mAbs isolated from a CMV-negative, male human. Antibodies recovered from sorted B cells were constructed in human IgG1 backbones and used for staining titration on both C-trap-stabilized and unstabilized HLA-E-VL9, HLA-E-RL9SIV, HLA-E-RL9HIV transfected 293T cells. EGFP expression indicates transfection efficiency. Transfected cells were stained with testing antibodies at the concentration of 2 μg/ml, followed by secondary antibody AF555-anti-human IgG staining. (D) Staining data of a representative antibody CA147 and a negative control antibody CA136. (E) Summary of the MFI of antibody binding data shown as bar chart. Data are representative from one of two independent experiments. (F) Cross-reactivities of human HLA-E-VL9 antibodies with rhesus Mamu-E-VL9 and mouse Qa-1b-VL9 complex. 293T cells were transfected with HLA-E-VL9, Mamu-E-VL9, two HLA-E/Mamu-E hybrids [HLA-E α1/Mamu-E α2 (Hα1/Mα2) and Mamu-E α1/HLA-E α2 (Mα1/Hα2)], and Qa-1b-VL9. Transfected cells were stained with human antibodies CA123, CA133, CA143, and CA147, followed by AF647 conjugated anti-mouse IgG(H+L) secondary antibody. Data are representative from one of three independent experiments. (G) Mapping of representative HLA-E-VL9-specific mAbs CA123, CA133, CA143 and CA147 on 293T cells transfected with HLA-E-VL9 peptide variants. 293T cells were transfected with HLA-E SCTs with VL9 peptides with single amino acid mutations at P1, then stained with human antibodies CA123, CA133, CA143, and CA147, followed by AF647 conjugated anti-mouse IgG(H+L) secondary antibody (dark blue). Cells were gated for EGFP positive subsets. MFI of the indicated antibody staining on wildtype VL9 peptide was set as 100%, and the percentages equals to (MFI of binding on each P1 variant)/(MFI of binding on wildtype VL9)×100%. (H) Affinity measurements of human HLA-E-VL9 antibodies binding to soluble HLA-E-VL9 complex. Human antibodies CA123 or CA147 on human IgG1 backbone was immobilized on CM5 sensor chips and soluble HLA-E-VL9 complex protein at the indicated concentrations was flowed over the antibody immobilized sensor chips. Rate constants (ka, kd) and dissociation constant KD were measured following curve fitting analysis. (I-J) NK cell cytotoxicity against CA123 IgG-treated target cells as assessed by 51Cr release assay. Human antibody CA123 was incubated with HLA-E-VL9 transfected 293T cells (I) and untransfected 293T cells (J) at final concentration of 30 μg/ml, g/ml or 3 μg/ml, and NK92 cells were added into the mixture as effector cells at effector: target (E:T) ratio of 20:1 and 6:1. Human antibody A32 were used as the isotype control. Dots represent the mean values of triplicate wells in five independent experiments. Statistical analysis was performed using mixed effects models.

FIGS. 21A-G. Gene usage and cross-reactivities of the HLA-E-VL9-specific antibodies. Related to FIG. 5. (A-D) J chain sequence analysis of HLA-E-VL9-specific antibodies (n=51). Reference VH-VL repertoires (n=198,148) from three healthy humans from a previous study (DeKosky et al., 2015) was used as a control. (A-B) Heavy chain (JH) gene usage shown as bar chart (A) and pie chart (B). (C-D) Kappa chain (Jk) and lambda chain (JL) gene usage shown as bar chart (C) and pie chart (D). (E) Human microbiome-derived peptides bound and stabilized HLA-E as indicated by upregulated HLA-E expression. The above described microbiome-derived peptides were loaded on K562 cells, and stained with HLA-E antibody 3D12 or isotype control mouse IgG at the concentration of g/ml. No peptide loaded cells were used as the negative control, and mtb44 peptide-loaded cells were set as the positive control. Data are representative from one of three independent experiments. (F-G) 3H4, CA143 and CA147 cross-reacted with human microbiome-derived peptides presented by HLA-E. The good HLA-E binders as shown in panel A were loaded on K562 cells, and stained with mouse antibodies (3H4 or isotype control mouse IgM TE4; panel F), as well as human antibodies (CA136, CA143 or CA147; panel G) at the concentration of 10 μg/ml. No peptide loaded cells were used as the negative control, and mtb44 peptide-loaded cells were set as the positive control. Data are representative from one of three independent experiments.

FIGS. 22A-E. HLA-E-VL9-specific antibodies isolated from humans. FIGS. 22A and B provide Mean Fluorescent Intensity data from binding specificity experiments.

FIGS. 22C and D provides for each antibody information on: Vh V-gene, D-gene, and J-gene family information; Vl V-gene and J-gene family information; CDR3 lengths; isotype; and mutation frequency information. The % mutation frequency is calculated from the mutation frequency in column U. FIG. 22E provide a correlation between the SEQ ID in the concurrently filed sequence listing for the nucleotide sequence for the Vh and Vl domains for each antibody.

FIG. 23. Microbiome database blast and HLA-E binding prediction for VL9-like epitopes. Prediction method: NetMHCpan EL 4.0. High Score=good binder. Candidates are ranked by HLA-E binding score from high to low. For microbiome-derived VL9-like peptides tested in this study, a VL9 peptide sequence was first searched by similarity in NIH Microbial database, and resultant microbial sequences were then analyzed using an MHC binding prediction tool for human HLA-E binding and mouse Qa1 binding (http://tools.immuneepitope.org/analyze/html/mhc_binding.html). Peptides that fulfilled the following criteria were selected for downstream validation experiments: 1) related to human pathogens, 2) 9 aa in length, and 3) with HLA-E binding score above 0.2. Related to FIG. 5.

FIGS. 24A and B show non-limiting embodiments of nucleic acid and amino acid sequences of antibodies (3H4 and CA147) designed to form hexamer. Any of the antibodies described herein could be designed to form hexamers. Non-limiting embodiments include any of the affinity matured antibodies described herein. FIG. 24B discloses SEQ ID NOS 337-341, respectively, in order of appearance.

FIGS. 25A-C shows expression of hexameric IgG forms of anti-HLA-E antibodies. (A) location of Fc mutations that generate IgG hexamers. (B-C) Two-dimensional class averages of negative stain electron microscopy images of (B) 3H4 or (C) CA147 IgG hexamers. Arrows indicate the antigen-binding fragment (Fab) and crystallizable fragment (Fc).

FIGS. 26A-C shows non-limiting embodiments of nucleic acid and amino acid sequences of antibody or fragment thereof sequences designed to form ferritin nanoparticles, or hexamers. FIG. 26A shows Amino acid sequence of full-length immunoglobulin genes (signal peptide is underlined; sortase acceptor sequence GGGGGSG is italicized). In FIG. 26A, signal peptide is underlined. For amino acids sequences and throughout the specification, a skilled artisan appreciates that recombinantly expressed protein do not include signal peptide which is cleaved during cellular processing. Double underlined is one non-limiting embodiment of a sortase donor/tag sequence LPETGG. FIG. 26A discloses SEQ ID NOS 342-345, 348-352, 350, 353, and 344, respectively, in order of appearance.

FIG. 26B shows Vh and Vl sequences of various antibodies. FIG. 26B shows amino acid sequences of only the variable regions of 3H4, CA147. FIG. 26B discloses SEQ ID NOS 128, 129, 184, 185, 184, 185, 128, and 129 respectively, in order of appearance. FIG. 26C shows non-limiting embodiments of nucleic acid sequencing encoding amino acids in FIGS. 26A-B. Any of the antibodies described herein could be designed to form nanoparticles and/or hexamers. Non-limiting embodiments include any of the affinity matured antibodies described herein.

FIGS. 27A-B shows negative stain electron microscopy images of (A) CA117 or (B) CA147 Fab nanoparticles. A and B. Raw images of Fab nanoparticles. The inset in B shows 3-D class average of the nanoparticle with Fab arrayed around the nanoparticle surface.

FIG. 28 shows that CA147 Fab nanoparticles recognize VL9:HLA-E complexes on the cell surface. See Example 3 for nanoparticle description. 293T cells were transfected HLA-E molecules loaded with VL9 (left) or tuberculosis peptide (right). Percentage cells bound by the Fab nanoparticles is shown for a dose range of Fab nanoparticle. Each row indicates binding at a different nanoparticle dose. Primary ab: Fab nanoparticles. Secondary ab: PE anti-His tag 1:50.

FIGS. 29A-B shows data concerning CA147 IgG with mutations within the Fc region form hexamers. CA147 IgG1 with three mutations (designated CA147_G1m3, with three mutations E345R/E430G/S440Y) were produced by transfected 293i and purified by Protein L IgK light chain beads. The Protein L purified products were further purified by Size exclusion chromatography (SEC) (A). The antibody before purification, the SEC purified peak A and B were used to stain HLA-E-VL9 or HLA-E-Mtb44 transfected 293T cells (B).

### DETAILED DESCRIPTION

The present invention relates to antibodies and antigen binding fragments thereof, including recombinant and/or derivative forms, that bind to HLA-E-peptide complexes. In some embodiments, the antibodies or fragments bind to epitopes on the HLA-E-VL9 peptide complex. The antibodies or fragments can have a binding specificity that is sensitive to the presence of a VL9 peptide as presented by HLA-E. The art defines the VL9 peptide as having a nine amino acid motif according to the following formula: VMAPRT(L/V)(V/L/I/F)L. In some respects, the antibodies of the invention specifically bind an HLA-E-VL9 complex where the peptide has an amino acid sequence according to the formula: VMAPRT(L/V)(V/L/I/F)L. In other respects, the antibodies of the invention bind an HLA-E-VL9 complex where the peptide has an amino acid sequence according to the following formula: (V/A/C/I/S/T/V/H/P)MAPRT(L/V)(V/L/I/F)L. In other aspects the peptide has an amino acid sequence variation at position P2 according to the following formula: (V/A/C/I/S/T/V/H/P)(M/L/Q/F)APRT(L/V)(V/L/I/F)L See e.g. Walters et al NATURE COMMUNICATIONS |(2018)9:3137 |DOI: 10.1038/s41467-018-05459—Thus, as used herein, a peptide with the motif, VMAPRTL(V/L/)L, VMA PRTV(L/I/F)L, (V/A/C/I/S/T)MAPRTLLL, V(M/T)APRTLLL, V(L/I/Q/T/V/S/A/F)APRTLLL, VMAPRTLL(L/F), or (V/A/C/I/S/T/V/H/P)(M/L/Q/F)APRT(L/V)(V/L/I/F)L, is referred to as a VL9 peptide or a VL9 variant. Non limiting embodiments of VL-9 peptides are listed in Table 4.

By binding to the HLA-E-VL9 complex, the antibodies can prevent intercellular signaling between HLA-E expressing cells and NKG2A expressing cells, i.e., the HLA-E-NKG2A pathway, which inhibits cytotoxic effector cell functions. Accordingly, the antibodies are useful in conditions where diseased or infected cells express HLA-E-VL9 complexes (or HLA-E-peptide complexes with peptides that do not confirm to the VL9 motifs recited above, including viral peptides and microbiome peptides, where an HLA-E-VL9 specific antibody of the invention is cross-reactive to the HLA-E-peptide complex) and where the condition would benefit from an increase in effector cell function against these cells.

### Recombinant Antibodies

Recombinant antibodies of the invention include antibodies derived from rearranged VDJ variable heavy chain (Vh) and/or rearranged VJ variable light chain (Vl) sequences from individual or clonal cells that express an antibody that specifically binds to HLA-E-VL9 (or other HLA-E-peptide complex of interest), and optionally is further able to prevent or inhibit binding between the HLA-E-VL9 complex and the NKG2A/CD94 heterodimeric complex. In some respects, the antibody is cross-reactive to HLA-E-peptide complexes where the peptide does not conform to VL9 motifs but where this HLA-E-non-VL9 peptide complex still engages with the NKG2A/CD94 complex such as with certain viral peptides and microbiome peptides. Antibodies are described in the accompanying examples, figures, and tables, and the invention includes antibodies comprising CDR sequences contained with the Vh and Vl amino acid sequences described herein. In certain embodiments, the invention provides monoclonal antibodies. In certain embodiments the monoclonal antibodies are produced by a clone of B-lymphocytes. In certain embodiments the monoclonal antibody is recombinant and is produced by a host cell into which an expression vector(s) encoding the antibody, or fragment thereof, has been transfected.

Methods for obtaining rearranged heavy and light chain sequences are well known in the art and often involve amplification-based-cloning and sequencing. Standard techniques of molecular biology may be used to prepare DNA sequences encoding the antibodies or antibody fragments of the present invention. Desired DNA sequences may be synthesized completely or in part using oligonucleotide synthesis techniques. Site-directed mutagenesis and polymerase chain reaction (PCR) techniques may be used as appropriate.

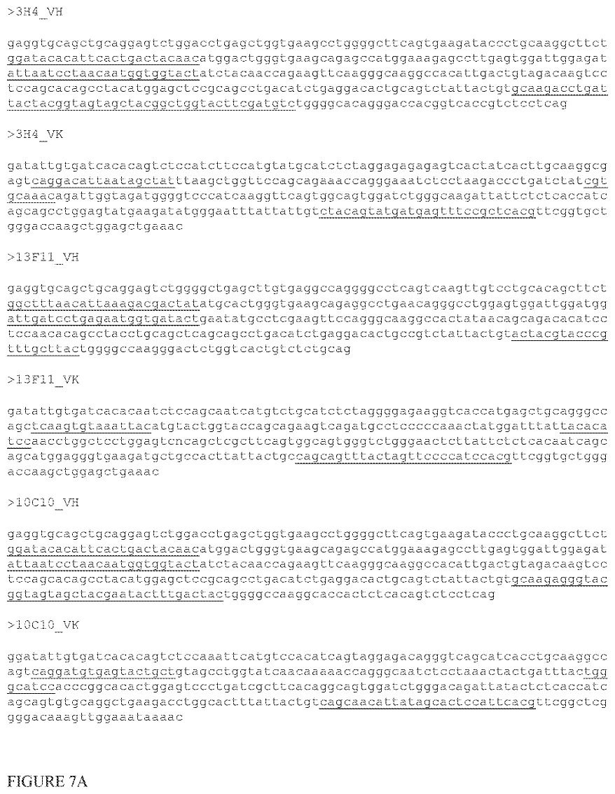

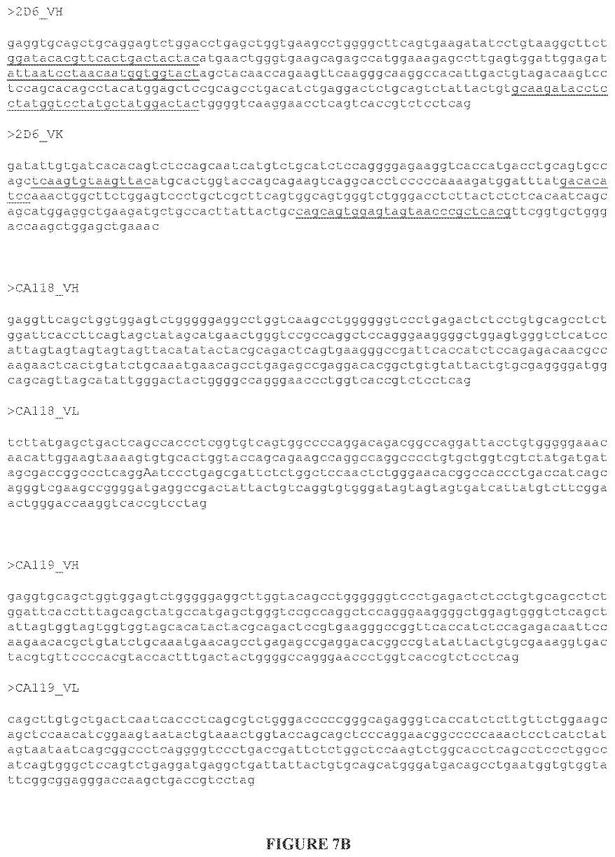

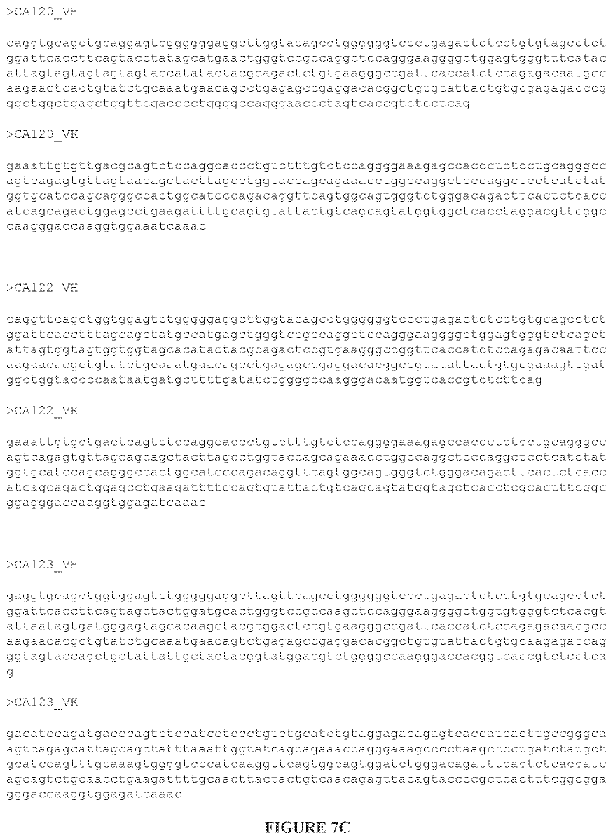

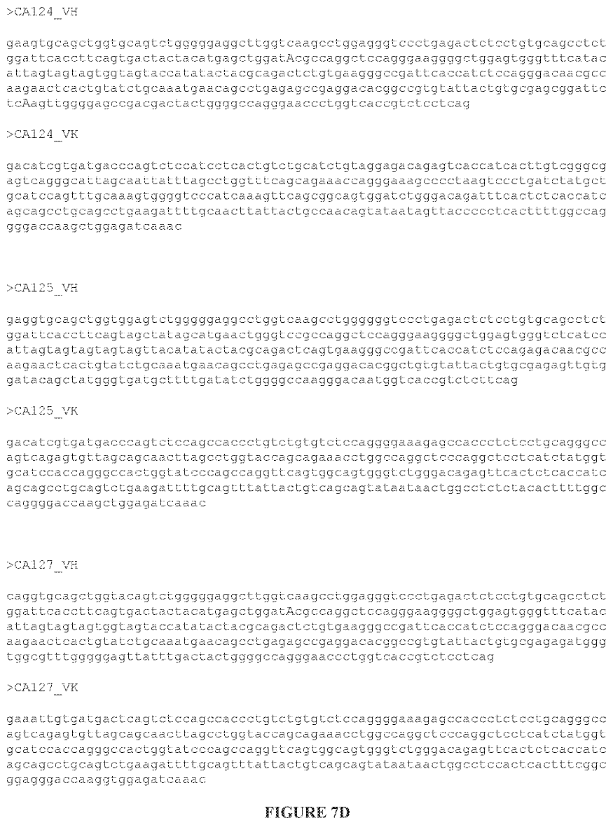

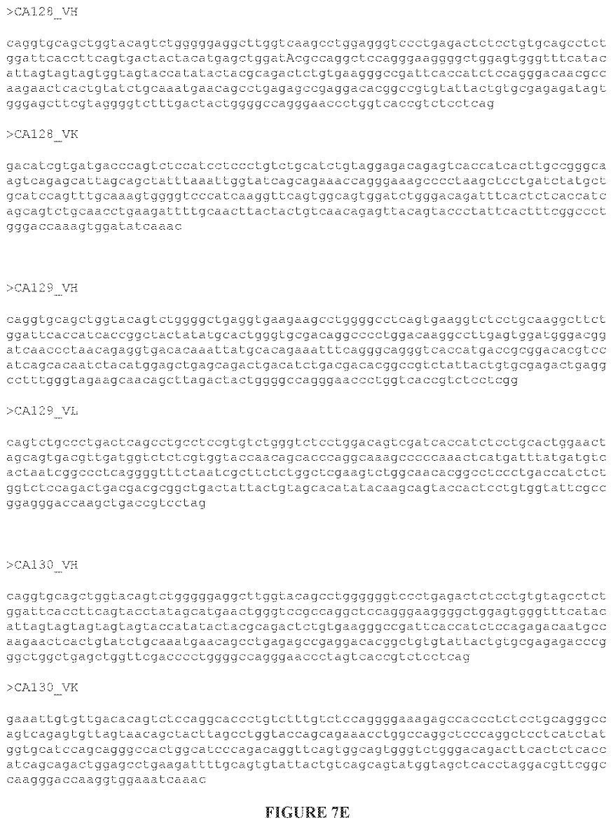

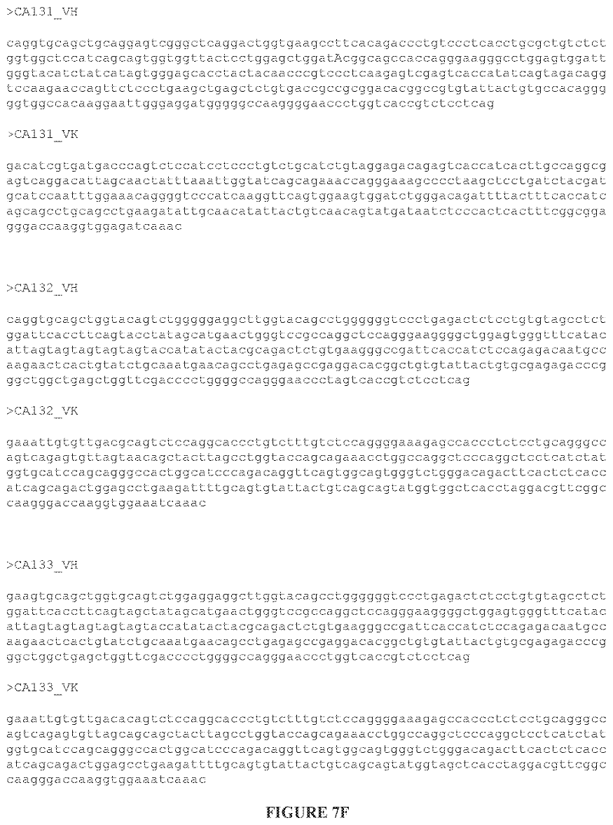

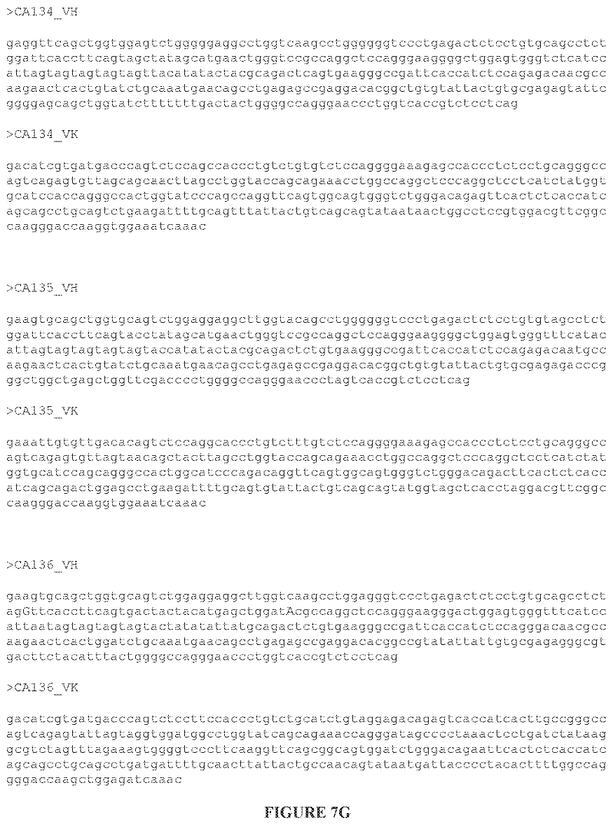

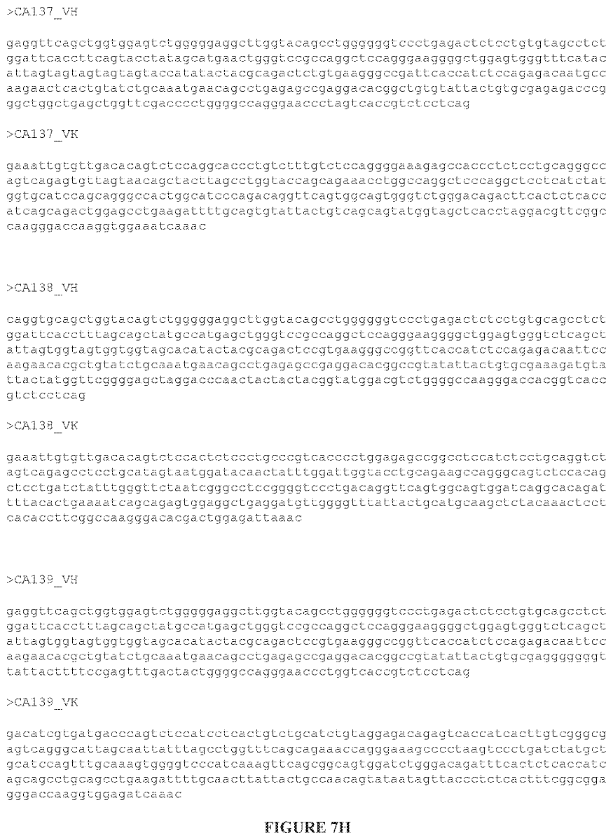

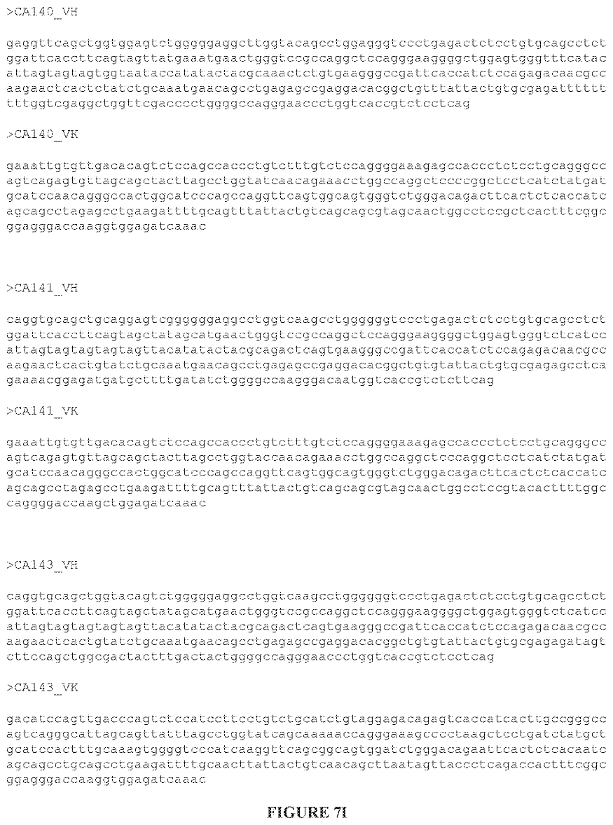

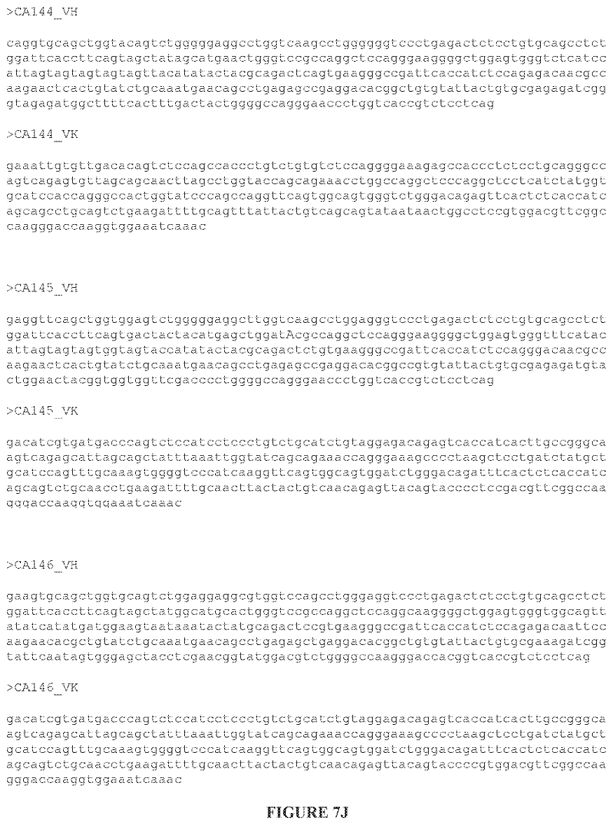

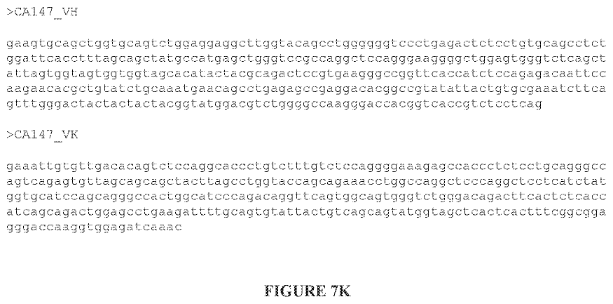

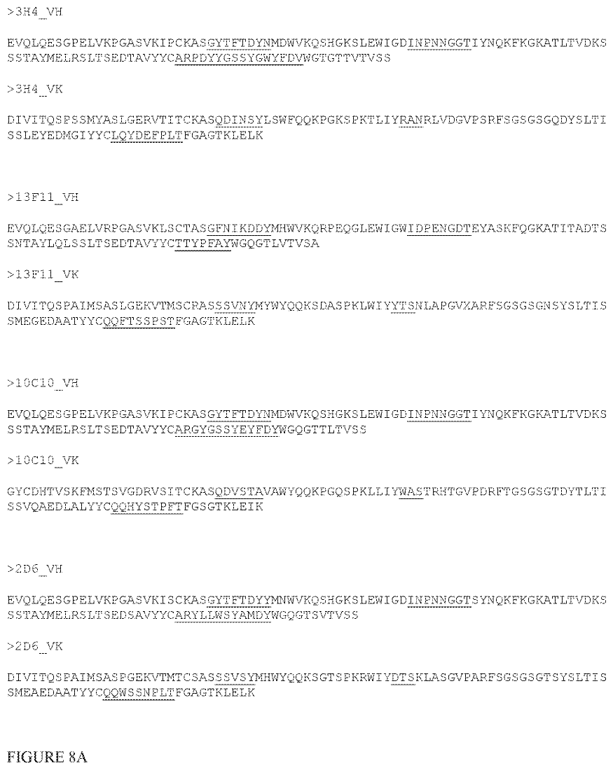

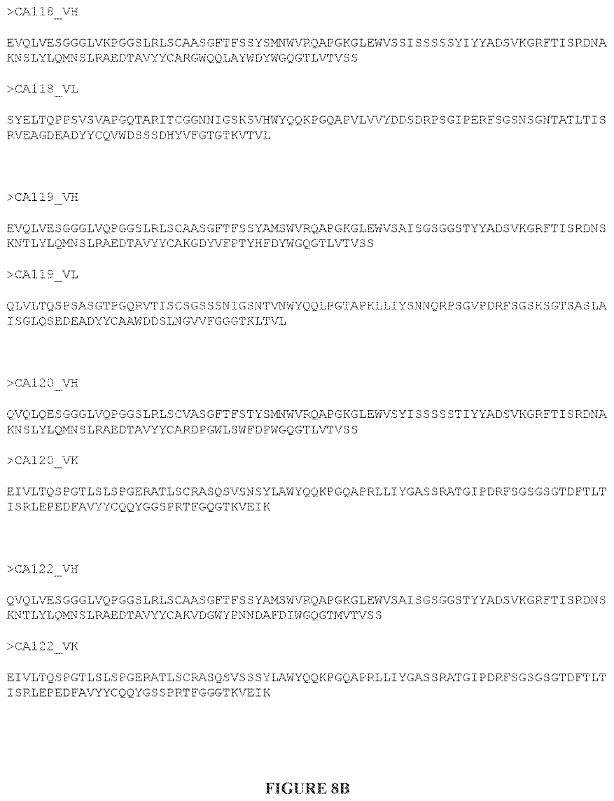

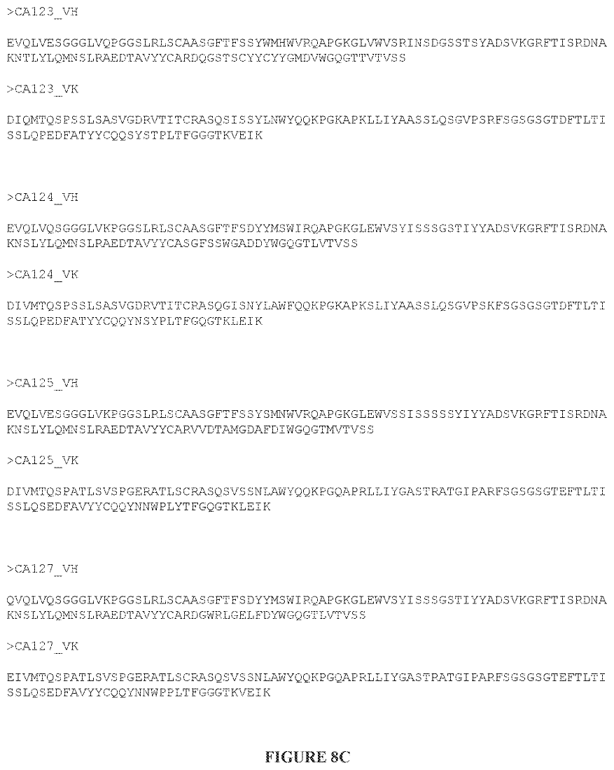

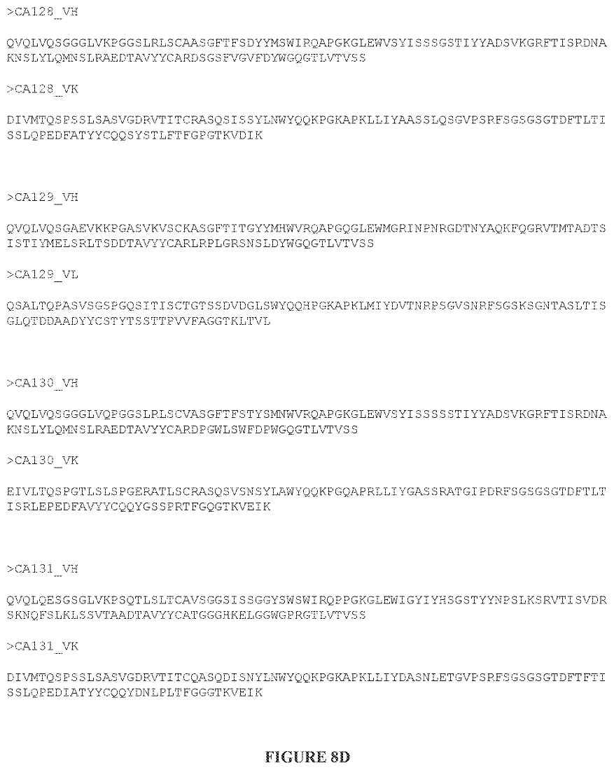

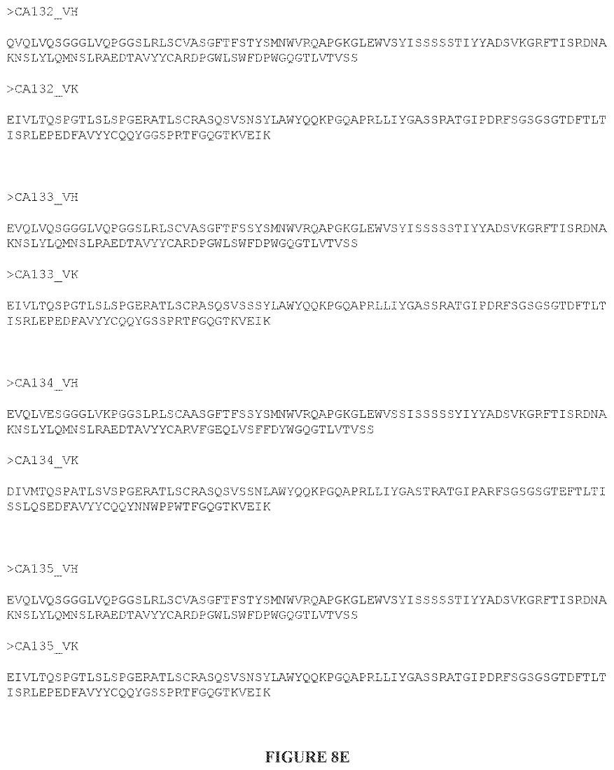

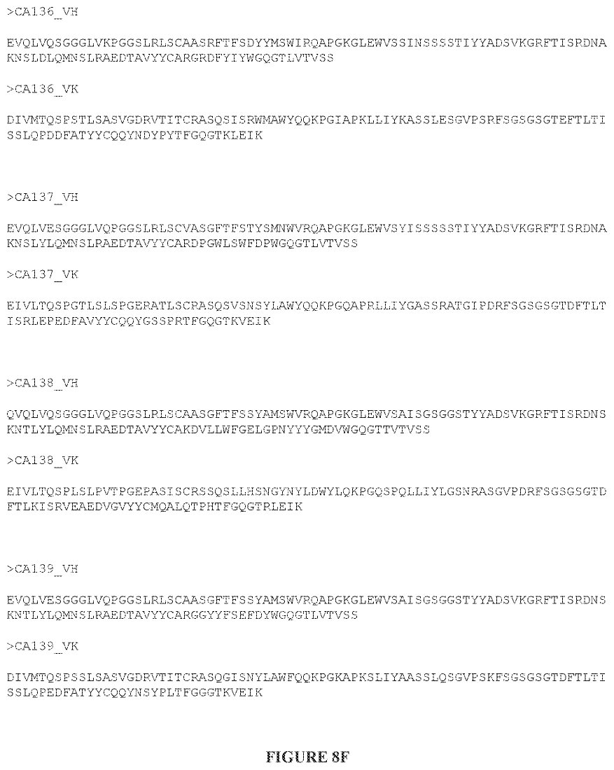

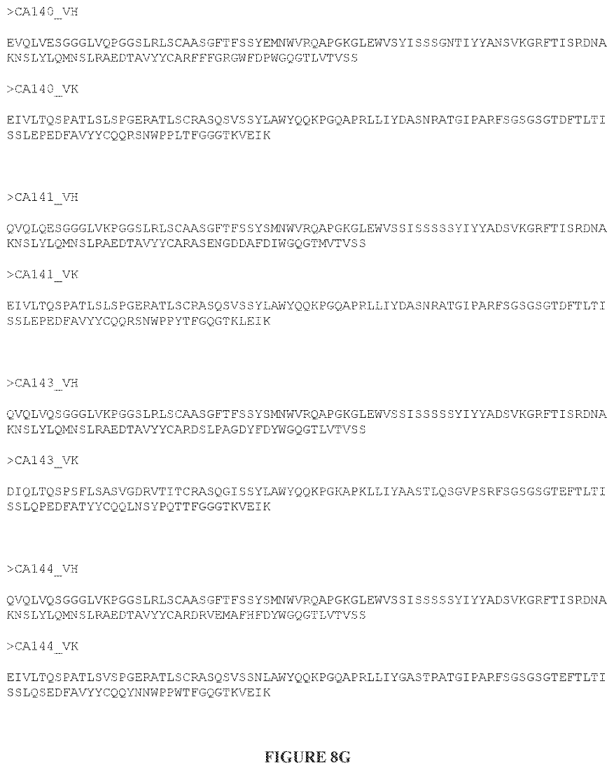

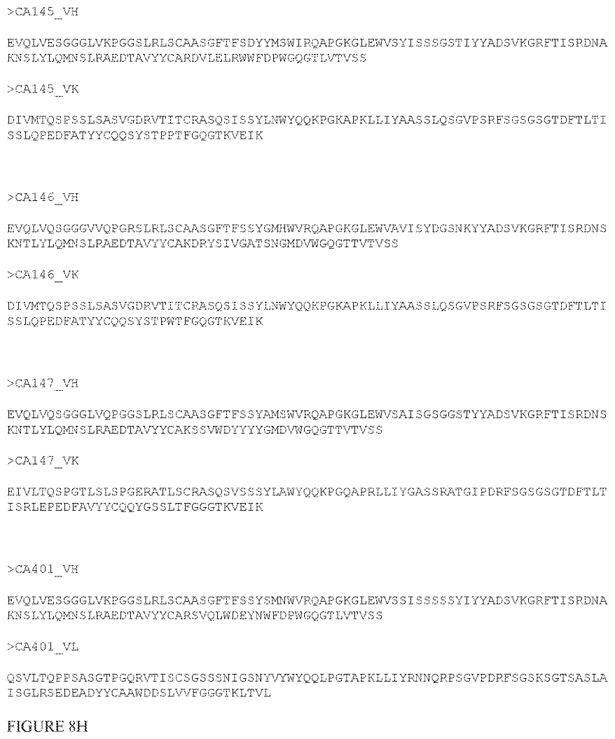

The invention encompasses antibodies which are 99%, 98%, 97%, 96%, 95%, 94%, 93%, 92%, 91%, 90%, 89%, 88%, 87%, 86%, 85%, 84%, 83%, 82%, 81%, 80%, 75% identical to the Vh and/or V1 variable domain amino acid sequences of the antibodies described herein in the Figures or Table 1. Further, the invention encompasses variants having one or mutations (99% et seq. per above) as compared to the sequences of the antibodies of the Figures or Table 1 with one or more of the additional requirements: (1) the variant maintains antigen binding specificity to the HLA-E-VL9 complex, and in some embodiments, maintains the ability to specifically bind an epitope that includes the part of Q2 domain of HLA-E and the amino terminal end of the VL9 peptide, (2) the variant does not have a decrease in binding affinity or avidity that is more than 10-fold, 5-fold, 2-fold, or 1-fold than the corresponding antibody of the Figures or Table 1, (3) the variant has a binding affinity or avidity that is an improvement of more than 100-fold, 10-fold, 5-fold, 2-fold, or 1-fold more than the corresponding antibody of the Figures or Table 1, (4) the variant does not have a decrease in promoting cytotoxic activity by NK cells or CD8+ cells that is more than 10-fold, 5-fold, 2-fold, or 1-fold as compared to the corresponding antibody of the Figures or Table 1, (5) the variant has an increase in in promoting cytotoxic activity by NK cells or CD8+ cells that is 10-fold, 5-fold, 2-fold, or 1-fold more as compared to the corresponding antibody of the Figures or Table 1, and (6) as compared to the antibodies listed in the Figures or Table 1, the variant has a V region with shared mutations compared to the germline, identical VDJ or VJ gene family usage, identical or the same or similar HCDR3 length, and the same VL and JL gene family usage. Generally, one or more of these six requirements are applicable to any antibody, including fragments (see below, Fab, Fv, et al.) or portions (Vh, Vl, one or more CDRs from a Vh/Vl pair) thereof, derived from the antibodies listed in Table 1 or the Figures. FIG. 7A provides the nucleotide sequence and FIG. 8 provides the amino acid sequence for the Vh and Vl domains of each of the antibodies listed in Table 1. FIG. 22E provides additional antibodies.

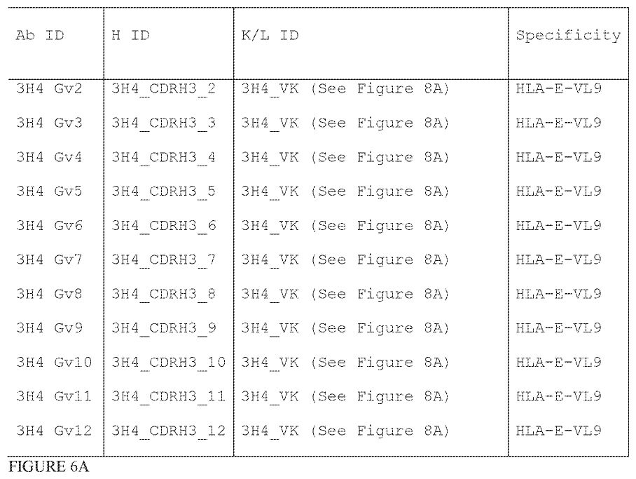

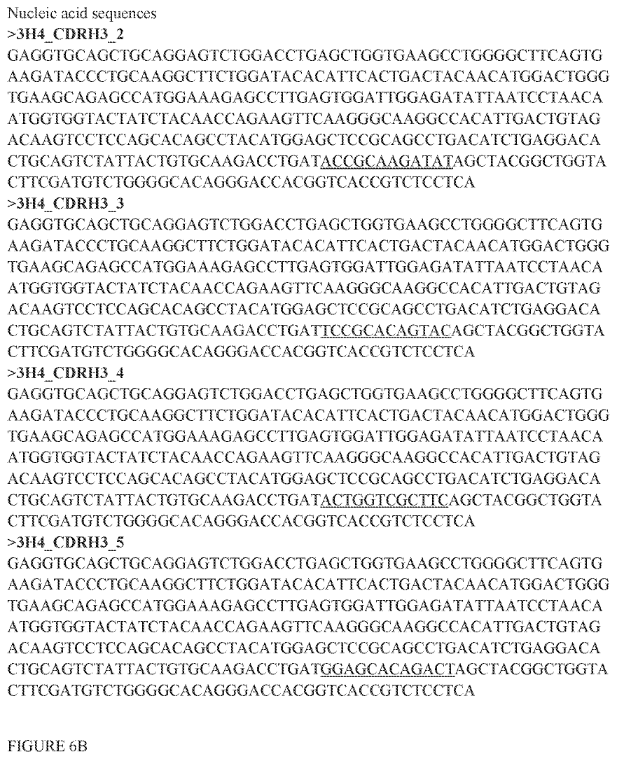

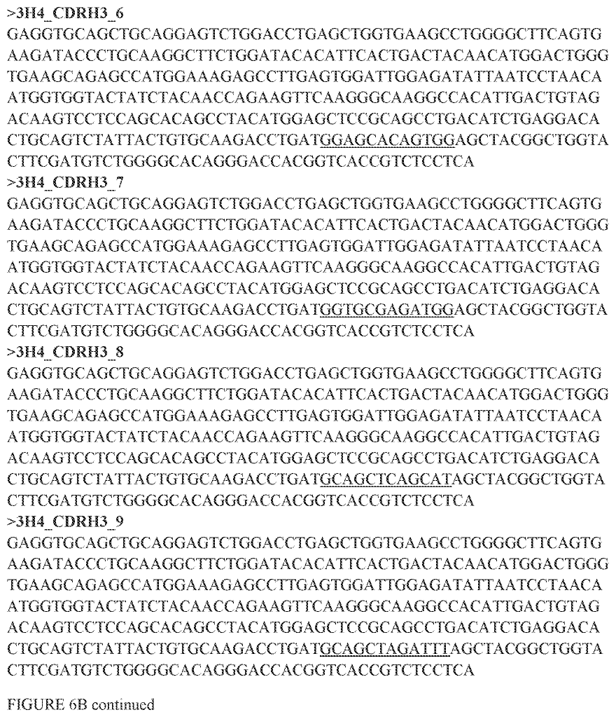

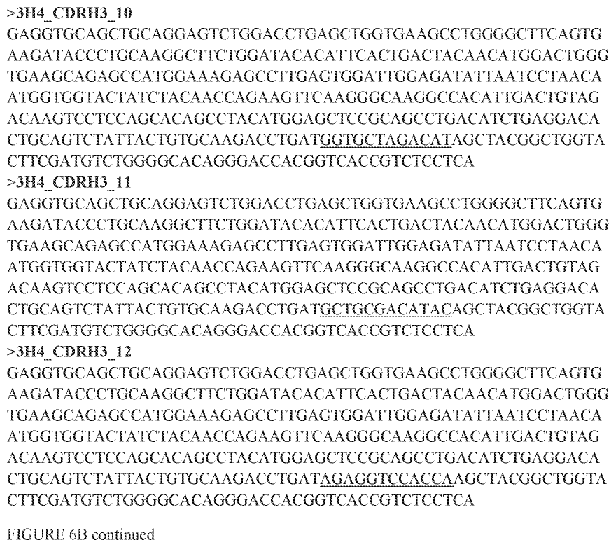

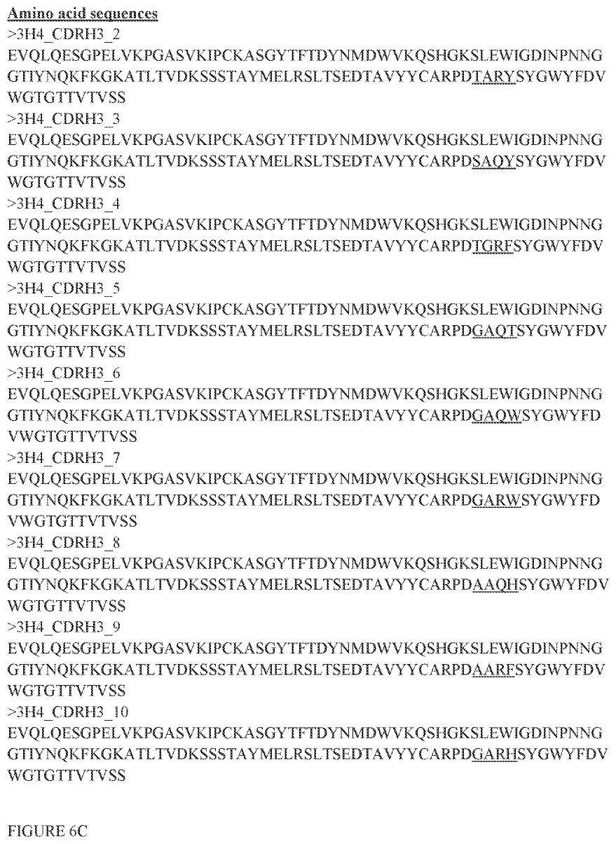

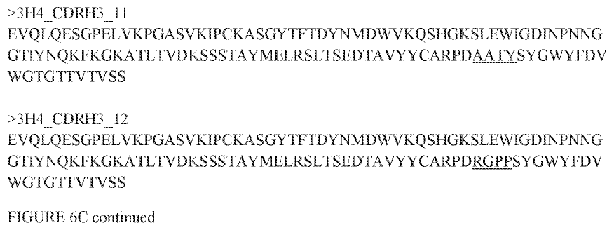

TABLE 1
List of Mouse and Certain Human Antibodies

| | | Light chain ID | |
|---|---|---|---|
| | | (kappa (K); | Species and |
| Ab ID | Heavy chain ID | lambda (L)) | Isotype |
| 3H4 | 3H4_VH | 3H4_VK | Mouse IgM |
| | FIG. 8A | FIG. 8A | |
| 13F11 | 13F11_VH | 13F11_VK | Mouse IgM |
| 10C10 | 10C10_VH | 10C10_VK | Mouse IgM |
| 2D6 | 2D6_VH | 2D6_VK | Mouse IgM |
| CA118 | CA118_VH | CA118_VL | Human IgM |
| CA119 | CA119_VH | CA119_VL | Human IgM |
| CA120 | CA120_VH | CA120_VK | Human IgM |
| CA122 | CA122_VH | CA122_VK | Human IgM |
| CA123 | CA123_VH | CA123_VK | Human IgM |
| CA124 | CA124_VH | CA124_VK | Human IgM |
| CA125 | CA125_VH | CA125_VK | Human IgM |
| CA127 | CA127_VH | CA127_VK | Human IgM |
| CA128 | CA128_VH | CA128_VK | Human IgM |
| CA129 | CA129_VH | CA129_VL | Human IgM |
| CA130 | CA130_VH | CA130_VK | Human IgM |
| CA131 | CA131_VH | CA131_VK | Human IgD |
| CA132 | CA132_VH | CA132_VK | Human IgM |
| CA133 | CA133_VH | CA133_VK | Human IgM |
| CA134 | CA134_VH | CA134_VK | Human IgM |
| CA135 | CA135_VH | CA135_VK | Human IgM |
| CA136 | CA136_VH | CA136_VK | Human IgM |
| CA137 | CA137_VH | CA137_VK | Human IgM |
| CA138 | CA138_VH | CA138_VK | Human IgM |
| CA139 | CA139_VH | CA139_VK | Human IgM |
| CA141 | CA141_VH | CA141_VK | Human IgM |
| CA143 | CA143_VH | CA143_VK | Human IgM |
| CA144 | CA144_VH | CA144_VK | Human IgM |
| CA145 | CA145_VH | CA145_VK | Human IgM |
| CA146 | CA146_VH | CA146_VK | Human IgM |
| CA147 | CA147_VH | CA147_VK | Human IgM |
| | FIG. 8H | FIG. 8H | |
| CA401 | CA401_VH | CA401_VL | Human IgM |
| 3H4 Gv2 | 3H4 CDRH3 2 | 3H4_VK | Mouse |
| | FIGS. 6B, C | | |
| 3H4 Gv3 | 3H4 CDRH3 3 | 3H4_VK | Mouse |
| | FIGS. 6B, C | | |
| 3H4 Gv4 | 3H4 CDRH3 4 | 3H4_VK | Mouse |
| | FIGS. 6B, C | | |
| 3H4 Gv5 | 3H4 CDRH3 5 | 3H4_VK | Mouse |
| | FIGS. 6B, C | | |
| 3H4 Gv6 | 3H4 CDRH3 6 | 3H4_VK | Mouse |
| | FIGS. 6B, C | | |
| 3H4 Gv7 | 3H4 CDRH3 7 | 3H4_VK | Mouse |
| | FIGS. 6B, C | | |
| 3H4 Gv8 | 3H4 CDRH3 8 | 3H4_VK | Mouse |
| | FIGS. 6B, C | | |
| 3H4 Gv9 | 3H4 CDRH3 9 | 3H4_VK | Mouse |
| | FIGS. 6B, C | | |
| 3H4 Gv10 | 3H4 CDRH3 10 | 3H4_VK | Mouse |
| | FIGS. 6B, C | | |
| 3H4 Gv11 | 3H4 CDRH3 11 | 3H4_VK | Mouse |
| | FIGS. 6B, C | | |
| 3H4 Gv12 | 3H4 CDRH3 12 | 3H4_VK | Mouse |
| | FIGS. 6B, C | | |
| CA147hexa | FIGS. 24B/26A | FIG. 26A | IgG as a |
| | | >CA147_FabH | hexamer |
| | | VL without | |
| | | signal peptide | |
| CA147sortaNP | FIG. 26A | FIG. 26A | Fab fragments |
| | >CA147_FabH_cSortA | >CA147_FabH | displayed on a |
| | VH without signal | VL without | nanoparticle |
| | peptide | signal peptide | |
| | Any sortase tag | | |
| 3H4hexa | FIG. 24B | FIG. 26A | IgG as a |
| | | >3H4_FabH | hexamer |
| | | VL without | |
| | | signal peptide | |
| 3H4sortaNP | FIG. 26A | FIG. 26A | Fab fragments |
| | >3H4_FabH_nSortA | >3H4_FabH | displayed on a |
| | VH without signal | VL without | nanoparticle |
| | peptide | signal peptide | |

With respect to the antibodies listed in Table 1, their corresponding sequences are provided in FIGS. 7 and 8 where CDR regions are underlined as determined by IN4GT (http://www.imgt.org/IMGT_vquest/). The boundaries of these CDR regions can be modified or substituted according to other CDR conventions as known in the art and as described herein. Further, there are additional human antibody sequences described in FIG. 22A-E that are not listed in Table 1. All embodiments described herein that refer to Table 1 may also encompass these additional human antibody sequences. Any one of the antibodies in Table 1 can be designed to form a hexamer. See Example 3 and FIGS. 24B, 25A, 26A. Any one of the antibodies in Table 1 can be designed to form a multimeric array of a full length antibodies, or fragments such as Fabs displayed on a nanoparticle, including without limitation ferritin nanoparticle. See Example 3, FIG. 26A. The constant heavy and light chains in the sequences referenced in the figures are non-limiting embodiments of constant heavy and light chain sequences. Any other suitable constant gene sequence could be used. The Fc portions of the full length antibodies could comprise any other mutations.

Binding specificity can be determined by any suitable assay in the art, for example but not limited competition binding assays, epitope mapping, etc. For example, as described in Example 1, epitope mapping can be conducted by using cells expressing HLA-E single chain trimers (SCTs) presenting different peptides including VL9 peptides with single amino acid mutations. The cells are incubated with an antibody to be tested and stained with secondary antibodies. Binding specificity is measured by counting the number of positively-stained cells using flow cytometry where specific binding to an HLA-E-peptide complex of interest is shown by differences in the number of positively stained cells as compared to experiments using cells that express HLA-E in complex with control and/or mutant peptides. For example, binding specificity can be determined by testing whether the antibody can bind to cells pulsed with a peptide of interest and that express HLA-E and is HLA class I negative; but not to the same cells pulsed with a negative control peptide or mutant peptides with sequences that differ from the peptide of interest. For example, in Example 1, antibody 3H4 was found to be able to preferentially bind HLA-E-peptide complexes with peptide variants of the classical VL9 motif, i.e., able to bind to complexes with peptides mutated at position 1 to alanine, cysteine, isoleucine, serine, threonine, valine, histidine, proline; but not to peptides mutated at position 1 to arginine, glutamate, glycine, lysine, methionine, asparagine, tryptophan, tyrosine, or phenylalanine. (See, Example 1.) Thus, in some embodiments, binding specificity can be determined in the context of epitope mapping where a peptide of interest is mutated and loaded into HLA-E complexes to test for differences in binding.

In some embodiments, an antibody or fragment of the invention has a binding specificity characterized by its ability to preferentially bind to an HLA-E-VL9 complex, where the peptide conforms to a VL9 motif as used herein or a variant thereof, but is not able to bind to a control HLA-E-peptide complex where the peptide is the RL9HIV peptide or the RL9SIV peptide. In some embodiments, an antibody or fragment preferentially binds to an HLA-E-viral peptide complex. In some embodiments, an antibody or fragment preferentially binds to an HLA-E-microbiome complex.

Another binding specificity assay can test whether the antibody can specifically bind to soluble HLA-E-peptide complexes using ELISA or SPR (or HLA-E-peptide complexes are immobilized and the antibody is soluble). (See, e.g., Example 1, where HLA-E-VL9 peptides were used.) Control antibodies that bind to HLA-E but are not specific to the HLA-E-VL9 complex are known in the art, for example, the pan-HLA-E mAb 3D12.

Affinity can be measured, for example, by surface plasmon resonance. It is well-known in the art how to conduct SPR for measuring antibody affinity to an antigen. SPR affinity measurements can provide the affinity constant KD of an antibody, which is based on the association rate constant kₒₙ divided by the disassociation rate constant k_{off}. Thus, in certain embodiments, comparing affinity between a variant and an antibody of Table 1 is based on KD. In other embodiments, the comparison is based only on k_{off}. When comparing affinity between antibodies, the antibodies should have the same valency, i.e., Fab vs. Fab, scFv vs. scFv, IgG v. IgG, IgM v. IgM, etc. Thus, when the comparison is between antibodies that are not monovalent, then affinity is a measure of functional affinity. In the art, functional affinity covers the binding strength of a bi- or polyvalent antibody to antigens that present more than one copy of an epitope, because they are multimeric or conjugated in multiple copies to a solid phase, thus allowing cross-linking by the antibody. It is known in the art that a monovalent antibody fragment (e.g., Fab) provides a measure of intrinsic affinity irrespective of the density of antigens (SPR often immobilizes antigen on a solid substrate and the antibody is flowed over the substrate thereby allowing kinetic measurements of antibody association and disassociation rates).

Avidity can also be measured by SPR. Avidity can be quantitatively expressed, for example, by the ratio of KD for a Fab over the multivalent form, e.g., IgG, IgM et al.

Potency can be measured, for example, by a NK cell or CD8+ T-cell cytotoxicity assay as known in the art. (See, e.g., Example 1, NK cell cytotoxicity assay.) Herein, the cytotoxicity assay utilizes different HLA-E-peptide complex expressing cells as the target cells. Target cells are incubated with antibodies and NKG2A+ NK cells or NKG2A+ CD8+ T-cells (effector cells) and pulsed with ⁵¹Cr (chromium). Cytotoxicity is measured as a percentage of specific lysis according to the following formula: (⁵¹Cr Release %)=[(Experimental Release−Spontaneous Release)/(Maximum Release−Spontaneous Release)]×100.

In certain embodiments, the invention provides antibodies with CDR amino acid sequences that are 99%, 98%, 97%, 96%, 95%, 94%, 93%, 92%, 91%, 90%, 89%, 88%, 87%, 86%, 85%, 84%, 83%, 82%, 81%, 80% identical to the CDR1, 2, and/or 3 of Vh (also referred to as CDRH1, CDRH2, and CDRH3) and/or CDR1, 2, and/or 3 of Vl (also referred to as CDRL1, CDRL2, and CDRL3) amino acid sequences of the antibodies of Table 1 or the amino acid sequences translated from the nucleotide sequences of the antibodies of FIG. 22E.

In certain embodiments, the invention provides antibodies with CDR amino acid sequences that are 99%, 98%, 97%, 96%, 95%, 94%, 93%, 92%, 91%, 90%, 89%, 88%, 87%, 86%, 85%, 84%, 83%, 82%, 81%, 80% identical to CDRs to an antibody of Table 1 or the amino acid sequences translated from the nucleotide sequences of the antibodies of FIG. 22E, where each CDR can have a different percent identity. For example, the antibody has at least 99%, 98%, 97%, 96%, or 95% identity for all CDRs as compared to the CDRs of an antibody listed in Table 1 or FIG. 22 except HCDR3 and LCDR3, which can allow for a lower percent identity, for example, 99% to 80%, 99% to 85%, 99% to 90%, or 99% to 95%.

In certain embodiments, the invention provides antibodies which can tolerate a larger percent variation in the sequences outside of the Vh and/Vl sequences of the antibodies. In certain embodiments, the invention provides antibodies which are 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65% identical, wherein the identity is outside of the Vh or V1 regions, or the CDRs of the Vh or V1 chains of the antibodies described herein.

In some aspects, the antibody or antigen binding fragment thereof, comprises a heavy chain comprising at least one CDRH1, at least one CDRH2 and at least one CDRH3 and a light chain comprising at least one CDRL1, at least one CDRL2 and at least one CDRL3, wherein at least one CDR, comprises, consists essentially of or consists of an amino acid sequence according to any of the sequences in FIG. 8 or FIG. 22 (translated from the nucleotide sequences), or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments, the functional variation is 80%, 81%, 82%, 83%, 84%, 85%, 86%, 87%, 88%, 89%, 90%, 91%, 92%, 93%, 94%, 95%, 96%, 97%, 98% or 99% identity. As stated herein, the person of ordinary skill in the art can select from one or more CDR conventions to identify the boundaries of the CDR regions.

In some aspects, the antibody or antigen binding fragment thereof, comprises a heavy chain comprising at least one CDRH1, at least one CDRH2 and at least one CDRH3 and a light chain comprising at least one CDRL1, at least one CDRL2 and at least one CDRL3, wherein at least one CDR, comprises, consists essentially of or consists of an amino acid sequence according to any of the sequences in FIG. 6, FIG. 8 or FIG. 22 (translated from the nucleotide sequences), or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments further wherein the antibody or antigen binding fragment thereof comprises framework regions corresponding to the heavy and light chain gene usage of the HLA-E-VL9-specific antibodies described herein. For human antibodies see FIG. 22C, columns R, S and T, 22D columns AA and AB, and for mouse antibodies see Table 5 for non-limiting embodiments of gene usage for human and mouse antibodies respectively. For example, in some non-limiting embodiments, the heavy chain of the antibody or antigen binding fragment thereof comprises a framework region corresponding to VH gene 3-21, 3-23, 3-48, 3-74, 3-11, 4-30-2, 3-30, 1-18, 1-69, 4-61, 3-30-3, 3-15, or 4-59. For example, in some non-limiting embodiments, the light chain of the antibody or antigen binding fragment thereof comprises a framework region corresponding to a kappa light chain. For example, in some non-limiting embodiments, the light chain of the antibody or antigen binding fragment thereof comprises a framework region corresponding to a lambda light chain. For example, in some non-limiting embodiments, the light chain of the antibody or antigen binding fragment thereof comprises a framework region corresponding to VL gene 3-21, 1-44, 2-14, 5-39, 2-8, or 2-23 or VK gene 3-20, 1-39, 1-16, 3-15, 1-33, 2-28, 3-11, 1-9, 2-14, 1-5, 1-8, 4-1, 1D-12, 6-21, 1-17. In some embodiments, the antibody or antigen binding fragment thereof comprises framework regions corresponding to the heavy and light chain gene and allele usage of the HLA-E-VL9-specific antibodies described herein. For example, in some non-limiting embodiments, the heavy chain of the antibody or antigen binding fragment thereof comprises a framework region corresponding to the *01, *04, *18, *05, *03, *06, or *12 allele of VH gene. For example, in some non-limiting embodiments, the light chain of the antibody or antigen binding fragment thereof comprises a framework region corresponding to the *01, *02, or *03 allele of the VL or VK gene.

In some aspects, the antibody or antigen binding fragment thereof, comprises, consists essentially of or consists of a Vh amino acid sequence or a Vl amino acid sequence in FIG. 8 or FIG. 22 or a functional sequence variant thereof having at least 70%, at least 75%, at least 80%, at least 85%, at least 88%, at least 90%, at least 92%, at least 95%, at least 96%, at least 97%, at least 98% or at least 99% sequence identity. In certain embodiments the functional variation is 80%, 81%, 82%, 83%, 84%, 85%, 86%, 87%, 88%, 89%, 90%, 91%, 92%, 93%, 94%, 95%, 96%, 97%, 98% or 99% identity.

In some respects, the antibody or antigen-binding fragment thereof, comprises, consists essentially of or consists of a Vh amino acid sequence and/or a Vl amino acid sequence according to FIG. 8 or translated from FIG. 22E.

Furthermore, the invention provides antibodies that are affinity matured in vitro. The affinity of an antibody to its antigen target can be modulated by identifying mutations introduced into the variable region generally or into targeted sub-regions. For example, it is known in the art that one can sequentially introduce mutations through each of the CDRs, optimizing one at a time, or to focus on CDRH3 and CDRL3, or CDRH3 alone, because it often forms the majority of antigen contacts. Alternatively, it is known in the art how to simultaneously mutagenize all six CDRs by generating large-scale, high-throughput expression and screening assays, such as by antibody phage display. Antibody-antigen complex structural information can also be used to focus affinity maturation to a small number of residues in the antibody binding site.

Various algorithms for sequence alignment are known in the art. For example, the NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

### CDRs and Frameworks

In some embodiments, the CDRs of the antibodies and fragments of the invention are defined according to the IMGT scheme. IMGT-defined CDR regions have been highlighted/underlined in the nucleotide and amino acid sequences for each of the Vh and Vl variable regions of the antibodies of Table 1. (See FIGS. 7 and 8.) IMGT sequence analysis tools will identify CDR and framework regions in the nucleotide sequence and translated amino acid sequence. See http://www.imgt.org/IMGT_vquest/analysis

In some embodiments, CDR and framework regions can be identified based on other classical variable region numbering and definition schemes or conventions, including the Kabat, Chothia, Martin, and Aho schemes. The ANARCI (Antigen receptor Numbering And Receptor Classification; see http: opig.stats.ox.ac.uk/webapps newsabdab sabpred/anarci/) online tool allows one to input amino acid sequences and to select an output with the IMGT, Kabat, Chothia, Martin, or AHo numbering scheme. With these numbering schemes, CDR and framework regions within the amino acid sequence can be identified. The person of ordinary skill is able to ascertain CDR and framework boundaries using one or more of several publicly available tools and guides.

For example, Table 2 below provides a general, not limiting guide, for the CDR regions as based on different numbering schemes (see http: www.bioinf.org.uk/abs/info.htm/#cdrid). In the Table, any of the numbering schemes can be used for these CDR definitions, except the Contact CDR definition uses the Chothia or Martin (Enhanced Chothia) numbering.

TABLE 2
General Guide of Selected CDR Definitions

| | Kabat | AbM | Chothia | Contact | IMGT |
|---|---|---|---|---|---|
| | CDR | CDR | CDR | CDR | CDR |
| CDR Loop | Definition | Definition | Definition | Definition | Definition |
| CDRL1 | L24-L34 | L24-L34 | L24-L34 | L30-L36 | L27-L32 |
| CDRL2 | L50-L56 | L50-L56 | L50-L56 | L46-L55 | L50-L51 |
| CDRL3 | L89-L97 | L89-L97 | L89-L97 | L89-L96 | L89-L97 |
| CDRH1 | H31-H35B | H26-H35B | H26- | H30-H35B | H26-H35B |
| (Kabat | | | H32..H34 | | |
| numbering) | | | | | |
| CDRH1 | H31-H35 | H26-H35 | H26-H32 | H30-H35 | H26-H33 |
| (Chothia | | | | | |
| numbering | | | | | |
| CDRH2 | H50-H65 | H50-H58 | H52-H56 | H47-H58 | H51-H56 |
| CDRH3 | H95-H102 | H95-H102 | H95-H102 | H93-H101 | H93-H102 |

In Table 2 above, for CDRH1 Kabat numbering using the Chothia CDR definition, the boundary is H26 to H32 or H34 because the Kabat numbering convention varies between H32 and H34 depending on the length of the loop. This is because the Kabat numbering scheme places insertions at H35A and H35B. If neither H35A nor H35B is present, CDRH2 ends at H32. If only H35A is present, the loop ends at H33. If both H35A and H35B are present, the loop ends at H34.

Different methods of identifying CDRs are well known and described in the art (e.g. Kabat, Chothia, IMGT). Delineating CDRs by any one of these methods would result in CDRs with specific boundaries within a VH or VL sequence as listed herein. CDRs identified by any one of the methods are specific and well defined. See, for example, Martin, A. C. R., “Chapter 3: Protein Sequence and Structure Analysis of Antibody Variable Domains,” Antibody Engineering, vol. 2 (2nd ed.), Springer-Verlag, Berlin Heidelberg pp. 33-51 (2010) (describing inter alia Kabat, Chothia, IMGT); and Munshaw, S. and Kepler, T. B., “SoDA2: a Hidden Markov Model approach for identification of immunoglobulin rearrangements,” Bioinformatics, vol. 26, No. 7, pp. 867-872 (February 2010) (describing SoDA2). Any of these methods for identifying CDRs may be used with the instant technology.

Alternatively or in combination, one can examine amino acid sequences and identify CDR and framework regions according to the following alternative general guideline of Table 3, which is non-limiting.

TABLE 3
General Guide for Demarcating CDR Boundaries

| CDR Region | Guidelines |
|---|---|
| LCDR1 | Start: approx. residue 24 |
| | Residue before: always a Cys |
| | Residue after: always a Trp. Typically Trp-Tyr-Gln, but also, Trp-Leu- |
| | Gln, Trp-Phe-Gln, Trp-Tyr-Leu |
| | Length: 10 to 17 residues |
| LCDR2 | Start: always 16 residues after the end of L1. Residues before generally |
| | Ile-Tyr, but also, Val-Tyr, Ile-Lys, Ile-Phe |
| | Length: always 7 residues (except NEW (7FAB) which has a deletion in |
| | this region) |
| LCDR3 | Start: always 33 residues after end of L2 (except NEW (7FAB) which has |
| | the deletion at the end of CDR-L2). Residue before always Cys. Residues |
| | after |
| | always Phe-Gly-XXX-Gly |
| | Length |
| | 7 to 11 residues |
| HCDR1 | Start |
| | Approx. residue 26 (always 4 after a Cys) [Chothia/AbM definition]; |
| | Kabat definition starts 5 residues later |
| | Residues before |
| | always Cys-XXX-XXX-XXX |
| | Residues after |
| | always a Trp. Typically Trp-Val, but also, Trp-Ile, Trp-Ala |
| | Length |
| | 10 to 12 residues [AbM definition]; |
| | Chothia definition excludes the last 4 residues |
| HCDR2 | Start |
| | always 15 residues after the end of Kabat/AbM definition) of CDR-H1 |
| | Residues before |
| | typically Leu-Glu-Trp-Ile-Gly, but a number of variations |
| | Residues after |
| | Lys/Arg-Leu/Ile/Val/Phe/Thr/Ala-Thr/Ser/Ile/Ala |
| | Length |
| | Kabat definition 16 to 19 residues; |
| | AbM (and recent Chothia) definition ends 7 residues earlier |
| HCDR3 | Start |
| | always 33 residues after end of CDR-H2 (always 2 after a Cys) |
| | Residues before |
| | always Cys-XXX-XXX (typically Cys-Ala-Arg) |
| | Residues after |
| | always Trp-Gly-XXX-Gly |
| | Length |
| | 3 to 25(!) residues |

In FIGS. 7 and 8, some CDR boundaries are underlined, and these boundaries are defined according to the IMGT scheme. Because framework (FR) regions constitute all of the variable domain sequence outside of the CDRs, once CDR boundaries are identified, framework regions are necessarily identified. The convention within the art is to label the framework regions as FR1 (sequence before CDR1), FR2 (sequence between CDR1 and CDR2), FR3 (sequence between CDR2 and CDR3), and FR4 (sequence after CDR3).

CDR and framework regions can also be demarcated using other numbering schemes and CDR definitions. The ABnum tool numbers the amino acid sequences of variable domains according to a large and regularly updated database called Abysis, which takes into account insertions of variable lengths and integrates sequences from Kabat, IMGT, and the PDB databases. The Honneger scheme is based on structural alignments of the 3D structures of immunoglobulin variable regions and allows one to define structurally conserved C□ positions and deduction of appropriate framework regions and CDR lengths (Honegger and Plückthun, J. Mol. Biol., 2001, 309:657-70). Similarly, Ofran et al. used a multiple structural alignment approach to identify the antigen binding residues of the variable regions called “Antigen Binding Regions (ABRs)” (Ofran et al., J. Imunol., 2008, 181:6230-5). ABRs can be identified using the Paratome online tool that identifies ABR by comparing the antibody sequence with a set of antibody-antigen structural complexes (Kunik et al., Nucleic Acids Res., 2012, 40:521-4). Another alternative tool is the proABC software, which estimates the probabilities for each residue to form an interaction with the antigen (Olimpieri et al., Bioinformatics, 2013, 29:2285-91).

In some embodiments, the CDRs of the antibodies of the invention are defined by the scheme or tool that provides the broadest or longest CDR sequence. In some embodiments, the CDRs are defined by a combination of schemes or tools that provides the broadest/longest CDRs. For example, from the Table of CDR Definitions above, CDRL1 would be L24-L36, CDRL2 would be L46-L56, CDR3 would be L89-L97, CDRH1 would be H26-H35/H35B, CDRH2 would be H47-H65, and CDRH3 would be H93-H102. In some embodiments, the CDRs are defined by the Anticalign software, which automatically identifies all hypervariable and framework regions in experimentally elucidated antibody sequences from an algorithm based on rules from the Kabat and Chothia conventions (Jarasch et al., Proteins Struct. Funct. Bioinforma, 2017, 85:65-71). In some embodiments, the CDRs are defined by a combination of the Kabat, IMGT, and Chothia CDR definitions. In some embodiments, the CDRs are defined by the Martin scheme in combination with the Kabat and IMGT schemes. In some embodiments, the CDRs are defined by a combination of the Martin and Honneger schemes. In some embodiments, the CDRs comprise the ABR residues identified by the Paratome tool.

The complete human immunoglobulin germline gene loci and alleles are available in the Immunogenetics Database (IMGT). Skilled artisan can readily determine the V, D, and/or J of the heavy and/or light sequences of various embodiments of antibodies of the invention of fragments thereof.

### Fragments and Other Engineered Antibody Forms

In certain embodiments the invention provides antibody fragments, which have the binding specificity and/or properties of the inventive antibodies. Recombinant fragments of the antibodies can be obtained by cloning and expression of part of the sequences of the heavy or light chains.

Antibody “fragments” include Fab, Fab′, F(ab′)2, F(ab)c, diabodies, Dabs, nanobodies, and Fv fragments. Also included are heavy or light chain monomers and dimers, single domain heavy chain antibodies, single domain light chain antibodies, (a single domain antibody, sdAb, is also referred to in the art as a nanobody) as well as single chain antibodies, e.g., single chain Fv in which the heavy and light chain variable domains are joined by a peptide linker. (See, e.g., Bird et al., Science 242:423-426, 1988; Huston et al., Proc. Natl. Acad. Sci. USA 85:5879-5883, 1988; McCafferty et al., Nature 348:552-554, 1990). Optionally, a cleavage site can be included in a linker, such as a furin cleavage site.

A recombinant antibody can also comprise a heavy chain variable domain from one antibody and a light chain variable domain from a different antibody. Further, the invention encompasses chimeric antigen receptors (CARs; chimeric T cell receptors) engineered from the variable domains of antibodies. (Chow et al, Adv. Exp. Biol. Med., 2012, 746:121-41). The Chimeric Antigen Receptor (CAR) consists of an antibody-derived targeting domain (including fragments such as scFv or Fab) fused with T-cell signaling domains that, when expressed by a T-cell, endows the T-cell with antigen specificity determined by the targeting domain of the CAR.

### Fc Domains

Whether full-length, or fragments engineered to have a Fc domain, (or particular constant domain portions thereof), the antibodies of the invention can be of any isotype or have any Fc (or portion thereof) of any isotype. It is well-known in the art how to engineer Fc domains or portions together with antibody fragments.

In certain embodiments, the antibodies of the invention can be used as IgG1, IgG2, IgG3, IgG4, whole IgG1 or IgG3s, whole monomeric IgAs, dimeric IgAs, secretory IgAs, IgMs as monomeric, pentameric, hexameric, or other polymer forms of IgM. The class of an antibody comprising the VH and VL chains described herein can be specifically switched to a different class of antibody by methods known in the art.

In some embodiments, the nucleic acid encoding the VH and VL can encode an Fc domain (immunoadhesin). The Fc domain can be an IgA, IgM or IgG Fc domain. The Fc domain can be an optimized Fc domain, as described in U.S. Published Patent Application No. 20100093979, incorporated herein by reference. In one example, the immunoadhesin is an IgG1 Fc. In one example, the immunoadhesin is an IgG3 Fc.

In one embodiment, the IgG constant region comprises the LS mutation. Additional variants of the Fc portion of the antibody are also contemplated by the invention. See Maeda et al. MAbs. 2017 July; 9(5): 844-853. Published online 2017 Apr. 7, PMID: 28387635; see also Booth et al. MAbs. 2018 October; 10(7): 1098-1110. Published online 2018 Jul. 26. doi: 10.1080/19420862.2018.1490119.

In certain embodiments the antibodies comprise amino acid alterations, or combinations thereof, for example in the Fc region outside of epitope binding, which alterations can improve their properties. Various Fc modifications are known in the art.

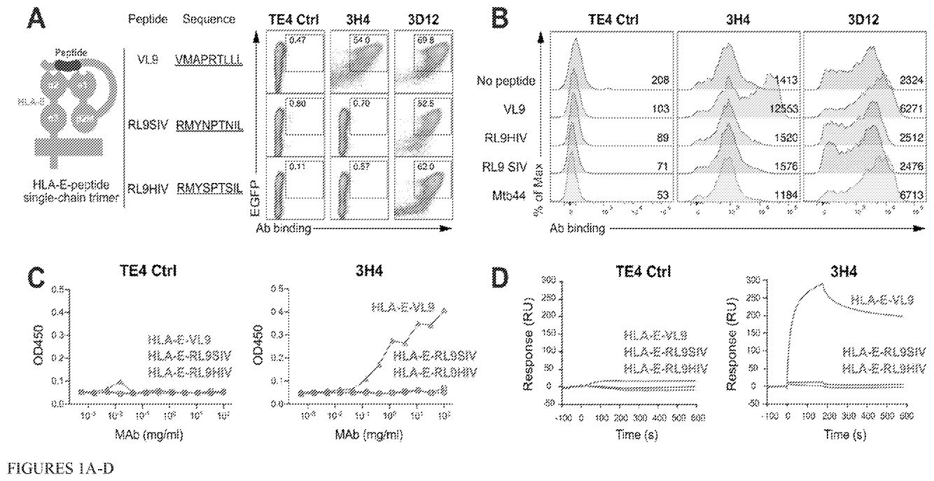

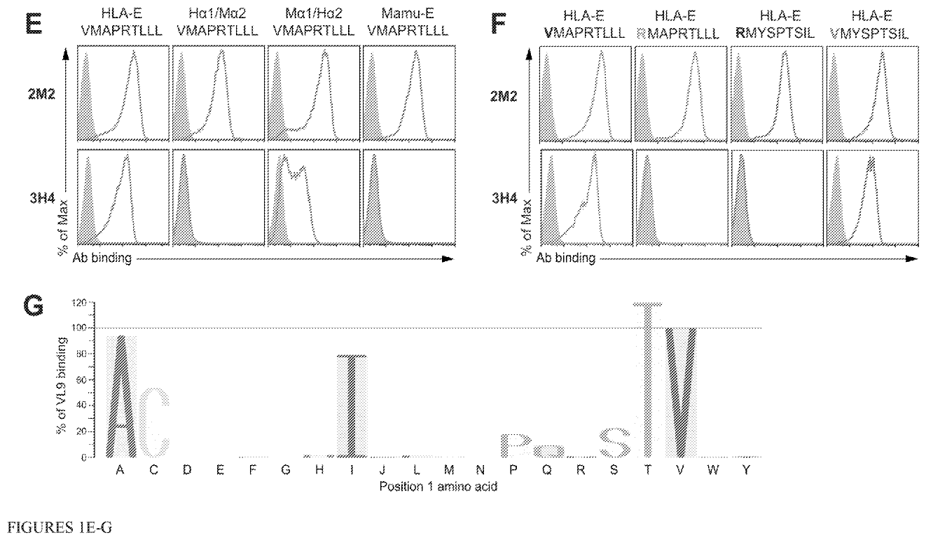

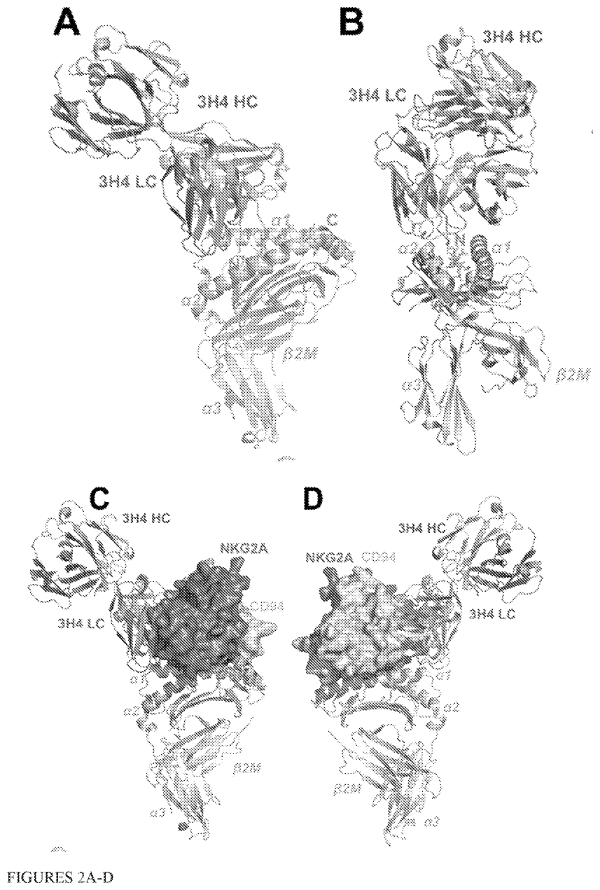

In some embodiments, the invention contemplates antibodies comprising mutations that affect neonatal Fc receptor (FcRn) binding, antibody half-life, and localization and persistence of antibodies at mucosal sites. See e.g. Ko S Y et al., Nature 514: 642-45, 2014, at FIG. 1a and citations therein; Kuo, T. and Averson, V., mAbs 3(5): 422-430, 2011, at Table 1, US Pub 20110081347 (an aspartic acid at Kabat residue 288 and/or a lysine at Kabat residue 435), US Pub 20150152183 for various Fc region mutation, incorporated by reference in their entirety.

In certain embodiments, the antibodies comprise AAAA substitution in and around the Fc region of the antibody that has been reported to enhance ADCC via NK cells (AAA mutations) containing the Fc region aa of S298A as well as E333A and K334A (Shields R I et al JBC, 276: 6591-6604, 2001) and the 4^{th} A (N434A) is to enhance FcR neonatal mediated transport of the IgG to mucosal sites (Shields R I et al. ibid).

Other antibody mutations have been reported to improve antibody half-life or function or both and can be incorporated in sequences of the antibodies. These include the DLE set of mutations (Romain G, et al. Blood 124: 3241, 2014), the LS mutations M428L/N434S, alone or in a combination with other Fc region mutations, (Ko S Y et al. Nature 514: 642-45, 2014, at FIG. 1a and citations therein; Zlevsky et al., Nature Biotechnology, 28(2): 157-159, 2010; US Pub 20150152183) for LS mutation see e.g. Gaudinski M R, Coates E E, Houser K V, Chen G L, Yamshchikov G, Saunders J G, et al. (2018) Safety and pharmacokinetics of the Fc-modified HIV-1 human monoclonal antibody VRC01LS: A Phase 1 open-label clinical trial in healthy adults. PLoS Med 15(1): e1002493. https://doi.org/10.1371/journal.pmed.100249; the YTE Fc mutations (Robbie G et al Antimicrobial Agents and Chemotherapy 12: 6147-53, 2013) as well as other engineered mutations to the antibody such as QL mutations, IHH mutations (Ko S Y et al. Nature 514: 642-45, 2014, at FIG. 1a and relevant citations; See also Rudicell R et al. J. Virol 88: 12669-82, 201).

In some embodiments, modifications, such as but not limited to antibody fucosylation, may affect interaction with Fc receptors (See e.g. Moldt, et al. JVI 86(11): 66189-6196, 2012). In some embodiments, the antibodies can comprise modifications, for example but not limited to glycosylation, which reduce or eliminate polyreactivity of an antibody. See e.g. Chuang, et al. Protein Science 24: 1019-1030, 2015.

In some embodiments the antibodies can comprise modifications in the Fc domain such that the Fc domain exhibits, as compared to an unmodified Fc domain enhanced antibody dependent cell mediated cytotoxicity (ADCC); increased binding to Fc.gamma.RIIA or to Fc.gamma.RIIIA; decreased binding to Fc.gamma.RIIB; or increased binding to Fc.gamma.RIIB. See e.g. US Pub 20140328836.

### Multivalent and Multispecific Antibodies

In certain embodiments the invention provides a multivalent and multispecific antibody. A multivalent antibody has at least two antigen-binding sites, i.e., at least two heavy/light chain pairs, or fragments thereof. When the heavy/light pairs of a multivalent antibody bind to different epitopes, whether on the same antigen or on different antigens, the antibody is considered to be multispecific. Antibody fragments may impart monovalent or multivalent interactions and be contained in a variety of structures as described above. For instance, monovalent scFv molecules may be synthesized to create a bivalent diabody, a trivalent “triabody” or a tetravalent “tetrabody.” The scFv molecules may include a domain of the Fc region resulting in bivalent minibodies. In addition, the sequences of the invention may be a component of multispecific molecules in which the sequences of the invention target the epitopes of the invention and other regions of the molecule bind to other targets. Exemplary molecules include, but are not limited to, bispecific Fab2, trispecific Fab3, bispecific scFv, and diabodies (Holliger and Hudson, 2005, Nature Biotechnology 9: 1126-1136).

In some embodiments, multivalent but not multispecific antibodies are provided, where the multispecific antibody comprises multiple identical Vh/Vl pairs, or the CDRs from the Vh and a Vl pairs. This type of multispecific antibody will serve to improve the avidity of an antibody. For example, a tetramer can comprise four identical scFvs where the scFv is based on the Vh/Vl pair from an antibody of Table 1 or FIG. 22.

In some embodiments, multivalent but not multispecific antibodies comprise multiple Vh/Vl pairs (or the CDRs from the pairs) where each pair binds to an overlapping epitope. Determining overlapping epitopes can be conducted, for example, by structural analysis of the antibodies and competitive binding assays as known in the art.

In some embodiments, multispecific antibodies comprise multiple Vh/Vl pairs (or the CDRs from the pairs) where each pair binds to a distinct epitope (not overlapping) on the HLA-E-VL9 complex.

In some embodiments, multispecific antibodies or fragments of the invention comprise at least a Vh and a Vl pair from Table 1, or the CDRs from the Vh and a Vl pair, in order to provide the multispecific antibody with binding specificity to the HLA-E-VL9 peptide complex. As described below, the multispecific antibody can have one or more additional binding specificities by further comprising antibody binding site fragments from antibodies that bind to different antigens. For example, a multispecific antibody can comprise a Vh/Vl pair that targets the HLA-E-VL9 peptide complex from Table 1 and one or more Vh/Vl pairs that target and block different inhibitory receptors. Other inhibitory receptors include, but are not limited to, NKG2A, CD94, NKG2A/CD94 heterodimer, LAG-3, TIM-3, TIGIT, BTLA, PD-1, and CTLA-4. In other embodiments, a multispecific antibody can comprise a Vh/Vl pair that targets the HLA-E-VL9 peptide complex from Table 1 and one or more Vh/Vl pairs that specifically bind and operate as agonists upon stimulatory receptors for effector cell function. Non-limiting examples of stimulatory receptors for effector cell function include NKG2C, NKG2D, 4-1BB (CD137), OX40 (CD134), TNFRSF7 (CD27), ICOS (CD278), TNFRSF8 (CD30), LFA-2 (CD2), DNAM-1 (CD226), CD3, CD16, CD32, and CD64.

In certain embodiments the invention provides a bispecific antibody. A bispecific or bifunctional/dual targeting antibody is an artificial hybrid antibody having two different heavy/light chain pairs and two different binding sites (see, e.g., Romain Rouet & Daniel Christ “Bispecific antibodies with native chain structure” Nature Biotechnology 32, 136-137 (2014); Garber “Bispecific antibodies rise again” Nature Reviews Drug Discovery 13, 799-801 (2014), FIG. 1a; Byrne et al. “A tale of two specificities: bispecific antibodies for therapeutic and diagnostic applications” Trends in Biotechnology, Volume 31, Issue 11, November 2013, Pages 621-632 Songsivilai and Lachmann, Clin. Exp. Immunol., 79:315-321 (1990); Kostelny et al., J. Immunol. 148:1547-53 (1992) (and references therein)). In certain embodiments the bispecific antibody is a whole antibody of any isotype. In other embodiments it is a bispecific fragment, for example but not limited to F(ab)₂ fragment. In some embodiments, the bispecific antibodies do not include Fc portion, which makes these diabodies relatively small in size and easy to penetrate tissues.

Non-limiting examples of multispecific antibodies also include: (1) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (2) a Tandab, which is a fusion of two single chain diabodies resulting in a tetravalent bispecific antibody that has two binding sites for each of the target antigens; (3) a flexibody, which is a combination of scFvs with a diabody resulting in a multivalent molecule; (4) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (5) a so-called Scorpion molecule, comprising, e.g., two scFvs fused to both termini of a human Fc-region. Examples of platforms useful for preparing bispecific antibodies include but are not limited to BiTE (Micromet), DART (MacroGenics) (e.g., U.S. Pat. No. 8,795,667; US Publications 20090060910; 20100174053), Fcab and Mab2 (F-star), Fc-engineered IgG1 (Xencor) or DuoBody (based on Fab arm exchange, Genmab).

In certain embodiments, the multispecific antibodies can include a Fc region. For example, Fc bearing DARTs are heavier, and could bind neonatal Fc receptor, increasing their circulating half-life. See Garber “Bispecific antibodies rise again” Nature Reviews Drug Discovery 13, 799-801 (2014), FIG. 1a; See US Pub 20130295121, incorporated by reference in their entirety.

In certain embodiments, the invention encompasses multispecific molecules comprising an Fc domain or portion thereof (e.g. a CH2 domain, or CH3 domain). The Fc domain or portion thereof may be derived from any immunoglobulin isotype or allotype including, but not limited to, IgA, IgD, IgG, IgE and IgM. In some embodiments, the Fc domain (or portion thereof) is derived from IgG. In some embodiments, the IgG isotype is IgG1, IgG2, IgG3 or IgG4 or an allotype thereof.

In some embodiments, the multispecific molecule comprises an Fc domain, which Fc domain comprises a CH2 domain and CH3 domain independently selected from any immunoglobulin isotype (i.e. an Fc domain comprising the CH2 domain derived from IgG and the CH3 domain derived from IgE, or the CH2 domain derived from IgG1 and the CH3 domain derived from IgG2, etc.). In some embodiments, the Fc domain may be engineered into a polypeptide chain comprising the multispecific molecule of the invention in any position relative to other domains or portions of the polypeptide chain (e.g., the Fc domain, or portion thereof, may be c-terminal to both the Vl and Vh domains of the polypeptide of the chain; may be n-terminal to both the Vl and Vh domains; or may be N-terminal to one domain and c-terminal to another (i.e., between two domains of the polypeptide chain)).

The present invention also encompasses molecules comprising a hinge domain. The hinge domain be derived from any immunoglobulin isotype or allotype including IgA, IgD, IgG, IgE and IgM. In preferred embodiments, the hinge domain is derived from IgG, wherein the IgG isotype is IgG1, IgG2, IgG3 or IgG4, or an allotype thereof. The hinge domain may be engineered into a polypeptide chain comprising the multispecific molecule together with an Fc domain such that the multispecific molecule comprises a hinge-Fc domain. In certain embodiments, the hinge and Fc domain are independently selected from any immunoglobulin isotype known in the art or exemplified herein. In other embodiments the hinge and Fc domain are separated by at least one other domain of the polypeptide chain, e.g., the Vl domain. The hinge domain, or optionally the hinge-Fc domain, may be engineered into a polypeptide of the invention in any position relative to other domains or portions of the polypeptide chain. In certain embodiments, a polypeptide chain of the invention comprises a hinge domain, which hinge domain is at the C-terminus of the polypeptide chain, wherein the polypeptide chain does not comprise an Fc domain. In yet other embodiments, a polypeptide chain of the invention comprises a hinge-Fc domain, which hinge-Fc domain is at the C-terminus of the polypeptide chain. In further embodiments, a polypeptide chain of the invention comprises a hinge-Fc domain, which hinge-Fc domain is at the N-terminus of the polypeptide chain.

In some embodiments, the invention encompasses multimers of polypeptide chains, each of which polypeptide chains comprise a Vh and a Vl domain, comprising CDRs as described herein. In certain embodiments, the Vl and Vh domains comprising each polypeptide chain have the same specificity, and the multimer molecule is bivalent and monospecific. In other embodiments, the Vl and Vh domains comprising each polypeptide chain have differing specificity and the multimer is bivalent and bispecific. In some embodiments, the polypeptide chains in multimers further comprise an Fc domain. Dimerization of the Fc domains leads to formation of a diabody molecule that exhibits immunoglobulin-like functionality, i.e., Fc mediated function (e.g., Fc-Fc.gamma.R interaction, complement binding, etc.).

One non-limiting approach to multimerize antibodies or fragments uses staphylococcus Sortase A transpeptidase ligation to conjugate antibodies or fragments, for e.g. but not limited to a nanoparticle. To conjugate antibody or fragment thereof to a nanoparticle via a sortase reaction, a C-terminal LPXTG(G) tag or a N-terminal pentaglycine repeat tag is added to the gene encoding antibody or fragment thereof, where X signifies any amino acid, such as Ala, Ser, Glu. A nanoparticle carrying the complementary tag is provided. Sortase A is then used to covalently bond the tagged antibody or fragment thereof to a nanoparticle.

The sortase A-tagged antibody or fragment thereof can also be conjugated to other peptides, proteins, or fluorescent labels. In non-limiting embodiments, the sortase A tagged antibody or fragment thereof are conjugated to ferritin to form nanoparticles. In non-limiting embodiments, ferritin is H. pylori ferritin. Any suitable ferritin can be used. In non-limiting embodiments, ferritin sequences are disclosed in WO/2018/005558.

In some embodiments sequences herein include c-terminal sortase A donor sequences to allow for site specific conjugation to multimerizing scaffolds expressing the n-terminal sortase A acceptor sequence. The donor sequence is a LPXTGG where the third amino acid can vary. In one embodiment X is E. The acceptor sequence is composed of 5 or more glycines appended to the N-terminus.

Any suitable ferritin can be used in the nanoparticles of the invention. In non-limiting embodiments, the ferritin is derived from Helicobacter pylori. In non-limiting embodiments, the ferritin is insect ferritin. In non-limiting embodiments, each nanoparticle displays 24 copies of the spike protein on its surface.

Another non-limiting approach to multimerize antibodies or fragments uses mutations E345R, E430G, S440Y in the Fc region of human gamma immunoglobulin (G1m3 allotype) that allow formation of hexamers (see Diebolder, C A et al Science 343:1260-63 2014).

In yet other embodiments, diabody molecules of the invention encompass tetramers of polypeptide chains, each of which polypeptide chain comprises a Vh and Vl domain. In certain embodiments, two polypeptide chains of the tetramer further comprise an Fc domain. The tetramer is therefore comprised of two ‘heavier’ polypeptide chains, each comprising a Vl, Vh and Fc domain, and two ‘lighter’ polypeptide chains, comprising a Vl and Vh domain. Interaction of a heavier and lighter chain into a bivalent monomer coupled with dimerization of the monomers via the Fc domains of the heavier chains will lead to formation of a tetravalent immunoglobulin-like molecule. In certain aspects the monomers are the same, and the tetravalent diabody molecule is monospecific or bispecific. In other aspects the monomers are different, and the tetravalent molecule is bispecific or tetraspecific.

Formation of a tetraspecific diabody molecule as described supra requires the interaction of four differing polypeptide chains. Such interactions are difficult to achieve with efficiency within a single cell recombinant production system, due to the many variants of potential chain mispairings. One solution to increase the probability of mispairings, is to engineer “knobs-into-holes” type mutations into the desired polypeptide chain pairs. Such mutations favor heterodimerization over homodimerization. For example, with respect to Fc-Fc-interactions, an amino acid substitution (preferably a substitution with an amino acid comprising a bulky side group forming a ‘knob’, e.g., tryptophan) can be introduced into the CH2 or CH3 domain such that steric interference will prevent interaction with a similarly mutated domain and will obligate the mutated domain to pair with a domain into which a complementary, or accommodating mutation has been engineered, i.e., ‘the hole’ (e.g., a substitution with glycine). Such sets of mutations can be engineered into any pair of polypeptides comprising the diabody molecule, and further, engineered into any portion of the polypeptides' chains of the pair. Methods of protein engineering to favor heterodimerization over homodimerization are well known in the art, in particular with respect to the engineering of immunoglobulin-like molecules, and are encompassed herein (see e.g., Ridgway et al. (1996) “Knobs-Into-Holes' Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270: 26-35, and Xie et al. (2005) “A New Format Of Bispecific Antibody: Highly Efficient Heterodimerization, Expression And Tumor Cell Lysis,” J. Immunol. Methods 296:95-101; each of which is hereby incorporated herein by reference in its entirety).

The invention also encompasses diabody molecules comprising variant Fc or variant hinge-Fc domains (or portion thereof), which variant Fc domain comprises at least one amino acid modification (e.g. substitution, insertion deletion) relative to a comparable wild-type Fc domain or hinge-Fc domain (or portion thereof). Molecules comprising variant Fc domains or hinge-Fc domains (or portion thereof) (e.g., antibodies) normally have altered phenotypes relative to molecules comprising wild-type Fc domains or hinge-Fc domains or portions thereof. The variant phenotype may be expressed as altered serum half-life, altered stability, altered susceptibility to cellular enzymes or altered effector function as assayed in an NK dependent or macrophage dependent assay. Fc domain variants identified as altering effector function are known in the art. For example International Application WO04/063351, U.S. Patent Application Publications 2005/0037000 and 2005/0064514.

The bispecific diabodies of the invention can simultaneously bind two separate and distinct epitopes. In certain embodiments the two separate epitopes are on different cells, e.g., HLA-E-VL9 epitope on one cell and a stimulatory receptor epitope on another cell. In certain embodiments, the two separate epitopes are on two different inhibitory receptors on the same cell. In certain embodiments the epitopes are from the same antigen. In other embodiments, the epitopes are from different antigens. In some embodiments, at least one epitope binding site is specific for a determinant expressed on an immune effector cell (e.g. CD3, CD16, CD32, CD64, etc.) which are expressed on T lymphocytes, natural killer (NK) cells or other mononuclear cells. In one embodiment, the diabody molecule binds to the effector cell determinant and also activates the effector cell. In this regard, diabody molecules of the invention may exhibit Ig-like functionality independent of whether they further comprise an Fc domain (e.g., as assayed in any effector function assay known in the art or exemplified herein (e.g., ADCC assay).

In certain embodiments the bispecific antibodies engage cells for Antibody-Dependent Cell-mediated Cytotoxicity (ADCC). In certain embodiments the bispecific antibodies engage natural killer cells, neutrophil polymorphonuclear leukocytes, monocytes and macrophages. In certain embodiments the bispecific antibodies are T-cell engagers. In certain embodiments, the bispecific antibody comprises an HLA-E-VL9 binding fragment and a CD3 binding fragment. Various CD3 antibodies are known in the art. See for example U.S. Pat. No. 8,784,821. The CD3 antibodies may be activating or non-activating to recruit CD8 T cells as effector cells (See for e.g. Sung et al. J Clin Invest. 2015; 125(11):4077-4090. https://doi.org/10.1172/JCI82314). In certain embodiments, the bispecific antibody comprises a HLA-E-VL9 binding fragment and CD16 binding fragment.

In certain embodiments the invention provides antibodies with dual targeting specificity. In certain aspects the invention provides bi-specific molecules that are capable of localizing an immune effector cell to an HLA-E over-expressing cell, such as a tumor cell or a virally infected cell, so as facilitate the killing of this cell. In this regard, bispecific antibodies bind with one “arm” to a surface antigen on target cells, and with the second “arm” to an activating, invariant component of the T cell receptor (TCR) complex or to an activating, invariant component of a different stimulatory receptor such as NKG2C on NK cells or other immune effector cells. The simultaneous binding of such an antibody to both of its targets will force a temporary interaction between target cell and effector cell, causing, for example, activation of any cytotoxic T cell or NK cell and subsequent lysis of the target cell. Hence, the immune response is re-directed to the target cells and may be independent of classical MHC class I peptide antigen presentation by the target cell or the specificity of the T cell as would be relevant for normal MHC-restricted activation of CTLs. In this context it is crucial that CTLs are only activated when a target cell is presenting the bispecific antibody to them, i.e. the immunological synapse is mimicked. Particularly desirable are bispecific antibodies that do not require lymphocyte preconditioning or co-stimulation in order to elicit efficient lysis of target cells.

Several bispecific antibody formats have been developed and their suitability for T cell mediated immunotherapy investigated. Out of these, the so-called BiTE (bispecific T cell engager) molecules have been very well characterized and already shown some promise in the clinic (reviewed in Nagorsen and Bauerle, Exp Cell Res 317, 1255-1260 (2011)). BiTEs are tandem scFv molecules wherein two scFv molecules are fused by a flexible linker. Further bispecific formats being evaluated for T cell engagement include diabodies (Holliger et al., Prot Eng 9, 299-305 (1996)) and derivatives thereof, such as tandem diabodies (Kipriyanov et al., J Mol Biol 293, 41-66 (1999)). DART (dual affinity retargeting) molecules are based on the diabody format that separates cognate variable domains of heavy and light chains of the two antigen binding specificities on two separate polypeptide chains but feature a C-terminal disulfide bridge for additional stabilization (Moore et al., Blood 117, 4542-51 (2011)). The invention also contemplates Fc-bearing DARTs. The so-called triomabs, which are whole hybrid mouse/rat IgG molecules and also currently being evaluated in clinical trials, represent a larger sized format (reviewed in Seimetz et al., Cancer Treat Rev 36, 458-467 (2010)).

The invention also contemplates bispecific molecules with enhanced pharmacokinetic properties. In some embodiments, such molecules are expected to have increased serum half-life. In some embodiments, these are Fc-bearing DARTs (see supra).

In certain embodiments, such bispecific molecules comprise one portion which targets HLA-E-VL9 and a second portion which binds a second target. In certain embodiments, the first portion comprises Vh and Vl sequences, or CDRs from the antibodies described herein. In certain embodiments, the second target could be, for example but not limited to an effector cell. In certain embodiments the second portion is a T-cell engager. In certain embodiments, the second portion comprises a sequence/paratope which targets CD3, CD16, or another suitable target. In certain embodiments, the second portion is an antigen-binding region derived from a CD3 antibody, optionally a known CD3 antibody. In certain embodiments, the anti-CD antibody induce T cell-mediated or NK-mediated killing. In certain embodiments, the bispecific antibodies are whole antibodies. In other embodiments, the dual targeting antibodies consist essentially of Fab fragments. In other embodiments, the dual targeting antibodies comprise a heavy chain constant region. In certain embodiments, the bispecific antibody does not comprise Fc region. In certain embodiments, the bispecific antibodies have improved effector function. In certain embodiments, the bispecific antibodies have improved cell killing activity. Various methods and platforms for design of bispecific antibodies are known in the art. See for example US Pub. 20140206846, US Pub. 20140170149, US Pub. 20090060910, US Pub 20130295121, US Pub. 20140099318, US Pub. 20140088295 which contents are herein incorporated by reference in their entirety.

The invention also provides trispecific antibodies comprising binding specificities of the invention antibodies. Non-limiting embodiments of trispecific format is described in Xu et al. Science 6 Oct. 2017, Vol. 358, Issue 6359, pp. 85-90.

The invention also provides CAR-T cell designs which comprise antigen binding portions or fragments incorporating portions of Vh and Vl sequences as described herein.

### Chimeric and Humanized Antibodies

Recombinant antibodies include chimeric and humanized forms of non-human Vh and Vl sequences, or portions thereof. For example, chimeric and humanized antibodies can be based on the murine antibodies listed in Table 1. For some of these antibodies, the CDR regions based on the IMGT convention are underlined. In particular, antibody variable regions of the Fab is a major focus of engineering because of its role in antigen or target binding. The antigen combining site is formed by the combination of the six CDR or hypervariable regions, three from the heavy chain and three from the light chain.

Chimeric antibodies are well-known in the art and have a design where the non-human Vh and Vl variable domain sequences are spliced together with human heavy chain and light chain constant domain sequences. Generally, humanized antibodies are created by combining at the genetic level (engineering, grafting), the CDR regions of a non-human antibody (usually murine) with the framework sequences of a human antibody variable domain.

As discussed below, many of the humanization techniques involve engineering one or more of the six non-human antibody CDRs onto different templates or frameworks. In some embodiments of the invention, all six CDRs are grafted, in others just CDRH3 and CDRL3. Depending on the humanization technique, it often involves retaining those non-human framework residues that influence antigen-binding activity.

In some embodiments, the humanized antibody comprises Vl domain framework regions that are derived from a human antibody having a Vl domain amino acid sequence that is most similar or identical to the Vl domain amino acid sequence of the murine antibody, and wherein the Vh domain framework regions are derived from a human antibody having a Vh domain amino acid sequence that is most similar or identical to the Vh domain amino acid sequence of the murine antibody.

In some embodiments, the humanized antibody comprises Vh domain framework regions are derived from a human antibody having a Vh domain that has the most similar three-dimensional structure to the Vh domain of the murine antibody, and wherein the Vl domain framework regions are derived from a human antibody having a Vl domain that has the most similar three-dimensional structure to the Vl domain of the murine antibody.

In some embodiments, the humanized antibody comprises Vh domain framework regions derived from IGHV3-21, IGHV3-11, IGHV3-23, IGHV1-69, or IGHV3-48.

In some embodiments, the humanized antibody comprises Vl domain framework regions are derived from IGKV3-15, IGKV3-20, IGKV1-39, IGKV3-11, or IGKV1-5.

There are several humanization approaches known in the art. CDR grafting is the traditional approach, where non-human CDRs are engineered onto human framework regions, while retaining only those murine framework residues deemed important for the integrity of the antigen-binding site. (Jones et al., Nature, 1986, 321:522-25.) There are several strategies for selecting the human framework. In the “fixed frameworks” strategy, the human framework is fixed regardless of the parental antibody or its sequence similarity. Usually the human myeloma antibodies REI for the light chain and NEW for the heavy chain are used. In the “best fit” or “homology matching” approach, human frameworks are selected based on shared sequence similarities to the parental antibody's variable regions. In the “consensus” approach, human frameworks are based on the consensus sequence of subgroups in the Kabat database. The consensus sequence of each Kabat subgroup is composed of the most frequent amino acid at each framework position. Consensus sequences for the Vh and Vl most similar to the non-human sequences are chosen for CDR grafting. In the “germline” approach, human immunoglobulin germline genes most similar to the non-human Vl and Vh sequences are selected. The complete human immunoglobulin germline gene loci and alleles are available in the Immunogenetics Database (IMGT). Regardless of the approach for selecting human frameworks, “backmutations” are often conducted, which involves changing one or more residues of the human framework back to non-human residues. Considerations for backmutations include whether framework residues can directly interact with antigen, affect packing and orientation of the Q-sheets of the variable domain that might affect the topography of the antigen binding site. All CDRs except HCDR3 have a limited repertoire of structural conformations and therefore are categorized into canonical classes. (Chothia & Lesk, J. Mol. Biol., 1987, 196:901-17.) A few critical residues in each class have been identified as being conserved in order to retain CDR conformation. Once the canonical class is identified for each non-human CDR, then backmutation of the critical/conserved residues in the human frameworks to their non-human counterpart is conducted as they usually are important for maintaining proper CDR conformation. (See also, SAbPred and PylgClassify for CDR structure prediction and classification; see proABC and Paratome for paratope identification that includes CDR residues and FR residues.) Backmutations are tested for their effect on antigen binding.

Specificity-determining residue (SDR) grafting is similar to CDR grating, but involves grafting a subset of residues in the CDRs that are more variable and are directly involved in the interaction with antigen as compared to more conserved residues in CDRs that maintain the conformations of CDR loops. An SDR-grafted humanized antibody is constructed by grafting the SDRs and the residues maintaining the conformations of the CDRs onto a human template/scaffold/framework. The choice of a human template can be based on selecting human antibody framework sequence(s) that exhibit the closest Vh/Vl angles compared to those in the parental non-human antibody for a correct positioning of the CDRs in the humanized construct. (See Abangle or PAPS software for angle analysis.) The SDRs are identified from the 3D structure of the antigen-antibody complex, computational analysis of three-dimensional structures of antibody:antigen complexes in databases, and/or by mutational analysis of the antibody-combining site. (De Pascalis et al., J. Immunol., 2002, 169:3076-84.) SDRs are mainly in CDRH1, in the N-terminal and middle regions of CDRH2, CDRH3 but not in the terminal region, C-terminal region of CDRL1, the first and sometimes middle parts of CDRL2, and in the middle region of CDRL3. (Padlan et al., FASEB J., 1995, 9:133-9.)

Reshaping or veneering involves replacing only the surface residues of the non-human variable regions with human residues while maintaining the non-human core and CDRs. (Pedersen et al., J. Mol. Biol., 1994, 235:959-73; Padlan, Mol. Immunol., 1991, 28:489-98.) Surface residues can be identified according to a defined set of positions in the heavy and light chain variable regions that are thought to describe the exposed framework surface of the Fv regions, and those residues that are non-human are subsequently backmutated. (Id.; see also Staelens et al., Mol. Immunol., 2006, 43:1243-57.)

Superhumanization is also a CDR-grafting approach, but it focuses on structural homologies between the non-human CDRs and human CDRs. Superhumanization involves selecting variable region framework sequences from human antibody genes by comparing canonical CDR structure types for CDR sequences of the variable region of a non-human antibody to canonical CDR structure types for corresponding CDRs from a library of human antibody sequences, preferably germline antibody gene segments. Human antibody variable regions having similar canonical CDR structure types to the non-human CDRs form a subset of member human antibody sequences from which to select human framework sequences. Human germline V genes are identified that have the same canonical structure class as the non-human antibody to be humanized, and those human gene segments are selected whose CDRs have the best residue-to-residue homology to the non-human antibody. In the selected sequences, non-homologous CDR residues in the human gene segments are converted to the non-human antibody sequence. (Tan et al., J. Immunol., 2002, 169:1119-1125; U.S. Pat. No. 7,732,578.)

Human string content (HSC) optimization quantifies humanness of a non-human antibody by counting 9-mer stretches in the non-human Fv region that perfectly matches corresponding stretches in human germline sequences. (Lazar et al., Mol. Immunol., 2007, 44:1986-98.) This approach utilizes the homology present in human germline sequences to make non-human to human substitutions that increase the human sequence content of the non-human Fv region. The humanness of the resulting Fv is derived from several discrete germline sequences, and positions that are not within or proximal to CDRs and the Vh/Vl interface are optimized in the process.

In “framework shuffling,” a non-human antibody is humanized by synthesizing a combinatorial library comprised of its six CDRs fused in frame to a pool of mixed and matched human germline frameworks. (Dall'Acqua et al., Methods, 2005, 36:43-60.) The human frameworks encompass all known heavy and light chain human germline genes. Libraries of the CDR-framework pools are cloned into phage expression vectors and screened for binding to the antigen. Framework shuffling does not require rational design from sequence analysis, structural information, or backmutations.

In “human framework adaptation (HFA),” human germline genes are selected based on sequence and structural considerations. (Fransson et al. 2010.) An expression library is first constructed by combining the binding site of the non-human antibody with human germline genes. The binding site includes CDRs (Kabat definition) and also hypervariable loops (Chothia definition). The size of the library is intentionally limited from sequence and structural considerations. Further, human genes for frameworks are not shuffled. Rather, only frameworks coming from the same human heavy or light chain genes are used.

### Production of Antibodies

Antibodies, preferably monoclonal antibodies, according to the invention can be made by any method known in the art.

In certain embodiments, plasma cells are cultured in limited numbers, or as single plasma cells in microwell culture plates. Antibodies can be isolated from the plasma cell cultures. Vh and Vl can be isolated from single cell sorted plasma cells. From the plasma cell, RNA can be extracted, and PCR can be performed using methods known in the art. The Vh and Vl regions of the antibodies can be amplified by RT-PCR (reverse transcriptase PCR), sequenced and cloned into intermediate vectors for further engineering or into an expression vector that is then transfected into HEK293T cells or other host cells as described below or known in the art. The cloning of nucleic acid in intermediate vectors, expression vectors, the transfection of host cells, the culture of the transfected host cells and the isolation of the produced antibody can be done using any methods known to one of skill in the art. Antibody isolation and purification techniques are known in the art, which can include filtration, centrifugation and various chromatographic methods such as HPLC or affinity chromatography. Techniques for purification of antibodies, e.g., monoclonal antibodies, including techniques for producing pharmaceutical-grade antibodies (and at a sufficiently high concentration or titer for therapeutic use), are well known in the art.

In some embodiments, the antibodies or fragments of the invention have an IgM Fc region or constant domains thereof. It is established that IgM can assume both pentameric and hexameric configurations, depending on the substitution of the J-chain with an additional Fab(2) monomer, which increases the number of Fabs on a single IgM from 10 to 12 (Hiramoto et al Sci. Adv. 2018; 4: eaaul 199; Moh E S et al J Am Soc Mass Spectrom. 2016 July; 27(7):1143-55). Methods for the recombinant production of polymeric IgM (both with and without J chain) have been described (Chromikova et al., Cytotechnology, 2015, 67:343-356; Gilmour et al. Transfusion Medicine, 2008. 18:167-174; Hennicke et al., PLoS ONE, 2020, 15(3):e0229992). Stable or transient IgM producing cells lines can be generated as described in Chromikova et al., 2015 and Hennicke et al., 2020, where two different pIRES expression vectors are used, one to express the heavy chain and the other to express the light chain and the J-chain sequence. IgM antibodies can be purified according to standard methods in the art, including IgM specific resins for use in affinity chromatography (e.g., POROS Capture Select IgM Affinity Matrix by ThermoFisherScientific.) Transmission electron microscopy (TEM) can be used to confirm pentameric and hexameric forms of IgM.

In addition. besides expression constructs that encode antibody fragments or elements, protein fragments of antibodies can be obtained by methods that include digestion with enzymes, such as pepsin or papain, and/or by cleavage of disulfide bonds by chemical reduction.

Any suitable host cell/vector system may be used for expression of the DNA sequences encoding the antibody molecules of the present invention or fragments thereof. Bacterial, for example E. coli, and other microbial systems may be used, in part, for expression of antibody fragments such as Fab and F(ab′)2 fragments, and especially Fv fragments and single chain antibody fragments, for example, single chain Fvs. eukaryotic, e.g., mammalian, host cell expression systems may be used for production of larger antibody molecules, including complete antibody molecules. Suitable mammalian host cells include, but are not limited to, CHO, HEK293T, PER.C6, NS0, myeloma or hybridoma cells. Mammalian cell lines suitable for expression of therapeutic antibodies are well known in the art.

The antibody molecule may comprise only a heavy or light chain polypeptide, in which case only a heavy chain or light chain polypeptide coding sequence needs to be used to transfect the host cells. For production of products comprising both heavy and light chains, the cell line may be transfected with two vectors, a first vector encoding a light chain polypeptide and a second vector encoding a heavy chain polypeptide. Alternatively, a single vector may be used, the vector including sequences encoding light chain and heavy chain polypeptides. Alternatively, antibodies according to the invention may be produced by (i) expressing a nucleic acid sequence according to the invention in a host cell, e.g. by use of a vector according to the present invention, and (ii) isolating the expressed antibody product. Additionally, the method may include (iii) purifying the isolated antibody. Transformed B cells and cultured plasma cells may be screened for those producing antibodies of the desired specificity or function.

The screening step may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

B cell clones or transfected host-cells of the invention can be used in various ways e.g., as a source of monoclonal antibodies, as a source of nucleic acid (DNA or mRNA) encoding a monoclonal antibody of interest, for research, etc.

Expression from recombinant sources is common for pharmaceutical purposes than expression from B cells or hybridomas e.g., for reasons of stability, reproducibility, culture ease, etc.

Thus the invention also provides a method for preparing a recombinant cell, comprising the steps of: (i) obtaining one or more nucleic acids (e.g., heavy and/or light chain mRNAs) from the B cell clone or the cultured plasma cells that encodes the antibody of interest; (ii) inserting the nucleic acid into an expression vector and (iii) transfecting the vector into a host cell in order to permit expression of the antibody of interest in that host cell.

Similarly, the invention provides a method for preparing a recombinant cell, comprising the steps of: (i) sequencing nucleic acid(s) from the B cell clone or the cultured plasma cells that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest, wherein the nucleic acids are nucleic acids that were derived from a cell sorted B cell or a cultured plasma cell of the invention.

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

Any suitable host cells could be used for transfection and production of the antibodies of the invention. The transfected host cell may be a eukaryotic cell, including yeast and animal cells, particularly mammalian cells (e.g., CHO cells, NS0 cells, human cells such as PER.C6 or HKB-11 cells, myeloma cells, or a human liver cell), as well as plant cells. In certain embodiments, expression hosts can glycosylate the antibody of the invention, particularly with carbohydrate structures that are not themselves immunogenic in humans. In one embodiment, the transfected host cell may be able to grow in serum-free media. In a further embodiment, the transfected host cell may be able to grow in culture without the presence of animal-derived products. The transfected host cell may also be cultured to give a cell line.

In general, protein therapeutics are produced from mammalian cells. The most widely used host mammalian cells are Chinese hamster ovary (CHO) cells and mouse myeloma cells, including NS0 and Sp2/0 cells. Two derivatives of the CHO cell line, CHO-K1 and CHO pro-3, gave rise to the two most commonly used cell lines in large scale production, DUKX-X11 and DG44. (Kim, J., et al., Appl. Microbiol. Biotechnol., 2012, 93:917-30.) Other mammalian cell lines for recombinant antibody expression include, but are not limited to, COS, HeLa, HEK293T, U2OS, A549, HT1080, CAD, P19, NIH 3T3, L929, N2a, HEK 293, MCF-7, Y79, SO-Rb50, HepG2, J558L, and BHK. If the aim is large-scale production, the most currently used cells for this application are CHO cells. Guidelines to cell engineering for mAbs production were also reported. (Costa et al., Eur J Pharm Biopharm, 2010, 74:127-38.) Using heterologous promoters, enhancers and amplifiable genetic markers, the yields of antibody and antibody fragments can be increased. Thus, in certain embodiments, the invention provides an antibody, or antibody fragment, that is recombinantly produced from a mammalian cell-line, including a CHO cell-line. In certain embodiments, the invention provides a composition comprising an antibody, or antibody fragment, wherein the antibody or antibody fragment was recombinantly produced in a mammalian cell-line, and wherein the antibody or antibody fragment is present in the composition at a concentration of at least 1, 10, 100, 1000 micrograms/mL, or at a concentration of at least 1, 2, 3, 4, 5, 6, 7, 8, 9, 10, 20, 50, or 100 milligrams/mL.

Furthermore, large-scale production of therapeutic-grade antibodies are much different than those for laboratory scale. There are extreme purity requirements for therapeutic-grade. Large-scale production of therapeutic-grade antibodies requires multiples steps, including product recovery for cell-culture harvest (removal of cells and cell debris), one or more chromatography steps for antibody purification, and formulation (often by tangential filtration). Because mammalian cell culture and purification steps can introduce antibody variants that are unique to the recombinant production process (i.e., antibody aggregates, N- and C-terminal variants, acidic variants, basic variants, different glycosylation profiles), there are recognized approaches in the art for analyzing and controlling these variants. (See, Fahrner, et al., Industrial purification of pharmaceutical antibodies: Development, operation, and validation of chromatography processes, Biotech. Gen. Eng. Rev., 2001, 18:301-327.) In certain embodiments of the invention, the antibody composition comprises less than 1, 2, 3, 4, 5, 10, 15, 20, 25, 30, 35, 50, or 100 nanograms of host cell protein (i.e., proteins from the cell-line used to recombinantly produce the antibody)). In other embodiments, the antibody composition comprises less than 1, 2, 3, 4, 5, 6, 7, 8, 9, 10, 15, 20, or 25 ng of protein A per milligram of antibody or antibody fragment (i.e., protein A is a standard approach for purifying antibodies from recombinant cell culture, but steps should be done to limit the amount of protein A in the composition, as it may be immunogenic). (See, e.g., U.S. Pat. No. 7,458,704, Reduced protein A leaching during protein A affinity chromatography; which is hereby incorporated-by-reference.)

In certain aspects the invention provides nucleic acids encoding the inventive antibodies. In non-limiting embodiments, the nucleic acids are mRNA, modified or unmodified, suitable for use any use, e.g. but not limited to use as pharmaceutical compositions. In certain embodiments, the nucleic acids are formulated in lipid, such as but not limited to LNPs.

Method for Making Recombinant HLA-E-VL9 Specific Antibodies from Circulating Single B-Cells

In certain embodiments, the invention provides a method for making recombinant HLA-E-VL9 specific antibodies by screening for very rare antibodies from a circulating B-cell antibody repertoire. The method has a sensitivity level where it can identify and isolate B-cells expressing HLA-E-VL9 specific antibodies that are present at a very low percentage as compared to the overall circulating B-cell population, i.e., 1 in 1 million, 1 in 2 million, 1 in 3 million, 1 in 4 million, 1 in 5 million, 1 in 10 million cells, or 1 in 100 million cells or more.

In certain embodiments, the method comprises the following steps: (1) Fold a VL9 peptide (or other test peptide) with HLA-E to make a stable complex; (2) Assemble the folded HLA-E-peptide as a tetramer; (3) Use the tetramer to stain B cells from peripheral blood of a human donor or an animal (the donor or animal may be pre-immunized or challenged; for example, mice can be immunized with HLA-E-peptide, or for example, if one is preparing an antibody to an HLA-E-pathogen peptide complex, then one can screen human donors infected or immunized with the pathogen or animals immunized with the HLA-E-pathogen peptide complex); (3) Sort tetramer binding B cells as single cells and clone DNA or mRNA for antibody heavy and light chains; (4) Express full length DNA for heavy and light chains in suitable cells (e.g., HEK293T) so that antibody is expressed and secreted (or express fragments or other derivative forms that can be secreted); (5) Determine specificity of antibody binding to HLA-E-peptide protein complexes expressed on cells transfected with DNA encoding single chain trimers of peptide-β2microblobulin-HLA-E (such as by flow cytometry) or to such complexes immobilized on a substrate (such as by ELISA by SPR (and with such assays, alternatively, the antibodies can be immobilized on the substrate and soluble trimers added); and (6) purify antibodies with requisite binding specificity.

In this method, standard approaches can be used for staining and sorting for B-cells that express antibodies specific to an HLA-E-peptide complex. For example, using fluorescent activated cells sorting (FACS; flow cytometry), numerous commercial reagents are available to stain cells with antibodies conjugated to different fluorescent colors, including different combinations of reagents to identify and sort B-cells and B-cell sub-populations such as naïve and memory B-cells. For identifying and sorting B-cells that bind to HLA-E-peptide tetramers, the tetramers are conjugated to fluorescent dyes according to standard methods in the art. A non-limiting example is provided in Example 1 herein, under Materials & Methods, Antigen-Specific Single B-cell Sorting.

### Pharmaceutical Compositions

The present invention also provides a pharmaceutical composition comprising one or more of: (i) the antibody, or the antibody fragment thereof, according to the present invention; (ii) the nucleic acid encoding the antibody, or antibody fragments according to the present invention; (iii) the vector comprising the nucleic acid according to the present invention; and/or (iv) the cell expressing the antibody according to the present invention or comprising the vector according to the present invention.

In certain aspects, the invention provides a pharmaceutical composition comprising the antibody, or the antigen binding fragment thereof, according to the present invention, the nucleic acid according to the present invention, the vector according to the present invention and/or the cell according to the present invention.

The pharmaceutical composition may also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In general, pharmaceutically acceptable carriers in a pharmaceutical composition according to the present invention may be active components or inactive components. In certain embodiments the pharmaceutically acceptable carrier in a pharmaceutical composition according to the present invention is not an active component in respect to coronavirus infection.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, capsules, liquids, gels, syrups, slurries and suspensions, for ingestion by the subject.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin™, for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody may be provided in kit form with sterile water or a sterile buffer.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH between 5.5 and 8.5, in some embodiments this may be between 6 and 8, and in other embodiments about 7. The pH may be maintained by the use of a buffer. The composition may be sterile and/or pyrogen free. The composition may be isotonic with respect to humans. In one embodiment pharmaceutical compositions of the invention are supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody molecule may be in dry form, for reconstitution before use with an appropriate sterile liquid. A vehicle is typically understood to be a material that is suitable for storing, transporting, and/or administering a compound, such as a pharmaceutically active compound, in particular the antibodies according to the present invention. For example, the vehicle may be a physiologically acceptable liquid, which is suitable for storing, transporting, and/or administering a pharmaceutically active compound, in particular the antibodies according to the present invention. Once formulated, the compositions of the invention can be administered directly to the subject. In one embodiment the compositions are adapted for administration to mammalian, e.g., human subjects.

The pharmaceutical compositions of this invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions of the invention. In certain embodiments, the pharmaceutical composition may be prepared for oral administration, e.g. as tablets, capsules and the like, for topical administration, or as injectable, e.g. as liquid solutions or suspensions. In certain embodiments, the pharmaceutical composition is an injectable. Solid forms suitable for solution in, or suspension in, liquid vehicles prior to injection are also contemplated, e.g. that the pharmaceutical composition is in lyophilized form.

For injection, e.g. intravenous, cutaneous or subcutaneous injection, or injection at the site of affliction, the active ingredient could be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is a polypeptide, peptide, or nucleic acid molecule, other pharmaceutically useful compound according to the present invention that is to be given to an individual, administration is in a “prophylactically effective amount” or a “therapeutically effective amount”, this being sufficient to show benefit to the individual. The actual amount administered, and rate and time-course of administration, will depend on the nature and severity of what is being treated. For injection, the pharmaceutical composition according to the present invention may be provided for example in a pre-filled syringe.

The inventive pharmaceutical composition as defined above may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the inventive transporter cargo conjugate molecule as defined above, is combined with emulsifying and suspending agents. If desired, certain sweetening, flavoring or coloring agents may also be added.

The inventive pharmaceutical composition may also be administered topically, especially when the target of treatment includes areas or organs readily accessible by topical application, e.g. including diseases of the skin or of any other accessible epithelial tissue. Suitable topical formulations are readily prepared for each of these areas or organs. For topical applications, the inventive pharmaceutical composition may be formulated in a suitable ointment, containing the inventive pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the inventive pharmaceutical composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

Suitable dose ranges can depend on the antibody (or fragment) and on the nature of the formulation and route of administration. For example, doses of antibodies in the range of 0.1-50 mg/kg, 1-50 mg/kg, 1-10 mg/kg, 1, 1.25, 1.5, 2, 2.5, 3, 3.5, 4, 4.5, 5, 5.5, 6, 6.5, 7, 7.5, 8, 8.5, 9, 9.5 or 10 mg/kg of antibody can be used. If antibody fragments are administered, then less antibody can be used (e.g., from 5 mg/kg to 0.01 mg/kg). In other embodiments, the antibodies of the invention can be administered at a suitable fixed dose, regardless of body size or weight. See Bai et al. Clinical Pharmacokinetics February 2012, Volume 51, Issue 2, pp 119-135.

Dosage treatment may be a single dose schedule or a multiple dose schedule. In particular, the pharmaceutical composition may be provided as single-dose product. In certain embodiments, the amount of the antibody in the pharmaceutical composition—in particular if provided as single-dose product—does not exceed 200 mg. In certain embodiments, the amount does not exceed 100 mg, and in certain embodiments, the amount does not exceed 50 mg.

In non-limiting embodiments, the antibodies of the invention could be used for non-therapeutic uses, such as but not limited to diagnostic assays.

### Administration of Antibody-Encoding Nucleic Acid Sequences

In some embodiments the antibodies are administered as nucleic acids, including but not limited to mRNAs which could be modified and/or unmodified. See US Pub 20180028645A1, US Pub 20090286852, US Pub 20130111615, US Pub 20130197068, US Pub 20130261172, US Pub 20150038558, US Pub 20160032316, US Pub 20170043037, US Pub 20170327842, U.S. Pat. Nos. 10,006,007, 9,371,511, 9,012,219, US Pub 20180265848, US Pub 20170327842, US Pub 20180344838A1 at least at paragraphs [0260]-[0281], WO/2017/182524 for non-limiting embodiments of chemical modifications, wherein each content is incorporated by reference in its entirety.

mRNAs delivered in LNP formulations have advantages over non-LNPs formulations. See US Pub 20180028645A1, WO/2018/081638, WO/2016/176330, wherein each content is incorporated by reference in its entirety. In certain embodiments the nucleic acid encoding an envelope is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides a composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding the polypeptide sequence of the sequences in in the instant application, or a variant thereof or a fragment thereof. Accordingly, in one embodiment, the invention provides an RNA molecule encoding one or more of inventive antibodies. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7mG(5′)ppp(5′)NlmpNp). This cap can enhance in vivo translation of the RNA. The 5′ nucleotide of an RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. In some embodiments, a RNA molecule useful with the invention may be single-stranded. In some embodiments, a RNA molecule useful with the invention may comprise synthetic RNA.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

### Therapeutic Methods

In certain aspects, the invention provides prophylactic methods comprising administering the antibodies of the invention. In certain embodiments, the methods lead to protection or treatment of infection or disease by blocking or otherwise inhibiting the intercellular signaling mediated by the engagement of an HLA-E-VL9 complex (or an HLA-E-peptide of interest complex such as a tumor or viral peptide) on an infected or diseased cell and the NKG2A receptor expressed on sub-populations of NK cells and CD8+ T-cells. Tumor cells and virally-infected cells have been known to co-opt this intercellular signaling pathway in order to inhibit the activation and concomitant killing by the NKG2A+NK cell and NKG2A+CD8+ T-cell sub-populations. As used herein, activated NKG2A+NK cell and NKG2A+ T-cells include such cells that downregulate and reduce or eliminate cell-surface expression of NKG2A+.

In one embodiment, the therapeutic method is for protection against cytomegalovirus HCMV, as an HLA-E-HCMV peptide complex has a peptide with an exact match to one of the VL9 peptide sequences VMAPRTLIL that is expressed and binds to HLA-E. In another embodiment, the therapeutic method comprises administration of an antibody having the binding specificity of the 3H4 antibody, is a chimeric version of 3H4, or is a humanized version of 3H4.

Thus, in certain embodiments, the therapeutic compositions and methods not only involve blocking the inhibitory HLA-E-VL9-NKG2A pathway in said NK cells and CD8+ T-cells, but also: (1) blocking other inhibitory receptors on these NK cells and CD8+ T-cells, and/or (2) promoting the activation of stimulatory receptors on these NK cells and CD8+ T-cells. The multiple targeting of receptors on NKG2A+NK cell and NKG2A+CD8+ T-cell sub-populations can be accomplished, for example, by the use of combination of different antibodies or agents each targeting a different receptor, or by recombinant multi-specific antibodies.

In certain aspects, the invention provides methods of treatment comprising administering the pharmaceutical composition of the invention. Generally, the methods are applicable to diseases or conditions that would benefit from an increase in the number of stimulated effector immune cells such as NK cells, CD8+ T-cells, and γδ T-cells (which can also mediate cytotoxic responses against tumors). Exemplary diseases or conditions include, but are not limited to, cancer and viral infection.

Such methods of treatment can relate to methods of immunostimulation comprising the steps of: administering to a subject in need of enhanced immune cytotoxic effector function a therapeutically effective amount of an antibody of the invention, which antibody specifically binds to at least an HLA-E-VL9 peptide complex (or other HLA-E-peptide of interest complexes) and increases the number of activated NK cells or activated CD8+ cells. An increase in the number of activated NK cells or activated CD8+ cells can be determined by testing for the ability of the antibody to increase the proportion of activated cells in a sample, such as a peripheral blood sample. It is known in the art that flow-cytometry methods can be used to detect and count the number activated NK cells or activated CD8+ cells. Cell-surface marker profiles that define NK cell CD8+ T cell, γδ T-cell, and other populations are known in the art. Generally, activated NK cells can be identified by a decrease in CD16 surface level and an increase in CD107a. (See, e.g., Veluchamy et al., Scientific Reports, 2017, 7:43873.) Generally, activated effector T-cells can be identified by a loss of L-selectin, a gain of VLA-4, higher levels of LFA-1 and CD2, and expression of CD45RO instead of CD45RA. Furthermore, as described, the ⁵¹Cr release assay provides an in vitro means to determine whether an antibody of the invention can cause an increase in NK cell activation.

In certain embodiments, the methods comprise administering additional therapeutic or prophylactic agents, including but not limited to additional antibodies that block inhibitory receptors on NK or T-cells, antibodies that work as agonists for stimulatory receptors on NK or T cells, small molecule therapeutics, or any other suitable agent. In some respects, the additional agent is an antibody that specifically binds to the NKG2A/CD94 complex on NK cells or CD8+ T-cells. In some respects, the additional agent is an antibody that specifically binds the CTLA-4 receptor on NK cells or CD8+ T-cells. In some respects, the additional agent is an antibody that specifically binds PD-1 on NK cells or CD8+ T-cells.

In certain embodiments the methods comprise administering one or more antibodies or antigen fragments thereof, including without limitation multimeric antibodies, of the invention in a combination treatment. In certain embodiments, the these are selected such that each antibody or antigen fragments thereof has at least one differential function compared to other antibody or antibodies in the combination. In a non-limiting embodiment, antibodies or antigen binding fragments in a combination treatment have non-overlapping epitopes. In non-limiting embodiments, antibodies or antigen fragments thereof in a combination treatment provide different effect on cellular pathways.

In some embodiments, a combination treatment comprising inventive antibodies could further comprise additional therapeutic or prophylactic agents.

TABLE 4
Non limiting embodiments of VL-9 peptides

| Natural/ | | | | | | | | | | |
|---|---|---|---|---|---|---|---|---|---|---|
| synthetic | P1 | P2 | P3 | P4 | P5 | P6 | P7 | P8 | P9 | ReF |
| Natural | V | M | A | P | R | T | L | L | L | 1 |
| | —(*) | — | — | — | — | — | — | V | — | 1 |
| | — | — | — | — | — | — | V | — | — | 1 |
| | — | — | — | — | — | — | — | I | — | 1 |
| | — | — | — | — | — | — | — | F | — | 1 |
| | — | T | — | — | — | — | — | — | — | 1 |
| Synthetic | A | — | — | — | — | — | — | — | — | 2 |
| | C | — | — | — | — | — | — | — | — | 2 |
| | I | — | — | — | — | — | — | — | — | 2 |
| | S | — | — | — | — | — | — | — | — | 2 |
| | T | — | — | — | — | — | — | — | — | 2 |
| | — | L | — | — | — | — | — | — | — | 3 |
| | — | I | — | — | — | — | — | — | — | 3 |
| | — | Q | — | — | — | — | — | — | — | 3 |
| | — | F | — | — | — | — | — | — | — | 3 |
| | — | T | — | — | — | — | — | — | — | 3 |
| | — | V | — | — | — | — | — | — | — | 3 |
| | — | S | — | — | — | — | — | — | — | 3 |
| | — | A | — | — | — | — | — | — | — | 3 |
| | — | — | — | — | — | — | — | — | F | 3 |

(*) A dash in the table indicates that the amino acid at that position is identical to the amino acid in the VMAPRTLLL peptide. References are as follows:
1 https://www.ebi.ac.uk/ipd/imgt/hla/;
2 See Example 1,
3 Walters L C, Harlos K, Brackenridge S, Rozbesky D, Barrett J R, Jain V, Walter T S, O'Callaghan C A, Borrow P, Toebes M, Hansen S G, Sacha J B, Abdulhaqq S, Greene J M, Fruh K, Marshall E, Picker L J, Jones E Y, McMichael A J, Gillespie G M. Pathogen-derived HLA-E bound epitopes reveal broad primary anchor pocket tolerability and conformationally malleable peptide binding. Nat Commun. 2018; 9(1): 3137. Epub 2018/08/09. doi: 10.1038/s41467-018-05459-z. PubMed PMID: 30087334; PMCID: PMC6081459. Both natural and a subset of the synthetic VL9 peptides have been shown to bind to HLA-E. A skilled artisan can readily determine which of the synthetic peptides can be loaded into HLA-E to form a complex.

### EXAMPLES

### Example 1: IgM Natural Antibodies Bind HLA-E-Leader Peptide Complexes and Modulate NK Cell Cytotoxicity—Including Non-Limiting Embodiments of Affinity Matured 3114 Antibodies

### Abstract

The non-classical class Ib molecule human leukocyte antigen E (HLA-E) has limited polymorphism and can bind HILA class Ia leader peptides (VL9). HILA-E-VL9 complexes interact with the natural killer (NIK) cell receptors NTKG2A-C/CD94 and regulate NIK cell-mediated cytotoxicity. Here we report the isolation of 3H4, a murine HILA-E-VL9-specific IgM antibody that enhanced killing of HLA-E-VL9-expressing cells by an NKG2A+NK cell line. Structural analysis revealed that 3H4 acts by preventing CD94/NKG2A docking on HLA-E-VL9. Upon in vitro maturation, an affinity-optimized IgG form of 3H4 showed enhanced NK killing of HLA-E-VL9-expressing cells. HLA-E-VL9-specific IgM antibodies similar in function to 3H4 were also isolated from naïve B cells of cytomegalovirus (CMV)-negative, healthy humans. Thus, a subset of natural antibodies that recognize VL9-bound HLA-E exist as part of the naïve Ig repertoire with the capacity to regulate NK cell function.

### Introduction

Natural killer (NK) cells play critical roles in immune surveillance by discriminating normal from altered cells, and function as effector cells by killing non-self malignant or pathogen-infected cells and by producing inflammatory cytokines (Chiossone et al., 2018; Raulet, 2006; Yokoyama and Kim, 2006). Specific recognition of abnormal cells by NK cells relies on a series of activating and inhibitory receptors, including the killer immunoglobulin-like receptor (KIR) family and NKG2/CD94 heterodimeric receptors (Andre et al., 2018; Chiossone et al., 2018). NK cell inhibitory receptors ligate human lymphocyte antigen (HLA) or major histocompatibility complex (MHC) class I molecules expressed on healthy cells as self. Conversely, cells lacking MHC class I are recognized by NK cells as “missing-self” and are sensitive to NK cell-mediated killing (Ljunggren and Karre, 1985, 1990). In humans, KIRs recognize classical human HLA class Ia molecules (Colonna and Samaridis, 1995; Karlhofer et al., 1992; Pende et al., 2019), whereas the inhibitory NKG2A/CD94 heterodimeric receptor interacts with the non-classical HLA class Ib molecule HLA-E and is balanced by an activating receptor NKG2C/CD94 (Braud et al., 1997; Braud et al., 1998; Brooks et al., 1997). While KIR expression is heterogeneous, NKG2A/CD94 is expressed on ˜40% of human NK cells (Andre et al., 1999; Mahapatra et al., 2017; Pende et al., 2019). Unlike classical HLA class I molecules, HLA-E has limited polymorphism with only two expressed variants, HLA-E*01:01 and HLA-E*01:03, that differ only in residue 107, which is outside the peptide-binding groove (Kraemer et al., 2014). The NKG2A/CD94/HLA-E pathway is considered as an important immune checkpoint target and has recently become a focus for NK cell-based immunotherapeutic strategies (Andre et al., 2018; Hu et al., 2019; Kim et al., 2019; Souza-Fonseca-Guimaraes et al., 2019; van Hall et al., 2019). A subset of CD8+ T cells also express NKG2A/CD94, and inhibition of NKG2A/CD94-HLA-E interaction similarly has application in CD8+ T cell-based immunotherapy (Andre et al., 2018; van Montfoort et al., 2018).

HLA-E engages with NKG2A/CD94 via a restricted subset of peptides VMAPRT(L/V) (V/L/I/F)L (designated VL9) that derive from the leader sequence of HLA-A, -C, -G and a third of HLA-B molecules (Braud et al., 1997; Braud et al., 1998; Lee et al., 1998a; Lee et al., 1998b). HLA-E binds VL9 peptides, which stabilize HLA-E surface expression (Braud et al., 1997; Braud et al., 1998) on healthy host cells in which HLA-Ia expression is not perturbed and initiate recognition by NKG2A/CD94 or NKG2C/CD94 on NK cells. The binding affinity of HLA-E-VL9 peptide complexes for NKG2A/CD94 is greater than that for NKG2C/CD94, such that the inhibitory signal dominates to suppress aberrant NK cell-mediated cytotoxicity and cytokine production (Aldrich et al., 1994; Braud et al., 1998; Kaiser et al., 2008; Llano et al., 1998; Rolle et al., 2018). In addition, HLA-E and its murine or rhesus macaque homologs are capable of binding to a range of other host peptides and pathogen-derived peptides, including heat-shock protein 60 (Hsp60)-derived peptides (Michaelsson et al., 2002), Mycobacterium tuberculosis (Mtb) peptides (Joosten et al., 2010; van Meijgaarden et al., 2015), and simian immunodeficiency virus (SIV) Gag peptides (Hansen et al., 2016; Walters et al., 2018). However, only VL9 peptide-loaded HLA-E can engage CD94/NKG2A and protect cells from NK cell cytotoxicity (Kraemer et al., 2015; Michaelsson et al., 2002; Sensi et al., 2009). Hence, leader sequence VL9 peptides are essential not only for stabilizing HLA-E surface expression but also for mediating the role of HLA-E/NKG2A/CD94 in regulating NK cell self-recognition. However, it remains unclear if interruption of this pathway by specifically targeting HLA-E-peptide complexes on target cells can enhance NK cell activity.

Natural antibodies are immunoglobulins that are present prior to simulation by cognate antigen, and provide the first line of defense against bacterial, fungal and viral infections (Holodick et al., 2017). They also suppress autoimmune, inflammatory and allergic responses, protect from atherosclerotic vascular injury, and mediate apoptotic cell clearance (New et al., 2016). Natural antibodies are generally near germline in sequence, have repertoire skewing of variable heavy chain (VH) and variable light chain (VL) genes, and respond to antigens with T cell independence (Holodick et al., 2017). However, specific roles of natural antibodies in regulation of natural killer (NK) cell function are unknown.

Here, we define a mechanism of natural antibody modulation of NK cell killing whereby a murine IgM monoclonal antibody (mAb) 3H4 bound to HLA-E-VL9 on target cells and enhanced NK cytotoxicity mediated by an NKG2A+NK cell line. X-ray crystallographic analysis of an HLA-E-VL9/3H4 antigen-binding fragment (Fab) co-complex indicated that due to steric clashes, 3H4 and CD94/NKG2A cannot simultaneously bind to what are essentially overlapping recognition surfaces on HLA-E-VL9. Ig V(D)J residues at the 3H4-HLA-E-VL9 binding interface were germline-encoded. While 3H4 mAb enhanced NK cytotoxicity as an IgM, the IgG form of the antibody showed no such functionality. To address this, we developed 3H4 IgG variants with enhanced HLA-E-VL9 binding by high throughput screening of antibody libraries. These optimized 3H4 IgG Abs contain mutations in their CDR-H3 loops, bind HLA-E/VL-9-220 times tighter than the WT mAb and showed robust enhancement of NK cytotoxicity. Finally, human HLA-E-VL9-reactive, near-germline IgMs were isolated from the human naïve B cell repertoire that also enhanced NK cell killing as IgG. Thus, a subset of natural IgM HLA-E-VL9 antibodies exist in vivo that have the potential to regulate NK cell cytotoxicity.

### Results

### Isolation of Murine HLA-E-VL9-Specific mAb 3114

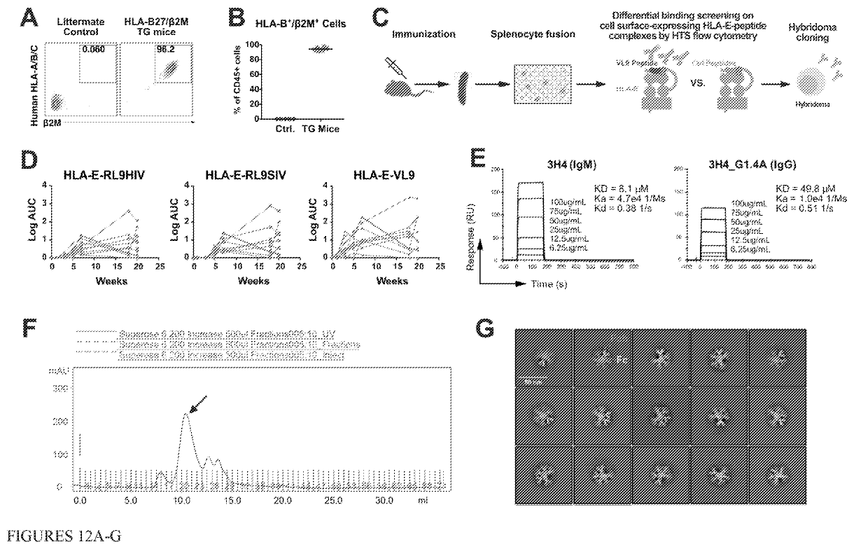

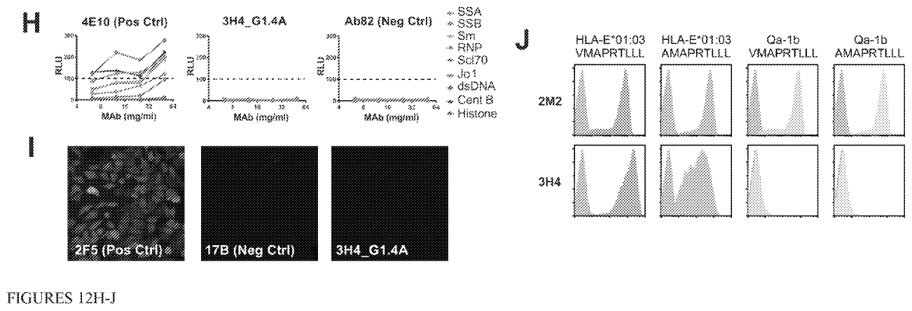

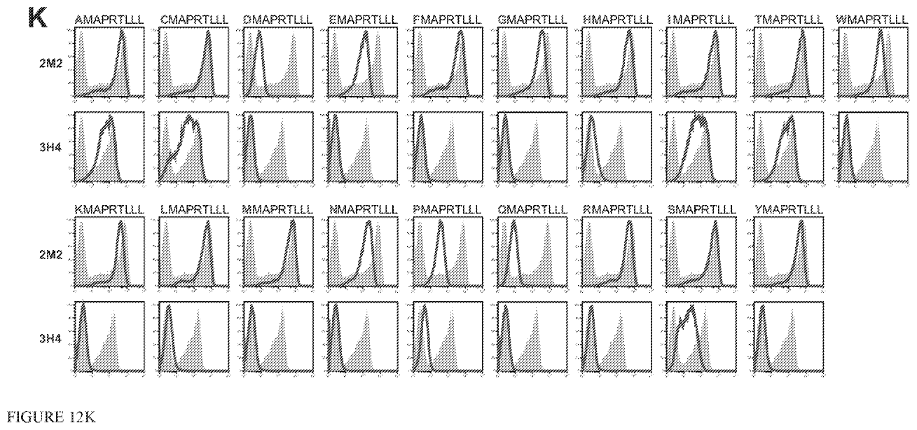

With the original intention of raising monoclonal antibodies to the HIV-1 Gag peptide RMYSPTSIL (RL9HIV) (the HIV counterpart of RL9SIV, one of the MHC-E binding SIV Gag epitope peptides identified by Hansen et al., 2016), we immunized human HLA-B27/β2-microglobulin (β2M) transgenic mice (Taurog et al., 1990) (FIGS. 12A-B) with 293T cells transfected with surface-expressed single-chain HLA-E-RL9HIV complexes (Yu et al., 2002) (FIG. 12C-D). We produced hybridomas, and screened culture supernatants for binding on a panel of 293T cells transfected with either single-chain HLA-E-RL9HIV peptide complexes, or with single-chain HLA-E-VL9 peptide complexes as a control. Unexpectedly, we isolated a subset of antibodies that specifically reacted with HLA-E-VL9 peptide, the most potent of which was the IgM mAb 3H4. Unlike the well-characterized pan-HLA-E mAb 3D12 (Marin et al., 2003), 3H4 reacted specifically with HLA-E-VL9 (VMAPRTLVL) and not with control, non-VL9 HLA-E-peptide complexes (FIG. 1A). Mab 3H4 also bound to VL9 peptide-pulsed HLA-class I negative K562 cells transfected with HLA-E (Lampen et al., 2013) (FIG. 1B) and also to soluble HLA-E refolded with synthetic VL9 peptide in both ELISA (FIG. 1C) and surface plasmon resonance (SPR) assays (FIG. 1D). SPR measurements showed that the 1:1 dissociation constants (KDs) of IgM 3H4 and human IgG1 backbone 3H4 for soluble HLA-E-VL9 were 8.1 and 49.8 μM, respectively (FIG. 12E).

### HLA-E-VL9-Specific mAb 3H4 is a Minimally Mutated Pentameric IgM

Sequence analysis of 3H4 mAb revealed 1.04% heavy chain variable region (VH) and 2.51% light chain viable region (VL) mutations (Table 5). We isolated 3 additional HLA-E-VL9 mouse mAbs from another two immunization studies (see Methods), and all four antibodies were minimally mutated IgMs (mean VH and VL mutations, 1.21% and 2.87%, respectively (Table 5). Negative stain electron microscopy showed that 3H4 was predominantly pentameric with a small proportion of hexamers (FIGS. 12F-G). In addition, 3H4 was not autoreactive in anti-nuclear antibody or clinical autoantibody assays (FIGS. 12H-I).

### 3H4 IgM Recognized the α1/α2 Domain of HLA-E and N-Terminus of the VL9 Peptide

To map the epitope on the HLA-E-VL9 complex recognized by 3H4, we tested 3H4 binding to VL9 peptide presented by HLA-E, the rhesus ortholog Mamu-E, as well as two HLA-E/Mamu-E hybrids—one with HLA-E α1/Mamu-E α2 (Hα1/Mα2), the other with Mamu-E α1/HLA-E α2 (Mα1/Hα2). 3H4 did not bind to Mamu-E/VL9 or Hα1/Mα2-VL9, and its staining of cells expressing Mα1/Hα2-VL9 was weak (FIG. 1E), suggesting that 3H4 recognition involved interaction with both α1 and α2 domains of HLA-E, and the epitope on α2 might be partially conserved between human and rhesus. 3H4 also did not cross-react with mouse ortholog Qa-1b (FIG. 12J). Moreover, VL9 mutations indicated that position 1 (P1) of the peptide was important for 3H4 binding (FIG. 1F), with strong antibody recognition of VL9 peptide P1 variants with alanine, cysteine, isoleucine, serine, threonine, weak binding to histidine and proline substitutions, but no interaction with arginine, glutamate, glycine, lysine, methionine, asparagine, tryptophan, tyrosine or phenylalanine (FIGS. 1G and 12K). These data suggested that mAb 3H4 made contacts with both the HLA-E α1/a2 domain and the amino-terminal end of the VL9 peptide.

### Co-Complex Crystal Structure of a 3H4 Fab Bound to HLA-E-VL9

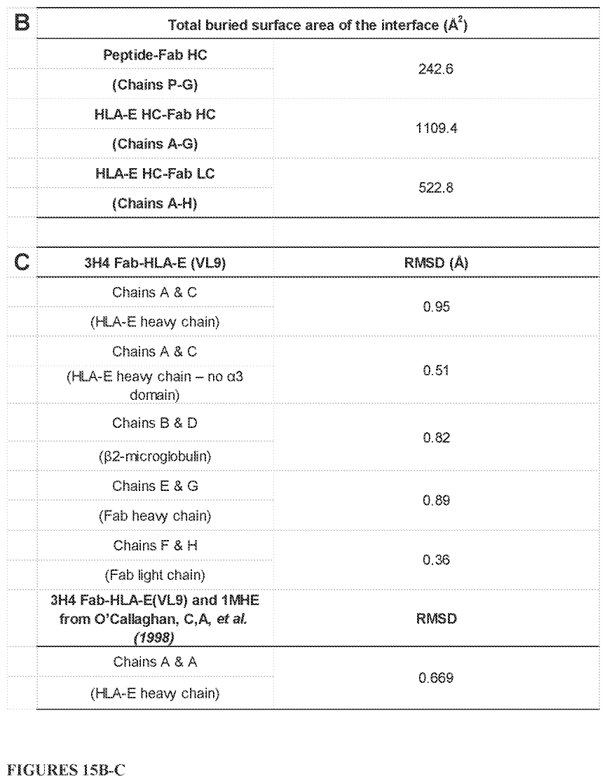

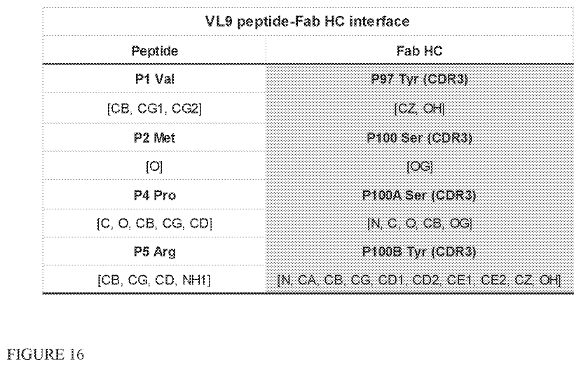

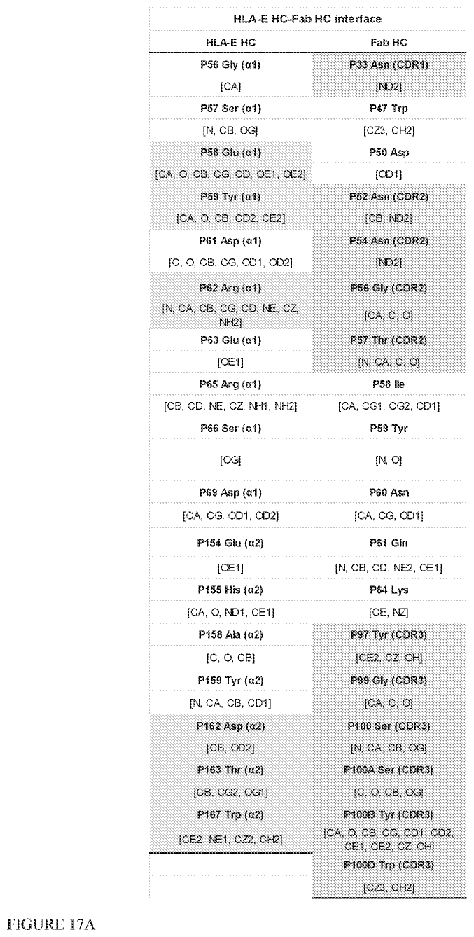

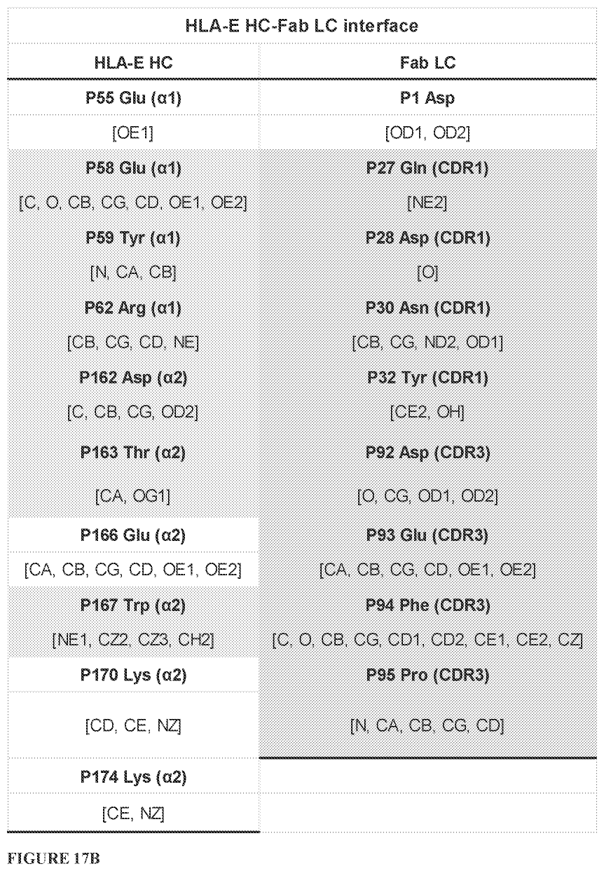

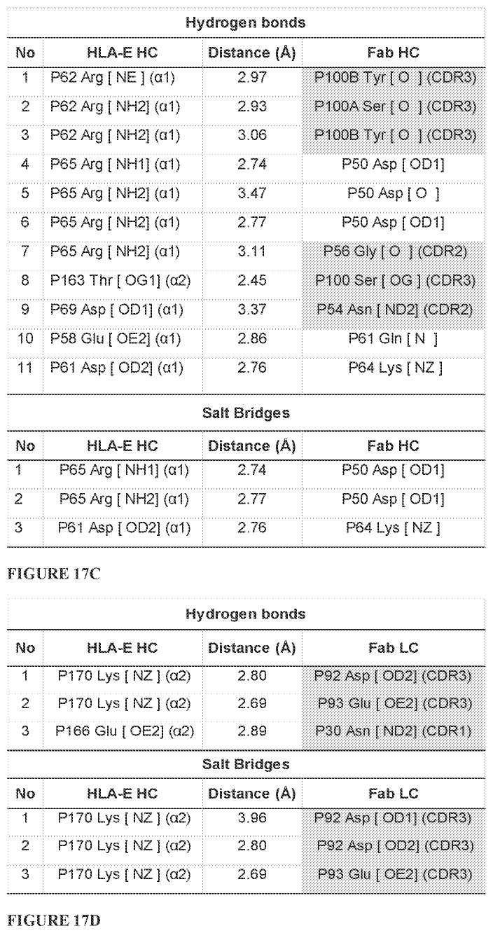

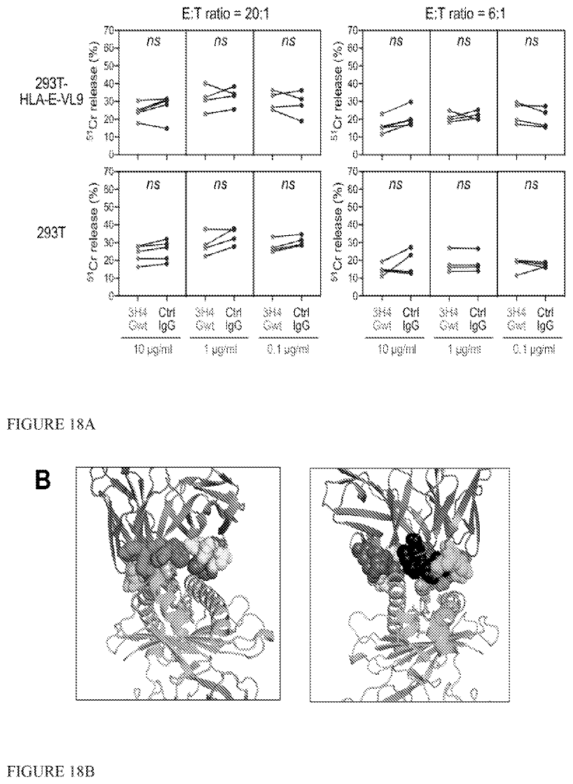

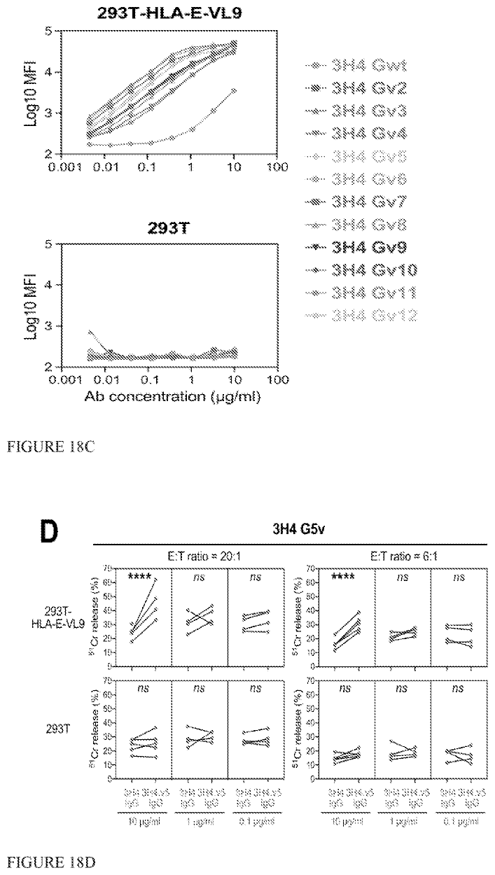

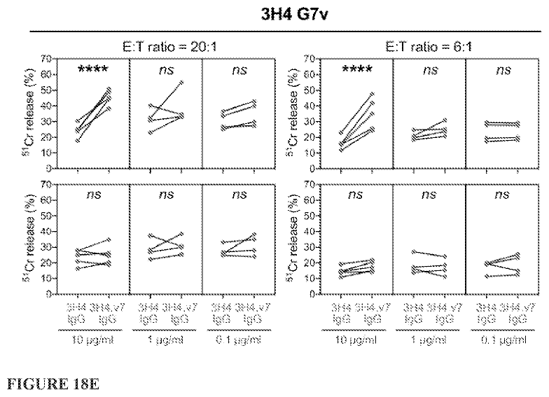

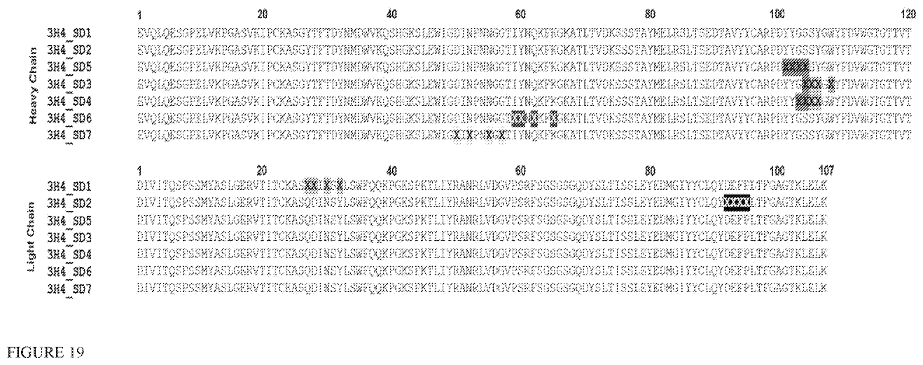

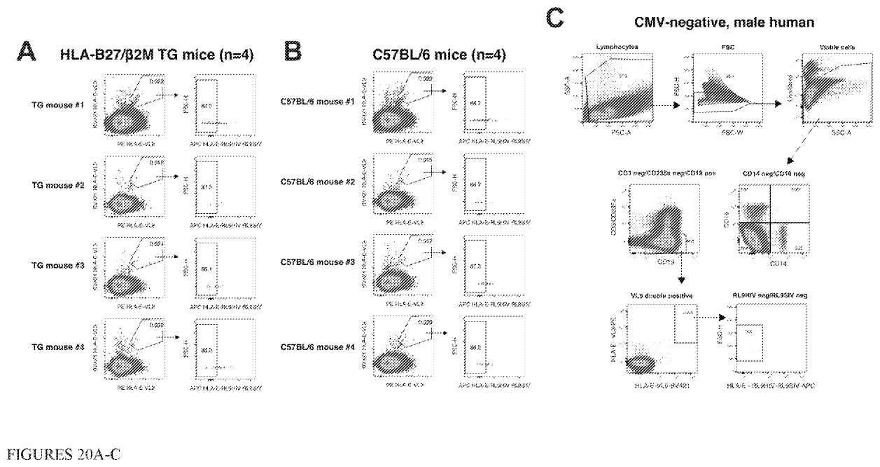

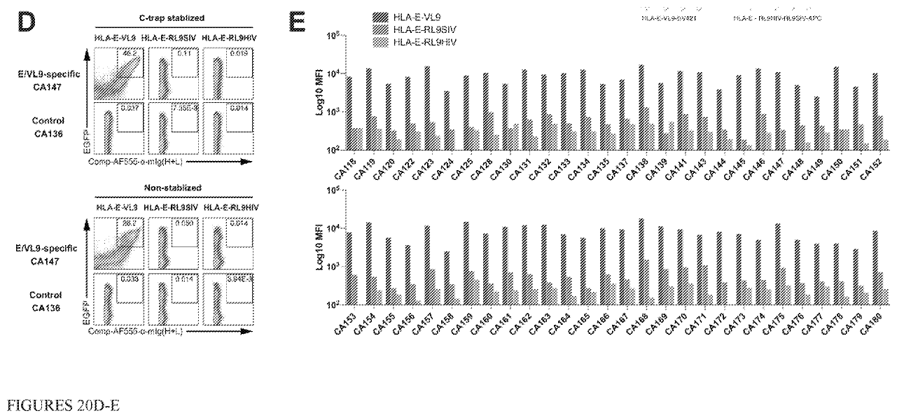

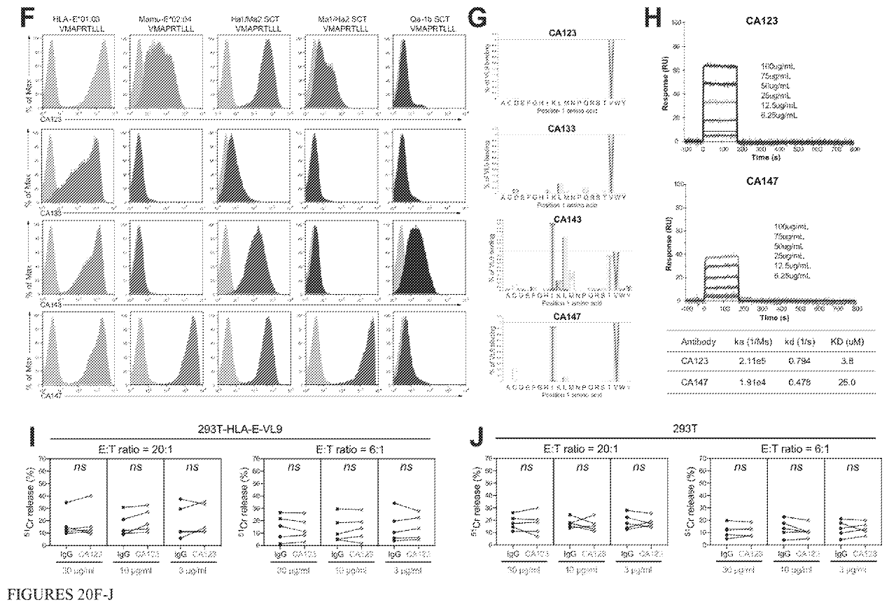

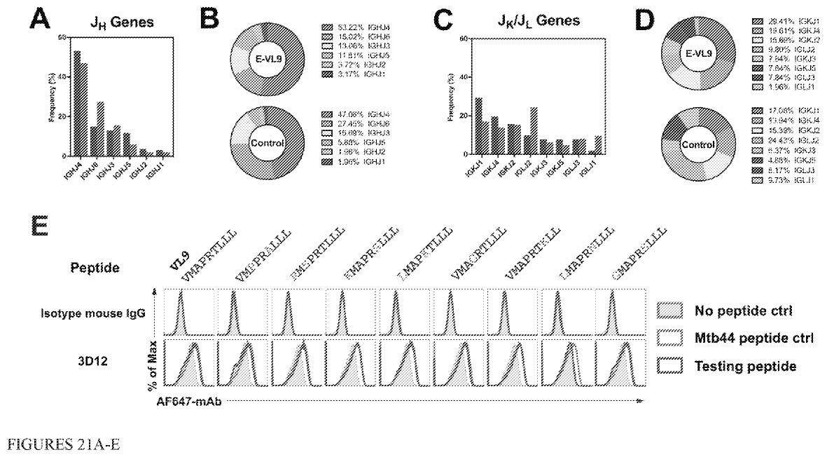

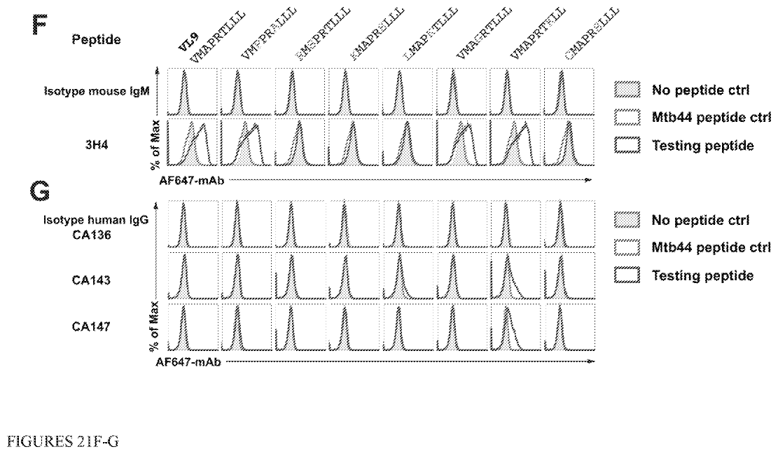

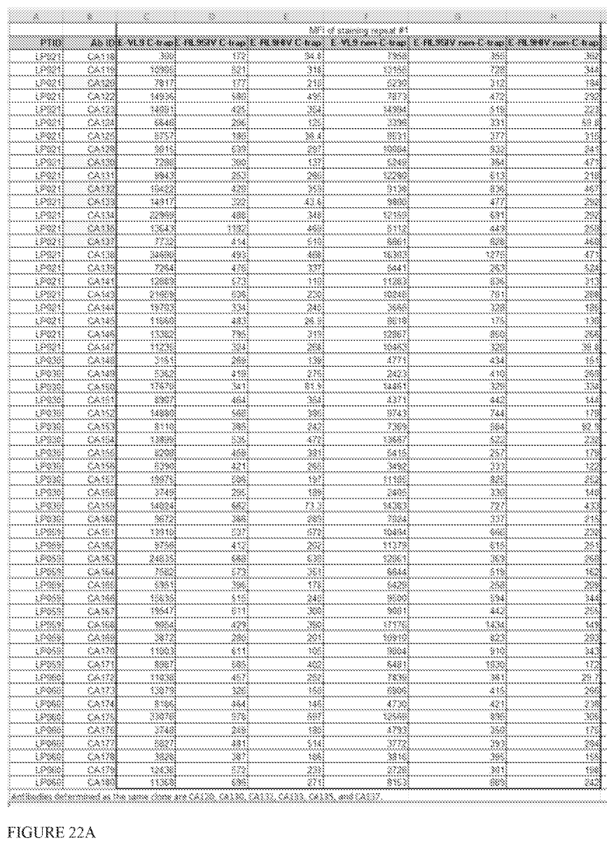

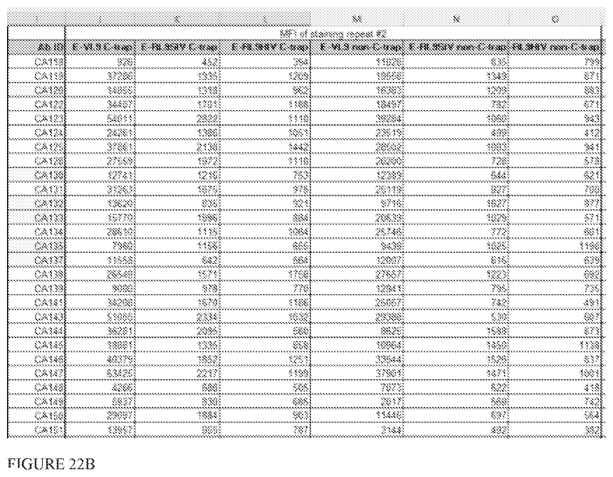

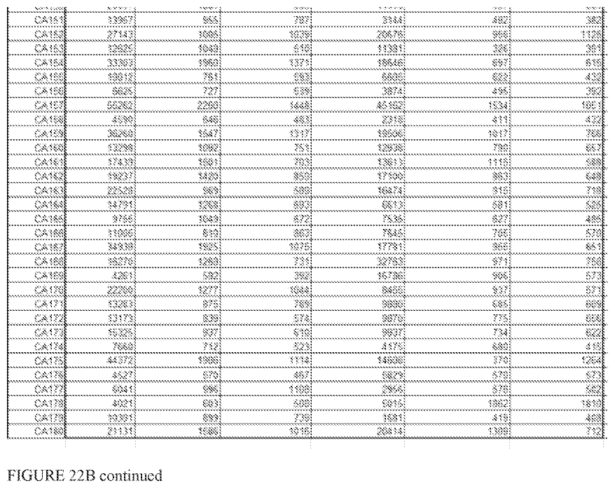

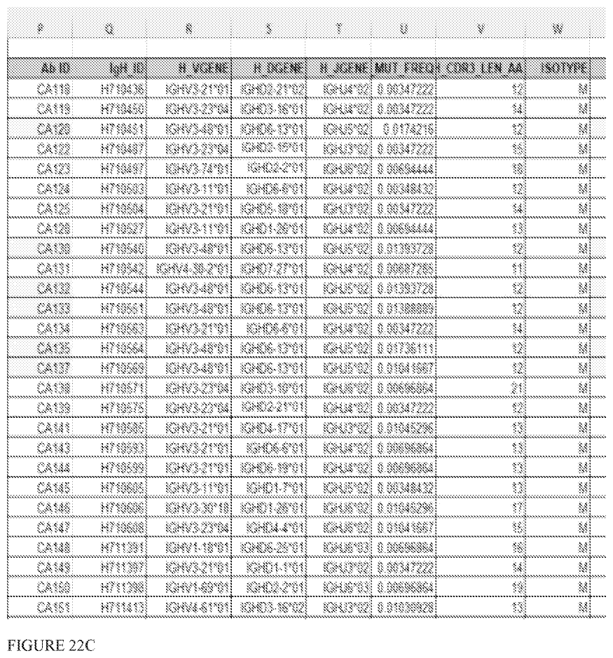

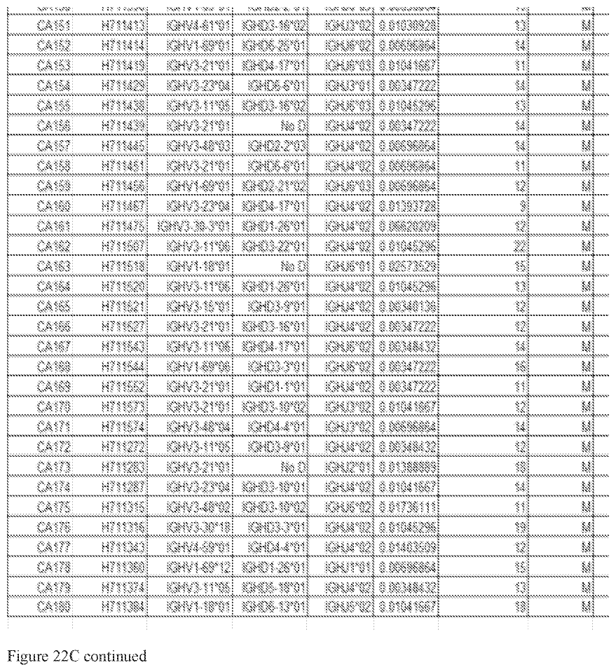

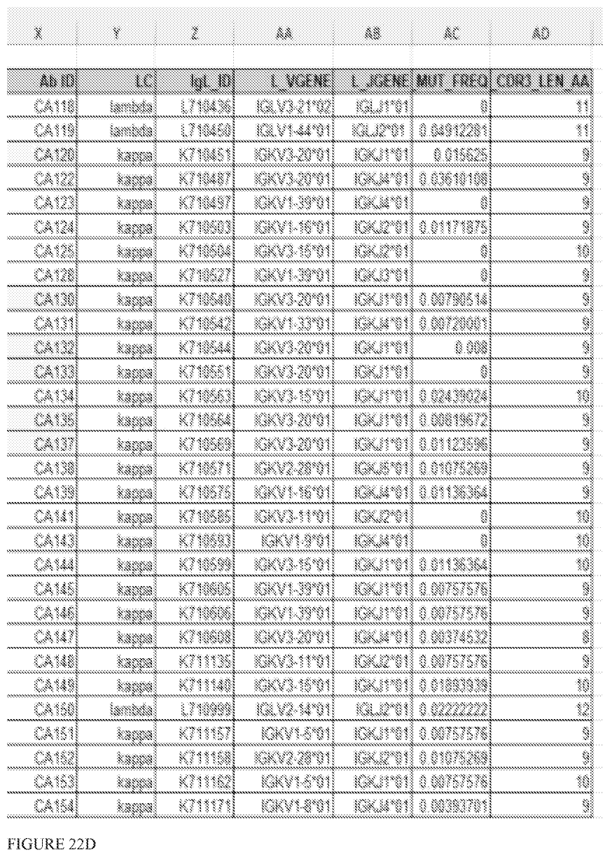

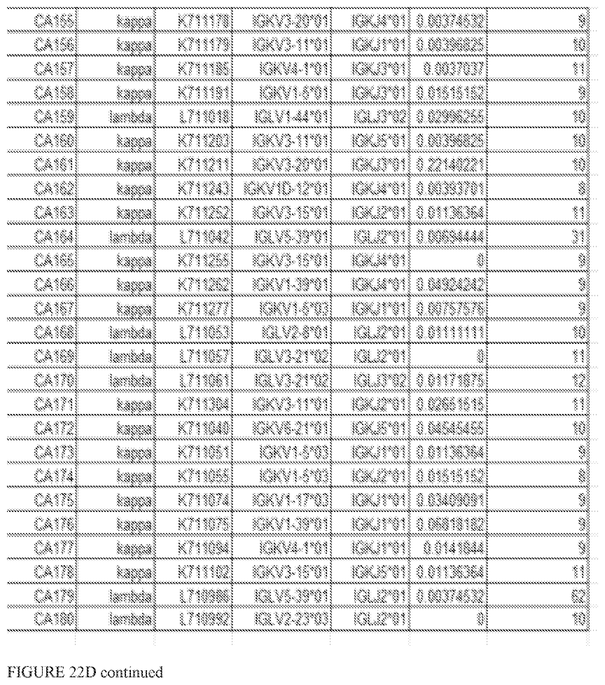

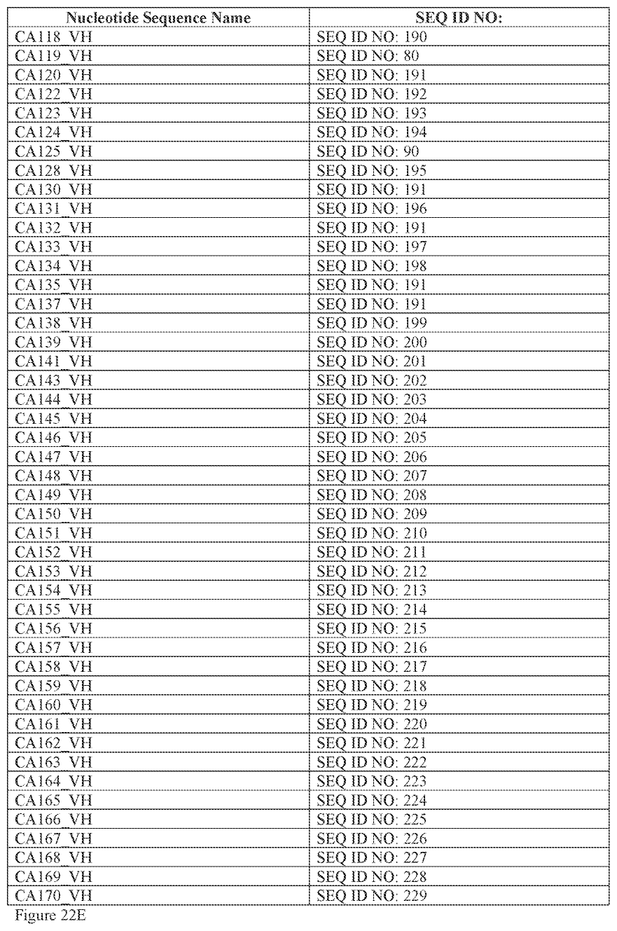

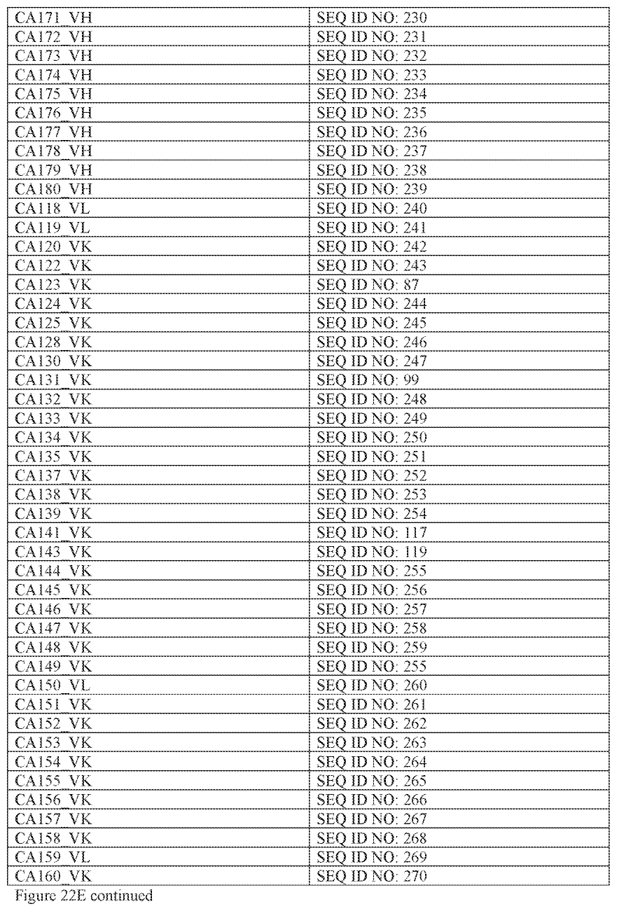

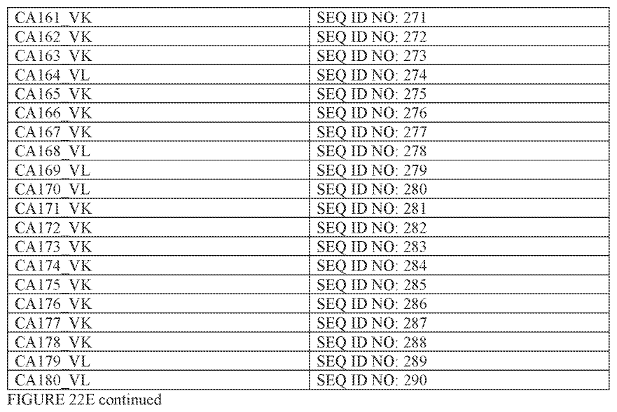

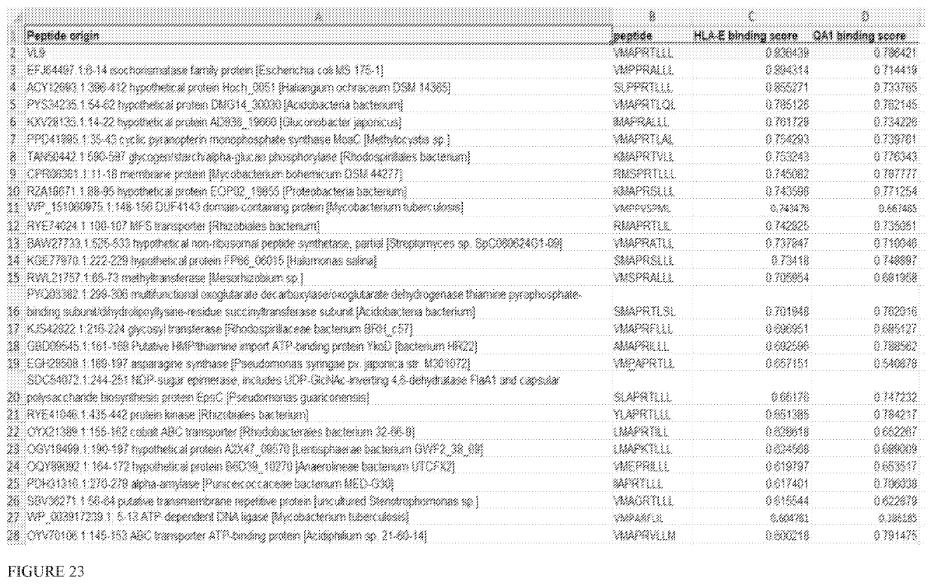

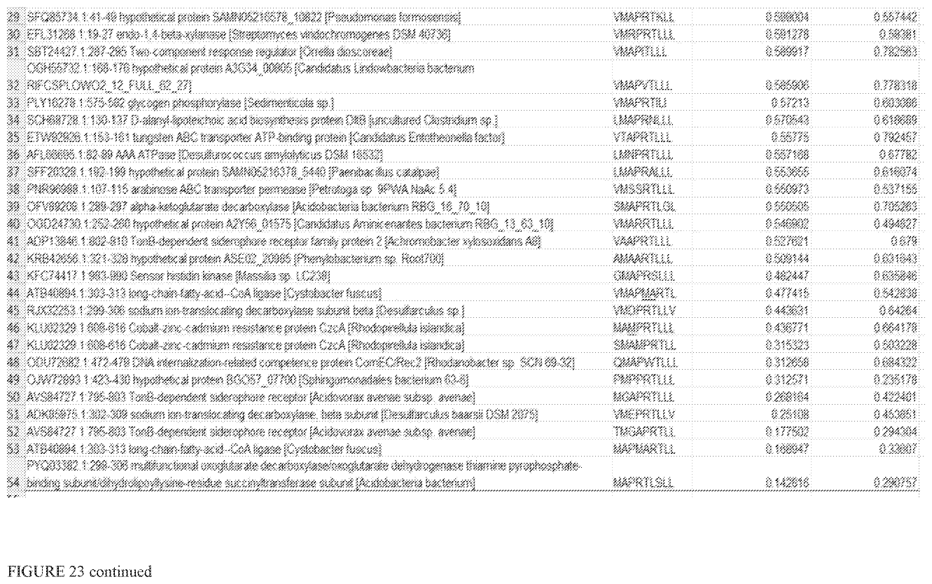

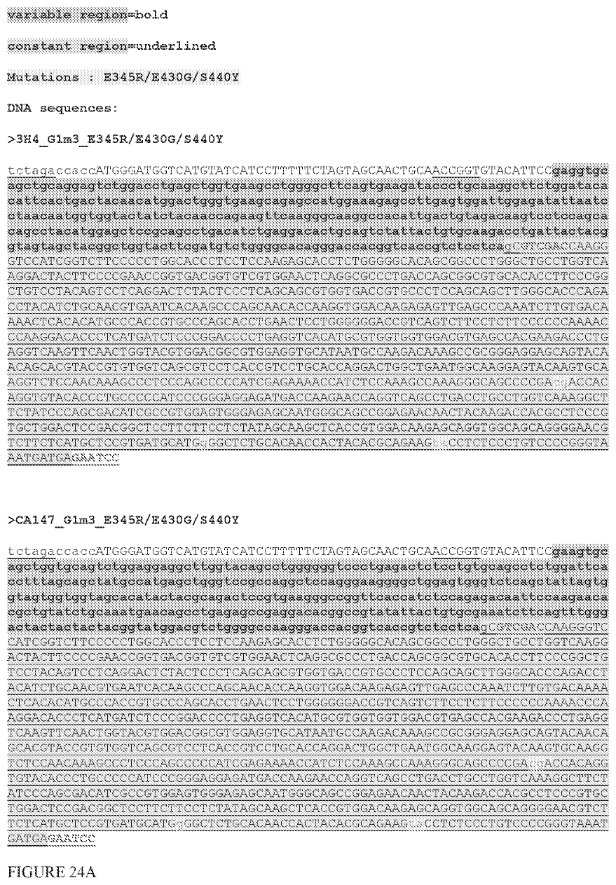

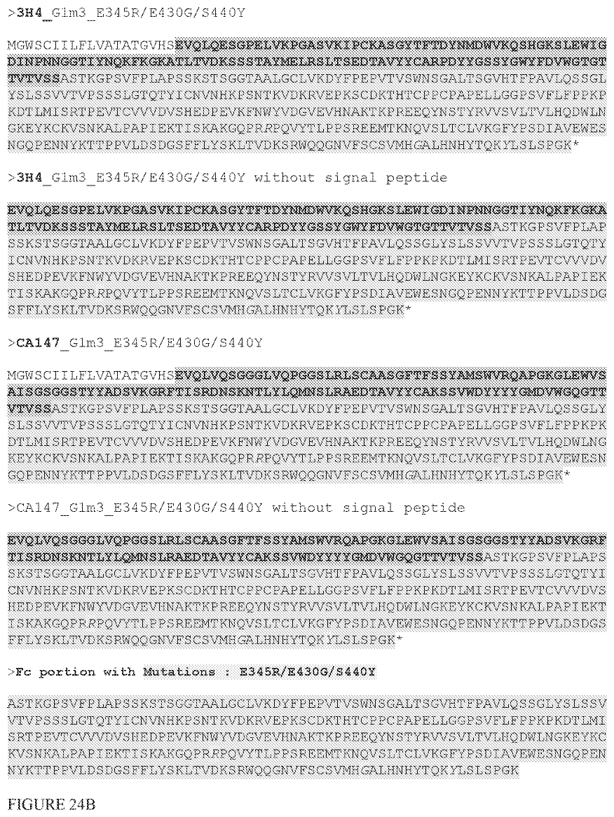

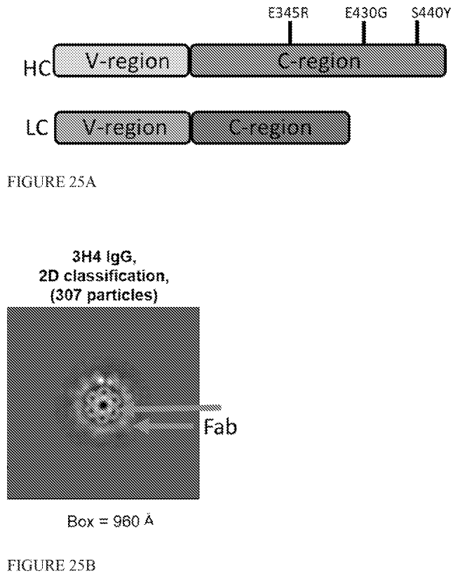

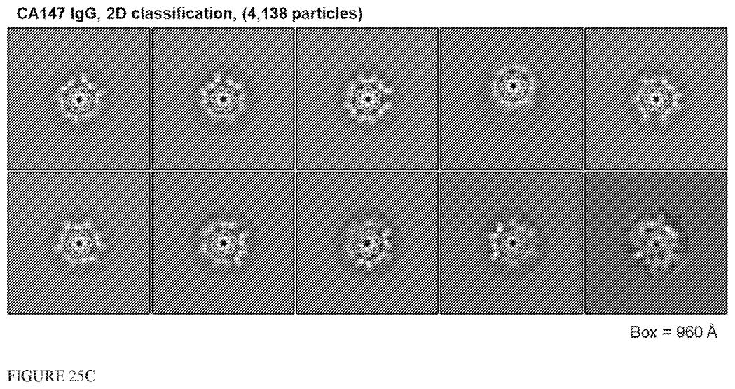

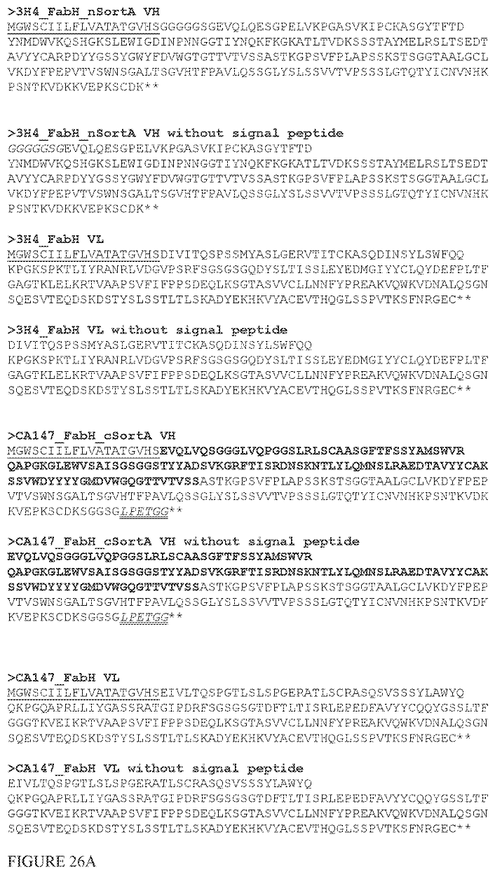

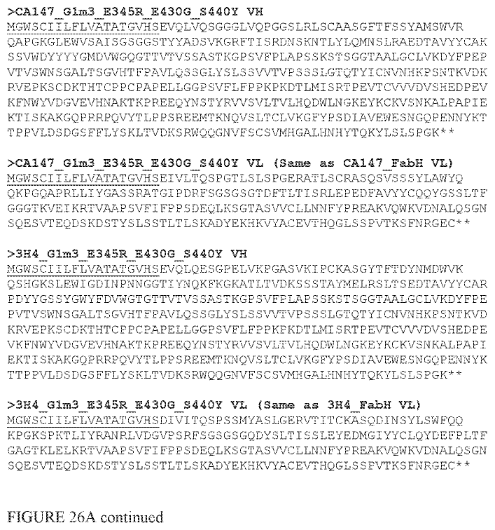

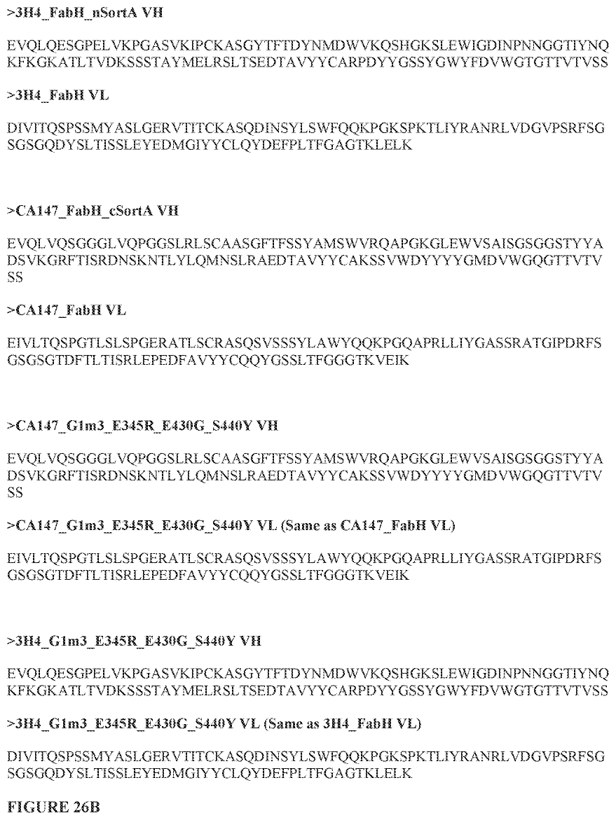

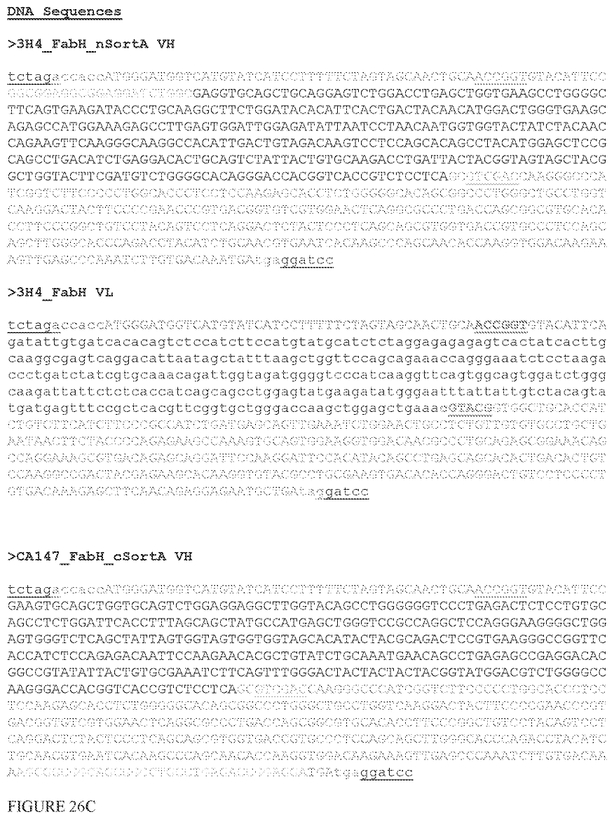

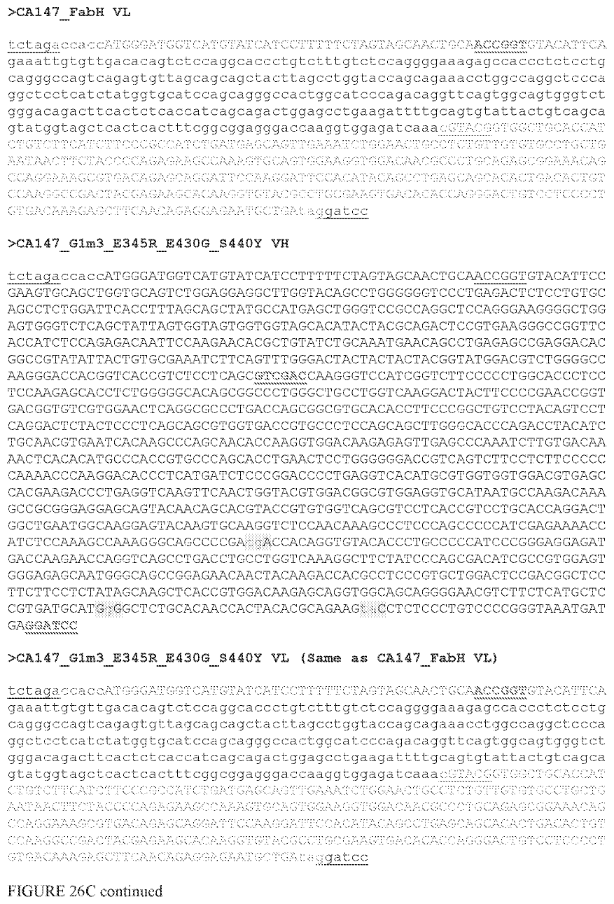

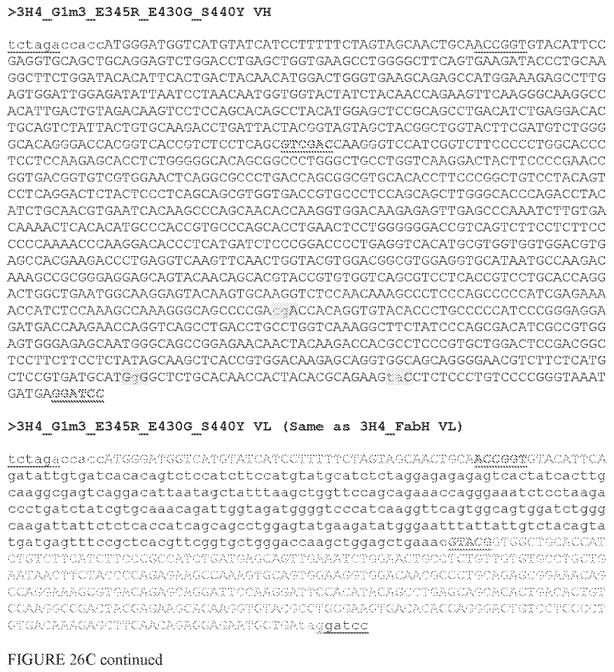

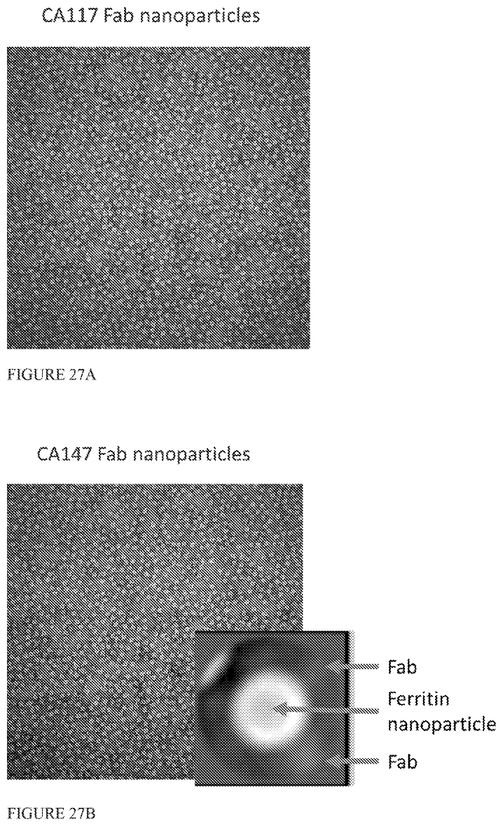

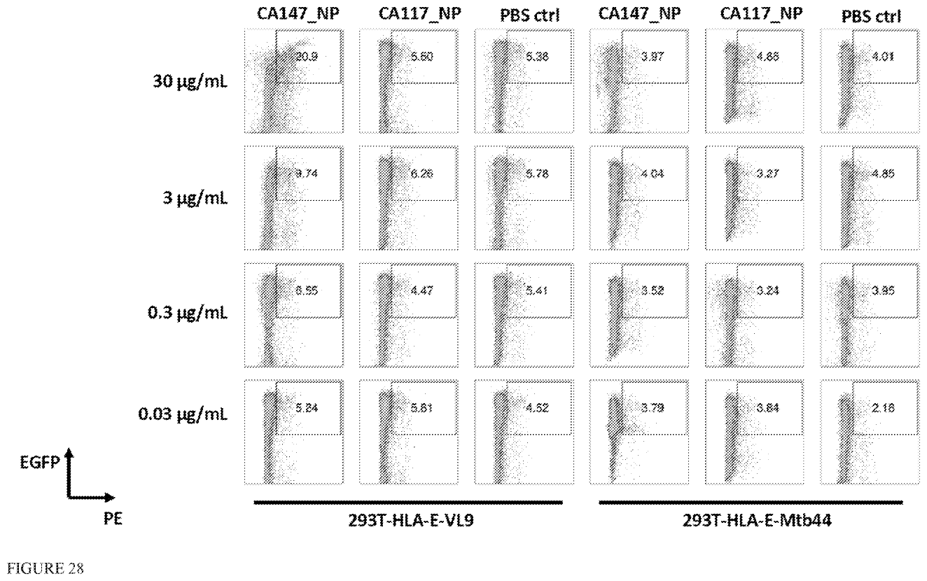

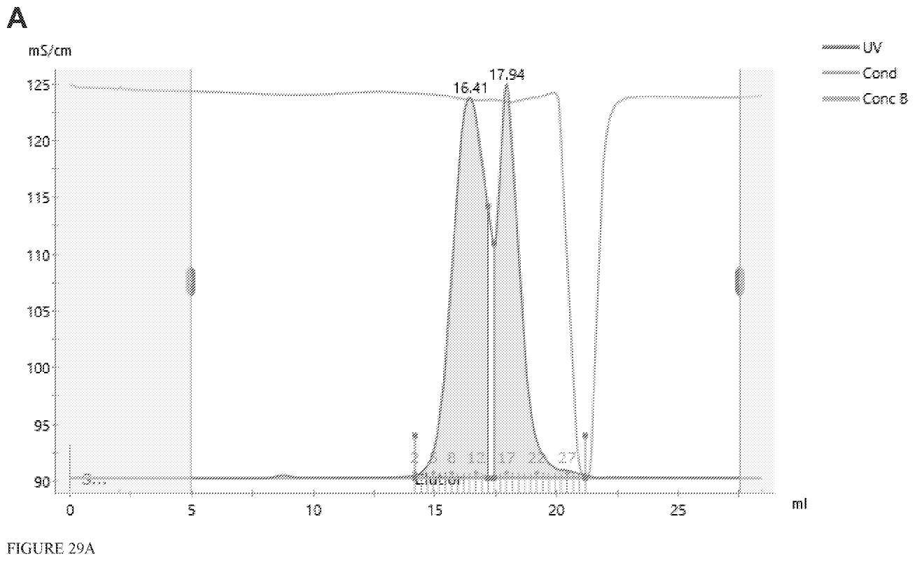

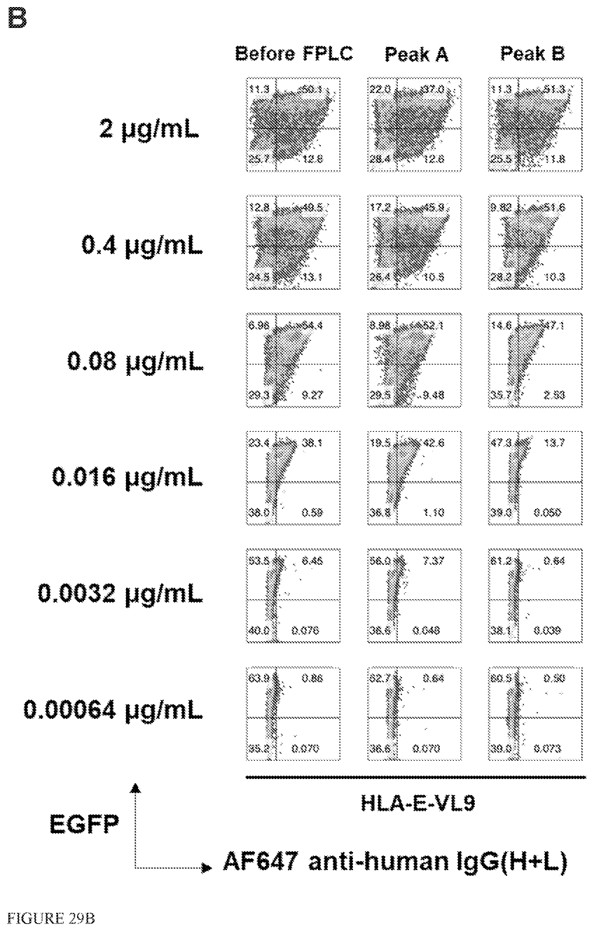

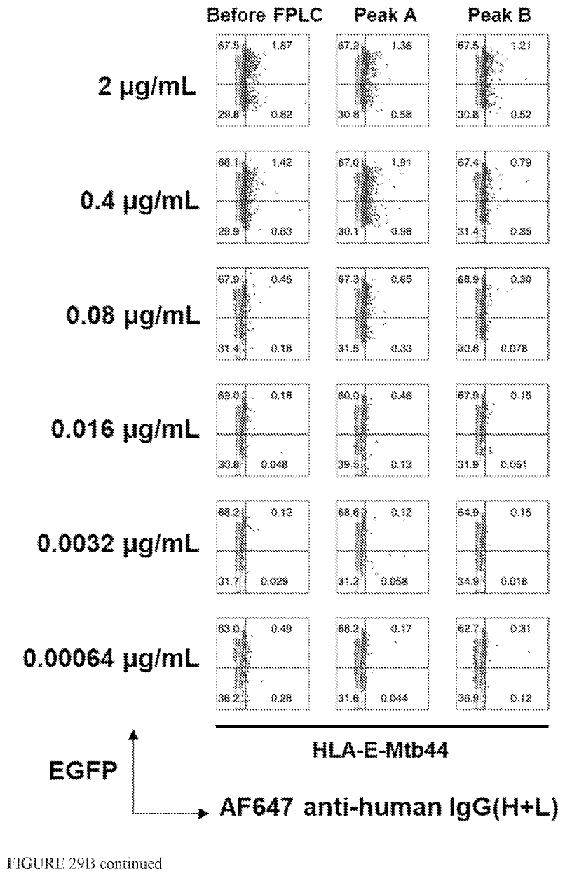

We obtained a co-complex crystal structure of the 3H4 Fab bound to VL9 peptide-loaded HLA-E, which packed in the C2 space group and diffracted to 1.8 Å(FIGS. 15-17). Although two copies of the co-complex were present in the asymmetric unit, a single copy constituted the focus of further discussion here since root-mean-square deviation (RMSD) calculations from Cα-atom pairwise alignment of the two copies indicated minimal repositioning of interfacing residues at the HLA-E-3H4 binding site (FIGS. 15-17). Additionally, pairwise alignment with the previously published non-receptor-bound HLA-E coordinates (PDB ID: 1MHE) (O'Callaghan et al., 1998) revealed minimal structural changes in HLA-E upon 3H4 engagement (FIGS. 15-17).

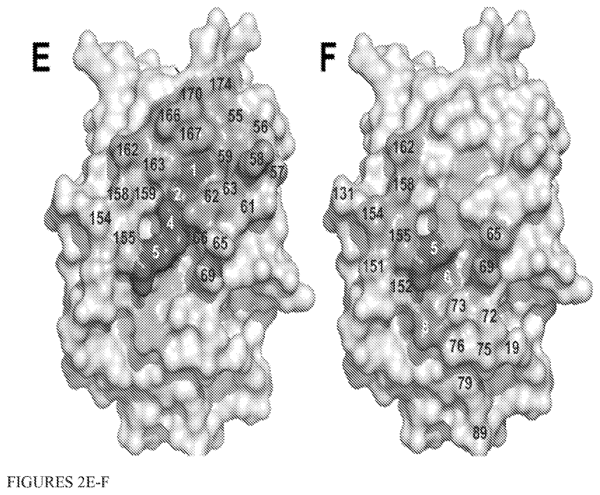

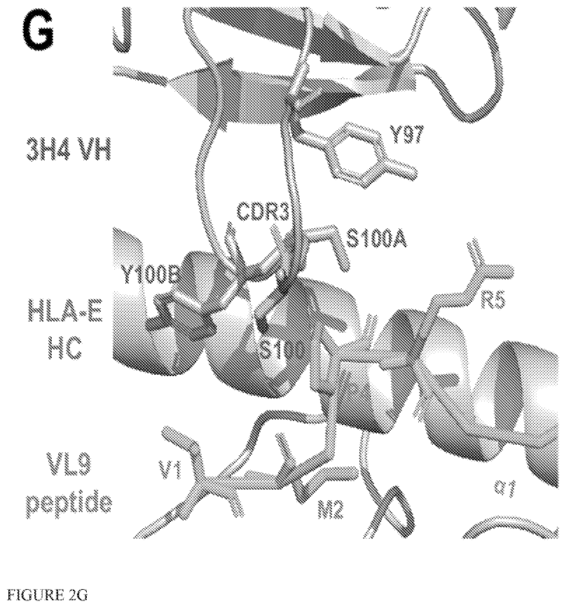

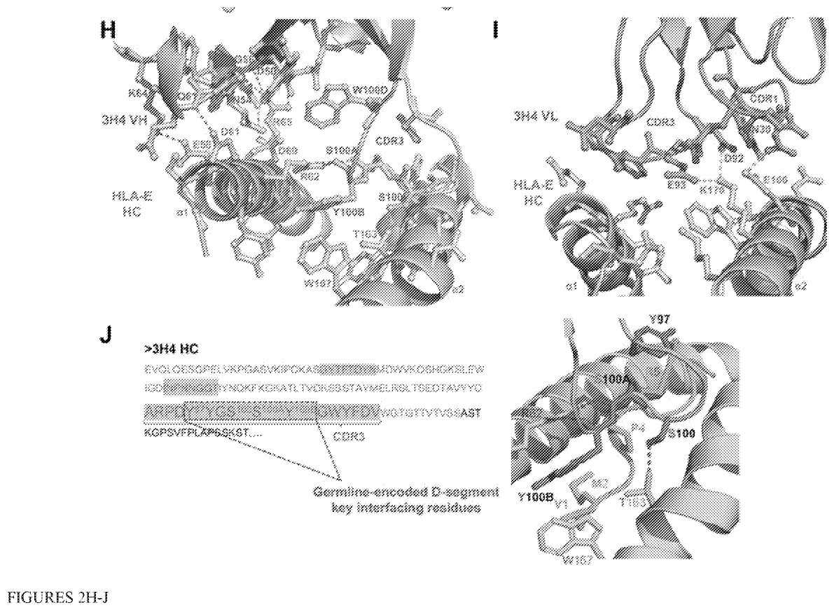

3H4 docked onto the N-terminal region of the HLA-E-peptide-binding groove making contacts with both α-helices of the HLA-E heavy chain in addition to residues 1-4 of the VL9 peptide (FIGS. 2A-B). The 3H4-HLA-E interface was mainly mediated via electrostatic interactions and was dominated by the 3H4 VH chain segment which created a total buried surface area of 1109.4 Å2 and formed ten hydrogen bonds (H-bonds) and three salt bridges with HLA-E residues of the al-helix and one H-bond with T163 of the HLA-E α2-helix. By contrast, the smaller 3H4 VL chain-HLA-E interface buried 522.8 Å2 and involved only three inter-molecular H-bonds and three salt bridges (FIGS. 15-17). Superposition of the 3H4-HLA-E-VL9 co-complex with a previously published HLA-E-bound CD94/NKG2A structure (Kaiser et al., 2008; Petrie et al., 2008) revealed steric clashes between the VH and VL domains of 3H4 and the CD94 and NKG2A subdomains, respectively (FIGS. 2C-D). Moreover, seven HLA-E heavy chain residues (positions 58, 59, 62, 63 on the al helix and 162, 163 and 167 on the α2 helix) are shared between the 3H4-HLA-E and CD94/NKG2A-HLA-E footprints (FIGS. 2E-F). Such steric clashes and overlapping footprints suggest simultaneous docking of these two HLA-E binding partners, 3H4 and NKG2A/CD94, would be disallowed.

Remarkably, all four of the 3H4-derived residues that interfaced with the VL9 peptide (Y97, S100, S100A and Y100B, Kabat numbering) resided within the VH CDR3 D-junction and were germline-encoded. This 3H4-VL9 interface was characterized by weak Van der Waals and hydrophobic contacts, such as those mediated between Y100B (3H4) and V1 or P4 (VL9) (FIG. 2G). Further, the positioning of the Y100B (3H4) side chain directly above V1 (VL9) in part explained the preference for small side chains at this position of the peptide and the dramatic reductions in 3H4 binding to HLA-E bound to VL9 variants with larger residues such as H or F at position 1 (FIG. 1G). Distinctive shape complementarity was also observed at the 3H4-VL9 interface with the side chains of S100 and S100A (3H4) wrapping around the cyclic side chain of P4 (VL9).

The germline-encoded VH CDR3 D-junction residues that formed the 3H4-VL9 interface (Y97, S100, S100A and Y100B), also mediated key contacts with the HLA-E heavy chain. The surface loop (residues A93-V102) containing these germline-encoded residues swept across the HLA-E-peptide-binding groove forming H-bonds with both the α1 and α2 helices; T163 of the HLA-E α2 helix formed an H-bond with S100 (3H4), and R62 of the HLA-E al-helix formed two H-bonds with the Y100B (3H4) mainchain and an additional H-bond with the main chain of S100A (3H4) (FIG. 2H). Moreover, Y100B (3H4) was involved in multiple polar pi stacking interactions. Not only was the Y100B side chain sandwiched between R62 and W167 of the HLA-E α1 and α2 helices, respectively, R62 (HLA-E al) was also positioned between the aromatic rings of Y100B and W100D of the VH CDR3 domain.

Key contacts outside the germline-encoded CDR3 D-junction region were also formed at the 3H4 VH-HLA-E or 3H4 VL-HLA-E interfaces. For 3H4 HC, the VH CDR2 region (residues I51-T57) was positioned above the HLA-E al-helix where a string of inter-molecular H-bonds were formed involving G56 and N54 of the VH CDR2 in addition to D50, Q61 and K64 of the framework VH chain region (FIG. 2H). Critically, R65 of the HLA-E al-helix formed four H-bonds with the 3H4 VH and also mediated polar pi stacking interactions with W100D of the VH CDR3 loop. For 3H4 LC, D92 and E93 of the VL CDR3 loop H-bond with K170 of the HLA-E α2-helix and N30 of the VL CDR1 loop formed an H-bond with the α2-helix residue, E166, of HLA-E (FIG. 2I). It is noteworthy that the four key interfacing residues of the 3H4 VH CDR3 D-junction (Y97, S100, S100A and Y100B) were germline-encoded (FIG. 2J). Since these residues interfaced with both the HLA-E heavy chain and VL9 peptide, the B cell receptor germline component played a central role in the recognition of VL9-bound HLA-E complexes by 3H4.

### 3H4 IgM Enhanced NK Cell Cytotoxicity Against HLA-E-VL9-Expressing Target Cells

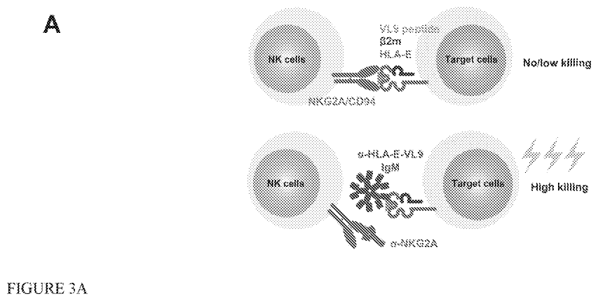

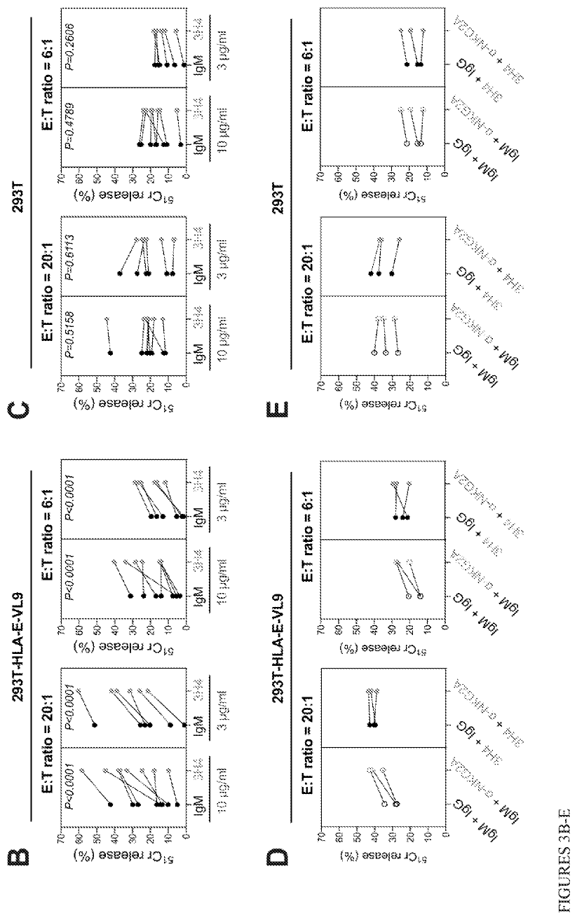

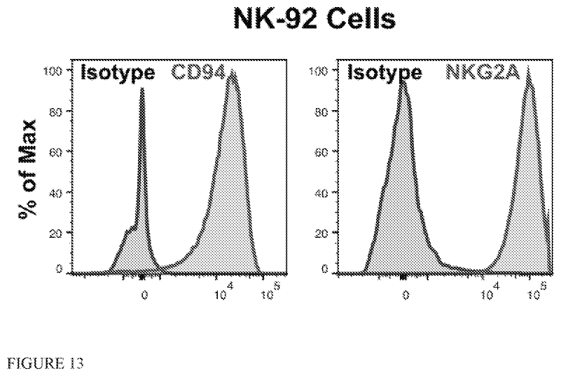

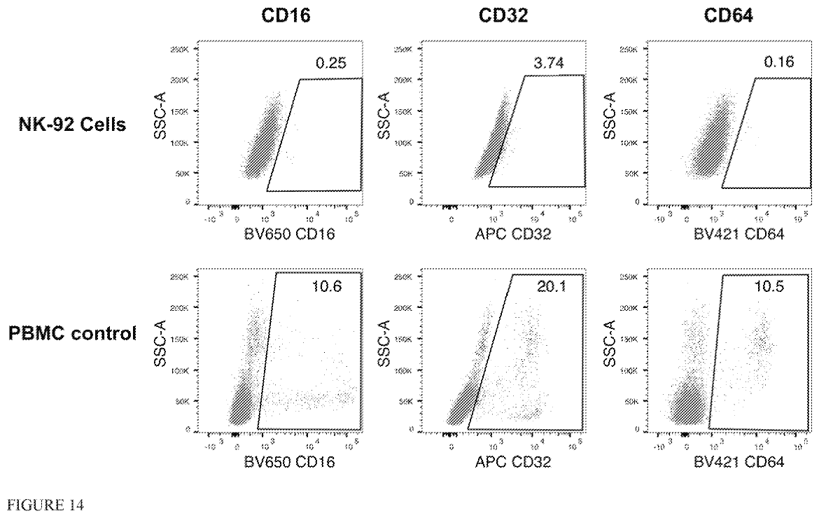

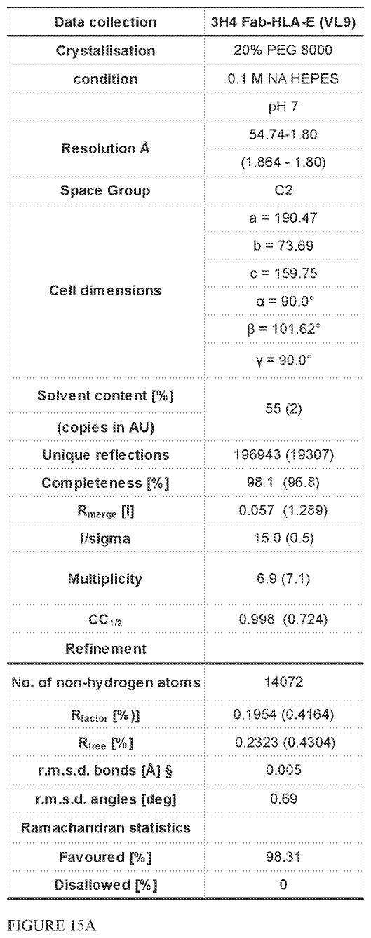

Given the suppressive role of the HLA-E-VL9/NKG2A/CD94 pathway in NK cell function, we tested whether the binding of mAb 3H4 to HLA-E-VL9 could enhance NK cell killing of target cells (FIG. 3A). An NKG2A/CD94-positive, CD16/CD32/CD64-negative human NK cell line, NK92 (FIGS. 13, 14), exhibited significantly increased cytotoxicity against HLA-E-VL9-transfected 293T cells (FIG. 3B; P<0.0001, mixed effects models) but not against non-HLA-E-expressing 293T cells (FIG. 3C) in the presence of 3H4 IgM compared to an isotype control IgM. In addition, we tested a combination of 3H4 with the NKG2A specific antibody, Z199. While Z199 alone enhanced NK killing against HLA-E-VL9 expressing cells, no additional elevation of killing was observed with the combination of mAbs 3H4 and Z199 (FIGS. 3D-E), suggesting that killing enhancement was maximal with either 3H4 or Z199 alone. These data demonstrated that HLA-E-VL9-specific IgM mAb 3H4 could enhance the killing capacity of NKG2A+NK cells by binding to HLA-E-VL9 on target cells.

The majority of multimeric IgM is restricted to serum and lymph and does not penetrate well into tissues (Sathe and Cusick, 2021). Thus, we constructed a recombinant 3H4 IgG in a human IgG1 backbone and tested it for ability to enhance NK92 cell killing of HLA-E-VL9 target cells. In contrast to 3H4 IgM, 3H4 IgG could not mediate enhancement of NK cell killing (FIG. 18A). Thus, either the affinity of the 3H4 Fab on an IgG was too low (FIG. 12E), or a multimeric antibody is needed for efficient blocking of HLA-E-VL9 binding to NKG2A/CD94 to enhance of NK killing.

### Rational Design of Affinity-Optimized 3H4 IgG

To distinguish between the need for higher affinity versus multimerization of the IgM antibody for enhanced NK killing activity, we aimed to obtain a 3H4 IgG with tighter binding and enhanced biological function. Therefore, we developed and analyzed 3H4 antibody libraries using high-throughput screening on the surface of yeast (FIG. 4A). A library was built that contained ˜1.1 million 3H4 scFv variants with amino acid diversity at sites that were determined by structural analysis to interact with HLA-E-VL9 (FIG. 4B). Seventeen total residues located in the CDR loops of 3H4 were randomized in groups of four based on their proximity, and all the possible combinations of amino acids were sampled at these sites (FIG. 18B). The resulting 3H4 scFv library was transformed into yeast and screened for three rounds by fluorescence-activated cell sorting (FACS) for binding to fluorescently labeled HLA-E-VL9 tetramer (FIG. 4C). All libraries were pooled and screened together, and eleven 3H4 variants were selected for experimental characterization as recombinant human IgGs from the highly represented clones remaining in the library upon the final selection round. These novel Abs (3H4 Gv1 to 3H4 Gv12) were mutated at positions 97-100 of the CDR H3 loop. Compared to the original 3H4 mAb, the optimized antibodies predominantly contained small amino acids at positions 97 and 98, a polar amino acid at position 99, and a large aromatic at position 100 that is closest to the HLA-E-VL9 (FIG. 4D). In non-limiting embodiments, position 97 includes T/S/G/A/R, position 98 includes A/G, position 99 includes R/Q/T/P, position 100 includes Y/F/T/W/H/P, or a combination thereof. See FIG. 4D.

We next expressed all eleven 3H4 Gv antibodies recombinantly as human IgGs and confirmed that they had higher binding than wild-type 3H4 IgG on cell surface expressed HLA-E-VL9 (FIGS. 4E and 18C). Two 3H4 variants, 3H4 G3v and 3H4 G6v, were selected for affinity and functional analysis. SPR measurements showed that the 1:1 dissociation constants (KDs) of the selected 3H4 variants for soluble HLA-E-VL9 were markedly improved compared to that of wild-type 3H4 that had a KD of 49.8 μM. 3H4 G3v showed the tightest HLA-E-VL9 binding, with a KD of 220 nM, representing a ˜226-fold improvement in affinity over the WT mAb (FIG. 4F). In the NK cytotoxicity assay, the optimized 3H4 mAbs enhanced NK-92 cell killing of HLA-E-VL9-transfected 293T cells at concentrations of 10 μg/ml and 1 μg/ml to levels comparable to those observed for 3H4 IgM (FIGS. 4G and 18D). Therefore, the higher affinity of affinity-optimized 3H4 IgG for HLA-E-VL9 could compensate for the need for avidity effect in the 3H4 IgM multimers to mediate NK enhancement.

### Germline-Encoding HLA-E-VL9-Specific Antibodies Exist in CMV-Negative, Healthy Humans

That HLA-E-VL9-specific antibodies were isolated from mice immunized with an unrelated peptide antigen (RL9HIV) implied that antibody 3H4 might be derived from the natural B cell pool rather than induced by immunization. Therefore, we assessed binding of HLA-E-VL9 fluorescent tetramers to B220+CD19+B cells from naïve HLA-B27/β2M TG mice and B6 mice and found that HLA-E-VL9-tetramer-binding B cells existed in unimmunized mice (FIGS. 20A-B). Additionally, all HLA-E-VL9-specific mouse antibodies were minimally mutated IgM antibodies (Table 5). These findings raised the hypothesis that HLA-E-VL9-specific antibodies were natural antibodies in mice.

We next asked if similar HLA-E-VL9 antibodies were present in the natural B cell pool in humans. Using HLA-E-VL9 tetramers as probes, we identified B cells expressing HLA-E-VL9-specific B cell receptors (BCRs) in four male, cytomegalovirus (CMV) seronegative human donors (FIGS. 5A and 18C, Table 6). We isolated 56 HLA-E-VL9-specific antibodies that reacted with HLA-E-VL9 complexes but not control HLA-E-peptide complexes (FIGS. 5B and 18D-E, FIG. 22); all were IgM (FIG. 22). By performing more in-depth analysis of the binding profiles of four representative HLA-E-VL9 antibodies—CA123, CA133, CA143 and CA147, we found that these antibodies exhibited differential cross-reactivities with rhesus Mamu-E-VL9 or mouse Qa-1-VL9 complexes (FIG. 20F) in addition to distinct binding specificities to VL9 peptide variants (FIG. 20G). The affinities (KD) of CA123 and CA147 on a human IgG1 backbone to soluble HLA-E-VL9 were 3.8 and 25.0 μM, respectively (FIG. 20H). Human HLA-E-VL9 antibodies CA147 and CA123 tested in functional NK killing assays as a recombinant human IgG1. CA147 enhanced NK-92 cell cytotoxicity to HLA-E-VL9-expressing target cells (FIG. 5C), whereas CA123 had no enhancing effect (FIGS. 20I-J), suggesting that NK killing-enhancement function of the HLA-E-VL9 antibodies was determined by factors beyond binding affinity. These data demonstrated that BCRs with diverse recognition patterns and NK cell cytotoxicity regulation function were present in male, CMV-seronegative humans.

In the four humans, the percentages of HLA-E-VL9-specific B cells in pan-B cells (CD3−CD235−CD14−CD16−CD19+) were 0.0009%-0.0023% (mean of 0.0014%) (FIG. 5D). HLA-E-VL9-specific B cells were IgD+IgM+/−B cells, in which four cell subsets were observed (FIG. 5E)—CD10−CD27−CD38+/−naïve B cells (71.4%), CD10+CD27−CD38++ immature or newly formed B cells (Giltiay et al., 2019) (10.7%), and CD10−CD27+CD38-non-class-switched memory cells, demonstrating that BCRs specifically targeting HLA-E-VL9 peptide existed in the naïve B cell repertoire of healthy humans.

V_{H}/V_{L} Gene Usage of HLA-E-VL9-Specific Antibodies

Natural antibodies demonstrate Ig repertoire skewing (Holodick et al., 2017; New et al., 2016). To characterize the human antibody gene usage of HLA-E-VL9 antibodies, we analyzed the paired heavy chain and light chain gene sequences of 56 human HLA-E-VL9 antibodies and found 1 multiple-member clone containing 6 antibodies in donor LP021 (Kepler et al., 2014) (FIG. 22). Next, we compared the 51 HLA-E-VL9-specific B cell clones with a reference human antibody repertoire (DeKosky et al., 2015). Over 45% of the heavy chain variable region (VH) genes were VH3-21 or VH3-11 in HLA-E-VL9 antibodies, whereas less than 7% of the control B cells used these two genes (FIG. 5F, FIG. 22). HLA-E-VL9 antibody light chain variable regions (Vx/VX) also were skewed and preferentially utilized IGKV3-15, IGKV1-39 and IGKV3-11 genes compared to controls (FIG. 5G, FIG. 22). No J chain gene usage preference was observed (FIGS. 21A-D). Moreover, HLA-E-VL9 antibodies showed a trend to have shorter heavy chain complementarity determining region 3 (CDR3) lengths than reference antibodies (FIG. 5H), while no difference was observed for light chain CDR3 (FIG. 5I). Given that HLA-E-VL9 antibodies were IgMs derived primarily from naïve or immature B cells, we compared the mutation frequencies of the 51 clones with a reference human antibody repertoire containing both naïve and antigen-experienced antibodies (DeKosky et al., 2016). Both HLA-E-VL9 antibody heavy and light chain variable region genes exhibited low somatic mutation rates that were similar to naïve B cell controls (FIGS. 5J-K). Thus, human HLA-E-VL9-specific antibodies were IgM, minimally mutated and displayed skewed usage of VH and Vx/VX genes.

HLA-E-VL9-Specific mAbs Recognize Microbiome-Derived VL9-Like Peptides Presented by HLA-E

We identified microbiome-derived VL9-like peptides from the NCBI microbiome database predicted by NetMHC to have HLA-E binding capacity (Andreatta and Nielsen, 2016; Nielsen et al., 2003) (FIG. 23). Eight peptides with the highest HLA-E binding scores were synthesized as 9 amino acid peptides, incubated with K562-E cells, and tested for mAb 3H4 HLA-E-VL9-specific antibody binding. Seven out of eight microbiome sequence-derived peptides showed strong HLA-E binding as indicated by the ability to stabilize and upregulate HLA-E expression, as determined by detecting with the HLA-E reactive antibody, 3D12 (FIGS. 5L and 21E). Notably, peptides with sequences very closely related to VL9 (VMAPRTLLL), VMPPRALLL (from Escherichia coli MS 175-1), VMAGRTLLL (from Stenotrophomonas sp.) and VMAPRTKLL (from Pseudomonas formosensis) were detected on K562-E cells by the HLA-E-peptide-antibody 3H4. Human HLA-E-VL9 antibodies CA143 and CA147 also reacted with Pseudomonas formosensis-derived peptide VMAPRTKLL bound to HLA-E (Lin et al., 2013) (FIGS. 5L and 21F-G). These data demonstrated that microbiome-derived peptides in complex with HLA-E were capable of binding to HLA-E-VL9-specific antibodies and raised the hypothesis that microbiome peptides may be one type of antigen capable of stimulating B cells with HLA-E-VL9 peptide specificity in vivo.

### Discussion

In this study, we have isolated and characterized antibodies reactive with HLA-E-VL9 peptide complexes, and found these antibodies were derived from the naïve IgM B cell BCR repertoire in mice as well as in non-immunized, HCMV seronegative male humans. Somatic mutations of these antibodies were minimal, and the affinities of these antibodies for HLA-E-VL9 were low. The lack of class-switching in HLA-E-VL9-specific antibodies may reflect self-tolerance of CD4 T cells and a lack of T cell help for affinity maturation of these antibodies. While the mouse antibodies were selected in the setting of HLA-E-unrelated peptide immunizations, they were minimally mutated IgM antibodies, as were the antibodies isolated from human CMV-negative, healthy males. Structural analysis of the HLA-E-VL9-3H4 Fab co-complex revealed that the 3H4 heavy chain made key contacts with HLA-E and the VL9 peptide using germline-encoded residues in the CDR-H3 (D) region. However, 3H4 is a mouse antibody that reacted with human HLA-E-VL9. The HLA-E equivalent in C57BL/6×SJL mice is Qa1b which presents a similar class Ia signal peptide AMAPRTLLL and 3H4 did not bind to this HLA-E-peptide complex. However, HLA-E-VL9-specific antibodies were identified in the naïve B cell pool of healthy humans and, like the mouse 3H4, the human CA147 HLA-E-VL9 antibody enhanced NK cytotoxicity of NKG2A+NK cells. Therefore, this type of natural antibody-producing B cell could play immunoregulatory roles in humans to enhance NK killing of pathogen infected cells in the early stages of a viral infection. If so, this might provide the selective force to maintain these enriched V genes in the germline.

Autoantibodies to HLA-Ia (Alberu et al., 2007; Morales-Buenrostro et al., 2008) and HLA-E heavy chains (Ravindranath et al., 2010a; Ravindranath et al., 2010b) have been detected in non-alloimmunized males, and contribute to allograft damage (Hickey et al., 2016; McKenna et al., 2000). It has been suggested that the HLA-E antibodies in non-alloimmunized humans could be elicited by autoantigens derived from viral, bacterial, or environmental agents cross-reactive with HLAs, or soluble HLA-E heavy chains that become immunogenic without the β2M subunit (Alberu et al., 2007; Hickey et al., 2016; Ravindranath et al., 2010a; Ravindranath et al., 2010b).

A recent study found that mouse gut microbial antigens shaped the BCR repertoire by contributing to BCR selection and affinity maturation (Chen et al., 2020). Therefore, we tested several potential HLA-E binding peptides with sequence similarities to VL9, derived from the human microbiome. Three of our HLA-E-VL9 antibodies recognized a subset of these HLA-E-presented microbiome-derived VL9-like peptides. These data imply that human microbial peptides presented by HLA-E, could potentially interact with HLA-E-VL9-bound naïve BCRs, and trigger expansion of B cells that express HLA-E-VL9-specific BCRs. Viruses, bacteria and other microbes stimulate such innate-adaptive immune interactions. The best known example is human cytomegalovirus (CMV), which encodes the VL9 sequence VMAPRTLIL in the leader sequence of its UL40 gene. This peptide is processed in a TAP independent manner and presented bound to HLA-E at the cell surface to inhibit NK cell killing and evade innate immune responses (Tomasec et al., 2000). This has not been reported to elicit antibody responses, but HLA-E-UL40 peptide-specific T cells have been described when the limited polymorphism in the HLA A, B and C sequences mismatches that of the virally-encoded VL9 peptide sufficiently to overcome self-tolerance (Sullivan et al., 2015). However, the subjects in our study were all HCMV seronegative, ruling out the possibility that these antibodies were HCMV-induced. Similarly, that they were male excluded pregnancy-induced priming.

Finally, harnessing NK cells to attack tumor cells has emerged as an attractive strategy for cancer immunotherapies (Guillerey et al., 2016; Lowry and Zehring, 2017). A promising target for therapeutic immune-modulation of NK cell functions is the NKG2A/CD94-HLA-E-VL9 interaction. Monalizumab, the first-in-class monoclonal antibody checkpoint inhibitor targeting NKG2A, enhances anti-tumor immunity by activating cytotoxic activities of effector CD8+ T cells and NK cells (Andre et al., 2018; Creelan and Antonia, 2019; van Hall et al., 2019). In our study, co-complex structural analysis revealed steric clashes between the 3H4 Fab and the NK inhibitory receptor NKG2A/CD94 when docked onto HLA-E-VL9, which explained the mechanism of 3H4 IgM enhancing NKG2A+NK cell killing. Notably, mouse 3H4 IgM, the affinity-optimized 3H4 IgG, and the recombinant IgG1 form of human CA147 both enhanced the cytotoxicity of an NKG2A+ human NK cell line NK92, which is a safe and established cell line for adoptive immunotherapy in phase I clinical trials (Klingemann et al., 2016). Thus, HLA-E-VL9-targeting antibodies 3H4 and CA147 could have therapeutic potential as NK checkpoint inhibitors.

In summary, our study has demonstrated a novel specificity of IgM natural antibodies, that of recognition of HLA-E-VL9 peptide complexes, which suggests an NK cell immunoregulatory role by a subset of natural antibodies.

### Methods

Methods are provided in the Example 3 Supplementary Materials.

### REFERENCES

Alberu, J., Morales-Buenrostro, L. E., de Leo, C., Vargas-Rojas, M. I., Marino-Vazquez, L. A., and Crispin, J. C. (2007). A non-allogeneic stimulus triggers the production of de novo HLA antibodies in healthy adults. Transpl Immunol 18, 166-171.
Aldrich, C. J., DeCloux, A., Woods, A. S., Cotter, R. J., Soloski, M. J., and Forman, J. (1994). Identification of a Tap-dependent leader peptide recognized by alloreactive T cells specific for a class Ib antigen. Cell 79, 649-658.
Andre, P., Brunet, C., Guia, S., Gallais, H., Sampol, J., Vivier, E., and Dignat-George, F. (1999). Differential regulation of killer cell Ig-like receptors and CD94 lectin-like dimers on NK and T lymphocytes from HIV-1-infected individuals. Eur J Immunol 29, 1076-1085.
Andre, P., Denis, C., Soulas, C., Bourbon-Caillet, C., Lopez, J., Arnoux, T., Blery, M., Bonnafous, C., Gauthier, L., Morel, A., et al. (2018). Anti-NKG2A mAb Is a Checkpoint Inhibitor that Promotes Anti-tumor Immunity by Unleashing Both T and NK Cells. Cell 175, 1731-1743 e1713.
Andreatta, M., and Nielsen, M. (2016). Gapped sequence alignment using artificial neural networks: application to the MHC class I system. Bioinformatics 32, 511-517.
Braud, V., Jones, E. Y., and McMichael, A. (1997). The human major histocompatibility complex class Ib molecule HLA-E binds signal sequence-derived peptides with primary anchor residues at positions 2 and 9. Eur J Immunol 27, 1164-1169.
Braud, V. M., Allan, D. S., O'Callaghan, C. A., Soderstrom, K., D'Andrea, A., Ogg, G. S., Lazetic, S., Young, N. T., Bell, J. I., Phillips, J. H., et al. (1998). HLA-E binds to natural killer cell receptors CD94/NKG2A, B and C. Nature 391, 795-799.
Brooks, A. G., Posch, P. E., Scorzelli, C. J., Borrego, F., and Coligan, J. E. (1997). NKG2A complexed with CD94 defines a novel inhibitory natural killer cell receptor. J Exp Med 185, 795-800.
Chen, H., Zhang, Y., Ye, A. Y., Du, Z., Xu, M., Lee, C. S., Hwang, J. K., Kyritsis, N., Ba, Z., Neuberg, D., et al. (2020). BCR selection and affinity maturation in Peyer's patch germinal centres. Nature 582, 421-425.
Chiossone, L., Dumas, P. Y., Vienne, M., and Vivier, E. (2018). Natural killer cells and other innate lymphoid cells in cancer. Nat Rev Immunol 18, 671-688.
Colonna, M., and Samaridis, J. (1995). Cloning of immunoglobulin-superfamily members associated with HLA-C and HLA-B recognition by human natural killer cells. Science 268, 405-408.
Creelan, B. C., and Antonia, S. J. (2019). The NKG2A immune checkpoint—a new direction in cancer immunotherapy. Nat Rev Clin Oncol 16, 277-278.
DeKosky, B. J., Kojima, T., Rodin, A., Charab, W., Ippolito, G. C., Ellington, A. D., and Georgiou, G. (2015). In-depth determination and analysis of the human paired heavy- and light-chain antibody repertoire. Nat Med 21, 86-91.
DeKosky, B. J., Lungu, O. I., Park, D., Johnson, E. L., Charab, W., Chrysostomou, C., Kuroda, D., Ellington, A. D., Ippolito, G. C., Gray, J. J., et al. (2016). Large-scale sequence and structural comparisons of human naive and antigen-experienced antibody repertoires. Proc Natl Acad Sci USA 113, E2636-2645.
Giltiay, N. V., Giordano, D., and Clark, E. A. (2019). The Plasticity of Newly Formed B Cells. J Immunol 203, 3095-3104.
Guillerey, C., Huntington, N. D., and Smyth, M. J. (2016). Targeting natural killer cells in cancer immunotherapy. Nat Immunol 17, 1025-1036.
Hansen, S. G., Wu, H. L., Burwitz, B. J., Hughes, C. M., Hammond, K. B., Ventura, A. B., Reed, J. S., Gilbride, R. M., Ainslie, E., Morrow, D. W., et al. (2016). Broadly targeted CD8(+) T cell responses restricted by major histocompatibility complex E. Science 351, 714-720.
Hickey, M. J., Valenzuela, N. M., and Reed, E. F. (2016). Alloantibody Generation and Effector Function Following Sensitization to Human Leukocyte Antigen. Front Immunol 7, 30.
Holodick, N. E., Rodriguez-Zhurbenko, N., and Hernandez, A. M. (2017). Defining Natural Antibodies. Front Immunol 8, 872.
Hu, W., Wang, G., Huang, D., Sui, M., and Xu, Y. (2019). Cancer Immunotherapy Based on Natural Killer Cells: Current Progress and New Opportunities. Front Immunol 10, 1205.
Joosten, S. A., van Meijgaarden, K. E., van Weeren, P. C., Kazi, F., Geluk, A., Savage, N. D., Drijfhout, J. W., Flower, D. R., Hanekom, W. A., Klein, M. R., et al. (2010). Mycobacterium tuberculosis peptides presented by HLA-E molecules are targets for human CD8 T-cells with cytotoxic as well as regulatory activity. PLoS Pathog 6, e1000782.
Kaiser, B. K., Pizarro, J. C., Kerns, J., and Strong, R. K. (2008). Structural basis for NKG2A/CD94 recognition of HLA-E. Proc Natl Acad Sci USA 105, 6696-6701.
Karlhofer, F. M., Ribaudo, R. K., and Yokoyama, W. M. (1992). MHC class I alloantigen specificity of Ly-49+IL-2-activated natural killer cells. Nature 358, 66-70.
Kepler, T. B., Munshaw, S., Wiehe, K., Zhang, R., Yu, J. S., Woods, C. W., Denny, T. N., Tomaras, G. D., Alam, S. M., Moody, M. A., et al. (2014). Reconstructing a B-Cell Clonal Lineage. II. Mutation, Selection, and Affinity Maturation. Front Immunol 5, 170.
Kim, N., Lee, H. H., Lee, H. J., Choi, W. S., Lee, J., and Kim, H. S. (2019). Natural killer cells as a promising therapeutic target for cancer immunotherapy. Arch Pharm Res 42, 591-606.
Klingemann, H., Boissel, L., and Toneguzzo, F. (2016). Natural Killer Cells for Immunotherapy—Advantages of the NK-92 Cell Line over Blood NK Cells. Front Immunol 7, 91.
Kraemer, T., Blasczyk, R., and Bade-Doeding, C. (2014). HLA-E: a novel player for histocompatibility. J Immunol Res 2014, 352160.
Kraemer, T., Celik, A. A., Huyton, T., Kunze-Schumacher, H., Blasczyk, R., and Bade-Doding, C. (2015). HLA-E: Presentation of a Broader Peptide Repertoire Impacts the Cellular Immune Response-Implications on HSCT Outcome. Stem Cells Int 2015, 346714.
Lampen, M. H., Hassan, C., Sluijter, M., Geluk, A., Dijkman, K., Tjon, J. M., de Ru, A. H., van der Burg, S. H., van Veelen, P. A., and van Hall, T. (2013). Alternative peptide repertoire of HLA-E reveals a binding motif that is strikingly similar to HLA-A2. Mol Immunol 53, 126-131.
Lee, N., Goodlett, D. R., Ishitani, A., Marquardt, H., and Geraghty, D. E. (1998a). HLA-E surface expression depends on binding of TAP-dependent peptides derived from certain HLA class I signal sequences. J Immunol 160, 4951-4960.
Lee, N., Llano, M., Carretero, M., Ishitani, A., Navarro, F., Lopez-Botet, M., and Geraghty, D. E. (1998b). HLA-E is a major ligand for the natural killer inhibitory receptor CD94/NKG2A. Proc Natl Acad Sci USA 95, 5199-5204.
Lin, S. Y., Hameed, A., Liu, Y. C., Hsu, Y. H., Lai, W. A., and Young, C. C. (2013). Pseudomonas formosensis sp. nov., a gamma-proteobacteria isolated from food-waste compost in Taiwan. Int J Syst Evol Microbiol 63, 3168-3174.
Ljunggren, H. G., and Karre, K. (1985). Host resistance directed selectively against H-2-deficient lymphoma variants. Analysis of the mechanism. J Exp Med 162, 1745-1759.
Ljunggren, H. G., and Karre, K. (1990). In search of the ‘missing self’: MHC molecules and NK cell recognition. Immunol Today 11, 237-244.
Llano, M., Lee, N., Navarro, F., Garcia, P., Albar, J. P., Geraghty, D. E., and Lopez-Botet, M. (1998). HLA-E-bound peptides influence recognition by inhibitory and triggering CD94/NKG2 receptors: preferential response to an HLA-G-derived nonamer. Eur J Immunol 28, 2854-2863.
Lowry, L. E., and Zehring, W. A. (2017). Potentiation of Natural Killer Cells for Cancer Immunotherapy: A Review of Literature. Front Immunol 8, 1061.
Mahapatra, S., Mace, E. M., Minard, C. G., Forbes, L. R., Vargas-Hernandez, A., Duryea, T. K., Makedonas, G., Banerjee, P. P., Shearer, W. T., and Orange, J. S. (2017). High-resolution phenotyping identifies NK cell subsets that distinguish healthy children from adults. PLoS One 12, e0181134.
Marin, R., Ruiz-Cabello, F., Pedrinaci, S., Mendez, R., Jimenez, P., Geraghty, D. E., and Garrido, F. (2003). Analysis of HLA-E expression in human tumors. Immunogenetics 54, 767-775.
McKenna, R. M., Takemoto, S. K., and Terasaki, P. I. (2000). Anti-HLA antibodies after solid organ transplantation. Transplantation 69, 319-326.
Michaelsson, J., Teixeira de Matos, C., Achour, A., Lanier, L. L., Karre, K., and Soderstrom, K. (2002). A signal peptide derived from hsp60 binds HLA-E and interferes with CD94/NKG2A recognition. J Exp Med 196, 1403-1414.
Morales-Buenrostro, L. E., Terasaki, P. I., Marino-Vazquez, L. A., Lee, J. H., El-Awar, N., and Alberu, J. (2008). “Natural” human leukocyte antigen antibodies found in nonalloimmunized healthy males. Transplantation 86, 1111-1115.
New, J. S., King, R. G., and Kearney, J. F. (2016). Manipulation of the glycan-specific natural antibody repertoire for immunotherapy. Immunol Rev 270, 32-50.
Nielsen, M., Lundegaard, C., Worning, P., Lauemoller, S. L., Lamberth, K., Buus, S., Brunak, S., and Lund, O. (2003). Reliable prediction of T-cell epitopes using neural networks with novel sequence representations. Protein Sci 12, 1007-1017.
O'Callaghan, C. A., Tormo, J., Willcox, B. E., Braud, V. M., Jakobsen, B. K., Stuart, D. I., McMichael, A. J., Bell, J. I., and Jones, E. Y. (1998). Structural features impose tight peptide binding specificity in the nonclassical MHC molecule HLA-E. Mol Cell 1, 531-541.
Pende, D., Falco, M., Vitale, M., Cantoni, C., Vitale, C., Munari, E., Bertaina, A., Moretta, F., Del Zotto, G., Pietra, G., et al. (2019). Killer Ig-Like Receptors (KIRs): Their Role in NK Cell Modulation and Developments Leading to Their Clinical Exploitation. Front Immunol 10, 1179.
Petrie, E. J., Clements, C. S., Lin, J., Sullivan, L. C., Johnson, D., Huyton, T., Heroux, A., Hoare, H. L., Beddoe, T., Reid, H. H., et al. (2008). CD94-NKG2A recognition of human leukocyte antigen (HLA)-E bound to an HLA class I leader sequence. J Exp Med 205, 725-735.
Raulet, D. H. (2006). Missing self recognition and self tolerance of natural killer (NK) cells. Semin Immunol 18, 145-150.
Ravindranath, M. H., Kaneku, H., El-Awar, N., Morales-Buenrostro, L. E., and Terasaki, P. I. (2010a). Antibodies to HLA-E in nonalloimmunized males: pattern of HLA-Ia reactivity of anti-HLA-E-positive sera. J Immunol 185, 1935-1948.
Ravindranath, M. H., Taniguchi, M., Chen, C. W., Ozawa, M., Kaneku, H., El-Awar, N., Cai, J., and Terasaki, P. I. (2010b). HLA-E monoclonal antibodies recognize shared peptide sequences on classical HLA class Ia: relevance to human natural HLA antibodies. Mol Immunol 47, 1121-1131.
Rolle, A., Meyer, M., Calderazzo, S., Jager, D., and Momburg, F. (2018). Distinct HLA-E Peptide Complexes Modify Antibody-Driven Effector Functions of Adaptive NK Cells. Cell Rep 24, 1967-1976 e1964.
Sathe, A., and Cusick, J. K. (2021). Biochemistry, Immunoglobulin M. In StatPearls (Treasure Island (FL)).
Sensi, M., Pietra, G., Molla, A., Nicolini, G., Vegetti, C., Bersani, I., Millo, E., Weiss, E., Moretta, L., Mingari, M. C., et al. (2009). Peptides with dual binding specificity for HLA-A2 and HLA-E are encoded by alternatively spliced isoforms of the antioxidant enzyme peroxiredoxin 5. Int Immunol 21, 257-268.
Souza-Fonseca-Guimaraes, F., Cursons, J., and Huntington, N. D. (2019). The Emergence of Natural Killer Cells as a Major Target in Cancer Immunotherapy. Trends Immunol 40, 142-158.
Sullivan, L. C., Westall, G. P., Widjaja, J. M., Mifsud, N. A., Nguyen, T. H., Meehan, A. C., Kotsimbos, T. C., and Brooks, A. G. (2015). The Presence of HLA-E-Restricted, CMV-Specific CD8+ T Cells in the Blood of Lung Transplant Recipients Correlates with Chronic Allograft Rejection. PLoS One 10, e0135972.
Taurog, J., Hammer, R., Maika, S., Sams, K., El-Zaatari, F., Stimpson, S., and Schwab, J. (1990). HLA-B27 transgenic mice as potential models of human disease. In Transgenic mice and mutants in MHC research (Springer), pp. 268-275.
Tomasec, P., Braud, V. M., Rickards, C., Powell, M. B., McSharry, B. P., Gadola, S., Cerundolo, V., Borysiewicz, L. K., McMichael, A. J., and Wilkinson, G. W. (2000). Surface expression of HLA-E, an inhibitor of natural killer cells, enhanced by human cytomegalovirus gpUL40. Science 287, 1031.
van Hall, T., Andre, P., Horowitz, A., Ruan, D. F., Borst, L., Zerbib, R., Narni-Mancinelli, E., van der Burg, S. H., and Vivier, E. (2019). Monalizumab: inhibiting the novel immune checkpoint NKG2A. J Immunother Cancer 7, 263.
van Meijgaarden, K. E., Haks, M. C., Caccamo, N., Dieli, F., Ottenhoff, T. H., and Joosten, S. A. (2015). Human CD8+ T-cells recognizing peptides from Mycobacterium tuberculosis (Mtb) presented by HLA-E have an unorthodox Th2-like, multifunctional, Mtb inhibitory phenotype and represent a novel human T-cell subset. PLoS Pathog 11, e1004671.
van Montfoort, N., Borst, L., Korrer, M. J., Sluijter, M., Marijt, K. A., Santegoets, S. J., van Ham, V. J., Ehsan, I., Charoentong, P., Andre, P., et al. (2018). NKG2A Blockade Potentiates CD8 T Cell Immunity Induced by Cancer Vaccines. Cell 175, 1744-1755 e1715.
Walters, L. C., Harlos, K., Brackenridge, S., Rozbesky, D., Barrett, J. R., Jain, V., Walter, T. S., O'Callaghan, C. A., Borrow, P., Toebes, M., et al. (2018). Pathogen-derived HLA-E bound epitopes reveal broad primary anchor pocket tolerability and conformationally malleable peptide binding. Nat Commun 9, 3137.
Yokoyama, W. M., and Kim, S. (2006). How do natural killer cells find self to achieve tolerance?Immunity 24, 249-257.
Yu, Y. Y., Netuschil, N., Lybarger, L., Connolly, J. M., and Hansen, T. H. (2002). Cutting edge: single-chain trimers of MHC class I molecules form stable structures that potently stimulate antigen-specific T cells and B cells. J Immunol 168, 3145-3149.

### Materials and Methods

### Cell Lines

K562-E cells (K562 cells stably expressing HLA-E) and K562-E/UL49.5 cells (with a TAP-inhibitor UL49.5) are kindly provided by Dr. Thorbald van Hall from Leiden University (Lampen et al., 2013). All the other cells used in this study are from ATCC. 293T cells (ATCC CRL-3216) were maintained in Dulbecco's Modified Eagle's Medium (DMEM; Gibco, Catalog #10564) supplemented with 10% fetal bovine serum (FBS; Gibco, Catalog #10099141) and 1% penicillin/streptomycin (Gibco, Catalog #10378016). K562 cells (ATCC CCL-243), K562-E cells and K562-E/UL49.5 cells were cultured in Iscove's Modified Dulbecco's Medium (IMDM; Hyclone, Catalog #SH30228.01) supplemented with 10% FBS. Jurkat, DU-4475 and U-937 cells were cultured in RPMI-1640 medium (Gibco, Catalog #72400) supplemented with 10% FBS. SiHa cells were cultured in Minimum Essential Medium (MEM; Gibco, Catalog #11095080) supplemented with 10% FBS. The NK-92 human cell line (ATCC CRL-2407) was cultured in Alpha Minimum Essential medium (α-MEM; Gibco, Catalog #12561072) supplemented with 2 mM L-glutamine, 0.2 mM inositol, 0.1 mM 2-mercaptoethanol, 0.02 mM folic acid, 100 U/ml recombinant IL-2 (Biolegend, Catalog #589108), 12.5% horse serum (Gibco, Catalog #16050122) and 12.5% FBS. All the cells were maintained at 37° C., 5% CO2 in humidified incubators.

### Animals

Transgenic mice carrying human β2-microglobulin (β2m) and HLA-B*27:05 genes were obtained from Jackson lab (B6.Cg-Tg(B2M, HLA-B*27:05)56-3Trg/DcrJ; stock #003428). Hemizygous mice were used in this experiment, as this strain is homozygous lethal. For hemizygous mice genotyping, peripheral blood lymphocytes (PBLs) were isolated and stained using mouse CD45 antibody (Biolegend, Catalog #103122), human HLA class I antibody (Biolegend, Catalog #311406) and human β2m antibody (Biolegend, Catalog #316312). All animal experiments were conducted with approved protocols from the Duke University Institutional Animal Care and Use Committee.

### Human Subjects

Human leukapheresis frozen vials were collected by the External Quality Assurance Program Oversight Laboratory (EQAPOL) (Sanchez et al., 2014a; Sanchez et al., 2014b). Samples from four male donors were used in this study. Table 6 shows the clinical characteristics of the individuals studied. All experiments that related to human subjects was carried out with the informed consent of trial participants and in compliance with Institutional Review Board protocols approved by Duke University Medical Center.

### Peptide Synthesis

The VL9 peptide (VMAPRTVLL) was synthesized to >85% purity via Fmoc (9-fluorenylmethoxy carbonyl) chemistry by Genscript USA and reconstituted to 200 mM in DMSO.

### HLA-E-Peptide Protein Refolding and Purification

β2-microglobulin, previously purified from inclusion bodies in a Urea-MES buffer, was added to a refolding buffer to achieve a final concentration of 2 μM. The refold buffer comprised 100 mM Tris pH8.0, 400 mM L-arginine monohydrochloride, 2 mM EDTA, 5 mM reduced glutathione and 0.5 mM oxidized Glutathione and was prepared in MiliQ water. A 20 μM concentration of VL9 peptide (VMAPRTVLL), previously reconstituted to 200 mM in DMSO, was added to the refolding buffer followed by HLA-E*0103 heavy chain, which was pulsed into the refold to a final concentration of 1 μM. Once the refold had incubated for 72 hours at 4° C. it was filtered through a 1.0 m cellular nitrate membrane and concentrated in the VivaFlow 50R and VivaSpin Turbo Ultrafiltration centrifugal systems with 10 kDa molecular weight cut-offs. The concentrated samples were injected onto a Superdex S75 16/60 column and refolded protein eluted according to size into phosphate buffered saline (PBS). Eluted protein complexes were validated by non-reducing SDS-PAGE electrophoresis on NuPAGE 12% Bis-Tris protein gels and further concentrated via VivaSpin Turbo Ultrafiltration centrifugal device to 1.1 mg/mL.

### HLA-E-Peptide Biotinylation and Tetramer Generation

HLA-E-peptide samples requiring biotinylation were subsequently buffered exchanged on Sephadex G-25 PD10 columns (GE Healthcare, UK) into 10 mM Tris buffer using commercially available BirA enzyme (Avidity, USA) following the manufacturer's instructions. Following overnight biotinylation, protein samples were subsequently purified into 20 mM Tris pH8,100 mM NaCl buffer or PBS on a HiLoad 16/600 Superdex 75 pg column using an AKTA size exclusion fast protein liquid chromatography (FPLC) system. Correctly folded β2m-HLA-E*01:03-peptide complexes were subsequently concentrated to 2 mg/mL and snap frozen.

HLA-E*01:03 tetramers were generated via conjugation to various fluorescent labels including Extravidin-PE (Sigma), Streptavidin-bound APC (Biolegend, San Diego) or BV421 (Biolegend, San Diego) at a Molar ratio of 4:1 as previously described (Braud et al., 1998).

### Immunization in HLA-B27/β2m Transgenic Mice

HLA-B27/β2m transgenic mice (n=23) were intramuscularly (i.m.) immunized with pooled HLA-E-RL9HIV complex (12.5 μg/animal) and HLA-E-RL9SIV complex (12.5 g/animal) adjuvanted with STR8S-C (Moody et al., 2014) at weeks 0, 2, 4, 6, 12 and 16. MAb 3H4 was isolated from this study. In another experiment, HLA-B27/β2m transgenic mice (n=10) were i.p. immunized with either HLA-E-RL9HIV single chain trimer (SCT) transfected 293T cells (2×106 cells/animal) or HLA-E-RL9SIV SCT transfected 293T cells (2×106 cells/animal) at weeks 0, 2, 4, 6, 17 and 19. MAb 13F11 was isolated from this study. In the third experiment, HLA-B27/β2m transgenic mice (n=10) were i.m. immunized with HLA-E-VL9 complex (25 μg/animal) adjuvanted with STR8S-C at Week 0, 2 and 4, following by intraperitoneally (i.p.) immunization with HLA-E-VL9 SCT transfected 293T cells (2×106 cells/animal) at Week 14, 16 and 18. MAb 10C10 and 2D6 were isolated from this study. Serum titers were monitored by ELISA Mice with high binding antibody titers were selected for the subsequent spleen cell fusion and B cell sorting experiments.

### Hybridoma Cell Line Generation and Monoclonal Antibody Production

Mice were boosted with the indicated priming antigen 3 days prior to fusion. Spleen cells were harvested and fused with NS0 murine myeloma cells using PEG1500 to generate hybridomas. After 2 weeks, supernatant of hybridoma clones were collected and screened by flow cytometry-based high throughput screening (HTS). Specifically, we tested for antibodies differentially binding 293T cells transiently transfected with plasmid DNA expressing single chain peptide-HLA-E-β2m trimers so that they expressed HLA-E-RL9HIV, HLA-E-RL9SIV or HLA-E-VL9 at the cell surface. Hybridomas cells that secreted HLA-E-VL9 antibodies were cloned by limiting dilution for at least 5 rounds until the phenotypes of all limiting dilution wells are identical. IgG mAbs were purified by protein G affinity chromatography, while IgM mAbs were purified by ammonium sulfate precipitation and by Superose 6 column size-exclusion chromatography in AKTA Fast Protein Liquid Chromatography (FPLC) system. The VH and VL sequences of mAbs were amplified from hybridoma cell RNA using primers reported previously (Tian et al., 2016; von Boehmer et al., 2016).

### Cell Surface Staining and High-Throughput Screening (HTS)

HLA-E SCT constructs encoding HLA-E-VL9, HLA-E-RL9HIV, or HLA-E-RL9SIV were transfected into 293T cells using GeneJuice transfection reagent (Novagen, Catalog #70967). For epitope mapping experiment, a panel of HLA-E-VL9 SCT constructs with single amino acid mutations were transfected into 293T cells using the same method. Cells were dissociated with 0.1% EDTA at 48 hours post-transfection and stained with a Fixable Near-IR Dead Cell Stain Kit (Thermo Fisher, Catalog #L34976). After washing, primary antibodies (supernatant from hybridoma cells, supernatant from transfected cells, or purified antibodies) were added and incubated with cells for 1 hour at 4° C., following by staining with 1:1000 diluted secondary antibodies for 30 mins at 4° C. For mouse primary antibodies, we used Alexa Fluor 555 (AF555) conjugated goat anti-mouse IgG (H+L) (Thermo Fisher, Catalog #A32727) or Alexa Fluor 647 (AF647) conjugated goat anti-mouse IgG (H+L) (Thermo Fisher, Catalog #A32728) as secondary antibodies; for human primary antibodies, we used AF555 conjugated goat anti-human IgG (H+L) (Thermo Fisher, Catalog #A-21433) or AF647 conjugated goat anti-human IgG (H+L) (Thermo Fisher, Catalog #A-21445) as secondary antibodies. Cells were then washed 3 times and resuspended in fixation buffer (1% formaldehyde in PBS, pH7.4). Data were acquired on a BD LSR II flow cytometer and analyzed using FlowJo version 10.

### 3H4 Fab Production

A humanized version of the 3H4 antibody (3H4-huIgG1) was digested to produce Fab fragments using the Pierce Fab Preparation kit (ThermoFisher SCIENTIFIC). 3H4 Fab-retrieved sample was further purified by size exclusion on a Superdex S75 16/60 column and eluted into PBS buffer. Following concentration to 1.1 mg/mL and SDS-PAGE gel-based validation, 3H4 Fab purified material was incubated for 1 hours on ice with freshly purified HLA-E-VL9. The combined 3H4:Fab-HLA-E-VL9 sample was concentrated to 7.5 mg/mL prior to crystallographic set-up.

### Crystallization Screening

Crystals were grown via sitting drop vapour-diffusion at 20° C. in a 200 nL drop with a 1:1 protein to reservoir ratio (Walter et al., 2005). The 3H4 Fab-HLA-E(VL9) co-complex crystallized in 20% PEG 8000, 0.1 M Na HEPES at pH 7, in the ProPlex sparse matrix screen. Crystals were cryo-preserved in 25% glycerol and diffraction data were collected at the 103 beamline of Diamond Light Source.

### Crystallographic Analysis

Diffraction data were merged and indexed in xia2 dials (Winter et al., 2018). Outer shell reflections were excluded from further analysis to ensure the CC1/2 value exceeded the minimum threshold (>0.5) in each shell (Karplus and Diederichs, 2012). Sequential molecular replacement was carried out in MolRep of the CCP4i suite using molecule one of the previously published Mtb44-bound HLA-E structure with the peptide coordinates deleted (PDB ID: 6GH4) and one molecule of the previously published anti-APP-tag Fab structure (PDB ID: 6HGU) as phasing models (Vagin and Teplyakov, 2010; Winn et al., 2011). Rigid body and retrained refinement were subsequently carried out by Phenix.refine (Afonine et al., 2012) in between manual model building in Coot (Emsley et al., 2010). Model geometry was validated by MolProbity (Chen et al., 2010) and structural interpretation was conducted using the PyMOL Molecular Graphics System, version 2.0 (Schrödinger, LLC) in addition to the PDBePISA (Krissinel and Henrick, 2007) and PDBeFOLD (Krissinel and Henrick, 2004) servers.

### Antigen-Specific Single B Cell Sorting

HLA-E-VL9-specific human B cells were sorted in flow cytometry using a three-color sorting technique. Briefly, the stabilized HLA-E-β2M-peptide complexes were made as tetramers and conjugated with different fluorophores. Human pan-B cells, including naïve and memory B cells, were isolated from PBMCs of healthy donors using human pan-B cell enrichment kit (STEMCELL, Catalog #19554). The isolated pan-B cells were then stained with IgM PerCp-Cy5.5 (Clone #G20-127, BD Biosciences, Catalog #561285), IgD FITC (Clone #IA6-2, BD Biosciences, Catalog #555778), CD3 PE-Cy5 (Clone #HIT3a, BD Biosciences, Catalog #555341), CD235a PE-Cy5 (Clone #GA-R2, BD Biosciences, Catalog #559944), CD10 PE-CF594 (Clone #HI10A, BD Biosciences, Catalog #562396), CD27 PE-Cy7 (Clone #0323, eBioscience, Catalog #25-0279), CD16 BV570 (Clone #3G8, Biolegend, Catalog #302035), CD14 BV605 (Clone #M5E2, Biolegend, Catalog #301834), CD38 APC-AF700 (Clone #LS198-4-2, Beckman Coulter, Catalog #B23489), CD19 APC-Cy7 (Clone #LJ25C1, BD Biosciences, Catalog #561743) and tetramers at 2 μg/million cells (including BV421-conjugated HLA-E-VL9 tetramer, PE-conjugated HLA-E-VL9 tetramer, APC-conjugated HLA-E-RL9SIV tetramer and APC-conjugated HLA-E-RL9HIV tetramer). The cells were then stained with a Fixable Aqua Dead Cell Stain Kit (Invitrogen, Catalog #L34957). HLA-E-VL9-specific B cells were sorted in BD FACSAria II flow cytometer (BD Biosciences) for viable CD3neg/CD14neg/CD16neg/CD235aneg/CD19pos/HLA-E-VL9double-pos/HLA-E-RL9HIVneg/HLA-E-RL9SIVneg subset as single cells in 96-well plates.

### PCR Amplification of Human Antibody Genes

The VHDHJH and VLJL genes were amplified by RT-PCR from the flow cytometry-sorted single B cells using the methods as described previously (Liao et al., 2009; Wrammert et al., 2008) with modification. The PCR-amplified genes were then purified and sequenced with 10 μM forward and reverse primers. Sequences were analyzed by using the human library in Clonalyst for the VDJ arrangements of the immunoglobulin IGHV, IGKV, and IGLV sequences and mutation frequencies (Kepler et al., 2014). Clonal relatedness of VHDHJH and VLJL sequences was determined as previously described (Liao et al., 2013).

Expression of VHDHJH and VLJL as Full-Length IgG Recombinant mAbs

Transient transfection of recombinant mAbs was performed as previously described (Liao et al., 2009). Briefly, purified PCR products were used for overlapping PCR to generate linear human antibody expression cassettes. The expression cassettes were transfected into 293i cells using ExpiFectamine (Thermo Fisher Scientific, Catalog #A14525). The supernatant samples containing recombinant antibodies were used for cell surface staining and HTS assay to measure the binding reactivities.

The selected human antibody genes were then synthesized and cloned (GenScript) in a human IgG1 backbone with 4A mutations (Saunders, 2019). Recombinant IgG mAbs were then produced in HEK293i suspension cells by transfection with ExpiFectamine and purified using Protein A resin. The purified mAbs were run in SDS-PAGE for Coomassie blue staining and western blot. Antibodies with aggregation were further purified in AKTA FPLC system using a Superdex 200 size-exclusion column.

### Surface Plasmon Resonance (SPR)

Surface plasmon resonance assays were performed on a BIAcore 3000 instrument, and data analysis was performed with BIAevaluation 3.0 software as previously described (Liao et al., 2006). Purified mAbs flowed over CM5 sensor chips at concentrations of 100 g/ml, and antibody binding was monitored in real-time at 25° C. with a continuous flow of PBS at 30 l/min. For SPR affinity measurements, antibody binding to HLA-E-VL9 complex protein was performed using a BIAcore S200 instrument (Cytiva, formerly GE Healthcare, DHVI BIA Core Facility, Durham, NC) in HBS-EP+ 1× running buffer. The antibodies were first captured onto CM5 sensor chip to a level of ˜9000 RU. The HLA-E-VL9 soluble proteins were injected over the captured antibodies at a flow rate of 30 uL/min. After dissociation, the antibodies were regenerated using a 30 second pulse of Glycine pH2.0. Results were analyzed using the Biacore S200 Evaluation software (Cytiva). Subsequent curve fitting analyses were performed using a 1:1 Langmuir model with a local Rmax. The reported binding curves are representative of two data sets.

### ELISA

Direct binding ELISAs were conducted in 384-well ELISA plates coated with 2 g/ml of C-trap-stabilized HLA-E-VL9, C-trap-stabilized HLA-E-RL9HIV or C-trap-stabilized HLA-E-RL9SIV in 0.1 M sodium bicarbonate overnight at 4° C. Plates were washed with PBS+0.05% Tween 20 and blocked with 3% BSA in PBS at room temperature for 1 h. MAb samples were incubated for 1 h in 3-fold serial dilutions starting at 100 μg/ml, followed by washing with PBS-0.05% Tween 20. HRP-conjugated goat anti-human IgG secondary Ab (SouthernBiotech, catalog #2040-05) was diluted to 1:10,000 in 1% BSA in PBS-0.05% Tween 20 and incubated at room temperature for 1 h. For sandwich ELISA, 384-well ELISA plates were coated with HLA-E-VL9 antibodies in a 3-fold dilution starting from 100 μg/mL in 0.1 M sodium bicarbonate overnight at 4° C. Plates were washed with PBS+0.05% Tween 20 and blocked with 3% BSA in PBS at room temperature for 1 h. C-trap-stabilized HLA-E-VL9, C-trap-stabilized HLA-E-RL9HIV, C-trap-stabilized HLA-E-RL9SIV, or diluent control were then added at 2 μg/mL and incubated at room temperature for 1 h. After washing, HRP-conjugated anti-human β2M antibody (Biolegend, catalog #280303) were added at 0.2 μg/mL and incubated at room temperature for 1 h. These plates were washed for 4 times and developed with tetramethylbenzidine substrate (SureBlue Reserve). The reaction was stopped with 1 M HCl, and optical density at 450 nm (OD450) was determined.

### Antibody Poly-Reactivity Assays

All mAbs isolated from mice and human were tested for ELISA binding to nine autoantigens—Sjogren's syndrome antigen A (SSA), Sjogren's syndrome antigen (SSB), Smith antigen (Sm), ribonucleoprotein (RNP), scleroderma 70 (Scl-70), Jo-1 antigen, double-stranded DNA (dsDNA), centromere B (Cent B), and histone as previously described (Han et al., 2017; Liao et al., 2011). Indirect immunofluorescence assay of mAbs binding to HEp-2 cells (Inverness Medical Professional Diagnostics, Princeton, NJ) was performed as previously described (Haynes et al., 2005; Liao et al., 2011). MAbs 2F5 (Yang et al., 2013) and 17B (Moore and Sodroski, 1996) were used as positive and negative controls, respectively. All antibodies were screened in two independent experiments.

### Negative Stain Electron Microscopy of IgM Antibodies

FPLC purified IgM antibodies were diluted to 0.08 mg/ml in HEPES-buffered saline (pH 7.4)+5% glycerol and stained with 2% uranyl formate. Images were obtained with a Philips EM420 electron microscope at 82,000 magnification and processed in Region 3.0.

### Microbiome-Derived Peptide Prediction

VL9 peptide sequence was first searched by similarity in NCBI microbial protein BLAST. The BLAST results were then analyzed for HLA-E binding epitope prediction using HLA class I peptide binding algorithms NetMHC 4.0 (Andreatta and Nielsen, 2016; Nielsen et al., 2003). Epitopes that have HLA-E binding prediction scores >0.1, length=9 aa, and are relative to human microbiome were synthesized for validation experiments.

### Peptide-Pulsing in K562-E Cells

K562-E cells and K562-E/UL49.5 cells were resuspended with fresh IMDM media with 10% FBS at 2×106 cells/ml. Peptides were added into cell suspension at a final concentration of 100 μM. The cell/peptide mixtures were incubated at 26° C. with 5% CO2 for 20-22 hours and were transferred to 37° C. for 2 hours with 5% CO2 before use. In the following mAb staining experiment, medium with 100 μM peptides was used to maintain peptide concentration.

### NK Cell Cytotoxicity Assay

NK Cell Cytotoxicity was measured by 51Cr release assay. A NKG2A-positive, CD16/CD32/CD64-negative NK-92 cells were used as effector cells in our study. Transfected or untransfected 293T cells were used as target cells. Target cells were counted, washed, resuspended in R10 at 1×107 cell/ml, and labeled with Na251CrO4 at 250 μCi/ml for 2 hours at 37° C. After washing three times using R10, cells were mixed with the testing antibody and effector cells in a final effector to target (E:T) ratio of 20:1 and 6:1 in triplicate wells in a flexible 96 well round bottom plates (PerkinElmer, Catalog #1450-401). The plates were inserted in flexible 96-well plate cassettes (PerkinElmer, Catalog #1450-101), sealed and incubated at 37° C. for 4 hours. After the incubation, cells were pelleted by centrifugation, and from the top of the well, add 25 ul of supernatant to a rigid 96 well isoplates (PerkinElmer, Catalog #1450-514) containing 150 ul of Ultima Gold LSC Cocktail (Sigma, Catalog #L8286). The plates were inserted in rigid 96-well plate cassettes (PerkinElmer, Catalog #1450-105), sealed and counted on Perkin Elmer Microbeta Triux 1450 counter. 51Cr labeled target cells without effector cells were set as a spontaneous release control, and 51Cr labeled target cells mixed with detergent (2% Triton X-100) were used as a maximum release control. The percentages of specific lysis were calculated with the formulation: The Percentages of Specific Lysis (51Cr Release %)=[(Experimental Release−Spontaneous Release)/(Maximum Release−Spontaneous Release)]×100.

Development and Screening of scFv Libraries on the Surface of Yeast

A library was built that contained ˜1.1 million 3H4 scFv variants with amino acid diversity at sites that were determined by structural analysis to interact with HLA-E-VL9. Seventeen residues (FIG. 18) located in the CDR loops of 3H4 were randomized in groups of four based on their proximity and all the possible combinations of amino acids were sampled at these sites. Library DNA was synthesized on a BioXP 3250 (Codex) system and amplified with High Fidelity Phusion polymerase (New England Biolabs). PCR products were gel extracted (Qiagen Gel Extraction kit) to select full length genes as per the manufacturer's protocol. 3H4 scFv variants were displayed in library format on the surface of yeast as previously described (Benatuil et al., 2010; Chao et al., 2006). Briefly, S. cerevisiae EBY100 cells were transformed by electroporation with a 3:1 ratio of 12 μg scFv library DNA and 4 μg pCTcon2 plasmid digested with BamHI, SalI, NheI (New England Biolabs). The size of the transformed library, determined by serial dilution on selective plates, was 5×107 individual colonies. Yeast Libraries were grown in SDCAA media (Teknova) supplemented with pen-strep at 30° C. and 225 rpm. 80% of the sequences recovered from the transformed libraries were confirmed to contain full length, in-frame genes by Sanger sequencing (Genewiz). scFv expression on the surface of yeast was induced by culturing the libraries in SGCAA (Teknova) media at a density of 1×107 cells/mL for 24-36 hours. Cells were washed twice in ice cold PBSA (0.01M sodium phosphate, pH 7.4, 0.137M sodium chloride, 1 g/L bovine serum albumin) and labeled with APC conjugated HLA-E-VL9 tetramer and 1:100 anti-c-myc:FITC (ICL) and incubated for 1 hour at 4 C. Initial selection was conducted with 50 mg/mL labeling concentration of HLA-E-VL9 tetramer; the second round of selection was done at 0.6 mg/mL tetramer. Cells were washed twice with PBSA after incubation with the fluorescently labeled probes and sorted on a BD FACS-DiVa. Double positive cells for APC and FITC were collected and expanded in SDCAA media supplemented with pen-strep before successive rounds of enrichment. FACS data was analyzed with Flowjo_v10.7 software (Becton, Dickinson & Company). All clones selected by FACS were expanded, and their DNA was extracted (Zymo Research) for analysis by Sanger sequencing (Genewiz). scFv encoding plasmids were recovered from yeast cultures by yeast miniprep with the Zymoprep yeast plasmid miniprep II kit (Zymo Research). Isolated DNA was transformed into NEB5a strain of E. coli (New England Biolabs) and the DNA of individual bacterial colonies was isolated (Wizard Plus SV Minipreps, Promega) and analyzed by Sanger sequencing (Genewiz).

### Statistics Analysis

Data were plotted using Prism GraphPad 8.0 or visualized using the ComplexHeatmap R package. SAS 9.4 (SAS Institute, Cary, NC) was used to perform the statistical analysis with a p-value<0.05 considered significant. For 51Cr release assays, mixed effects models were used to make comparisons of antibody to control using a random intercept for the triplicates run within each experiment and fixed effects of E:T ratio, type (antibody or control), and the interaction of E:T ratio by type. For human antibody gene usage analysis, chi-square test of independence was used to compare differences between groups.

### REFERENCES

Afonine, P. V., Grosse-Kunstleve, R. W., Echols, N., Headd, J. J., Moriarty, N. W., Mustyakimov, M., Terwilliger, T. C., Urzhumtsev, A., Zwart, P. H., and Adams, P. D. (2012). Towards automated crystallographic structure refinement with phenix.refine. Acta Crystallogr D Biol Crystallogr 68, 352-367.
Andreatta, M., and Nielsen, M. (2016). Gapped sequence alignment using artificial neural networks: application to the MHC class I system. Bioinformatics 32, 511-517.
Benatuil, L., Perez, J. M., Belk, J., and Hsieh, C. M. (2010). An improved yeast transformation method for the generation of very large human antibody libraries. Protein Eng Des Sel 23, 155-159.
Braud, V. M., Allan, D. S., O'Callaghan, C. A., Soderstrom, K., D'Andrea, A., Ogg, G. S., Lazetic, S., Young, N. T., Bell, J. I., Phillips, J. H., et al. (1998). HLA-E binds to natural killer cell receptors CD94/NKG2A, B and C. Nature 391, 795-799.
Chao, G., Lau, W. L., Hackel, B. J., Sazinsky, S. L., Lippow, S. M., and Wittrup, K. D. (2006). Isolating and engineering human antibodies using yeast surface display. Nature Protocols 1, 755-768.
Chen, V. B., Arendall, W. B., 3rd, Headd, J. J., Keedy, D. A., Immormino, R. M., Kapral, G. J., Murray, L. W., Richardson, J. S., and Richardson, D. C. (2010). MolProbity: all-atom structure validation for macromolecular crystallography. Acta Crystallogr D Biol Crystallogr 66, 12-21.
Emsley, P., Lohkamp, B., Scott, W. G., and Cowtan, K. (2010). Features and development of Coot. Acta Crystallogr D Biol Crystallogr 66, 486-501.
Han, Q., Williams, W. B., Saunders, K. O., Seaton, K. E., Wiehe, K. J., Vandergrift, N., Von Holle, T. A., Trama, A. M., Parks, R. J., Luo, K., et al. (2017). HIV DNA-Adenovirus Multiclade Envelope Vaccine Induces gp41 Antibody Immunodominance in Rhesus Macaques. J Virol 91.
Haynes, B. F., Fleming, J., St Clair, E. W., Katinger, H., Stiegler, G., Kunert, R., Robinson, J., Scearce, R. M., Plonk, K., Staats, H. F., et al. (2005). Cardiolipin polyspecific autoreactivity in two broadly neutralizing HIV-1 antibodies. Science 308, 1906-1908.
Karplus, P. A., and Diederichs, K. (2012). Linking crystallographic model and data quality. Science 336, 1030-1033.
Kepler, T. B., Munshaw, S., Wiehe, K., Zhang, R., Yu, J. S., Woods, C. W., Denny, T. N., Tomaras, G. D., Alam, S. M., Moody, M. A., et al. (2014). Reconstructing a B-Cell Clonal Lineage. II. Mutation, Selection, and Affinity Maturation. Front Immunol 5, 170.
Krissinel, E., and Henrick, K. (2004). Secondary-structure matching (SSM), a new tool for fast protein structure alignment in three dimensions. Acta Crystallogr D Biol Crystallogr 60, 2256-2268.
Krissinel, E., and Henrick, K. (2007). Inference of macromolecular assemblies from crystalline state. J Mol Biol 372, 774-797.
Lampen, M. H., Hassan, C., Sluijter, M., Geluk, A., Dijkman, K., Tjon, J. M., de Ru, A. H., van der Burg, S. H., van Veelen, P. A., and van Hall, T. (2013). Alternative peptide repertoire of HLA-E reveals a binding motif that is strikingly similar to HLA-A2. Mol Immunol 53, 126-131.
Liao, H. X., Chen, X., Munshaw, S., Zhang, R., Marshall, D. J., Vandergrift, N., Whitesides, J. F., Lu, X., Yu, J. S., Hwang, K. K., et al. (2011). Initial antibodies binding to HIV-1 gp41 in acutely infected subjects are polyreactive and highly mutated. J Exp Med 208, 2237-2249.
Liao, H. X., Levesque, M. C., Nagel, A., Dixon, A., Zhang, R., Walter, E., Parks, R., Whitesides, J., Marshall, D. J., Hwang, K. K., et al. (2009). High-throughput isolation of immunoglobulin genes from single human B cells and expression as monoclonal antibodies. J Virol Methods 158, 171-179.
Liao, H. X., Lynch, R., Zhou, T., Gao, F., Alam, S. M., Boyd, S. D., Fire, A. Z., Roskin, K. M., Schramm, C. A., Zhang, Z., et al. (2013). Co-evolution of a broadly neutralizing HIV-1 antibody and founder virus. Nature 496, 469-476.
Liao, H. X., Sutherland, L. L., Xia, S. M., Brock, M. E., Scearce, R. M., Vanleeuwen, S., Alam, S. M., McAdams, M., Weaver, E. A., Camacho, Z., et al. (2006). A group M consensus envelope glycoprotein induces antibodies that neutralize subsets of subtype B and C HIV-1 primary viruses. Virology 353, 268-282.
Moody, M. A., Santra, S., Vandergrift, N. A., Sutherland, L. L., Gurley, T. C., Drinker, M. S., Allen, A. A., Xia, S. M., Meyerhoff, R. R., Parks, R., et al. (2014). Toll-like receptor 7/8 (TLR7/8) and TLR9 agonists cooperate to enhance HIV-1 envelope antibody responses in rhesus macaques. J Virol 88, 3329-3339.
Moore, J. P., and Sodroski, J. (1996). Antibody cross-competition analysis of the human immunodeficiency virus type 1 gp120 exterior envelope glycoprotein. J Virol 70, 1863-1872.
Nielsen, M., Lundegaard, C., Worning, P., Lauemoller, S. L., Lamberth, K., Buus, S., Brunak, S., and Lund, O. (2003). Reliable prediction of T-cell epitopes using neural networks with novel sequence representations. Protein Sci 12, 1007-1017.
Sanchez, A. M., DeMarco, C. T., Hora, B., Keinonen, S., Chen, Y., Brinkley, C., Stone, M., Tobler, L., Keating, S., Schito, M., et al. (2014a). Development of a contemporary globally diverse HIV viral panel by the EQAPOL program. J Immunol Methods 409, 117-130.
Sanchez, A. M., Rountree, W., Berrong, M., Garcia, A., Schuetz, A., Cox, J., Frahm, N., Manak, M., Sarzotti-Kelsoe, M., D'Souza, M. P., et al. (2014b). The External Quality Assurance Oversight Laboratory (EQAPOL) proficiency program for IFN-gamma enzyme-linked immunospot (IFN-gamma ELISpot) assay. J Immunol Methods 409, 31-43.
Saunders, K. O. (2019). Conceptual Approaches to Modulating Antibody Effector Functions and Circulation Half-Life. Front Immunol 10, 1296.
Tian, M., Cheng, C., Chen, X., Duan, H., Cheng, H. L., Dao, M., Sheng, Z., Kimble, M., Wang, L., Lin, S., et al. (2016). Induction of HIV Neutralizing Antibody Lineages in Mice with Diverse Precursor Repertoires. Cell 166, 1471-1484 e1418.
Vagin, A., and Teplyakov, A. (2010). Molecular replacement with MOLREP. Acta Crystallogr D Biol Crystallogr 66, 22-25.
von Boehmer, L., Liu, C., Ackerman, S., Gitlin, A. D., Wang, Q., Gazumyan, A., and Nussenzweig, M. C. (2016). Sequencing and cloning of antigen-specific antibodies from mouse memory B cells. Nat Protoc 11, 1908-1923.
Walter, T. S., Diprose, J. M., Mayo, C. J., Siebold, C., Pickford, M. G., Carter, L., Sutton, G. C., Berrow, N. S., Brown, J., Berry, I. M., et al. (2005). A procedure for setting up high-throughput nanolitre crystallization experiments. Crystallization workflow for initial screening, automated storage, imaging and optimization. Acta Crystallogr D Biol Crystallogr 61, 651-657.
Winn, M. D., Ballard, C. C., Cowtan, K. D., Dodson, E. J., Emsley, P., Evans, P. R., Keegan, R. M., Krissinel, E. B., Leslie, A. G., McCoy, A., et al. (2011). Overview of the CCP4 suite and current developments. Acta Crystallogr D Biol Crystallogr 67, 235-242.
Winter, G., Waterman, D. G., Parkhurst, J. M., Brewster, A. S., Gildea, R. J., Gerstel, M., Fuentes-Montero, L., Vollmar, M., Michels-Clark, T., Young, I. D., et al. (2018). DIALS: implementation and evaluation of a new integration package. Acta Crystallogr D Struct Biol 74, 85-97.
Wrammert, J., Smith, K., Miller, J., Langley, W. A., Kokko, K., Larsen, C., Zheng, N. Y., Mays, I., Garman, L., Helms, C., et al. (2008). Rapid cloning of high-affinity human monoclonal antibodies against influenza virus. Nature 453, 667-671.
Yang, G., Holl, T. M., Liu, Y., Li, Y., Lu, X., Nicely, N. I., Kepler, T. B., Alam, S. M., Liao, H. X., Cain, D. W., et al. (2013). Identification of autoantigens recognized by the 2F5 and 4E10 broadly neutralizing HIV-1 antibodies. J Exp Med 210, 241-256.

All non-patent references cited herein are hereby incorporated-by-reference for their background teachings per context of citation.

TABLE 5
Gene usage and mutation rate of four HLA-E-VL9-
specific mAbs and one pan-HLA-E
mAb isolated from immunized transgenic mice.

| | | | VH | | VK |
|---|---|---|---|---|---|
| | | | Mutation | | Mutation |
| Ab ID | Isotype | VH gene | % | VK gene | % |
| 3H4 | IgM | 1-18*01 F | 1.040 | 14-111*01 | 2.510 |
| | | | | F | |
| 13F11 | IgM | 6-6*01 F | 1.361 | 6-32*01 F | 0.000 |
| 10C10 | IgM | 1-18*01 F | 1.040 | 6-25*01 F | 7.168 |
| 2D6 | IgM | 1-26*01 F | 1.390 | 4-59*01 F | 1.810 |

TABLE 6
Information of human subjects used in this study. “PIDs” are anonymous
patient IDs.

| | PID 0873-031 | PID 0873-099 | PID 0873-127 | PID 0873-133 |
|---|---|---|---|---|
| LeukoPak ID | PPD LP 021 | PPD LP 030 | PPD LP 059 | PPD LP 060 |
| Age at time of donation | 27 | 22 | 21 | 22 |
| Gender | M | M | M | M |
| Race | W | W | W | W |
| HIV-1 Ab Test | Neg | Neg | Neg | Neg |
| CMV IgG [Expected < | <4 | <4 | <4 | <4 |
| 4 AU/mL, | | | | |
| Positive >= 6] | | | | |
| EBV IgG | 69 | 2155 | 2596 | 1281 |
| Ab[Expected = Neg < 100 | | | | |
| AU/mL, Equiv 100- | | | | |
| 120, Positive > 120 | | | | |
| AU/ml] | | | | |
| Hep B S—Ag | Neg | | | |
| CD3+45+ % | 63 | 81 | 71 | 87 |
| CD3+8+45+ % | 20 | 30 | 23 | 27 |
| CD3+4+45+ % | 40 | 49 | 42 | 56 |
| CD3-16+56+45+% | 23 | 9 | 15 | 5 |
| CD19+45+ % | 13 | 8 | 13 | 7 |
| HLA-A | 0101 | 0301 | 0101 | 0201 |
| HLA-A | 3002 | 1101 | — | 2402 |
| HLA-B | 0801 | 1501 | 0801 | 2705 |
| HLA-B | 1302 | 4002 | — | 4402 |
| HLA-C | 06020 | 0202 | 0701 | 0202 |
| HLA-C | 0701 | 0401 | — | 0501 |

HLA-E-VL9-specific antibodies isolated from human are also described in FIG. 22E.

### Example 2: Optimization of HLA-E-VL9 Binding Antibodies

In order to optimize HLA-E-VL9 binding antibodies, e.g. without limitation for clinical use, the following antibody properties could be targeted for improvement: 1) Binding affinity for the HLA-E/VL9 peptide with an improved dissociation constant, e.g. in the 10-100 nM range; 2) Specificity for the VL9 peptide; at least 10 times more specific for the HLA-E/VL9 complex compared to other naturally occurring HLA-E-peptide complexes; 3) reduced off target binding/polyspecificity, for example below clinically accepted levels; 4) stability and solubility suitable for pharmaceutical grade antibody production.

In some embodiments, the invention contemplates computational optimization methods which could include without limitation the following: Identify mutations to increase binding affinity, alter CDR loop length/sequence for increased affinity and/or target specificity.

Antibody libraries could include without limitation single site libraries, CDR loops libraries, saturation libraries, directed libraries, structurally informed and/or computationally informed optimization libraries.

Any suitable library screening platform could be used. Platforms include without limitation yeast display which allows for screening of scFv, but has limited ability to select for stability/solubility. Platforms include without limitation mammalian display which allows for screening of IgG, assessing expression levels which could correlate with stability and solubility.

In non-limiting embodiments, screening for optimized antibodies include strategies to efficiently select for improved affinity, specificity, reduced polyreactivity, and improved stability.

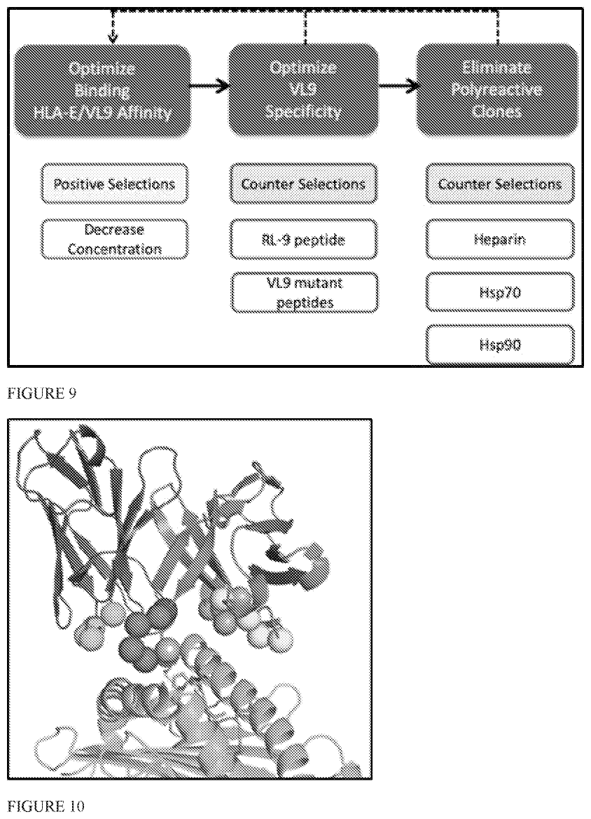

The HLA-E-VL9 binding antibodies will be subjected to multiple rounds of optimization using high throughput screening. In some embodiments, libraries of antibody variants, constructed as scFVs and displayed on the surface of yeast will be screened and sorted by FACS in succession as described in FIG. 9: 1) five positive selections rounds for binding to the HLA-E/VL9 complex present at a concentration of 500 nM in the first round, and two fold dilutions afterwards (250 nM, 125 nM, 62.5 nM, 31.2 nM and 16 nM respectively); 2) two counter selection rounds for specificity in the presence of the HLA-E/VL9 complex at a concentration of 10 nM and HLA-E/RL-9 competitor peptide present at 100 nM; 3) two counter selection rounds to eliminate clones that bind heparin, Hsp70 and Hsp90 to eliminate polyreactive clones.

Different type of libraries containing antibody variants will be designed, constructed and screened. In one embodiment, the library is a single site CDR loop saturation library, e.g. designed to contain all the twenty amino acids variants at each position in the CDR loops, mutated one at a time. In another embodiment, the libraries are site directed libraries that explores all possible amino acid variants only at the antibody sites involved in binding interactions with HLA-E/VL9, for example as informed from the crystal structure of the complex.

The yeast display platform is well suited to analyze 10⁷ antibody variants at a time; therefore all the possible amino acids combinations will be simultaneously tested at groups of four antibody residues to ensure that the number of resulting antibody variants to be screened experimentally is below 10⁷.

### 3H4 Optimization and Libraries Based on HLA-E-VL9/3H4 Complex Structure

The 3H4 antibody has four key contacts with the HLA-E/VL9 peptide complex. See FIG. 2. These contacts are Y97, S100, S100A and Y100B.

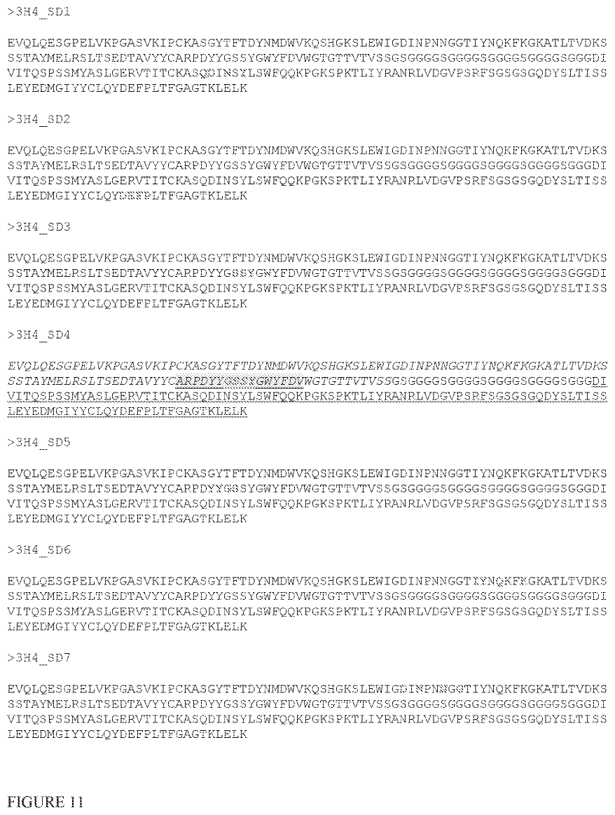

Additional antibody residues/sites were chosen for modification and antibody optimization. Residues that are chosen to be randomized as part of directed libraries are shown in the structure to contact HLA-E. Based on the analysis of the structure we chose to randomize 7 groups of 4 residues. The groups of four residues are picked such they are close in space and contact the same site of the HLA-E/VL9 complex. The four residues are selected such that they interact with the same region(s) of the epitope and thus may have combined binding effects (FIG. 10). Four residues are randomized at a time because this is the number of variants that can be tested by yeast display. Some of the residues in the libraries are part of the CDR H3, but other residues beyond the CDR H3 will also be tested.

In some embodiments, not all the amino acids in a group will need to be changed during the optimization. In some embodiments any one residue from a group of residues or a combination thereof could be changed in an optimized variant. In some embodiments the combination is a combination of residues within a group. In some embodiments the combination is a combination of residues from different groups.

Based on structural analysis, seven scFv different directed libraries that sample all the amino acid variants at four residues are generated. These libraries aim to test all the 20 amino acid combinations at the four residues shown in bold and red in the sequence in FIG. 11. 3H4 SD4 sequence shows one embodiment of a scFv fragment where VH is italicized and VL is double underlined, and HCDR3 sequence is underlined. by alignment to the other variants in FIG. 11, the VH, VL, and HCDR3 regions can be determined. The linker between the VH and VL sequence could be any suitable linker of varying length and/or sequence.

FIG. 11 shows non-limiting embodiments of scFv variants comprising contact residues which could be modified in an optimized variant. Based on structural analysis, seven scFv different directed libraries that sample all the amino acid variants at four sites were designed. The four sites are shown in bold and red in the sequence. Sequence changes identified from these libraries that improve the properties of 3H4 mAb will be further combined in additional libraries that will be screened as in FIG. 9 to identified sets of mutations that are cumulative towards the optimization 3H4. Each sequence shows one embodiment of a scFv fragment where VH is italicized and VL is double underlined, and HCDR3 sequence is underlined.

The linker between the VH and VL sequence could be any suitable linker of varying length and/or sequence.

In non-limiting embodiments, the optimized antibody sequences will be tested for their binding. Binding affinity will be determined. Binding assays include without limitation cell surface staining, SPR and ELISA.

Cell Surface Staining and High-Throughput Screening (HTS). HLA-E SCT constructs encoding HLA-E-VL9, HLA-E-RL9HIV, or HLA-E-RL9SIV are transfected into 293T cells using GeneJuice transfection reagent (Novagen, Catalog #70967). For epitope mapping experiment, a panel of HLA-E-VL9 SCT constructs with single amino acid mutations are transfected into 293T cells using the same method. Cells are dissociated with 0.1% EDTA at 48 hours post-transfection and stained with a Fixable Near-IR Dead Cell Stain Kit (Thermo Fisher, Catalog #L34976). After washing, primary antibodies (supernatant from hybridoma cells, supernatant from transfected cells, or purified antibodies) are added and incubated with cells for 1 hour at 4° C., following by staining with 1:1000 diluted secondary antibodies for 30 mins at 4° C. For mouse primary antibodies, Alexa Fluor 555 (AF555) conjugated goat anti-mouse IgG (H+L) (Thermo Fisher, Catalog #A32727) is used or Alexa Fluor 647 (AF647) conjugated goat anti-mouse IgG (H+L) (Thermo Fisher, Catalog #A32728) as secondary antibodies; for human primary antibodies, AF555 conjugated goat anti-human IgG (H+L) (Thermo Fisher, Catalog #A-21433) is used or AF647 conjugated goat anti-human IgG (H+L) (Thermo Fisher, Catalog #A-21445) as secondary antibodies. Cells were then washed 3 times and resuspended in fixation buffer (1% formaldehyde in PBS, pH7.4). Data are acquired on a BD LSR II flow cytometer and analyzed using FlowJo version 10.

Surface Plasmon Resonance (SPR). Surface plasmon resonance assays are performed on a BIAcore 3000 instrument, and data analysis was performed with BIAevaluation 3.0 software as previously described (Liao et al., 2006). Briefly, streptavidin is directly immobilized to CM5 sensor chips, then biotinylated HLA-E-peptide complexes (HLA-E-VL9, HLA-E-RL9SIV, HLA-E-RL9HIV and mock control) were bound to the immobilized streptavidin. Purified mAbs flowed over CM5 sensor chips at concentrations of 100 μg/ml, and antibody binding was monitored in real-time at 25° C. with a continuous flow of PBS at 30 l/min.

ELISA. Direct binding ELISAs were conducted in 384-well ELISA plates coated with 2 μg/ml of C-trap-stabilized HLA-E-VL9, C-trap-stabilized HLA-E-RL9HIV or C-trap-stabilized HLA-E-RL9SIV in 0.1 M sodium bicarbonate overnight at 4° C. Plates were washed with PBS+0.05% Tween 20 and blocked with 3% BSA in PBS at room temperature for 1 h. MAb samples were incubated for 1 h in 3-fold serial dilutions starting at 100 μg/ml, followed by washing with PBS-0.05% Tween 20. HRP-conjugated goat anti-human IgG secondary Ab (SouthernBiotech, catalog #2040-05) was diluted to 1:10,000 in 1% BSA in PBS-0.05% Tween 20 and incubated at room temperature for 1 h. For sandwich ELISA, 384-well ELISA plates were coated with HLA-E-VL9 antibodies in a 3-fold dilution starting from 100 μg/mL in 0.1 M sodium bicarbonate overnight at 4° C. Plates were washed with PBS+0.05% Tween 20 and blocked with 3% BSA in PBS at room temperature for 1 h. C-trap-stabilized HLA-E-VL9, C-trap-stabilized HLA-E-RL9HIV, C-trap-stabilized HLA-E-RL9SIV, or diluent control were then added at 2 μg/mL and incubated at room temperature for 1 h. After washing, HRP-conjugated anti-human β2M antibody (Biolegend, catalog #280303) were added at 0.2 μg/mL and incubated at room temperature for 1 h. These plates were washed for 4 times and developed with tetramethylbenzidine substrate (SureBlue Reserve). The reaction was stopped with 1 M HCl, and optical density at 450 nm (OD₄₅₀) was determined.

### Cell Killing Assays

To test if an optimized antibody has higher killing function, we will perform NK Cell Cytotoxicity Assay by ⁵¹Cr release assay. Human NK-92 cells are used as effector cells in our study. Transfected 293T cells are used as target cells. Target cells are counted, washed, resuspended in R10 at 1×10⁷ cell/ml, and labeled with Na₂⁵¹CrO4 at 250 μCi/ml for 2 hours at 37° C. After washing three times using R10, cells are mixed with effector cells in a final effector to target (E:T) ratio of 60:1 and 6:1 in triplicate wells in a flexible 96 well round bottom plates (PerkinElmer, Catalog #1450-401). The plates are inserted in flexible 96-well plate cassettes (PerkinElmer, Catalog #1450-101), sealed and incubated at 37° C. for 4 hours. After the incubation, cells are pelleted by centrifugation, and from the top of the well, add 25 ul of supernatant to a rigid 96 well isoplates (PerkinElmer, Catalog #1450-514) containing 150 ul of Ultima Gold LSC Cocktail (Sigma, Catalog #L8286). The plates are inserted in rigid 96-well plate cassettes (PerkinElmer, Catalog #1450-105), sealed and counted on Perkin Elmer Microbeta Triux 1450 counter. ⁵¹Cr labeled target cells without effector cells are set as a spontaneous release control, and ⁵¹Cr labeled target cells mixed with detergent (2% Triton X-100) were used as a maximum release control. The percentages of specific lysis were calculated with the formulation: The Percentages of Specific Lysis (⁵¹Cr Release %)=[(Experimental Release−Spontaneous Release)/(Maximum Release−Spontaneous Release)]×100.

To test off target binding, including autoreactivity, of an optimized antibody, we will screen the antibodies in AtheNA assay, Hep-2 cell staining, and any other suitable assay.

AtheNA assay. All mAbs isolated from mice and human are tested for ELISA binding to nine autoantigens—Sjogren's syndrome antigen A (SSA), Sjogren's syndrome antigen (SSB), Smith antigen (Sm), ribonucleoprotein (RNP), scleroderma 70 (Scl-70), Jo-1 antigen, double-stranded DNA (dsDNA), centromere B (Cent B), and histone as previously described (Han et al., 2017; Liao et al., 2011).

Indirect immunofluorescence assay of mAbs binding to HEp-2 cells (Inverness Medical Professional Diagnostics, Princeton, NJ) is performed as previously described (Haynes et al., 2005; Liao et al., 2011). MAbs 2F5 (Yang et al., 2013) and 17B (Moore and Sodroski, 1996) are used as positive and negative controls, respectively. All antibodies were screened in two independent experiments.

Membrane Proteome Array is an array of membrane proteins detecting off target binding.

Stability Assay. Melting temperature using Differential Scanning Fluorimetry (Tm with DSF) Assay: This assay tests the thermal stability of the antibody. The higher the thermal stability, the less likely the protein will spontaneously unfold and become immunogenic. The antibody will be mixed with a dye that fluoresces when in contact with hydrophobic regions, such as SPYRO orange. The mixture will then be taken through a range of temperatures (e.g. 40° C.->95° C. at a rate of 0.5° C./2 min). As the protein begins to unfold, buried hydrophobic residues will become exposed and the level of fluorescence will suddenly increase. The value of T when the increase in fluorescence intensity is greatest gives us a Tm value.

### Solubility/Aggregation Propensity:

a. Size-exclusion Chromatography (SEC) Assay: Antibodies will be flowed through a column consisting of spherical beads with miniscule pores. Non-aggregated antibodies will be small enough to get trapped in the pores, whereas aggregated antibodies will flow through the column more rapidly. Percentage aggregation can be worked out from the concentrations of the different fractions.
b. Clone Self-interaction by Bio-layer Interferometry (CSI-BLI) Assay: A more high-throughput method that uses a label-free technology to measure self-interaction. Antibodies will be loaded onto the biosensor tip and white light is shone down the instrument to yield an internal reflection interference pattern. Then the tip is inserted into a solution of the same antibody, and if self-interaction occurs, then the interference pattern shifts by an amount proportional to the change in thickness of the biological layer.
c. Affinity-capture Self-interaction Nanoparticle Spectroscopy (AC-SINS) Assay: This assay tests how likely an antibody is to interact with itself. It uses gold nanoparticles that are coated with anti-Fc antibodies. When a dilute solution of antibodies is added, they rapidly become immobilised on the gold beads. If these antibodies subsequently attract one another, it leads to shorter interatomic distances and longer absorption wavelengths that can be detected by spectroscopy.

See e.g. Jain et al. | PNAS | Jan. 31, 2017 | vol. 114 | no. 5 p. 944-949, and summarized here:

In non-limiting embodiments, optimized sequences based on mouse VH and VL chains will be humanized.

See Example 1, and Table 1, for non-limiting embodiments of affinity matured antibodies based on 3H4 antibody sequence.

### Optimization and Libraries Based on HLA-E-VL9 Complex Structure

Additional antibody residues/sites are chosen for modification and antibody optimization. Residues that are chosen to be randomized as part of directed libraries are residue that contact HLA-E/VL9 complex, and/or residues which are in proximity to contact site residues. Such contact and proximal residues could be organized in groups of 4 residues. The groups of four residues are picked such they are close in space and contact the same site of the HLA-E/VL9 complex. The four residues are selected such that they interact with the same region(s) of the epitope and thus may have combined binding effects). Four residues are randomized at a time because this is the number of variants that can be tested by yeast display. Some of the residues in the libraries are part of the CDR H3, but other residues beyond the CDR H3 will also be tested.

In some embodiments, not all the amino acids in a group will need to be changed during the optimization. In some embodiments any one residue from a group of residues or a combination thereof could be changed in an optimized variant. In some embodiments the combination is a combination of residues within a group. In some embodiments the combination is a combination of residues from different groups.

Based on structural analysis, scFv different directed libraries that sample all the amino acid variants at four residues are generated. These libraries aim to test all the 20 amino acid combinations at the four residues shown in FIG. 11. Residues could be selected from VH and/or VL residues. Residues could be within CDRs or outside of the CDRs. In scFv designs, the VH and VL are connected by a linker. The linker between the VH and VL sequence could be any suitable linker of varying length and/or sequence. See e.g. Chao et al. Nat Protoc. 2006; 1(2):755-68. doi: 10.1038/nprot.2006.94.

Sequence changes identified from these libraries that improve the properties of an antibody will be further combined in additional libraries that will be screened as in FIG. 9 to identified sets of mutations that are cumulative towards the optimization of an antibody.

The linker between the VH and VL sequence could be any suitable linker of varying length and/or sequence.

scFv Libraries Currently being Built and/or Screened in Yeast:
CA147 (anti-HLA-E/VL9) single-site saturation (NNK) library of all CDR loops
CA123 (anti-HLA-E/VL9) single-site saturation (NNK) library of all CDR loops

Any other human HLA-E/VL-9 antibody could be optimized.

### Example 3: Multimeric Antibodies

In certain embodiments antibodies can have low affinity for their cognate antigen (Neuberger et al. 2008. Immunol Cell Biol Volume 86, Issue 2 Feb. 2008; Pages 124-132). In nature, the binding strength between antibodies and their cognate antigen is increased by providing avidity to the interaction with either by the presence of multiple B cell receptor on the surface of B cells or through the formation of dimers, pentamers, and hexamers of secreted immunoglobulin (Czajkowsky et al. Proc Natl Acad Sci USA. 2009; Kumar et al. Science. 2020). Similar principles can be applied to antibody biologics. Through avidity, we sought to increase the binding strength of these antibodies thereby enhancing their ability to mediate killing of infected cells.

In one non-limiting approach, we generated hexamers of IgG by introducing mutations E345R, E430G, S440Y in the Fc region of human gamma immunoglobulin (G1m3 allotype) (FIG. 25A, Diebolder, C A et al Science 343:1260-63 2014). These mutations could be introduced in any other suitable human gamma immunoglobulin gene. Any other mutations could be introduced in the Fc portion of the IgG molecule. Non-limiting embodiments heaxamer designs are shown in FIG. 24B, and FIG. 26A.

Each engineered antibody was expressed by transient transfection of Expi293F cells and purified by anti-kappa or anti-lambda constant region affinity resin. SDS-PAGE analysis showed the presence of heavy and light chains. To assess the presence of hexamer formation, we performed negative stain electron microscopy on each protein preparation. Electron microscopy confirmed the presence of some monomeric IgG, but also hexameric IgG (FIG. 25B, C). The hexameric IgG was purified by size exclusion chromatography to enrich for IgG hexamers. These results demonstrate proof-of-concept for multimeric anti-HLA-E antibodies hexamers and forms the rationale for using the same approach for other antibodies targeting MHC:peptide complexes. These antibodies may be targeted to non-classical or classical MHC molecules. The peptides presented by the MHC molecules can be derived from self antigens or pathogens. The antibodies may originate from any species.

Another non-limiting approach to form multimeric antibody, is to generate IgG antibodies or fragments thereof arrayed in multiple copies on the surface of nanoparticles, including without limitation protein nanoparticles. In a non-limiting embodiment, the nanoparticle is a ferritin nanoparticle. These specific ferritin nanoparticles would have the potential to array up to 24 copies of the antibody of interest. Antibody nanoparticles have been explored using single gene constructs (Rujas et al. Nat Commun. 2021; Divine et al. Science. 2021).

Antibody nanoparticles were generated as conjugate nanoparticles by adding a sortase A donor peptide (also referred to a sortase tag or linker) to the C-terminus of the heavy chain constant region and by adding a sortase acceptor sequence to the N-terminus of each ferritin nanoparticle subunit. In another embodiments, a sortase acceptor is added to the N-terminus of the heavy or light chain constant region and a sortase donor peptide sequence to the C-terminus of each ferritin nanoparticle subunit. Any suitable heavy chain constant region can be used. The donor peptide can vary at the third position, but A, E, and S tend to be the most common amino acids used. The acceptor sequence can be 5 or more glycines.

In one embodiment, the multimer antibody was designed as a full-length IgG. In another embodiment the multimer was designed as an antigen binding fragments (Fabs). In the Fab design, in one embodiment, the sortase tag is added to the C-terminus end of the Fab heavy chain sequence (FIG. 26). In another embodiment, the sortase tag is added to the C-terminus of the Fab light chain sequence. Any suitable constant gene could be used for the design of full length IgG, or the Fab fragment. One non-limiting embodiment of a Fab design is shown in FIG. 26A.

The Fabs of each antibody expressed well in Expi293 cells. The sortase A-tagged Fab molecule was conjugated to ferritin nanoparticles overnight at room temperature in the presence of 100 μM recombinant sortase A. Size exclusion chromatography was used to separate Fab-nanoparticle conjugates. SDS-PAGE analysis of Fab conjugate nanoparticles confirmed the Fab molecule was conjugated to ferritin subunits. Furthermore, negative stain electron microscopy of the purified Fab conjugate nanoparticles showed Fab molecules were conjugated to the surface of the nanoparticle (FIG. 27A, B). To determine whether anti-HLA-E-peptide Fabs were still functional on the surface of the nanoparticle, we assessed binding of nanoparticles to cells expressing HLA-E in complex with VL9 peptide or a negative control peptide. CA147 Fab conjugate nanoparticles bound to cells expressing HLA-E in complex with VL9 but did not show binding to cells expressing HLA-E in complex with a negative control peptide (FIG. 28). This approach can be used for Fabs derived from any species as long as the Fab is sortase A tagged.

### Materials & Methods

### Human IgG Hexameric Antibody Production

The CA147 human antibody heavy chain gene was synthesized and cloned (GenScript) in a human IgG1 backbone with the E345R/E430G/S440Y mutations (termed G1m3). Recombinant IgG mAbs were then produced in HEK293i suspension cells by transfection the heavy chain CA147_VH_G1m3 gene and the light chain CA147_VK gene with ExpiFectamine and purified using Protein L resin. The purified mAbs were further purified in AKTA FPLC system using a Superose 6 size-exclusion column.

### Cell Surface Staining and High-Throughput Screening (HTS)

HLA-E single-chain trimer (SCT) constructs encoding HLA-E-VL9 or HLA-E-Mtb44 were transiently transfected into 293T cells using GeneJuice transfection reagent (EMD Millipore, Catalog #70967). Two days post transfection, cells were diassociated with 1 mM EDTA and were washed and resuspended in 1×PBS pH 7.4. Next, primary antibodies (supernatant from hybridoma cells or purified antibodies) were added and incubated with cells for 30 minutes at 4° C., followed by staining with secondary antibody Goat anti-human IgG (H+L) Highly Cross-Adsorbed Secondary Antibody, Alexa Fluor Plus 647 (Thermo Fisher, Catalog #A-21445). Cells were then stained with LIVE/DEAD Fixable Aqua Dead Cell Stain Kit (Invitrogen, Catalog #L34957). Then, cells were washed and resuspended in fixation buffer (1% paraformaldehyde in PBS, pH 7.4). Data were acquired on a BD LSR II flow cytometer with HTS and analyzed using FlowJo version 10.

The multimeric antibodies or fragments thereof will be tested in any suitable assay to characterize their properties, including without limitation any of the assays described herein.

1-60. (canceled)

61. A recombinant antibody or fragment thereof selected from the group consisting of:
3H4, CA147, 13F11, 10C10, 2D6, CA118, CA119, CA120, CA122, CA123, CA124, CA125, CA127, CA128, CA129, CA130, CA131, CA132, CA133, CA134, CA135, CA136, CA137, CA138, CA139, CA140, CA141, CA143, CA144, CA145, CA146, CA401, 3H4 Gv2, 3H4 Gv3, 3H4 Gv4, 3H4 Gv5, 3H4 Gv6, 3H4 Gv7, 3H4 Gv8, 3H4 Gv9, 3H4 Gv10, 3H4 Gv11, 3H4 Gv12, CA147hexa, CA147sortaNP, 3H4hexa, and 3H4sortaNP;
wherein 3H4 comprises a heavy chain variable region (VH) comprising an amino acid sequence at least 80% identical to SEQ ID NO: 128 and a light chain variable region (VL) comprising an amino acid sequence at least 80% identical to SEQ ID NO: 129;
wherein CA147 comprises a VH comprising an amino acid sequence at least 80% identical to SEQ ID NO: 184 and a VL comprising an amino acid sequence at least 80% identical to SEQ ID NO: 185;
wherein 13F11 comprises a VH comprising an amino acid sequence at least 80% identical to SEQ ID NO: 130 and a VL comprising an amino acid sequence at least 80% identical to SEQ ID NO: 131;
wherein 10C10 comprises a VH comprising an amino acid sequence at least 80% identical to SEQ ID NO: 132 and a VL comprising an amino acid sequence at least 80% identical to SEQ ID NO: 133;
wherein 2D6 comprises a VH comprising an amino acid sequence at least 80% identical to SEQ ID NO: 134 and a VL comprising an amino acid sequence at least 80% identical to SEQ ID NO: 135;
wherein CA118 comprises a VH comprising an amino acid sequence at least 80% identical to SEQ ID NO: 136 and a VL comprising an amino acid sequence at least 80% identical to SEQ ID NO: 137;
wherein CA119 comprises a VH comprising an amino acid sequence at least 80% identical to SEQ ID NO: 138 and a VL comprising an amino acid sequence at least 80% identical to SEQ ID NO: 139;
wherein CA120 comprises a VH comprising an amino acid sequence at least 80% identical to SEQ ID NO: 140 and a VL comprising an amino acid sequence at least 80% identical to SEQ ID NO: 141;
wherein CA122 comprises a VH comprising an amino acid sequence at least 80% identical to SEQ ID NO: 142 and a VL comprising an amino acid sequence at least 80% identical to SEQ ID NO: 143;
wherein CA123 comprises a VH comprising an amino acid sequence at least 80% identical to SEQ ID NO: 144 and a VL comprising an amino acid sequence at least 80% identical to SEQ ID NO: 145;
wherein CA124 comprises a VH comprising an amino acid sequence at least 80% identical to SEQ ID NO: 146 and a VL comprising an amino acid sequence at least 80% identical to SEQ ID NO: 147;
wherein CA125 comprises a VH comprising an amino acid sequence at least 80% identical to SEQ ID NO: 148 and a VL comprising an amino acid sequence at least 80% identical to SEQ ID NO: 149;
wherein CA127 comprises a VH comprising an amino acid sequence at least 80% identical to SEQ ID NO: 150 and a VL comprising an amino acid sequence at least 80% identical to SEQ ID NO: 151;
wherein CA128 comprises a VH comprising an amino acid sequence at least 80% identical to SEQ ID NO: 152 and a VL comprising an amino acid sequence at least 80% identical to SEQ ID NO: 153;
wherein CA129 comprises a VH comprising an amino acid sequence at least 80% identical to SEQ ID NO: 154 and a VL comprising an amino acid sequence at least 80% identical to SEQ ID NO: 155;
wherein CA130 comprises a VH comprising an amino acid sequence at least 80% identical to SEQ ID NO: 156 and a VL comprising an amino acid sequence at least 80% identical to SEQ ID NO: 157;
wherein CA131 comprises a VH comprising an amino acid sequence at least 80% identical to SEQ ID NO: 158 and a VL comprising an amino acid sequence at least 80% identical to SEQ ID NO: 159;
wherein CA132 comprises a VH comprising an amino acid sequence at least 80% identical to SEQ ID NO: 156 and a VL comprising an amino acid sequence at least 80% identical to SEQ ID NO: 141;
wherein CA133 comprises a VH comprising an amino acid sequence at least 80% identical to SEQ ID NO: 160 and a VL comprising an amino acid sequence at least 80% identical to SEQ ID NO: 161;
wherein CA134 comprises a VH comprising an amino acid sequence at least 80% identical to SEQ ID NO: 162 and a VL comprising an amino acid sequence at least 80% identical to SEQ ID NO: 163;
wherein CA135 comprises a VH comprising an amino acid sequence at least 80% identical to SEQ ID NO: 164 and a VL comprising an amino acid sequence at least 80% identical to SEQ ID NO: 157;
wherein CA136 comprises a VH comprising an amino acid sequence at least 80% identical to SEQ ID NO: 165 and a VL comprising an amino acid sequence at least 80% identical to SEQ ID NO: 166;
wherein CA137 comprises a VH comprising an amino acid sequence at least 80% identical to SEQ ID NO: 167 and a VL comprising an amino acid sequence at least 80% identical to SEQ ID NO: 157;
wherein CA138 comprises a VH comprising an amino acid sequence at least 80% identical to SEQ ID NO: 168 and a VL comprising an amino acid sequence at least 80% identical to SEQ ID NO: 169;
wherein CA139 comprises a VH comprising an amino acid sequence at least 80% identical to SEQ ID NO: 170 and a VL comprising an amino acid sequence at least 80% identical to SEQ ID NO: 171;
wherein CA140 comprises a VH comprising an amino acid sequence at least 80% identical to SEQ ID NO: 172 and a VL comprising an amino acid sequence at least 80% identical to SEQ ID NO: 173;
wherein CA141 comprises a VH comprising an amino acid sequence at least 80% identical to SEQ ID NO: 174 and a VL comprising an amino acid sequence at least 80% identical to SEQ ID NO: 175;
wherein CA143 comprises a VH comprising an amino acid sequence at least 80% identical to SEQ ID NO: 176 and a VL comprising an amino acid sequence at least 80% identical to SEQ ID NO: 177;
wherein CA144 comprises a VH comprising an amino acid sequence at least 80% identical to SEQ ID NO: 178 and a VL comprising an amino acid sequence at least 80% identical to SEQ ID NO: 179;
wherein CA145 comprises a VH comprising an amino acid sequence at least 80% identical to SEQ ID NO: 180 and a VL comprising an amino acid sequence at least 80% identical to SEQ ID NO: 181;
wherein CA146 comprises a VH comprising an amino acid sequence at least 80% identical to SEQ ID NO: 182 and a VL comprising an amino acid sequence at least 80% identical to SEQ ID NO: 183;
wherein CA401 comprises a VH comprising an amino acid sequence at least 80% identical to SEQ ID NO: 186 and a VL comprising an amino acid sequence at least 80% identical to SEQ ID NO: 187;
wherein 3H4 Gv2 comprises a VH comprising an amino acid sequence at least 80% identical to SEQ ID NO: 35 and a VL comprising an amino acid sequence at least 80% identical to SEQ ID NO: 129;
wherein 3H4 Gv3 comprises a VH comprising an amino acid sequence at least 80% identical to SEQ ID NO: 36 and a VL comprising an amino acid sequence at least 80% identical to SEQ ID NO: 129;
wherein 3H4 Gv4 comprises a VH comprising an amino acid sequence at least 80% identical to SEQ ID NO: 37 and a VL comprising an amino acid sequence at least 80% identical to SEQ ID NO: 129;
wherein 3H4 Gv5 comprises a VH comprising an amino acid sequence at least 80% identical to SEQ ID NO: 38 and a VL comprising an amino acid sequence at least 80% identical to SEQ ID NO: 129;
wherein 3H4 Gv6 comprises a VH comprising an amino acid sequence at least 80% identical to SEQ ID NO: 39 and a VL comprising an amino acid sequence at least 80% identical to SEQ ID NO: 129;
wherein 3H4 Gv7 comprises a VH comprising an amino acid sequence at least 80% identical to SEQ ID NO: 40 and a VL comprising an amino acid sequence at least 80% identical to SEQ ID NO: 129;
wherein 3H4 Gv8 comprises a VH comprising an amino acid sequence at least 80% identical to SEQ ID NO: 41 and a VL comprising an amino acid sequence at least 80% identical to SEQ ID NO: 129;
wherein 3H4 Gv9 comprises a VH comprising an amino acid sequence at least 80% identical to SEQ ID NO: 42 and a VL comprising an amino acid sequence at least 80% identical to SEQ ID NO: 129;
wherein 3H4 Gv10 comprises a VH comprising an amino acid sequence at least 80% identical to SEQ ID NO: 43 and a VL comprising an amino acid sequence at least 80% identical to SEQ ID NO: 129;
wherein 3H4 Gv1 1 comprises a VH comprising an amino acid sequence at least 80% identical to SEQ ID NO: 44 and a VL comprising an amino acid sequence at least 80% identical to SEQ ID NO: 129;
wherein 3H4 Gv12 comprises a VH comprising an amino acid sequence at least 80% identical to SEQ ID NO: 45 and a VL comprising an amino acid sequence at least 80% identical to SEQ ID NO: 129;
wherein CA147hexa comprises a VH comprising an amino acid sequence at least 80% identical to all consecutive amino acids after the signal peptide of SEQ ID NO: 340 and a VL comprising an amino acid sequence at least 80% identical to SEQ ID NO: 351;
wherein CA147sortaNP comprises a VH comprising an amino acid sequence at least 80% identical to SEQ ID NO: 349 and a VL comprising an amino acid sequence at least 80% identical to SEQ ID NO: 351;
wherein 3H4hexa comprises a VH comprising an amino acid sequence at least 80% identical to SEQ ID NO: 338 and a VL comprising an amino acid sequence at least 80% identical to SEQ ID NO: 345; and
wherein 3H4sortaNP comprises a VH comprising an amino acid sequence at least 80% identical to SEQ ID NO: 343 and a VL comprising an amino acid sequence at least 80% identical to SEQ ID NO: 345.

62. The recombinant antibody or fragment thereof of claim 61, wherein the recombinant antibody or fragment thereof binds to an HLA-E-VL9 complex.

63. The recombinant antibody or fragment thereof of claim 61, wherein the recombinant antibody or fragment thereof is selected from the group consisting of 3H4 and CA147, and
wherein 3H4 comprises a VH comprising an amino acid sequence at least 90% identical to SEQ ID NO: 128 and a VL comprising an amino acid sequence at least 90% identical to SEQ ID NO: 129; and
wherein CA147 comprises a VH comprising an amino acid sequence at least 90% identical to SEQ ID NO: 184 and a VL comprising an amino acid sequence at least 90% identical to SEQ ID NO: 185.

64. The recombinant antibody or fragment thereof of claim 63, wherein heavy chain complementarity determining region (HCDR)1, HCDR2 and HCDR3 sequences of the VH collectively have no more than 10 amino acid variations as compared to the corresponding HCDR1, HCDR2 and HCDR3 sequences of SEQ ID NO: 128 or SEQ ID NO: 184; and light chain complementary determining region (LCDR)1, LCDR2 and LCDR3 sequences of the VL collectively have no more than 10 amino acid variations as compared to the corresponding LCDR1, LCDR2 and LCDR3 sequences of SEQ ID NO: 129 or SEQ ID NO: 185.

65. The recombinant antibody or fragment thereof of claim 61, wherein the recombinant antibody or fragment thereof is selected from the group consisting of 3H4, 13F11, 10C10, 2D6, 3H4 Gv2, 3H4 Gv3, 3H4 Gv4, 3H4 Gv5, 3H4 Gv6, 3H4 Gv7, 3H4 Gv8, 3H4 Gv9, 3H4 Gv10, 3H4 Gv11, 3H4 Gv12, 3H4hexa, and 3H4sortaNP wherein the recombinant antibody or fragment thereof is humanized.

66. The recombinant antibody or fragment thereof of claim 65, wherein the VH and VL each comprise framework regions that each have no more than 20 amino acid variations derived from framework regions of a human antibody.

67. The recombinant antibody or fragment thereof of claim 65, wherein the VH comprises framework regions derived from SEQ ID NOs: 136, 138, 140, 142, 144, 146, 148, 150, 152, 154, 156, 158, 160, 162, 164, 165, 167, 168, 170, 172, 174, 176, 178, 180, 182, 184, 186, 340 or 349; and the VL comprises framework regions derived from SEQ ID NO: 137, 139, 141, 143, 145, 147, 149, 151, 153, 155, 157, 159, 161, 163, 166, 169, 171, 173, 175, 177, 179, 181, 183, 185, 187 or 351.

68. The recombinant antibody or fragment thereof of claim 63, wherein HCDR1, HCDR2 and HCDR3 sequences of the VH collectively have no more than 10 amino acid variations as compared to the corresponding HCDR1, HCDR2 and HCDR3 of SEQ ID NO: 128 or SEQ ID NO: 184;
LCDR1, LCDR2 and LCDR3 sequences of the VL collectively have no more than 10 amino acid variations as compared to the corresponding LCDR1, LCDR2 and LCDR3 of SEQ ID NO: 129 or SEQ ID NO: 185; and
the framework regions of the VH and VL are derived from a human antibody.

69. The recombinant antibody or fragment thereof of claim 61, wherein the recombinant antibody or fragment thereof is chimeric.

70. A humanized antibody or fragment thereof selected from the group consisting of:
3H4, 13F11, 10C10, 2D6, 3H4 Gv2, 3H4 Gv3, 3H4 Gv4, 3H4 Gv5, 3H4 Gv6, 3H4 Gv7, 3H4 Gv8, 3H4 Gv9, 3H4 Gv10, 3H4 Gv11, 3H4 Gv12, 3H4hexa, and 3H4sortaNP;
wherein 3H4 comprises a VH comprising HCDR1, HCDR2 and HCDR3 sequences at least 80% identical to the HCDR1, HCDR2 and HCDR3 sequences of SEQ ID NO: 128 and a VL comprising LCDR1, LCDR2 and LCDR3 sequences at least 80% identical to the LCDR1, LCDR2 and LCDR3 sequences of SEQ ID NO: 129;
wherein 13F11 comprises VH comprising HCDR1, HCDR2 and HCDR3 sequences at least 80% identical to the HCDR1, HCDR2 and HCDR3 sequences of SEQ ID NO: 130 and a VL comprising LCDR1, LCDR2 and LCDR3 sequences at least 80% identical to the LCDR1, LCDR2 and LCDR3 sequences of SEQ ID NO: 131;
wherein 10C10 comprises a VH comprising HCDR1, HCDR2 and HCDR3 sequences at least 80% identical to the HCDR1, HCDR2 and HCDR3 sequences of SEQ ID NO: 132 and a VL comprising LCDR1, LCDR2 and LCDR3 sequences at least 80% identical to the LCDR1, LCDR2 and LCDR3 sequences of SEQ ID NO: 133;
wherein 2D6 comprises a VH comprising HCDR1, HCDR2 and HCDR3 sequences at least 80% identical to the HCDR1, HCDR2 and HCDR3 sequences of SEQ ID NO: 134 and a VL comprising LCDR1, LCDR2 and LCDR3 sequences at least 80% identical to the LCDR1, LCDR2 and LCDR3 sequences of SEQ ID NO: 135;
wherein 3H4 Gv2 comprises a VH comprising HCDR1, HCDR2 and HCDR3 sequences at least 80% identical to the HCDR1, HCDR2 and HCDR3 sequences of SEQ ID NO: 35 and a VL comprising LCDR1, LCDR2 and LCDR3 sequences at least 80% identical to the LCDR1, LCDR2 and LCDR3 sequences of SEQ ID NO: 129;
wherein 3H4 Gv3 comprises a VH comprising HCDR1, HCDR2 and HCDR3 sequences at least 80% identical to the HCDR1, HCDR2 and HCDR3 sequences of SEQ ID NO: 36 and a VL comprising LCDR1, LCDR2 and LCDR3 sequences at least 80% identical to the LCDR1, LCDR2 and LCDR3 sequences of SEQ ID NO: 129;
wherein 3H4 Gv4 comprises a VH comprising HCDR1, HCDR2 and HCDR3 sequences at least 80% identical to the HCDR1, HCDR2 and HCDR3 sequences of SEQ ID NO: 37 and a VL comprising LCDR1, LCDR2 and LCDR3 sequences at least 80% identical to the LCDR1, LCDR2 and LCDR3 sequences of SEQ ID NO: 129;
wherein 3H4 Gv5 comprises a VH comprising HCDR1, HCDR2 and HCDR3 sequences at least 80% identical to the HCDR1, HCDR2 and HCDR3 sequences of SEQ ID NO: 38 and a VL comprising LCDR1, LCDR2 and LCDR3 sequences at least 80% identical to the LCDR1, LCDR2 and LCDR3 sequences of SEQ ID NO: 129;
wherein 3H4 Gv6 comprises a VH comprising HCDR1, HCDR2 and HCDR3 sequences at least 80% identical to the HCDR1, HCDR2 and HCDR3 sequences of SEQ ID NO: 39 and a VL comprising LCDR1, LCDR2 and LCDR3 sequences at least 80% identical to the LCDR1, LCDR2 and LCDR3 sequences of SEQ ID NO: 129;
wherein 3H4 Gv7 comprises a VH comprising HCDR1, HCDR2 and HCDR3 sequences at least 80% identical to the HCDR1, HCDR2 and HCDR3 sequences of SEQ ID NO: 40 and a VL comprising LCDR1, LCDR2 and LCDR3 sequences at least 80% identical to the LCDR1, LCDR2 and LCDR3 sequences of SEQ ID NO: 129;
wherein 3H4 Gv8 comprises a VH comprising HCDR1, HCDR2 and HCDR3 sequences at least 80% identical to the HCDR1, HCDR2 and HCDR3 sequences of SEQ ID NO: 41 and a VL comprising LCDR1, LCDR2 and LCDR3 sequences at least 80% identical to the LCDR1, LCDR2 and LCDR3 sequences of SEQ ID NO: 129;
wherein 3H4 Gv9 comprises a VH comprising HCDR1, HCDR2 and HCDR3 sequences at least 80% identical to the HCDR1, HCDR2 and HCDR3 sequences of SEQ ID NO: 42 and a VL comprising LCDR1, LCDR2 and LCDR3 sequences at least 80% identical to the LCDR1, LCDR2 and LCDR3 sequences of SEQ ID NO: 129;
wherein 3H4 Gv10 comprises a VH comprising HCDR1, HCDR2 and HCDR3 sequences at least 80% identical to the HCDR1, HCDR2 and HCDR3 sequences of SEQ ID NO: 43 and a VL comprising LCDR1, LCDR2 and LCDR3 sequences at least 80% identical to the LCDR1, LCDR2 and LCDR3 sequences of SEQ ID NO: 129;
wherein 3H4 Gv1 1 comprises a VH comprising HCDR1, HCDR2 and HCDR3 sequences at least 80% identical to the HCDR1, HCDR2 and HCDR3 sequences of SEQ ID NO: 44 and a VL comprising LCDR1, LCDR2 and LCDR3 sequences at least 80% identical to the LCDR1, LCDR2 and LCDR3 sequences of SEQ ID NO: 129;
wherein 3H4 Gv12 comprises a VH comprising HCDR1, HCDR2 and HCDR3 sequences at least 80% identical to the HCDR1, HCDR2 and HCDR3 sequences of SEQ ID NO: 45 and a VL comprising LCDR1, LCDR2 and LCDR3 sequences at least 80% identical to the LCDR1, LCDR2 and LCDR3 sequences of SEQ ID NO: 129;
wherein 3H4hexa comprises a VH comprising HCDR1, HCDR2 and HCDR3 sequences at least 80% identical to the HCDR1, HCDR2 and HCDR3 sequences of SEQ ID NO: 338 and a VL comprising LCDR1, LCDR2 and LCDR3 sequences at least 80% identical to the LCDR1, LCDR2 and LCDR3 sequences of SEQ ID NO: 345; and
wherein 3H4sortaNP comprises a VH comprising HCDR1, HCDR2 and HCDR3 sequences at least 80% identical to the HCDR1, HCDR2 and HCDR3 sequences of SEQ ID NO: 343 and a VL comprising LCDR1, LCDR2 and LCDR3 sequences at least 80% identical to the LCDR1, LCDR2 and LCDR3 sequences of SEQ ID NO: 345

71. The humanized antibody or fragment thereof claim 70, wherein the humanized antibody or fragment thereof binds to an HLA-E-VL9 complex.

72. The humanized antibody or fragment thereof of claim 70, wherein the HCDR1, HCDR2 and HCDR3 sequences collectively have no more than 20 amino acid variations as compared to the corresponding HCDR1, HCDR2 and HCDR3 sequences of SEQ ID NOs: 35-45, 128, 130, 132, 134, 338, or 343; and
the LCDR1, LCDR2 and LCDR3 sequences collectively have no more than 20 amino acid variations as compared to the corresponding LCDR1, LCDR2 and LCDR3 sequence of SEQ ID NO: 129, 131, 133, 135, or 345.

73. The humanized antibody or fragment thereof of claim 70, wherein the humanized antibody or fragment thereof is 3H4 and the VH comprises HCDR1, HCDR2 and HCDR3 sequences of SEQ ID NO: 128; and the VH comprises LCDR1, LCDR2, and LCDR3 sequences of SEQ ID NO: 129.

74. The humanized antibody or fragment thereof of claim 70, wherein the humanized antibody or fragment thereof comprises a paratope, and wherein the paratope comprises the same contact residues as 3H4.

75. The humanized antibody or fragment thereof of claim 70, wherein the VH comprises framework regions that are derived from a human antibody and wherein the human antibody comprises a VH at least 90% identical to SEQ ID NO: 35-45, 128, 130, 132, 134, 338, or 343; and
the light chain variable region comprises framework regions that are derived from a human antibody and wherein the human antibody comprises a light chain variable region has an overall sequence identity of at least 90% of SEQ ID NO: 129, 131, 133, 135, or 345.

76. The humanized antibody or fragment thereof of claim 70, wherein
the VH comprises framework regions that are derived from a human antibody and wherein the human antibody comprises the most similar three-dimensional structure to the VH of SEQ ID NO: 35-45, 128, 130, 132, 134, 338, or 343; and
the VL comprises framework regions that are derived from a human antibody and wherein the human antibody comprises the most similar three-dimensional structure to the VL of SEQ ID NO: 129, 131, 133, 135, or 345.

77. The humanized antibody or fragment thereof of claim 70, wherein the VH comprises framework regions that are derived from IGHV3-21, IGHV3-11, IGHV3-23, IGHV1-69, or IGHV3-48.

78. The humanized antibody or fragment thereof of claim 70, wherein the VL comprises framework regions that are derived from IGKV3-15, IGKV3-20, IGKV1-39, IGKV3-11, or IGKV1-5.

79. The recombinant antibody or fragment thereof of claim 62, wherein the HLA-E-VL9 complex bound by the recombinant antibody or fragment thereof comprises a peptide with an amino acid sequence of SEQ ID NO: 4 or SEQ ID NO:1.

80. The recombinant antibody or fragment thereof of claim 79, wherein the recombinant antibody or fragment binds to two or more epitopes on both the HLA-E α2 domain and the amino terminal end of the peptide.

81. The recombinant antibody or fragment thereof of claim 62, wherein the recombinant antibody or fragment has an affinity or avidity for the HLA-E-VL9 complex greater than the affinity or avidity between the HLA-E-VL9 complex and NKG2A.

82. The recombinant antibody or fragment thereof of claim 81, wherein the recombinant antibody or fragment increases the cytotoxic activity of NKG2A+NK cells, NKG2A+CD8+ T-cells, or NKG2A+γδ T-cells.

83. The recombinant antibody or fragment thereof of claim 61, wherein the recombinant antibody or fragment comprises an Fc moiety.

84. The recombinant antibody or fragment thereof of claim 83, wherein the recombinant antibody or fragment comprises at least one mutation in the Fc moiety and wherein the mutated Fc moiety reduces binding of the recombinant antibody or fragment to an Fc receptor and/or increases the half-life of the recombinant antibody or fragment.

85. The recombinant antibody or fragment thereof of claim 61, wherein the recombinant antibody or fragment is a purified antibody, a single chain antibody, Fab, Fab′, F(ab′)2, Fv or scFv.

86. The recombinant antibody or fragment thereof of claim 61, wherein the recombinant antibody or fragment is of any isotype.

87. The recombinant antibody or fragment thereof of claim 61, wherein the recombinant antibody or fragment is for use as a medicament.

88. The recombinant antibody or fragment thereof of claim 61, wherein the recombinant antibody or fragment is for use in the prevention and/or treatment of a tumor comprising tumor cells that overexpress HLA-E.

89. A nucleic acid molecule comprising a polynucleotide sequence encoding the recombinant antibody or fragment thereof of claim 61.

90. A ribonucleic acid (RNA) comprising a nucleic acid sequence encoding the recombinant antibody or fragment thereof of claim 61.

91. A vector comprising the nucleic acid molecule according to claim 89.

92. A cell comprising the vector of claim 91.

93. A cell expressing the recombinant antibody or fragment thereof of claim 61.

94. A pharmaceutical composition comprising the recombinant antibody or fragment thereof of claim 61.

95. A pharmaceutical composition comprising the nucleic acid of claim 89.

96. A pharmaceutical composition comprising the vector of claim 91.

97. A pharmaceutical composition comprising the cell of claim 92.

98. A pharmaceutical composition comprising the cell of claim 93.

99. The pharmaceutical composition according to claim 94 further comprising a pharmaceutically acceptable excipient, diluent or carrier.
